(19) United States
(12) Patent Application Publication
(10) Pub. No.: US 2024/0035076 A1
(43) Pub. Date: Feb. 1, 2024

(54) METHOD OF AGEING FISH OR REPTILES

(71) Applicant: Commonwealth Scientific and Industrial Research Organisation, Acton, Australian Capital Territory (AU)

(72) Inventors: Oliver Berry, Acton (AU); Simon Jarman, Perth (AU); Benjamin Mayne, Acton (AU)

(21) Appl. No.: 18/246,006

(22) PCT Filed: Sep. 23, 2021
(86) PCT No.: PCT/AU2021/051117

§ 371 Date: Mar. 20, 2023

(30) Foreign Application Priority Data
Sep. 23, 2020 (AU) 2020903422
Mar. 16, 2021 (AU) 2021900750

(51) Int. Cl. C12Q 1/6858; C12Q 1/6888

(52) U.S. Cl. CPC C12Q 1/6858; C12Q 1/6888; C12Q 2600/154; C12Q 2600/16; C12Q 2600/124; C12Q 1/6869

(57) ABSTRACT

The present invention relates to age-associated CpG sites which can be used to estimate the age of the fish or reptile and to methods for identifying age-associated CpG sites for a fish or reptile. The present invention also relates to methods for estimating the age of a fish or reptile using the age-associated CpG sites.

### CROSS-REFERENCE TO RELATED APPLICATIONS

This application is a National Stage Application under 35 U.S.C. § 371 of International Application No. PCT/AU2021/051117 filed Sep. 23, 2021, which claims the benefit of priority to Australian Patent Application No. 2021900750 filed Mar. 16, 2021 and Australian Patent Application No. 2020903422 filed Sep. 23, 2020, the disclosures of which are hereby incorporated by reference in their entireties.

### FIELD OF THE INVENTION

The present disclosure relates to methods for estimating the age of a fish or reptile. The present disclosure also relates to age-associated CpG sites which can be used to estimate the age of the fish or reptile and to methods for identifying age-associated CpG sites for a fish or reptile.

### BACKGROUND OF THE INVENTION

Being able to determine the age of a fish is important for understanding the life cycle of a fish species. Knowing how fast they grow, how old they are when they reproduce and how long they live provides information that can be used to assess the status of a fish population and the sustainability of current and future fishing practices.

The method for estimating the age of a fish currently recommended by the Australian Department of Agriculture of Fisheries involves the use of otoliths to estimate age. Otoliths (a fish inner ear structure) are composed of a form of calcium carbonate and protein which is laid down at different rates throughout a fish's life. This process leaves alternating opaque and translucent bands on the otolith which can be used, like the growth rings in a tree, to estimate the age of the fish (Campana, 2001). Although widely used by temperate fisheries this methodology has several limitations. First, recovering the otolith from a fish is a time-consuming, expensive and a lethal process (Fowler, 2009). This methodology often relies on multiple operators which introduces a subjectivity to the test, requires that the otolith is undamaged while being removed and cannot be automated (Worthington et al., 2011). Second, the reliability of otolith-based ageing is also confounded by sources of variation including the size, age, sex, year class differences and environmental factors (Cadrin and Friedland, 1999). For example, in tropical fish species, environmental conditions are constant and distinct layers of growth increments are not observed. For these species, the otolith is simply weighed to estimate age. Finally, otolith ageing cannot be effectively used with low stock numbers or for conservation purposes as it requires killing a subset of fish.

Other methods of ageing fish involve measurements of anatomical structures such as fins, vertebra, eye lens and/or scales. The reliance on measuring a physical structure, such as an otolith, fin or scales, from the fish can cause under- and over-estimations of age depending on the species.

Accordingly, there is a need for an improved method of ageing fish or at least an alternative to otolith ageing or ageing relying on measuring a physical structure. Preferably, the method should be non-lethal, have the potential to be automated and/or cost-effective.

### SUMMARY OF THE INVENTION

The inventors have identified that the level of methylated cytosine at certain CpG sites within the fish and reptile genome varies as the fish or reptile ages and that these sites may be used to estimate the age of the fish or reptile.

Accordingly, the present application provides a method for estimating the age of a fish or reptile comprising estimating the age of the fish or reptile based on analysis of DNA obtained from the fish or reptile for the presence of a methylated cytosine at age-associated CpG sites. In some embodiments, the present application provides a method for estimating the age of a fish or reptile comprising analysing DNA obtained from a fish or reptile for the presence of a methylated cytosine at age-associated CpG sites; and estimating the age of the fish or reptile based on methylated cytosine levels at the age-associated CpG sites. In some embodiments, the age-associated CpG sites are selected from (i) Table 1, 2 or 3 or a homolog of one or more thereof, (ii) Table 7 or a homolog of one or more thereof, (iii) Table 8 or 9 or a homolog of one or more thereof, (iv) Table 12 or a homolog of one or more thereof, (v) Table 16 or a homolog of one or more thereof, or (vi) Table 19 or 20 or a homolog of one or more thereof. In some embodiments, the age-associated CpG sites are selected from (i) Table 1, 2 or 3 or a homolog of one or more thereof, (ii) Table 8 or 9 or a homolog of one or more thereof, (iii) Table 12 or a homolog of one or more thereof, (iv) Table 16 or a homolog of one or more thereof, or (v) Table 19 or 20 or a homolog of one or more thereof. In some embodiments, there is provided a method for estimating the age of a fish comprising analysing DNA obtained from a fish for the presence of a methylated cytosine at age-associated CpG sites; and estimating the age of the fish based on methylated cytosine levels at the age-associated CpG sites, wherein the age-associated CpG sites are selected from (i) Table 1, 2 or 3 or a homolog of one or more thereof; (ii) Table 12 or a homolog of one or more thereof, or (iii) Table 16 or a homolog of one or more thereof. In some embodiments, the age-associated CpG sites are comprised within an amplicon listed in Table 5. In some embodiments, the age-associated CpG sites are located within a nucleic acid sequence set forth in any one or more of SEQ ID NO: 53 to SEQ ID NO: 78.

In some embodiments, the age-associated CpG sites are selected from Table 1, 2 or 3 or a homolog of one or more thereof. In an embodiment, the age-associated CpG sites are selected from Table 2 or a homolog of one or more thereof. In an embodiment, the age-associated CpG sites are selected from Table 3 or a homolog of one or more thereof.

In some embodiments, the age-associated CpG sites are selected from Table 7 or a homolog of one or more thereof.

In some embodiments, the age-associated CpG sites are selected from Table 8 or 9 or a homolog of one or more thereof. In some embodiments, the age-associated CpG sites are selected from Table 8 or a homolog of one or more thereof. In some embodiments, the age-associated CpG sites are selected from Table 9 or a homolog of one or more thereof. In some embodiments, the age-associated CpG sites are selected from Table 12 or a homolog of one or more thereof. In some embodiments, the age-associated CpG sites are selected from Table 16 or a homolog of one or more thereof. In some embodiments, the age-associated CpG sites are selected from Table 19, Table 20 or a homolog of one or more thereof. In some embodiments, the age-associated CpG sites are selected from Table 20 or a homolog of one or more thereof.

In some embodiments, the presence of methylated cytosine is analysed at five or more, 10 or more, 15 or more, 20 or more, or 25 or more of the age-associated CpG sites. In an embodiment, the presence of each of the age-associated CpG sites in Table 3 or a homolog of one or more thereof is analysed. In an embodiment, the presence of each of the age-associated CpG sites in Table 8 or a homolog of one or more thereof is analysed. In an embodiment, the presence of each of the age-associated CpG sites in Table 9 or a homolog of one or more thereof is analysed. In an embodiment, the presence of each of the age-associated CpG sites in Table 12 or a homolog of one or more thereof is analysed. In an embodiment, the presence of each of the age-associated CpG sites in Table 16 or a homolog of one or more thereof is analysed. In an embodiment, the presence of each of the age-associated CpG sites in Table 19, Table 20 or a homolog of one or more thereof is analysed. In an embodiment, the presence of each of the age-associated CpG sites in Table 20 or a homolog of one or more thereof is analysed.

In some embodiments, analysing DNA comprises multiplex PCR. In some embodiments, analysing DNA comprises DNA sequencing. In some embodiments, analysing DNA comprises multiplex PCR and DNA sequencing.

In some embodiments, the multiplex PCR uses primer pairs configured to amplify a region of the DNA comprising the age-associated CpG sites. In some embodiments, the multiplex PCR uses two or more primer pairs configured to amplify a region of the DNA comprising the age-associated CpG sites. In some embodiments, at least one of the primers (i) is selected from Table 4; and/or (ii) can be used to amplify the same CpG site as the primers of (i). In some embodiments, at least one of the primers hybridizes to a region of the DNA within 100 or 50 or 20 base-pairs of a primer of (i). In some embodiments, one or more or all of the primers pairs provided in Table 4 are used. In some embodiments, at least one of the primers (i) is selected from Table 11; and/or (ii) can be used to amplify the same CpG site as the primers of (i). In some embodiments, at least one of the primers hybridizes to a region of the DNA within 100 or 50 or 20 base-pairs of a primer of (i). In some embodiments, one or more or all of the primers pairs provided in Table 11 are used. In some embodiments, at least one of the primers (i) is selected from Table 15; and/or (ii) can be used to amplify the same CpG site as the primers of (i). In some embodiments, at least one of the primers hybridizes to a region of the DNA within 100 or 50 or 20 base-pairs of a primer of (i). In some embodiments, one or more or all of the primers pairs provided in Table 15 are used.

In some embodiments, analysing DNA comprises determining the methylation beta value of the age associated CpG sites. In some embodiments, estimating the age of the fish or reptile comprises comparing to an age correlated reference population. In some embodiments, estimating the age of the fish or reptile comprises determining a methylation profile. In some embodiments, the methylation profile is the sum of raw summed methylation beta values for the age-associated CpG sites.

In some embodiments, estimating the age of the fish or reptile comprises comparing the methylation profile for the DNA to a methylation profile from an age correlated reference population determined using the same age-associated CpG sites.

In some embodiments, the methods described herein are non-lethal. In other words, the fish or reptile is not sacrificed prior to obtaining DNA from the fish or reptile.

In some embodiments, the method further comprises obtaining a biological sample comprising the DNA from the fish or reptile. In some embodiments, the DNA analysed is from caudal fin. In some embodiments, the DNA analysed is from skin biopsy.

In some embodiments, the correlation between chronological age and estimated age is at least 90%, or at least 95%.

The present application also provides use of two or more primer pairs for amplifying one or more age-associated CpG sites listed in Table 1, 2, 3, 7, 8, 9, 12, 16, 19 or 20 or a homolog thereof. The present application also provides use of two or more primer pairs for amplifying one or more age-associated CpG sites listed in Table 1, 2, 3, 8 or 9 or a homolog thereof. The present application also provides use of two or more primer pairs for amplifying one or more age-associated CpG sites listed in Table 1, 2 or 3 or a homolog thereof. The present application also provides use of two or more primer pairs for amplifying one or more age-associated CpG sites listed in Table 7 or a homolog thereof. The present application also provides use of two or more primer pairs for amplifying one or more age-associated CpG sites listed in Table 8 or 9 or a homolog thereof. The present application also provides use of two or more primer pairs for amplifying one or more age-associated CpG sites listed in Table 12 or a homolog thereof. The present application also provides use of two or more primer pairs for amplifying one or more age-associated CpG sites listed in Table 16 or a homolog thereof. The present application also provides use of two or more primer pairs for amplifying one or more age-associated CpG sites listed in Table 19 or 20 or a homolog thereof. The present application also provides use of two or more primer pairs for amplifying one or more age-associated CpG sites listed in Table 20 or a homolog thereof.

In some embodiments, there is provided a method for estimating the age of reptile comprising:
analysing DNA obtained from a reptile for the presence of a methylated cytosine at age-associated CpG sites; and
estimating the age of the reptile based on methylated cytosine levels at the age-associated CpG sites.

In some embodiments, the age-associated CpG sites are selected from Table 19 or 20 or a homolog of one or more thereof. In some embodiments, the age-associated CpG sites are selected from Table 20 or a homolog of one or more thereof. In some embodiments, the presence of methylated cytosine is analysed at five or more, 10 or more, 15 or more, 20 or more, 25 or more or all of the age-associated CpG sites listed in Table 20. In some embodiments, the reptile is a marine turtle. In some embodiments, the marine turtle is selected from the group consisting of Green sea turtle, Flatback turtle, Hawksbill turtle, Leatherback turtle, Loggerhead turtle and Olive Ridley turtle.

The present application also provides a method for identifying age-associated CpG sites for a species of fish or reptile comprising analysing DNA obtained from the species of fish or reptile of different chronological ages for the presence of methylated cytosine at CpG sites; and using a statistical algorithm to identify age-associated CpG sites.

In some embodiments, analysing DNA comprises reduced representation bisulfite sequencing. In some embodiments, the statistical algorithm is elastic net regression model.

The present inventors have also surprisingly found that the age associated CpG sites identified for one species fish or reptile can be used to identify age associated CpG sites for a second species of fish or reptile. Accordingly, the present application also provides a method of identifying an age-associated CpG site for a second species of fish comprising (i) analysing DNA of the second fish species for a candidate age-associated CpG site corresponding to an age-associated CpG site identified for a first species of fish; (ii) analysing the methylation patterns of a candidate age-associated CpG site identified in (i) in different ages of the second species of fish to determine if it is an age-associated CpG site in that second fish species. In some embodiments, step (i) comprises a pairwise analysis of the DNA of the first fish species with zebrafish the DNA of the second fish species. In some embodiments, the first fish species is zebrafish and step (i) comprises analysing DNA of the second fish species for a candidate age-associated CpG site corresponding to an age-associated CpG site listed in Table 1, 2 or 3. In some embodiments, the second fish species is a member of the infraclass Teleostei. In some embodiments, the first fish species is a shark and step (i) comprises analysing DNA of the second fish species for a candidate age-associated CpG site corresponding to an age-associated CpG site listed in Table 8 or 9. In some embodiments, the second fish species is a shark species.

The present application also provides a method of identifying an age-associated CpG site for a second species of reptile comprising (i) analysing DNA of the second reptile species for a candidate age-associated CpG site corresponding to an age-associated CpG site identified for a first species of reptile; (ii) analysing the methylation patterns of a candidate age-associated CpG site identified in (i) in different ages of the second species of reptile to determine if it is an age-associated CpG site in that second reptile species. In some embodiments, step (i) comprises a pairwise analysis of the DNA of the first reptile species with the DNA of the second reptile species. In some embodiments, the first reptile species is green sea turtle and step (i) comprises analysing DNA of the second reptile species for a candidate age-associated CpG site corresponding to an age-associated CpG site listed in Table 19 or 20. In some embodiments, the second reptile species is a marine turtle. In some embodiments, the marine turtle is selected from the group consisting of Flatback turtle, Hawksbill turtle, Leatherback turtle, Loggerhead turtle and Olive Ridley turtle.

In some embodiments, the fish is a member of the infraclass Teleostei. In some embodiments, the fish is a Grouper (Epinephelus spp.), Tuna, Cobia, Sturgeon, Mahi-mahi, Bonito, Dhufish, Murray cod, Barramundi, Herring, Tra catfish, Mekong giant catfish, Cod, Pilchard, Pollock, Turbot, Hake, Anchovy, Haddock, Black carp, Grass carp, Eels, Koi Carp, Giant gourami, zebrafish, Mackerel, Australian lungfish, Mary river cod, Salmon or trout. In some embodiments, the fish is zebrafish, yellow fin tuna, skipjack tuna, Atlantic cod, Atlantic herring, Alaska pollock, Australian lungfish, Mary River Cod or Atlantic Salmon. In some embodiments, the fish is zebrafish. In some embodiments, the fish is an Atlantic Salmon.

In some embodiments, the fish is a member of the subclass Elasmobranchii. Accordingly, the present application further provides a method for estimating the age of a fish which is a member of the subclass Elasmobranchii, the method comprising:
analysing DNA obtained from the fish for the presence of a methylated cytosine at age-associated CpG sites; and
estimating the age of the fish based on methylated cytosine levels at the age-associated CpG sites. In some embodiments, the age-associated CpG sites are selected from Table 8 or 9 or a homolog of one or more thereof. the age-associated CpG sites are identified by analysing DNA obtained from the species of fish of different chronological ages for the presence of methylated cytosine at CpG sites; and using a statistical algorithm to identify age-associated CpG sites. In some embodiments, analysing DNA comprises reduced representation bisulfite sequencing. In some embodiments, the statistical algorithm is elastic net regression model.

In some embodiments, the fish is a shark. In some embodiments, the shark is a school shark.

In some embodiments, the presence of methylated cytosine is analysed at five or more, 10 or more, 15 or more, 20 or more, 25 or more or 30 of the age-associated CpG sites. In some embodiments, analysing DNA comprises multiplex PCR. In some embodiments, analysing DNA comprises multiplex PCR and DNA sequencing. In some embodiments, the multiplex PCR uses two or more primer pairs configured to amplify a region of the DNA comprising the age-associated CpG sites.

In some embodiments, the method is used to estimate the age of a reptile. In some embodiments, the reptile is a marine turtle. In some embodiments, the marine turtle is selected from the group consisting of Green sea turtle, Flatback turtle, Hawksbill turtle, Leatherback turtle, Loggerhead turtle and Olive Ridley turtle.

The present application also provides a kit for estimating the age of a fish or reptile comprising one or more primer pairs or probes for detecting the presence of a methylated cytosine at age-associated CpG sites. In some embodiments, the age-associated CpG sites are selected from Table 1, 2 or 3 or a homolog of one or more thereof. In some embodiments, the age-associated CpG sites are selected from Table 8 or 9 or a homolog of one or more thereof. In some embodiments, the age-associated CpG sites are selected from Table 12 or a homolog of one or more thereof. In some embodiments, the age-associated CpG sites are selected from Table 16 or a homolog of one or more thereof. In some embodiments, the age-associated CpG sites are selected from Table 19 or 20 or a homolog of one or more thereof. In some embodiments, the age-associated CpG sites are selected from Table 20 or a homolog of one or more thereof. In some embodiments, at least one of the primers (i) is selected from Table 4; and/or (ii) can be used to amplify the same CpG site as the primers of (i). In some embodiments, at least one of the primers (i) is selected from Table 11; and/or (ii) can be used to amplify the same CpG site as the primers of (i). In some embodiments, at least one of the primers (i) is selected from Table 15; and/or (ii) can be used to amplify the same CpG site as the primers of (i).

In some embodiments, there is also provided a computer-readable medium which comprises a training data set comprising one or more or all of the CpG sites listed in Tables 1, 2, 3, 7, 8, 9, 12, 16, 19 or 20 or a homolog of one or more thereof. In some embodiments, the training data set comprises any of the CpG sites listed in Table 1 or at least 5, 10, 25, 50, 100, 150, 200, 250, 300, 350, 400, 450, 550, 600, 650, 700, 750, 800, 850, 900, 950, 1000, 1050, 1100, 1150, 1200, 1250, 1300 or all of the 1311 CpG sites listed in Table 1 or a homolog of one or more thereof. In some embodiments, the training data set comprises any of the CpG sites listed in Table 19 or at least 5, 10, 15, 20, 25, 30, 35, 40, 45, 50, 60, 70, 80, 90, 100, 110 or all of the 119 CpG sites listed in Table 19 or a homolog of one or more thereof.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples.

### BRIEF DESCRIPTION OF DRAWINGS

FIG. 1: A method of estimating the age of a Zebrafish in accordance with an embodiment of the present application. The exemplified method estimated the age of Zebrafish in a test data set from levels of methylated cytosine at 29 CpG sites. FIG. 1A shows performance of the model in the training data set (cor=0.95, p-value<2.20×10⁻¹⁶) FIG. 1B shows performance of the model in the testing data set (cor=0.92, p-value=9.56×10⁻¹¹). Colour represents the sample sex in the correlation plots. FIG. 1C shows boxplots showing the absolute error rate in the training and testing data sets. FIG. 1D shows unsupervised clustering of samples using the 29 CpG sites show separation based on age in the first principle component.

FIG. 2: Principle component analysis on an embodiment of the present application displaying no separation of sample sex.

FIG. 3: FIG. 3A shows weighting and directionality of each of 29 age associated CpG sites in accordance with an embodiment of the present application. FIG. 3B shows distribution of the performance of 10,000 age-estimation models in the form of median absolute error (weeks).

FIG. 4: Methylation-sensitive PCR was used to estimate age in zebrafish. FIG. 4A shows correlation between the chronological and predicted age (cor=0.62, p-value 0.00028). FIG. 4B shows the absolute error rate in age estimation (average MAE=13.4 weeks, Error relative to maximum age=17.2%).

FIG. 5: A method of estimating the age of a Zebrafish in accordance with an embodiment of the present application using multiplex PCR and DNA sequencing. Performance of age estimation by multiplex PCR in accordance with embodiments described herein showing the absolute error rate for 96 samples in triplicate.

FIG. 6: A method of estimating the age of a Zebrafish in accordance with an embodiment of the present application using multiplex PCR and DNA sequencing. Correlation between the chronological and predicted age in zebrafish. Samples were run in triplicate. FIG. 6A is a graph showing cor=0.97, p-value<2.20×10⁻¹⁶. FIG. 6B is a graph showing cor=0.96, p-value<2.20×10⁻¹⁶. FIG. 6C is a graph showing cor=0.97, p-value<2.20×10⁻¹⁶.

FIG. 7: Absolute error rate of samples by multiplex PCR over increasing age. The consistent absolute error rate over the lifespan of a Zebrafish shows the precision of the assay.

FIG. 8: Age estimation in school sharks (Galeorhinus galeus) in accordance with an embodiment of the present application. The exemplified model analysed DNA methylation at 30 CpG sites using a great white shark reference genome. FIG. 8A shows performance of the model in the training data set (cor=0.83, p-value=3.29×10⁻¹⁶). FIG. 8B shows performance of the model in the testing data set (cor=0.81, p-value=5.54×10⁻⁷). FIG. 8C shows boxplots showing the absolute error rate in the training and testing data sets using the great white shark reference genome. The median absolute error rate in the training samples was 0.80 years and 1.31 years in the testing samples.

FIG. 9: Age estimation in school sharks (Galeorhinus galeus) in accordance with an embodiment of the present application. The exemplified model analysed DNA methylation at 23 CpG sites using the whale shark reference genome (ASM164234v2). FIG. 9A shows performance of the model in the training data set (cor=0.74, p-value=1.03×10⁻¹²). FIG. 9B shows performance of the model in the testing data set (cor=0.61, p-value=0.00105). FIG. 9C shows boxplots showing the absolute error rate in the training and testing data sets using the whale shark reference genome. The median absolute error rate in the training samples was 1.69 years and 1.82 years in the testing samples.

FIG. 10: Age estimation by DNA methylation in the Australian Lungfish. FIG. 10A shows correlation plots between the chronological and predicted age in the training data set (Pearson correlation=0.98, p-value=2.92×10⁻⁷⁶). FIG. 10B shows correlation plots between the chronological and predicted age in the testing data set (Pearson correlation=0.98, p-value=1.39×10⁻³²). FIG. 10C shows boxplots showing the absolute error rate in age estimation in both the training and testing data sets.

FIG. 11: Age estimation by DNA methylation in the Murray cod and Mary River cod. FIG. 11A shows correlation plots between the chronological and predicted age in the training data set (Pearson correlation=0.92, p-value=1.36×10⁻²⁰). FIG. 11B shows correlation plots between the chronological and predicted age in the testing data set (Pearson correlation=0.92, p-value=1.36×10⁻¹³). FIG. 11C shows boxplots showing the absolute error rate in age estimation in both the training and testing data sets.

FIG. 12: Age estimation by DNA methylation in Green sea turtle (Chelonia mydas) using the 29 CpG sites from Table 20. FIG. 12A shows correlation plots between the chronological and predicted age in the training data set (Pearson correlation=0.93, p-value=<2.20×10⁻¹⁶). FIG. 12B shows correlation plots between the chronological and predicted age in the testing data set (Pearson correlation=0.90, p-value=7.54×10⁻⁷).

FIG. 12C shows boxplots showing the absolute error rate between the chronological and predicted age for the Green sea turtles. No statistical difference was found between the training (median=1.81 years) and testing (median=2.57 years) absolute error rates (t-test, two-tailed, p-value=0.143).

### KEY TO SEQUENCE LISTING

SEQ ID NO: 1-52: primers for multiplex PCR in accordance with Example 2.
SEQ ID NO: 53-78: amplicon amplified by primers listed in Table 4.
SEQ ID NO: 79-194: primers for msPCR in accordance with Example 2.
SEQ ID NO: 195-224: 300 bp amplicon comprising CpG site as described in Table 8.
SEQ ID NO: 225-334: primers for PCR in accordance with Example 7.
SEQ ID NO: 335-389: gDNA amplicon amplified by the primers defined in Example 7.
SEQ ID NO: 390-485: primers for PCR in accordance with Example 8.
SEQ ID NO: 486-533: gDNA amplicon amplified by the primers defined in Example 8.
SEQ ID NO: 534-562: 600 bp amplicon comprising CpG site as described in Table 20.

### DETAILED DESCRIPTION OF THE INVENTION

### General Techniques and Selected Definitions

Unless specifically defined otherwise, all technical and scientific terms used herein shall be taken to have the same meaning as commonly understood by one of ordinary skill in the art (for example, in epigenetics, biochemistry, molecular biology, fish ecology, and zoology). The following definitions apply to the terms as used throughout this specification, unless otherwise limited in specific instances.

As used herein, the term “about”, unless stated to the contrary, refers to +/−10%, +/−5%, or +/−1%, of the specified value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “consists of”, or variations such as “consisting of”, refers to the inclusion of any stated element, integer or step, or group of elements, integers or steps, that are recited in context with this term, and excludes any other element, integer or step, or group of elements, integers or steps, that are not recited in context with this term.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Throughout the present specification, various aspects and components of the disclosure can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 2, from 1 to 3, from 1 to 4, from 2 to 3, from 2 to 4, from 2 to 5, from 3 to 4 etc., as well as individual and partial numbers within the recited range, for example, 1, 2, 3, 4, and 5. This applies regardless of the breadth of the disclosed range. Where specific values are required, these will be indicated in the specification.

As used herein, the term “subject” refers to a fish or reptile. For example, the subject can be any fish (e.g., Atlantic salmon, blue fin tuna, zebrafish) or reptile (e.g. marine turtle, land turtle, lizard). In one example, the subject is a fish. In one embodiment, the fish is a member of the subclass Elasmobranchii (e.g. shark or ray). In one embodiment, the subject is a reptile. In one embodiment, the reptile is a turtle.

### Method for Estimating the Age of Fish or Reptile

The present inventors have surprisingly found that certain CpG sites, referred to herein as age-associated CpG sites, can be used to estimate the age of a fish or reptile. The present inventors have also demonstrated that the age-associated CpG sites for one species (e.g. zebrafish, school shark or green sea turtle) can be used to identify age-associated CpG sites for a second species. These age associated CpG sites can then be used to estimate the age of the second species. Accordingly, the present application provides a method of estimating the age of a fish or reptile. In some embodiments, there is provided a method for estimating the age of a fish or reptile comprising estimating the age of the fish or reptile based on analysis of DNA obtained from the fish or reptile for the presence of a methylated cytosine at age-associated CpG sites.

### Age-Associated CpG Sites

The method of estimating the age of a fish or reptile described herein comprises analysing DNA obtained from the fish or reptile for the presence of methylated cytosine at age-associated CpG sites.

As used herein a “methylated cytosine” refers to a cytosine derivative that comprises a methyl moiety at a position where a methyl moiety is not present in a cytosine. For example, cytosine does not contain a methyl moiety on its pyrimidine ring, but the methylated cytosine, 5-methylcytosine, contains a methyl moiety at position 5 of its pyrimidine ring.

As used herein, “CpG” (also referred to as “CG”) is shorthand for 5′-C-phosphate-G-3′ (i.e., cytosine and guanine separated by a single phosphate group) and refers to regions of nucleic acid where a cytosine nucleotide is followed by a guanine nucleotide in the linear sequence of bases along a 5′ to 3′ direction. The nucleic acid is typically DNA. The cytosine nucleotide can optionally contain a methyl moiety, hydroxymethyl moiety or hydrogen moiety at position 5 of the pyrimidine ring. The term “CpG site” is used interchangeably with “methylation site” and is a site in a nucleic acid where methylation has occurred, or has the possibility of occurring.

As used herein, the term “age-associated CpG site” (or age-associated methylation site) refers to a CpG site whose methylation status changes as the fish or reptile ages. In other words, age-associated CpG sites are susceptible to methylation or demethylation as the fish or reptile ages. A change in methylation status can include an increase in methylation of the cytosine at the CpG site or a decrease in methylation of the cytosine at the CpG site. In some embodiments, an age-associated CpG site has a significant Pearson correlation with age (e.g. p<0.05).

In some embodiments, for example where the fish is a bony fish, the age-associated CpG sites are selected from any of the CpG sites listed in Tables 1, 2 or 3 or a homolog of one or more thereof.

In some embodiments, the age-associated CpG sites are selected from any of the CpG sites listed in Table 1 or a homolog of one or more thereof. In some embodiments, the age associated CpG sites comprise at least 1%, at least 2%, at least 3%, at least 4%, at least 5%, at least 6%, at least 7%, at least 8%, at least 9%, at least 10%, at least 110, at least 12%, at least 13%, at least 14%, at least 15%, at least 16%, at least 17%, at least 18%, at least 19%, at least 20%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more of the CpG sites listed in Table 1, or a homolog of one or more thereof. In still a further embodiment, the method comprises from 1-1,311 (and any whole number there between), e.g., 1-2, 3-4, 5-10, 10-20, 20-29, 30-49, 50-100, 101-150, 151-200, 201-250, 251-300, 301-400, 401-500, 501-600, 601-700, 701-800, 801-900, 901-1,000, 1,001-1,100, 1,101-1,200, 1,201-1,300 or 1,301-1,311 CpG sites of Table 1 or a homolog of one or more thereof.

In some embodiments, the age-associated CpG sites comprise any of the 29 CpG sites listed in Table 2 or a homolog of one or more thereof. In some embodiments, the presence of methylated cytosine is analysed at 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the age-associated CpG sites listed in Table 2 or a homolog of one or more thereof. In some embodiments, the presence of methylated cytosine is analysed at all of the age-associated CpG sites listed in Table 2.

In some embodiments, the age-associated CpG sites comprise any of the 26 CpG sites listed in Table 3 or a homolog of one or more thereof. In some embodiments, the presence of methylated cytosine is analysed at all of the age-associated CpG sites listed in Table 3. Although the ageing model exemplified herein made use of the 26 CpG sites listed in Table 3 it will be appreciated that a smaller subset of the sites may be used to provide an effective ageing model. In some embodiments, the presence of methylated cytosine is analysed at 1, 2, 3, 4, 5, 6, 7, 8, 9, 10, 11, 12, 13, 14, 15, 16, 17, 18, 19, 20, 21, 22, 23, 24, 25, or 26 of the age-associated CpG sites listed in Table 3 or a homolog of one or more thereof.

In some embodiments, the age-associated CpG sites comprise one or more of the CpG sites listed in Table 7 or a homolog of one or more thereof. Although an ageing model may use all of the CpG sites listed in Table 7 it will be appreciated that a smaller subset of the sites may be used to provide an effective ageing model. In some embodiments, the method comprises from 1-131 (and any whole number there between), e.g., 1-2, 3-4, 5-10, 10-20, 20-29, 30-48, 49-60, 61-100, 101-131 of the CpG sites of Table 7 or a homolog of one or more thereof.

In some embodiments, for example where the fish is a member of the subclass Elasmobranchii (e.g. shark or ray), the age-associated CpG sites are selected from any of the CpG sites listed in Tables 8 or 9 or a homolog of one or more thereof.

As will be appreciated by the person skilled in the art the CpG sites provided in Table 7 are homologs of one or more of the CpG sites provided in Tables 1, 2 or 3 (e.g. Table 1).

In some embodiments, the age-associated CpG sites comprise any of the 30 CpG sites listed in Table 8 or a homolog of one or more thereof. In some embodiments, the presence of methylated cytosine is analysed at all of the age-associated CpG sites listed in Table 8. Although the ageing model exemplified herein made use of the 30 CpG sites listed in Table 8 it will be appreciated that a smaller subset of the sites may be used to provide an effective ageing model. In some embodiments, the presence of methylated cytosine is analysed at 1, 2, 3, 4, 5, 6, 7, 8, 9, 10, 11, 12, 13, 14, 15, 16, 17, 18, 19, 20, 21, 22, 23, 24, 25, 26, 27, 29, 29 or 30 of the age-associated CpG sites listed in Table 8 or a homolog of one or more thereof.

In some embodiments, the age-associated CpG sites comprise any of the 23 CpG sites listed in Table 9 or a homolog of one or more thereof. In some embodiments, the presence of methylated cytosine is analysed at all of the age-associated CpG sites listed in Table 9. Although the ageing model exemplified herein made use of the 23 CpG sites listed in Table 9 it will be appreciated that a smaller subset of the sites may be used to provide an effective ageing model. In some embodiments, the presence of methylated cytosine is analysed at 1, 2, 3, 4, 5, 6, 7, 8, 9, 10, 11, 12, 13, 14, 15, 16, 17, 18, 19, 20, 21, 22 or 23 of the age-associated CpG sites listed in Table 9 or a homolog of one or more thereof.

In some embodiments, the age-associated CpG sites comprise any of the 31 CpG sites listed in Table 12 or a homolog of one or more thereof. In some embodiments, the presence of methylated cytosine is analysed at all of the age-associated CpG sites listed in Table 12. Although the ageing model exemplified herein made use of the 31 CpG sites listed in Table 12 it will be appreciated that a smaller subset of the sites may be used to provide an effective ageing model. In some embodiments, the presence of methylated cytosine is analysed at 1, 2, 3, 4, 5, 6, 7, 8, 9, 10, 11, 12, 13, 14, 15, 16, 17, 18, 19, 20, 21, 22, 23, 24, 25, 26, 27, 29, 30 or 31 of the age-associated CpG sites listed in Table 12 or a homolog of one or more thereof.

In some embodiments, the age-associated CpG sites comprise any of the 26 CpG sites listed in Table 16 or a homolog of one or more thereof. In some embodiments, the presence of methylated cytosine is analysed at all of the age-associated CpG sites listed in Table 16. Although the ageing model exemplified herein made use of the 26 CpG sites listed in Table 16 it will be appreciated that a smaller subset of the sites may be used to provide an effective ageing model. In some embodiments, the presence of methylated cytosine is analysed at 1, 2, 3, 4, 5, 6, 7, 8, 9, 10, 11, 12, 13, 14, 15, 16, 17, 18, 19, 20, 21, 22, 23, 24, 25 or 26 of the age-associated CpG sites listed in Table 16 or a homolog of one or more thereof.

As will be appreciated by the person skilled in the art the CpG sites provided in Table 12 and 16 are homologs of one or more of the CpG sites provided in Tables 1, 2 or 3 (e.g. Table 1).

In some embodiments, the age-associated CpG sites comprise any of the 119 CpG sites listed in Table 19 or a homolog of one or more thereof. In some embodiments, the presence of methylated cytosine is analysed at all of the age-associated CpG sites listed in Table 19. Although the ageing model exemplified herein made use of the 119 CpG sites listed in Table 19 it will be appreciated that a smaller subset of the sites may be used to provide an effective ageing model. In some embodiments, the presence of methylated cytosine is analysed at 15, 10, 15, 20, 25, 30, 35, 40, 45, 50, 60, 70, 80, 90, 100, 110 or all of the 119 of the age-associated CpG sites listed in Table 19 or a homolog of one or more thereof.

In some embodiments, the age-associated CpG sites comprise any of the 29 CpG sites listed in Table 20 or a homolog of one or more thereof. In some embodiments, the presence of methylated cytosine is analysed at all of the age-associated CpG sites listed in Table 20. Although the ageing model exemplified herein made use of the 29 CpG sites listed in Table 20 it will be appreciated that a smaller subset of the sites may be used to provide an effective ageing model. In some embodiments, the presence of methylated cytosine is analysed at 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the age-associated CpG sites listed in Table 20 or a homolog of one or more thereof.

It will be appreciated by the person skilled in the art that homologs of the age-associated CpG sites identified in Tables 1, 2, 3, 8, 9, 12, 16, 19 or 20 includes CpG sites from a different species identified based on homology (e.g. sequence homology) with the CpG sites listed in Tables 1, 2, 3, 8, 9, 12, 16, 19 or 20 or a subset thereof. For example, homologs of the CpG sites described herein may be identified using prediction software, such as ClustalW (Thompson et al., 1994; available at www.genome.jp/tools-bin/clustalw), LASTZ (Harris 2007; available at www.bx.psu.edu/miller_lab/dist/README.lastz-1.02.00/README.lastz-1.02.00a.html#intro) or HISAT2 (Kim et al., 2015), to align the sequences of pairs of species and homologous CpG sites identified using suitable bioinformatics tools, e.g., by applying the Perl module Bio::AlignIO. In some embodiments, potential error due to misalignment may be removed, by further filtering the sites by requiring that the two flanking nucleotides (immediately upstream and downstream of each focal CpG) also are identical between the pair of species. In some embodiments, the genomic sequence for the fish or reptile of interest is aligned against a reference genome. In some embodiments, RNA sequence data is aligned against a reference genome. In some embodiments, the reference genome is the zebrafish reference genome (danRer10, Illumina iGenomes). As exemplified in Examples 8 and 9, the identification of homologous sites from other species is well within the capability of the skilled person. In some embodiments, homologs of one or more of the age-associated CpG sites identified in Tables 1, 2 or 3 comprise one or more of the age-associated CpG sites identified in Tables 7, 12 and/or 16. In some embodiments, homologs of one or more of the age-associated CpG sites identified in Tables 1, 2 or 3 comprise one or more of the age-associated CpG sites identified in Tables 12 and/or 16.

In some embodiments, the method does not analyse age-associated CpG sites in one or more or all of the genes selected from amh-r2, fsh-r, nr3c1 and sox9. In some embodiments, the method does not analyse age-associated CpG sites in one or more or all of the genes selected from 3bhsd, amh, amhr2, cyp11a, cyp17a1, cyp19a1a, cyp26a1, dnmt3a, erb1, er-b2, fshr, igf1, lhr, myf6, myhm86-1, mylz2, myod, nr3c1, sox19a, sox9, vasa and wnt1. In some embodiments, the method does not analyse CpG sites in the amh-r2, fsh-r, nr3c1 and sox9 genes.

The present inventors have found that use of the CpG sites (or homogs thereof) as described herein provides one or more advantages over the use of CpG sites that are selected based on based on a known function or property. In some embodiments, these advantages include increased sensitivity, accuracy and/or reproducibility; reduced cost; and/or decreased invasiveness; and/or flexibility in the choice of biological sample used to estimate age. The present inventors have also shown that, advantageously, the CpG sites as described herein allow for the prediction age across multiple species of fish or reptiles. As a result, the methods described herein are particularly suited for estimating the age of endangered fish or reptiles or for fish or reptiles where a population of known age is not readily available.

TABLE 1
Age-associated CpG sites as exemplified herein. The genomic
coordinates are from the Zebrafish genome version danRer10.

| CpG site | | | CpG site | | | CpG site | | |
|---|---|---|---|---|---|---|---|---|
| chr | position | strand | chr | position | strand | chr | position | strand |
| chr1 | 609687 | − | chr10 | 42116261 | + | chr18 | 29231439 | + |
| chr1 | 717733 | + | chr10 | 42989253 | + | chr18 | 30707659 | + |
| chr1 | 718954 | + | chr10 | 45397101 | + | chr18 | 31628946 | + |
| chr1 | 719012 | − | chr10 | 45904489 | + | chr18 | 32787896 | + |
| chr1 | 2362308 | + | chr10 | 47084931 | + | chr18 | 32849316 | + |
| chr1 | 2797554 | + | chr10 | 47212501 | + | chr18 | 33114540 | + |
| chr1 | 2861423 | + | chr10 | 47957540 | + | chr18 | 33333414 | + |
| chr1 | 3046678 | + | chr10 | 49515873 | + | chr18 | 33934994 | + |
| chr1 | 3161317 | + | chr10 | 50298364 | − | chr18 | 33978245 | + |
| chr1 | 4757457 | + | chr10 | 50663172 | + | chr18 | 34030652 | + |
| chr1 | 6966699 | + | chr10 | 51696425 | + | chr18 | 35650423 | + |
| chr1 | 8608715 | + | chr10 | 52813817 | + | chr18 | 35720876 | + |
| chr1 | 8611626 | + | chr10 | 53747435 | + | chr18 | 35996868 | + |
| chr1 | 10065929 | + | chr10 | 54701064 | + | chr18 | 36022526 | + |
| chr1 | 12079006 | − | chr10 | 55309153 | + | chr18 | 36035786 | + |
| chr1 | 13755578 | − | chr11 | 393042 | + | chr18 | 36108032 | + |
| chr1 | 14524421 | + | chr11 | 690188 | + | chr18 | 36532280 | + |
| chr1 | 14572540 | + | chr11 | 741151 | + | chr18 | 37795395 | + |
| chr1 | 16681099 | + | chr11 | 4073004 | + | chr18 | 38278671 | + |
| chr1 | 16981800 | + | chr11 | 4550447 | + | chr18 | 38530830 | + |
| chr1 | 18054888 | + | chr11 | 5574992 | + | chr18 | 38667904 | + |
| chr1 | 19939201 | + | chr11 | 6601708 | + | chr18 | 38698782 | + |
| chr1 | 20345119 | + | chr11 | 6847454 | + | chr18 | 39187355 | + |
| chr1 | 21051510 | + | chr11 | 7834897 | + | chr18 | 39282718 | + |
| chr1 | 22457088 | + | chr11 | 8153725 | + | chr18 | 42179720 | + |
| chr1 | 22860201 | + | chr11 | 8956608 | + | chr18 | 42785426 | + |
| chr1 | 23386154 | + | chr11 | 9472216 | + | chr18 | 43221811 | + |
| chr1 | 23386230 | + | chr11 | 9752400 | + | chr18 | 44460522 | + |
| chr1 | 23598870 | + | chr11 | 12499927 | + | chr19 | 17439 | + |
| chr1 | 25177107 | + | chr11 | 12771368 | + | chr19 | 992163 | + |
| chr1 | 25299286 | − | chr11 | 14003393 | + | chr19 | 1014167 | + |
| chr1 | 25497432 | + | chr11 | 14640010 | + | chr19 | 1772414 | − |
| chr1 | 26110391 | + | chr11 | 15504809 | + | chr19 | 2980826 | − |
| chr1 | 26418282 | + | chr11 | 16665027 | + | chr19 | 3021977 | + |
| chr1 | 26881784 | + | chr11 | 17091585 | + | chr19 | 3203348 | − |
| chr1 | 26947853 | + | chr11 | 18116053 | + | chr19 | 4215673 | + |
| chr1 | 26947925 | + | chr11 | 18255431 | + | chr19 | 4293521 | + |
| chr1 | 27318023 | − | chr11 | 21775622 | + | chr19 | 4417670 | + |
| chr1 | 27606184 | + | chr11 | 23689310 | + | chr19 | 5432194 | + |
| chr1 | 27826360 | + | chr11 | 24091046 | − | chr19 | 8757255 | + |
| chr1 | 27863431 | + | chr11 | 24549312 | + | chr19 | 9022776 | − |
| chr1 | 28162821 | − | chr11 | 24856318 | + | chr19 | 10306003 | − |
| chr1 | 29184136 | − | chr11 | 25612172 | − | chr19 | 11669054 | + |
| chr1 | 29618324 | + | chr11 | 27232431 | + | chr19 | 12422993 | + |
| chr1 | 32241543 | + | chr11 | 28388248 | + | chr19 | 13856844 | + |
| chr1 | 35652447 | + | chr11 | 28611493 | + | chr19 | 14308846 | − |
| chr1 | 35678182 | + | chr11 | 28904513 | + | chr19 | 15116860 | + |
| chr1 | 37590968 | + | chr11 | 29101209 | + | chr19 | 15174116 | + |
| chr1 | 37619595 | + | chr11 | 29843848 | + | chr19 | 17943579 | + |
| chr1 | 38669969 | + | chr11 | 29855938 | + | chr19 | 18035101 | + |
| chr1 | 38839196 | + | chr11 | 32281234 | + | chr19 | 18291981 | + |
| chr1 | 39636232 | + | chr11 | 32649161 | + | chr19 | 19030749 | + |
| chr1 | 40796018 | − | chr11 | 33329722 | + | chr19 | 19358703 | + |
| chr1 | 42957940 | + | chr11 | 33361680 | + | chr19 | 19406405 | + |
| chr1 | 43259461 | + | chr11 | 33895387 | − | chr19 | 19868851 | + |
| chr1 | 43480815 | − | chr11 | 34700048 | + | chr19 | 20700533 | + |
| chr1 | 44702461 | + | chr11 | 38089872 | − | chr19 | 21102192 | + |
| chr1 | 44740481 | + | chr11 | 38382637 | − | chr19 | 21323751 | + |
| chr1 | 48752075 | + | chr11 | 39036667 | + | chr19 | 22396428 | + |
| chr1 | 49449291 | + | chr11 | 39654246 | + | chr19 | 23051056 | + |
| chr1 | 50094203 | − | chr11 | 42283229 | − | chr19 | 27102641 | + |
| chr1 | 50638360 | + | chr11 | 42803207 | + | chr19 | 27405472 | + |
| chr1 | 51192910 | + | chr11 | 43567397 | + | chr19 | 28269341 | + |
| chr1 | 51241960 | + | chr11 | 45465564 | + | chr19 | 28304027 | − |
| chr1 | 51253087 | + | chr11 | 48311255 | + | chr19 | 28358425 | + |
| chr1 | 51494414 | + | chr11 | 50887169 | + | chr19 | 29937760 | + |
| chr1 | 52110013 | + | chr11 | 51225049 | − | chr19 | 30545634 | + |
| chr1 | 55636504 | + | chr11 | 52557575 | + | chr19 | 31264119 | + |
| chr1 | 57919215 | + | chr11 | 52623927 | + | chr19 | 32417517 | + |
| chr1 | 58251860 | + | chr11 | 52641016 | + | chr19 | 32920576 | + |
| chr2 | 74684 | + | chr11 | 52723656 | + | chr19 | 33344700 | + |
| chr2 | 3468012 | + | chr11 | 52836575 | + | chr19 | 33737223 | − |
| chr2 | 4495478 | + | chr11 | 52836692 | + | chr19 | 38927919 | − |
| chr2 | 4771734 | + | chr11 | 53162559 | + | chr19 | 40625110 | + |
| chr2 | 4979991 | + | chr12 | 54678 | − | chr19 | 41593463 | + |
| chr2 | 5267529 | + | chr12 | 1180912 | + | chr19 | 42102727 | − |
| chr2 | 5318674 | − | chr12 | 4489202 | + | chr20 | 832448 | + |
| chr2 | 5331787 | + | chr12 | 5004547 | + | chr20 | 1147259 | − |
| chr2 | 9524076 | + | chr12 | 5004559 | − | chr20 | 1523744 | + |
| chr2 | 9649515 | + | chr12 | 6096256 | + | chr20 | 1810569 | + |
| chr2 | 9875401 | + | chr12 | 9037848 | + | chr20 | 2540405 | + |
| chr2 | 10919169 | + | chr12 | 11393067 | + | chr20 | 2973456 | + |
| chr2 | 10919235 | + | chr12 | 12323254 | + | chr20 | 5215641 | + |
| chr2 | 12508862 | + | chr12 | 13966407 | + | chr20 | 6461988 | + |
| chr2 | 15145200 | + | chr12 | 14840019 | + | chr20 | 7744368 | + |
| chr2 | 15837480 | + | chr12 | 14910110 | + | chr20 | 8728622 | + |
| chr2 | 21108545 | + | chr12 | 15025564 | + | chr20 | 10043148 | + |
| chr2 | 21424233 | + | chr12 | 15384747 | + | chr20 | 10202641 | + |
| chr2 | 21847229 | + | chr12 | 16434087 | − | chr20 | 11154998 | + |
| chr2 | 22357790 | + | chr12 | 17821994 | + | chr20 | 11827122 | + |
| chr2 | 22870956 | + | chr12 | 19178812 | + | chr20 | 12664040 | + |
| chr2 | 24401666 | + | chr12 | 19513343 | + | chr20 | 13518026 | + |
| chr2 | 25382953 | + | chr12 | 19660240 | + | chr20 | 13541130 | + |
| chr2 | 27112961 | + | chr12 | 19988060 | + | chr20 | 14631230 | + |
| chr2 | 27112975 | − | chr12 | 20423747 | + | chr20 | 14831093 | + |
| chr2 | 32029173 | + | chr12 | 21141984 | + | chr20 | 16313450 | + |
| chr2 | 32610913 | + | chr12 | 22388034 | + | chr20 | 16967937 | + |
| chr2 | 33686767 | + | chr12 | 22528244 | + | chr20 | 17056388 | + |
| chr2 | 35421769 | + | chr12 | 22844926 | + | chr20 | 17060159 | + |
| chr2 | 37324919 | + | chr12 | 23269195 | + | chr20 | 17364443 | + |
| chr2 | 37556376 | + | chr12 | 25936740 | + | chr20 | 17543726 | + |
| chr2 | 37653753 | + | chr12 | 28505289 | − | chr20 | 18993534 | + |
| chr2 | 39066654 | + | chr12 | 29416858 | + | chr20 | 19937660 | + |
| chr2 | 40996065 | − | chr12 | 31649639 | + | chr20 | 20199458 | + |
| chr2 | 42252679 | + | chr12 | 31763330 | + | chr20 | 20280532 | + |
| chr2 | 43094971 | + | chr12 | 33386146 | + | chr20 | 20476371 | + |
| chr2 | 43577043 | + | chr12 | 33525870 | − | chr20 | 21134702 | + |
| chr2 | 43837384 | + | chr12 | 35786206 | + | chr20 | 23455052 | + |
| chr2 | 43905240 | + | chr12 | 38107080 | + | chr20 | 25222747 | + |
| chr2 | 43924435 | + | chr12 | 38774780 | + | chr20 | 26278372 | − |
| chr2 | 44263107 | + | chr12 | 40205918 | + | chr20 | 26917360 | + |
| chr2 | 44325098 | + | chr12 | 40673257 | + | chr20 | 28364425 | − |
| chr2 | 44441391 | + | chr12 | 44114297 | + | chr20 | 28687482 | − |
| chr2 | 44498534 | + | chr12 | 45395736 | + | chr20 | 28736308 | + |
| chr2 | 44527467 | + | chr12 | 45997733 | + | chr20 | 28911827 | + |
| chr2 | 44730433 | + | chr12 | 47309497 | + | chr20 | 28993097 | − |
| chr2 | 44891379 | + | chr12 | 47388933 | + | chr20 | 29267447 | + |
| chr2 | 45094517 | + | chr12 | 48115888 | + | chr20 | 30535152 | + |
| chr3 | 17101 | + | chr12 | 48956657 | + | chr20 | 31225439 | + |
| chr3 | 23947 | + | chr12 | 49225304 | + | chr20 | 31845930 | + |
| chr3 | 303332 | + | chr12 | 50617327 | + | chr20 | 33265728 | + |
| chr3 | 686832 | + | chr12 | 50617496 | − | chr20 | 33381440 | + |
| chr3 | 1186369 | + | chr12 | 50711551 | + | chr20 | 33462107 | − |
| chr3 | 2016738 | + | chr12 | 50792250 | + | chr20 | 33659733 | + |
| chr3 | 8633915 | + | chr13 | 172703 | + | chr20 | 33670106 | + |
| chr3 | 10278381 | + | chr13 | 2010929 | + | chr20 | 33670361 | + |
| chr3 | 10844380 | + | chr13 | 2020126 | + | chr20 | 33670423 | + |
| chr3 | 11010626 | + | chr13 | 2184395 | + | chr20 | 34411058 | + |
| chr3 | 11813422 | + | chr13 | 3110686 | − | chr20 | 34628466 | + |
| chr3 | 11906244 | + | chr13 | 3257967 | + | chr20 | 34635669 | − |
| chr3 | 12371074 | + | chr13 | 3577722 | + | chr20 | 34929808 | + |
| chr3 | 13133647 | + | chr13 | 3858772 | + | chr20 | 34997523 | + |
| chr3 | 15327720 | + | chr13 | 3943128 | + | chr20 | 35313264 | + |
| chr3 | 17986892 | + | chr13 | 4169378 | + | chr20 | 35417650 | + |
| chr3 | 20067178 | + | chr13 | 4656622 | + | chr20 | 35817603 | + |
| chr3 | 23725851 | + | chr13 | 5365862 | + | chr20 | 36612071 | + |
| chr3 | 23990731 | + | chr13 | 6988917 | + | chr20 | 36872756 | + |
| chr3 | 24589836 | + | chr13 | 7235986 | + | chr21 | 1421297 | + |
| chr3 | 24959860 | + | chr13 | 8826617 | + | chr21 | 1423737 | + |
| chr3 | 25539262 | + | chr13 | 10909245 | + | chr21 | 3278933 | + |
| chr3 | 26373329 | + | chr13 | 11207697 | + | chr21 | 4064153 | + |
| chr3 | 27296387 | + | chr13 | 12455165 | + | chr21 | 6568740 | + |
| chr3 | 28037677 | + | chr13 | 14570388 | + | chr21 | 7873973 | + |
| chr3 | 32570378 | + | chr13 | 15253799 | + | chr21 | 8011117 | + |
| chr3 | 33671430 | − | chr13 | 16441651 | + | chr21 | 12038483 | + |
| chr3 | 35317955 | + | chr13 | 17998644 | + | chr21 | 14708524 | + |
| chr3 | 36822568 | + | chr13 | 18310375 | + | chr21 | 14807851 | + |
| chr3 | 36984668 | + | chr13 | 19361296 | + | chr21 | 14981176 | + |
| chr3 | 38645108 | + | chr13 | 19808183 | + | chr21 | 15480463 | + |
| chr3 | 38711068 | + | chr13 | 20077224 | + | chr21 | 15757027 | + |
| chr3 | 39260307 | + | chr13 | 20147055 | + | chr21 | 17530229 | + |
| chr3 | 40184938 | − | chr13 | 20184356 | + | chr21 | 17729887 | + |
| chr3 | 40519388 | + | chr13 | 20199908 | + | chr21 | 19072276 | + |
| chr3 | 40606783 | + | chr13 | 21021316 | + | chr21 | 20832257 | + |
| chr3 | 40654180 | + | chr13 | 21512175 | + | chr21 | 21949879 | + |
| chr3 | 41224102 | − | chr13 | 21616655 | + | chr21 | 22458169 | + |
| chr3 | 41246979 | + | chr13 | 21708489 | + | chr21 | 22899610 | − |
| chr3 | 41500407 | + | chr13 | 21984807 | + | chr21 | 22923627 | + |
| chr3 | 43612544 | + | chr13 | 21991319 | + | chr21 | 23443864 | + |
| chr3 | 43881457 | + | chr13 | 22225191 | + | chr21 | 23465726 | + |
| chr3 | 43952852 | − | chr13 | 22870011 | + | chr21 | 23529270 | + |
| chr3 | 44037008 | + | chr13 | 25430596 | + | chr21 | 23616782 | + |
| chr3 | 44440020 | − | chr13 | 29867330 | + | chr21 | 23721145 | + |
| chr3 | 44508416 | + | chr13 | 31974492 | + | chr21 | 25140919 | + |
| chr4 | 89896 | + | chr13 | 32252919 | + | chr21 | 25650389 | + |
| chr4 | 292184 | − | chr13 | 34090412 | + | chr21 | 25670889 | + |
| chr4 | 459840 | + | chr13 | 34427779 | + | chr21 | 26155708 | + |
| chr4 | 1897746 | − | chr13 | 35461349 | + | chr21 | 26155718 | − |
| chr4 | 1909929 | + | chr13 | 36722082 | + | chr21 | 26296507 | + |
| chr4 | 2190802 | + | chr13 | 36919632 | + | chr21 | 26298958 | + |
| chr4 | 4201004 | + | chr13 | 40178631 | + | chr21 | 29583279 | + |
| chr4 | 10283862 | + | chr13 | 40278985 | + | chr21 | 29803813 | + |
| chr4 | 10955632 | + | chr13 | 40360120 | + | chr21 | 29804025 | + |
| chr4 | 11300427 | + | chr13 | 40598707 | + | chr21 | 30330422 | + |
| chr4 | 12366612 | + | chr13 | 43940582 | + | chr21 | 31433861 | + |
| chr4 | 14240746 | + | chr13 | 44397429 | + | chr21 | 32234964 | + |
| chr4 | 14413041 | + | chr13 | 44908413 | + | chr21 | 32583381 | + |
| chr4 | 14937754 | + | chr13 | 45943788 | + | chr21 | 33631167 | + |
| chr4 | 19607449 | + | chr13 | 46567515 | − | chr21 | 34082010 | + |
| chr4 | 21308425 | + | chr13 | 47559355 | + | chr21 | 34984314 | + |
| chr4 | 21540399 | + | chr13 | 47835179 | + | chr21 | 35472605 | + |
| chr4 | 22322523 | + | chr13 | 48406692 | + | chr21 | 35944097 | + |
| chr4 | 23818596 | + | chr14 | 93146 | + | chr21 | 37459124 | + |
| chr4 | 25519001 | + | chr14 | 446923 | + | chr21 | 37461493 | + |
| chr4 | 26185329 | + | chr14 | 944559 | + | chr21 | 37472110 | + |
| chr4 | 27026434 | + | chr14 | 944659 | + | chr21 | 38946002 | + |
| chr4 | 27932596 | + | chr14 | 2111641 | + | chr21 | 39530945 | + |
| chr4 | 28044082 | + | chr14 | 2425814 | + | chr21 | 40602618 | + |
| chr4 | 30076409 | + | chr14 | 3077211 | + | chr21 | 40880949 | + |
| chr4 | 30879174 | + | chr14 | 4367487 | + | chr21 | 41348990 | + |
| chr4 | 31003406 | + | chr14 | 4582555 | + | chr21 | 41369420 | + |
| chr4 | 31094534 | + | chr14 | 5047475 | + | chr21 | 42587400 | + |
| chr4 | 31777269 | + | chr14 | 6351706 | + | chr21 | 42887804 | + |
| chr4 | 32131249 | + | chr14 | 7370224 | − | chr21 | 42997681 | + |
| chr4 | 32465449 | + | chr14 | 8207957 | + | chr21 | 43133779 | + |
| chr4 | 32541425 | + | chr14 | 8280826 | + | chr21 | 44361938 | + |
| chr4 | 33253253 | + | chr14 | 9468000 | − | chr21 | 44383573 | + |
| chr4 | 34711088 | − | chr14 | 9935031 | + | chr21 | 46454864 | + |
| chr4 | 35057312 | + | chr14 | 10395395 | + | chr21 | 47698707 | + |
| chr4 | 35144510 | + | chr14 | 12128221 | + | chr21 | 49593249 | + |
| chr4 | 35432443 | + | chr14 | 14686082 | + | chr21 | 49624225 | + |
| chr4 | 36185065 | + | chr14 | 16121268 | + | chr21 | 52025877 | + |
| chr4 | 37001008 | + | chr14 | 20230428 | + | chr21 | 52894670 | + |
| chr4 | 41184118 | + | chr14 | 22102701 | + | chr21 | 53211624 | + |
| chr4 | 42571790 | − | chr14 | 22102760 | + | chr21 | 53859380 | − |
| chr4 | 42665340 | + | chr14 | 24551724 | + | chr21 | 55413055 | + |
| chr4 | 42989774 | − | chr14 | 24909170 | + | chr21 | 55489740 | + |
| chr4 | 43189257 | + | chr14 | 24963818 | + | chr21 | 55509405 | + |
| chr4 | 43757425 | + | chr14 | 25739992 | + | chr21 | 56573005 | + |
| chr4 | 44506693 | + | chr14 | 25776000 | + | chr21 | 57459751 | − |
| chr5 | 713889 | + | chr14 | 28672109 | + | chr21 | 57703915 | + |
| chr5 | 1626555 | + | chr14 | 32417155 | + | chr21 | 58685773 | + |
| chr5 | 1798541 | + | chr14 | 33293011 | + | chr21 | 59876087 | + |
| chr5 | 1897921 | − | chr14 | 34033357 | + | chr21 | 61064785 | + |
| chr5 | 1962719 | + | chr14 | 37185367 | + | chr21 | 62165137 | − |
| chr5 | 2346076 | + | chr14 | 37327874 | − | chr22 | 301182 | − |
| chr5 | 3309556 | − | chr14 | 38165650 | + | chr22 | 683201 | + |
| chr5 | 3824160 | + | chr14 | 38165658 | − | chr22 | 1320597 | + |
| chr5 | 4719773 | − | chr14 | 40674052 | + | chr22 | 1434444 | + |
| chr5 | 5518461 | + | chr14 | 40810800 | + | chr22 | 1939730 | + |
| chr5 | 5762835 | + | chr14 | 41269137 | + | chr22 | 2765126 | + |
| chr5 | 6008546 | + | chr14 | 41269456 | + | chr22 | 3059126 | + |
| chr5 | 7169136 | + | chr14 | 41635470 | + | chr22 | 3381123 | + |
| chr5 | 9110526 | + | chr14 | 41686867 | + | chr22 | 4008591 | + |
| chr5 | 10667023 | + | chr14 | 42279274 | + | chr22 | 4446115 | + |
| chr5 | 11744067 | + | chr14 | 42510538 | + | chr22 | 4675128 | + |
| chr5 | 11996110 | + | chr14 | 43083626 | + | chr22 | 5293538 | + |
| chr5 | 13893859 | + | chr14 | 43697062 | + | chr22 | 6168760 | − |
| chr5 | 14845188 | + | chr14 | 44457874 | + | chr22 | 6299812 | + |
| chr5 | 15334342 | + | chr14 | 45624815 | + | chr22 | 6469466 | + |
| chr5 | 17280376 | + | chr14 | 47054259 | + | chr22 | 6775288 | + |
| chr5 | 17310710 | + | chr14 | 47528675 | + | chr22 | 8318680 | − |
| chr5 | 17339498 | + | chr14 | 48452415 | − | chr22 | 8830038 | + |
| chr5 | 17506997 | + | chr14 | 53233671 | + | chr22 | 9481667 | + |
| chr5 | 18302264 | + | chr14 | 55213308 | + | chr22 | 9821305 | + |
| chr5 | 18947288 | + | chr14 | 57019506 | + | chr22 | 10201387 | + |
| chr5 | 19004461 | + | chr14 | 57903359 | + | chr22 | 10269069 | + |
| chr5 | 19400334 | + | chr15 | 140354 | + | chr22 | 10303606 | + |
| chr5 | 21578737 | − | chr15 | 1938725 | + | chr22 | 11561243 | + |
| chr5 | 22177645 | + | chr15 | 2717003 | + | chr22 | 12930754 | + |
| chr5 | 23879913 | + | chr15 | 2946070 | + | chr22 | 13123586 | + |
| chr5 | 24123828 | + | chr15 | 9440006 | + | chr22 | 13277388 | + |
| chr5 | 25410381 | + | chr15 | 10504330 | + | chr22 | 14192551 | + |
| chr5 | 25704250 | + | chr15 | 11318990 | + | chr22 | 14373473 | + |
| chr5 | 25787963 | + | chr15 | 11533198 | + | chr22 | 14718812 | − |
| chr5 | 25912278 | + | chr15 | 11670867 | + | chr22 | 14791998 | + |
| chr5 | 27739562 | − | chr15 | 13047361 | + | chr22 | 15241363 | + |
| chr5 | 28571442 | + | chr15 | 13302833 | + | chr22 | 15350051 | + |
| chr5 | 29201108 | + | chr15 | 14213492 | + | chr22 | 16626990 | + |
| chr5 | 31180246 | + | chr15 | 14507779 | + | chr22 | 16915783 | − |
| chr5 | 32016402 | + | chr15 | 16228906 | + | chr22 | 17045529 | + |
| chr5 | 32329750 | − | chr15 | 16323326 | + | chr22 | 17141709 | + |
| chr5 | 32894277 | + | chr15 | 16578711 | − | chr22 | 17690807 | + |
| chr5 | 33423631 | + | chr15 | 17299059 | + | chr22 | 17890769 | + |
| chr5 | 33870710 | + | chr15 | 18636559 | + | chr22 | 18675145 | + |
| chr5 | 33928032 | − | chr15 | 19177786 | + | chr22 | 19026766 | + |
| chr5 | 34415277 | + | chr15 | 19522680 | + | chr22 | 19045571 | + |
| chr5 | 35729177 | + | chr15 | 20738451 | + | chr22 | 20363297 | + |
| chr5 | 36420014 | + | chr15 | 21624045 | + | chr22 | 21058681 | + |
| chr5 | 36420101 | + | chr15 | 23526808 | + | chr22 | 21266818 | + |
| chr5 | 37669937 | + | chr15 | 23771997 | + | chr22 | 21471145 | + |
| chr5 | 38083896 | − | chr15 | 25424470 | + | chr22 | 21792514 | + |
| chr5 | 38582448 | + | chr15 | 26523373 | + | chr22 | 23198457 | + |
| chr5 | 39811067 | + | chr15 | 26718218 | + | chr22 | 23261221 | + |
| chr5 | 40372906 | + | chr15 | 26789705 | + | chr22 | 28916625 | − |
| chr5 | 40689392 | + | chr15 | 26932240 | + | chr22 | 28958472 | + |
| chr5 | 41697989 | + | chr15 | 27464070 | − | chr22 | 36791860 | + |
| chr5 | 41886038 | + | chr15 | 27655687 | + | chr22 | 38505883 | + |
| chr5 | 42835139 | + | chr15 | 27965173 | + | chr22 | 42615822 | + |
| chr5 | 45445466 | + | chr15 | 28724012 | + | chr22 | 43680961 | − |
| chr5 | 46043241 | + | chr15 | 28928268 | + | chr22 | 43830665 | + |
| chr5 | 46257029 | + | chr15 | 29436141 | + | chr22 | 48459090 | + |
| chr5 | 46625334 | + | chr15 | 30607773 | + | chr22 | 50440462 | + |
| chr5 | 47476951 | − | chr15 | 34183179 | + | chr22 | 52382373 | + |
| chr5 | 48940250 | + | chr15 | 34806032 | + | chr22 | 55665353 | + |
| chr5 | 49075283 | + | chr15 | 37045704 | + | chr22 | 62479693 | − |
| chr5 | 49628692 | − | chr15 | 37130927 | + | chr22 | 62904702 | + |
| chr5 | 51453503 | + | chr15 | 37881695 | + | chr22 | 64094230 | + |
| chr6 | 192119 | − | chr15 | 39771234 | + | chr22 | 65294542 | + |
| chr6 | 675005 | + | chr15 | 42572751 | + | chr22 | 70611943 | + |
| chr6 | 1152856 | + | chr15 | 42782528 | − | chr22 | 71933948 | + |
| chr6 | 2027094 | + | chr15 | 42847880 | + | chr22 | 72794074 | + |
| chr6 | 2205469 | + | chr15 | 44157676 | + | chr22 | 73450003 | + |
| chr6 | 2515364 | + | chr15 | 44165255 | + | chr22 | 74856015 | + |
| chr6 | 2589550 | + | chr15 | 44632628 | + | chr22 | 75312158 | + |
| chr6 | 2653068 | + | chr15 | 46541512 | − | chr22 | 75536897 | + |
| chr6 | 4579261 | + | chr15 | 46690402 | + | chr22 | 76040833 | + |
| chr6 | 5071280 | + | chr15 | 46795660 | + | chr23 | 21171 | − |
| chr6 | 5389181 | + | chr15 | 47470154 | + | chr23 | 1131200 | + |
| chr6 | 7316260 | + | chr15 | 47470231 | + | chr23 | 1655381 | + |
| chr6 | 7459996 | + | chr15 | 48772764 | + | chr23 | 3172623 | + |
| chr6 | 7586070 | + | chr15 | 50972049 | + | chr23 | 4599962 | + |
| chr6 | 7797807 | − | chr15 | 51585624 | + | chr23 | 8347406 | + |
| chr6 | 11276417 | + | chr15 | 52367865 | + | chr23 | 9617351 | + |
| chr6 | 11701880 | + | chr15 | 53560520 | + | chr23 | 11173849 | + |
| chr6 | 12832664 | + | chr15 | 55282516 | + | chr23 | 11347789 | + |
| chr6 | 13094248 | + | chr16 | 41981 | + | chr23 | 11627670 | + |
| chr6 | 13325910 | + | chr16 | 176223 | + | chr23 | 12401904 | + |
| chr6 | 16303034 | + | chr16 | 389163 | + | chr23 | 13184611 | + |
| chr6 | 16596586 | + | chr16 | 516643 | + | chr23 | 13763512 | + |
| chr6 | 20013104 | + | chr16 | 1207561 | + | chr23 | 13941998 | + |
| chr6 | 21508989 | + | chr16 | 1551825 | + | chr23 | 14716822 | + |
| chr6 | 21816580 | + | chr16 | 1609368 | + | chr23 | 15594584 | + |
| chr6 | 23945534 | + | chr16 | 1629781 | + | chr23 | 17357120 | + |
| chr6 | 25204318 | + | chr16 | 3006430 | + | chr23 | 19399901 | + |
| chr6 | 26405956 | + | chr16 | 3093954 | + | chr23 | 19862686 | + |
| chr6 | 29601201 | + | chr16 | 3246744 | + | chr23 | 20879495 | + |
| chr6 | 30029158 | + | chr16 | 4004580 | + | chr23 | 22599588 | + |
| chr6 | 30236168 | + | chr16 | 4076127 | + | chr23 | 22676337 | + |
| chr6 | 32403420 | + | chr16 | 4107833 | + | chr23 | 23007733 | + |
| chr6 | 32543646 | + | chr16 | 4917213 | + | chr23 | 25274048 | + |
| chr6 | 33145868 | − | chr16 | 5532226 | + | chr23 | 25380707 | + |
| chr6 | 34563778 | + | chr16 | 5644421 | + | chr23 | 27491047 | + |
| chr6 | 35381004 | + | chr16 | 6357693 | + | chr23 | 28597488 | + |
| chr6 | 35671336 | + | chr16 | 7098641 | + | chr23 | 29051686 | + |
| chr6 | 35674786 | + | chr16 | 7798828 | − | chr23 | 29146923 | + |
| chr6 | 35888592 | + | chr16 | 7842821 | + | chr23 | 29350218 | + |
| chr6 | 38174359 | + | chr16 | 9220272 | + | chr23 | 29565664 | + |
| chr6 | 38455793 | − | chr16 | 9729867 | + | chr23 | 29576346 | + |
| chr6 | 39217224 | + | chr16 | 10999665 | + | chr23 | 29987613 | + |
| chr6 | 39434335 | + | chr16 | 11290597 | + | chr23 | 34429512 | + |
| chr6 | 42632616 | + | chr16 | 13903863 | + | chr23 | 38434750 | + |
| chr6 | 42632777 | + | chr16 | 15365342 | + | chr23 | 38579661 | + |
| chr6 | 43852188 | + | chr16 | 16150066 | + | chr23 | 38599965 | + |
| chr6 | 43910751 | + | chr16 | 20553790 | + | chr23 | 38826605 | + |
| chr6 | 45005348 | + | chr16 | 20744729 | + | chr23 | 39803999 | + |
| chr6 | 45203191 | + | chr16 | 21520782 | + | chr23 | 41722874 | + |
| chr6 | 45387151 | + | chr16 | 21748172 | + | chr23 | 42884954 | + |
| chr6 | 45576970 | + | chr16 | 22476512 | − | chr23 | 44399126 | + |
| chr6 | 46156976 | + | chr16 | 22702424 | − | chr23 | 45597013 | + |
| chr6 | 46407025 | + | chr16 | 23189603 | − | chr23 | 46171754 | + |
| chr6 | 47139597 | + | chr16 | 23231786 | + | chr23 | 48342196 | + |
| chr6 | 48177345 | + | chr16 | 24026411 | + | chr23 | 48948517 | + |
| chr6 | 50806458 | + | chr16 | 25150743 | + | chr23 | 49233937 | + |
| chr6 | 51637360 | − | chr16 | 25295174 | + | chr23 | 49292496 | + |
| chr6 | 51693828 | + | chr16 | 25581327 | + | chr23 | 49366147 | + |
| chr7 | 3011002 | + | chr16 | 25652061 | + | chr23 | 49636145 | + |
| chr7 | 3302601 | + | chr16 | 25879266 | + | chr23 | 49669466 | + |
| chr7 | 3519006 | + | chr16 | 26017278 | + | chr23 | 49813423 | + |
| chr7 | 3714423 | + | chr16 | 26186665 | + | chr23 | 51679905 | + |
| chr7 | 4721065 | + | chr16 | 27449446 | − | chr23 | 53174164 | + |
| chr7 | 5197295 | + | chr16 | 29994824 | + | chr23 | 53367147 | + |
| chr7 | 7539575 | + | chr16 | 30090059 | + | chr23 | 53474560 | − |
| chr7 | 8762308 | + | chr16 | 32572242 | + | chr23 | 57109665 | + |
| chr7 | 10998337 | + | chr16 | 32639103 | + | chr23 | 58883979 | + |
| chr7 | 14225531 | + | chr16 | 33163523 | + | chr23 | 58884193 | + |
| chr7 | 16387551 | − | chr16 | 37922572 | − | chr23 | 60901416 | + |
| chr7 | 16509488 | + | chr16 | 37922669 | + | chr23 | 60949088 | + |
| chr7 | 17074083 | + | chr16 | 40327111 | − | chr23 | 61912587 | + |
| chr7 | 17085187 | − | chr16 | 42983281 | + | chr23 | 62069425 | + |
| chr7 | 17110105 | + | chr16 | 43175829 | + | chr23 | 64100534 | + |
| chr7 | 17497699 | + | chr16 | 43422584 | + | chr23 | 67269365 | + |
| chr7 | 18128891 | + | chr16 | 44681412 | + | chr23 | 67928489 | + |
| chr7 | 18327428 | + | chr17 | 569609 | + | chr23 | 69175437 | + |
| chr7 | 19211181 | + | chr17 | 1121017 | + | chr23 | 69347831 | + |
| chr7 | 20532976 | + | chr17 | 1236202 | + | chr23 | 70279117 | − |
| chr7 | 20561329 | + | chr17 | 1415089 | − | chr23 | 71617715 | + |
| chr7 | 20800005 | + | chr17 | 2174716 | + | chr24 | 119883 | + |
| chr7 | 21981347 | + | chr17 | 6171504 | + | chr24 | 278593 | + |
| chr7 | 24557679 | + | chr17 | 7448014 | + | chr24 | 402493 | + |
| chr7 | 28266232 | + | chr17 | 8057823 | + | chr24 | 475666 | − |
| chr7 | 28550384 | + | chr17 | 9862747 | + | chr24 | 579327 | + |
| chr7 | 29738083 | + | chr17 | 11197727 | + | chr24 | 581500 | + |
| chr7 | 29836453 | + | chr17 | 11887158 | + | chr24 | 922035 | + |
| chr7 | 29960366 | + | chr17 | 12519081 | + | chr24 | 2214054 | + |
| chr7 | 30051854 | + | chr17 | 12882931 | + | chr24 | 3204528 | + |
| chr7 | 30591627 | + | chr17 | 13719827 | + | chr24 | 5537908 | + |
| chr7 | 31521939 | + | chr17 | 14601269 | + | chr24 | 5545996 | + |
| chr7 | 33621746 | + | chr17 | 15162953 | + | chr24 | 6061648 | + |
| chr7 | 35704072 | + | chr17 | 15254052 | + | chr24 | 6071611 | + |
| chr7 | 35790621 | + | chr17 | 18648905 | + | chr24 | 6357799 | + |
| chr7 | 36230771 | + | chr17 | 18717160 | + | chr24 | 6379891 | − |
| chr7 | 36318357 | + | chr17 | 19325887 | + | chr24 | 8224394 | + |
| chr7 | 37040441 | + | chr17 | 23133603 | + | chr24 | 8370384 | + |
| chr7 | 37054395 | + | chr17 | 23236563 | + | chr24 | 10354359 | + |
| chr7 | 37552738 | + | chr17 | 24404412 | + | chr24 | 10676406 | + |
| chr7 | 37665328 | + | chr17 | 25289432 | + | chr24 | 11729426 | + |
| chr7 | 38319635 | + | chr17 | 26269538 | + | chr24 | 12228553 | + |
| chr7 | 38774632 | + | chr17 | 28699388 | − | chr24 | 12230053 | + |
| chr7 | 38799211 | + | chr17 | 30933599 | + | chr24 | 14193552 | − |
| chr7 | 39461210 | + | chr17 | 31889281 | + | chr24 | 14624910 | − |
| chr7 | 40117390 | + | chr17 | 32861480 | + | chr24 | 15316097 | + |
| chr7 | 40221339 | + | chr17 | 33157311 | + | chr24 | 15906534 | + |
| chr7 | 40406805 | + | chr17 | 33847127 | + | chr24 | 16043415 | − |
| chr7 | 41062359 | + | chr17 | 34213700 | + | chr24 | 16434464 | + |
| chr7 | 41412953 | − | chr17 | 34815926 | + | chr24 | 17457293 | + |
| chr7 | 42274068 | + | chr17 | 35666354 | + | chr24 | 17835882 | + |
| chr7 | 42274231 | + | chr17 | 36949439 | + | chr24 | 17989616 | + |
| chr7 | 42509521 | + | chr17 | 37438153 | + | chr24 | 18219185 | + |
| chr7 | 43109699 | + | chr17 | 38661061 | + | chr24 | 20062135 | + |
| chr7 | 44180657 | + | chr17 | 38829769 | + | chr24 | 21679164 | − |
| chr7 | 46802670 | + | chr17 | 39195559 | + | chr24 | 24984647 | + |
| chr10 | 14747 | + | chr18 | 159796 | + | chr24 | 26310574 | + |
| chr10 | 288180 | + | chr18 | 2252467 | + | chr24 | 26369044 | + |
| chr10 | 331027 | − | chr18 | 3357064 | + | chr24 | 29451113 | + |
| chr10 | 386158 | + | chr18 | 5123215 | − | chr24 | 30143396 | + |
| chr10 | 543609 | + | chr18 | 5123465 | + | chr24 | 30246045 | + |
| chr10 | 672095 | + | chr18 | 5517947 | + | chr24 | 30441603 | + |
| chr10 | 1885878 | − | chr18 | 6148515 | + | chr24 | 30700911 | + |
| chr10 | 5426054 | + | chr18 | 7118205 | − | chr24 | 33552555 | + |
| chr10 | 6060675 | + | chr18 | 7441876 | + | chr24 | 36573824 | + |
| chr10 | 7377979 | + | chr18 | 7697627 | + | chr24 | 36613466 | + |
| chr10 | 7745347 | + | chr18 | 8543333 | + | chr24 | 36649061 | + |
| chr10 | 9439299 | + | chr18 | 10193704 | + | chr24 | 36786461 | + |
| chr10 | 12289109 | − | chr18 | 10318966 | + | chr24 | 37643459 | − |
| chr10 | 12289574 | + | chr18 | 10461943 | + | chr24 | 39869749 | + |
| chr10 | 12560759 | + | chr18 | 11723895 | + | chr24 | 41019217 | + |
| chr10 | 18577064 | + | chr18 | 13735740 | + | chr24 | 41310184 | + |
| chr10 | 18724485 | + | chr18 | 15378097 | + | chr24 | 41623033 | + |
| chr10 | 19154562 | + | chr18 | 16088847 | + | chr24 | 42369837 | + |
| chr10 | 19677574 | + | chr18 | 16983153 | + | chr24 | 42498949 | + |
| chr10 | 19727427 | + | chr18 | 17288225 | + | chr24 | 42709442 | − |
| chr10 | 22036040 | + | chr18 | 17461354 | + | chr24 | 43172857 | + |
| chr10 | 23896615 | − | chr18 | 18606564 | + | chr24 | 43256474 | + |
| chr10 | 24283728 | + | chr18 | 18796264 | + | chr24 | 43451179 | + |
| chr10 | 24652020 | + | chr18 | 19140895 | + | chr24 | 43852797 | + |
| chr10 | 25654675 | + | chr18 | 19204274 | + | chr24 | 44633353 | + |
| chr10 | 25800213 | + | chr18 | 19554155 | + | chr24 | 44802673 | + |
| chr10 | 25858817 | + | chr18 | 20406982 | + | chr24 | 45886223 | + |
| chr10 | 26778022 | + | chr18 | 20522478 | − | chr24 | 49090031 | + |
| chr10 | 26996725 | + | chr18 | 21145442 | + | chr24 | 49209873 | + |
| chr10 | 27432995 | + | chr18 | 23065589 | + | chr24 | 51362291 | + |
| chr10 | 28921053 | − | chr18 | 23072589 | + | chr24 | 53242745 | + |
| chr10 | 29289430 | + | chr18 | 23346656 | + | chr24 | 53859246 | + |
| chr10 | 32333400 | − | chr18 | 23471124 | + | chr24 | 54630278 | + |
| chr10 | 32358801 | + | chr18 | 23721435 | + | chr24 | 56293014 | + |
| chr10 | 33231435 | + | chr18 | 23772971 | + | chr24 | 56781407 | + |
| chr10 | 33231506 | + | chr18 | 24220625 | + | chr24 | 57094973 | + |
| chr10 | 33430897 | + | chr18 | 24670866 | + | chr24 | 57624420 | + |
| chr10 | 34043561 | + | chr18 | 25074869 | + | chr24 | 59820979 | + |
| chr10 | 34342918 | − | chr18 | 25224868 | + | chr24 | 60040845 | + |
| chr10 | 35836139 | + | chr18 | 25397727 | − | chr25 | 3057773 | + |
| chr10 | 37844065 | + | chr18 | 25680312 | + | chr25 | 3503983 | + |
| chr10 | 38730888 | + | chr18 | 25680379 | + | chr25 | 6220745 | + |
| chr10 | 39458712 | + | chr18 | 27753062 | + | chr25 | 6294237 | + |
| chr10 | 39611410 | + | chr18 | 27985358 | + | chr25 | 6796891 | − |
| chr10 | 39972581 | + | chr18 | 29066926 | + | chr25 | 6816366 | + |

TABLE 2
Age-associated CpG sites as exemplified herein. The genomic coordinates are from the
Zebrafish genome version danRer10. The weight is also referred to as coefficient.

| CpG site | | | Association with age | | | Closest Feature | | | | |
|---|---|---|---|---|---|---|---|---|---|---|
| chr | position | strand | Weight | Correlation | p-value | Gene | feature | start | end | strand |
| Intercept | NA | NA | 3.261736 | NA | NA | NA | NA | NA | NA | NA |
| chr12 | 21540399 | + | −0.06868 | −0.456308633 | 3.80E−06 | mrpl27 | exon | 21563072 | 21563114 | + |
| chr12 | 35432443 | + | 0.041155 | 0.425636828 | 1.90E−05 | chmp6b | exon | 35487001 | 35487160 | + |
| chr13 | 31180246 | + | 0.422877 | 0.49406794 | 4.18E−07 | mettl18 | exon | 31259863 | 31260037 | + |
| chr13 | 38582448 | + | 0.287827 | 0.518416373 | 8.70E−08 | zgc:153049 | exon | 38688631 | 38688754 | + |
| chr14 | 38455793 | − | −0.34896 | −0.404759937 | 5.20E−05 | csnk1a1 | exon | 38442661 | 38443287 | − |
| chr14 | 45387151 | + | −0.22242 | −0.432519711 | 1.34E−05 | sncb | exon | 45619305 | 45619341 | + |
| chr17 | 52836692 | + | 0.089225 | 0.44142766 | 8.45E−06 | meis2a | exon | 52833657 | 52835083 | + |
| chr18 | 38107080 | + | −0.40695 | −0.407236996 | 4.63E−05 | nucb2b | exon | 38210387 | 38210462 | + |
| chr18 | 50792250 | + | −0.3449 | −0.434582509 | 1.21E−05 | reln | CDS | 50795737 | 50795848 | + |
| chr19 | 20077224 | + | 0.013643 | 0.428542532 | 1.64E−05 | hibadha | CDS | 20079490 | 20079646 | + |
| chr1 | 23386154 | + | 0.267495 | 0.462650352 | 2.67E−06 | mab21l2 | CDS | 23385795 | 23386871 | + |
| chr1 | 43259461 | + | −0.28726 | −0.419849369 | 2.53E−05 | cabp2a | exon | 43425989 | 43426036 | + |
| chr20 | 16578711 | − | 0.007467 | 0.459510595 | 3.18E−06 | ches1 | CDS | 16578582 | 16579053 | − |
| chr20 | 21624045 | + | 0.310809 | 0.383202718 | 0.000138 | jag2b | exon | 21573904 | 21575945 | + |
| chr20 | 26523373 | + | 0.436491 | 0.468930078 | 1.87E−06 | zbtb2b | exon | 26504936 | 26508142 | + |
| chr20 | 28928268 | + | 0.050606 | 0.492313214 | 4.66E−07 | fntb | exon | 28924424 | 28924877 | + |
| chr21 | 23231786 | + | −0.09242 | −0.406502544 | 4.79E−05 | alg8 | exon | 22864361 | 22864801 | + |
| chr21 | 25150743 | + | −0.33385 | −0.541055377 | 1.80E−08 | sycn.2 | exon | 25189953 | 25190586 | + |
| chr24 | 19868851 | + | −0.22858 | −0.559326016 | 4.64E−09 | LOC100334155 | exon | 20073262 | 20073368 | + |
| chr24 | 4215673 | + | 0.06477 | 0.410284892 | 4.01E−05 | wdr37 | exon | 3494784 | 3495510 | + |
| chr25 | 14631230 | + | 0.217506 | 0.420374681 | 2.46E−05 | mpped2 | CDS | 14637373 | 14637488 | + |
| chr25 | 16313450 | + | 0.307822 | 0.482149108 | 8.63E−07 | tead1a | CDS | 16315617 | 16315681 | + |
| chr25 | 36872756 | + | −0.17805 | −0.360931599 | 0.000352 | chmp1a | exon | 36871083 | 36871567 | + |
| chr25 | 6461988 | + | 0.453596 | 0.408996487 | 4.26E−05 | snx33 | exon | 6351734 | 6353787 | + |
| chr2 | 8207957 | + | 0.258846 | 0.462933479 | 2.63E−06 | chst2a | exon | 8314444 | 8316603 | + |
| chr3 | 23616782 | + | −0.27465 | −0.451308167 | 4.99E−06 | hoxb3a | exon | 23616752 | 23617534 | + |
| chr4 | 17690807 | + | −0.26411 | −0.603855727 | 1.17E−10 | gnptab | exon | 17690788 | 17690922 | + |
| chr4 | 18675145 | + | −0.20748 | −0.461522112 | 2.84E−06 | slc26a4 | CDS | 18793563 | 18793599 | + |
| chr5 | 51679905 | + | 0.034253 | 0.386666431 | 0.000118 | slc14a2 | exon | 51529758 | 51531231 | + |

TABLE 3
Age-associated CpG sites as exemplified herein. The genomic coordinates are from the
Zebrafish genome version danRer10. The weight is also referred to as coefficient.

| CpG site | | | Association with age | | | Closest Feature | | | | |
|---|---|---|---|---|---|---|---|---|---|---|
| chr | position | strand | Weight | Correlation | p-value | Gene | feature | start | end | strand |
| Intercept | NA | NA | 3.261736 | NA | NA | NA | NA | NA | NA | NA |
| chr12 | 35432443 | + | 0.041155 | 0.425636828 | 1.90E−05 | chmp6b | exon | 35487001 | 35487160 | + |
| chr13 | 31180246 | + | 0.422877 | 0.49406794 | 4.18E−07 | mettl18 | exon | 31259863 | 31260037 | + |
| chr13 | 38582448 | + | 0.287827 | 0.518416373 | 8.70E−08 | zgc:153049 | exon | 38688631 | 38688754 | + |
| chr14 | 45387151 | + | −0.22242 | −0.432519711 | 1.34E−05 | sncb | exon | 45619305 | 45619341 | + |
| chr17 | 52836692 | + | 0.089225 | 0.44142766 | 8.45E−06 | meis2a | exon | 52833657 | 52835083 | + |
| chr18 | 38107080 | + | −0.40695 | −0.407236996 | 4.63E−05 | nucb2b | exon | 38210387 | 38210462 | + |
| chr18 | 50792250 | + | −0.3449 | −0.434582509 | 1.21E−05 | Ireln | CDS | 50795737 | 50795848 | + |
| chr19 | 20077224 | + | 0.013643 | 0.428542532 | 1.64E−05 | hibadha | CDS | 20079490 | 20079646 | + |
| chr1 | 23386154 | + | 0.267495 | 0.462650352 | 2.67E−06 | mab2112 | CDS | 23385795 | 23386871 | + |
| chr1 | 43259461 | + | −0.28726 | −0.419849369 | 2.53E−05 | cabp2a | exon | 43425989 | 43426036 | + |
| chr20 | 16578711 | − | 0.007467 | 0.459510595 | 3.18E−06 | ches1 | CDS | 16578582 | 16579053 | − |
| chr20 | 21624045 | + | 0.310809 | 0.383202718 | 0.000138 | jag2b | exon | 21573904 | 21575945 | + |
| chr20 | 26523373 | + | 0.436491 | 0.468930078 | 1.87E−06 | zbtb2b | exon | 26504936 | 26508142 | + |
| chr20 | 28928268 | + | 0.050606 | 0.492313214 | 4.66E−07 | fntb | exon | 28924424 | 28924877 | + |
| chr21 | 23231786 | + | −0.09242 | −0.406502544 | 4.79E−05 | alg8 | exon | 22864361 | 22864801 | + |
| chr21 | 25150743 | + | −0.33385 | −0.541055377 | 1.80E−08 | sycn.2 | exon | 25189953 | 25190586 | + |
| chr24 | 19868851 | + | −0.22858 | −0.559326016 | 4.64E−09 | LOC100334155 | exon | 20073262 | 20073368 | + |
| chr25 | 14631230 | + | 0.217506 | 0.420374681 | 2.46E−05 | mpped2 | CDS | 14637373 | 14637488 | + |
| chr25 | 16313450 | + | 0.307822 | 0.482149108 | 8.63E−07 | tead1a | CDS | 16315617 | 16315681 | + |
| chr25 | 36872756 | + | −0.17805 | −0.360931599 | 0.000352 | chmp1a | exon | 36871083 | 36871567 | + |
| chr25 | 6461988 | + | 0.453596 | 0.408996487 | 4.26E−05 | snx33 | exon | 6351734 | 6353787 | + |
| chr2 | 8207957 | + | 0.258846 | 0.462933479 | 2.63E−06 | chst2a | exon | 8314444 | 8316603 | + |
| chr3 | 23616782 | + | −0.27465 | −0.451308167 | 4.99E−06 | hoxb3a | exon | 23616752 | 23617534 | + |
| chr4 | 17690807 | + | −0.26411 | −0.603855727 | 1.17E−10 | gnptab | exon | 17690788 | 17690922 | + |
| chr4 | 18675145 | + | −0.20748 | −0.461522112 | 2.84E−06 | slc26a4 | CDS | 18793563 | 18793599 | + |
| chr5 | 51679905 | + | 0.034253 | 0.386666431 | 0.000118 | slc14a2 | exon | 51529758 | 51531231 | + |

While the age-associated CpG sites disclosed herein (for example, in Tables 1, 2, 3, 7, 8, 9, 12, 16, 19 or 20) are described by reference to a reference genome or database, the person skilled in the art would be able to determine the corresponding age-associated sites in an updated reference genome or database or related genome or database using known techniques. In this situation, a related genome or database can include RNA sequence databases (which, in some embodiments, can be used as a substitute for genomic data), genomes or databases for the same species prepared using different sequencing techniques or by different research groups or proprietary genomes or databases. Databases include, but are not limited to, NCBI Genomes (available at www.ncbi.nlm.nih.gov/genome/), Short Read Archive (SRA) (available at www.ncbi.nlm.nih.gov/sra), Ensembl Genomes (available at asia.ensembl.org/index.html) and the like.

### Methylation Status

As used herein, the terms “methylation status”, “methylation level” or “the degree of methylation” are used interchangeably and refer to the presence or absence of a methylated cytosine (for example, 5-methylcytosine) at one or more CpG sites within a DNA sequence. For example, a CpG site containing a methylated cytosine is considered methylated (for example, the methylation status of the CpG site is methylated). A CpG site that does not contain a methylated cytosine is considered unmethylated.

As will be appreciated by a person skilled in the art not all copies of a CpG site in a sample will be methylated or unmethylated. In some embodiments, the methylation status can be represented or indicated by a “methylation value” (e.g., a methylation frequency, fraction, ratio, percent, etc.). A methylation value can be generated, for example, by comparing amplification profiles after bisulfite reaction or by comparing sequences of bisulfite-treated and untreated nucleic acids. Accordingly, a methylation value, represents the methylation status and can be used as a quantitative indicator of the level of methylation at an age-associated CpG site. This is of particular use when it is desirable to compare the methylation status of a one or more CpG sites in a sample to a reference value (e.g. the methylation status of one or more CpG sites in an age-correlated reference population).

In some embodiments, the methylation status of an age-associated CpG site can be represented as the fraction of ‘C’ bases out of ‘C’+‘U’ total bases at the age-associated CpG site “i” following the bisulfite treatment. In some embodiments, the methylation status of an age-associated CpG site can be represented as the fraction of ‘C’ bases out of ‘C’+‘T’ total bases at the age-associated CpG site “i” following the bisulfite treatment and subsequent PCR.

In some embodiments, analysing DNA comprises determining the methylation beta value of one or more age associated CpG sites. As used herein, the “methylation beta value” is the fraction of methylated cytosine at a CpG site. The methylation beta value is often calculated using the equation:

Beta=M/(M+U+a)

where M and U refer to the amount of methylated and unmethylated cytosine respectively (measured, for example, by signal intensities) and ‘a’ is an optional offset (often set to 100) which is added to help stabilise beta values when both M and U are small. The methylation beta-value is typically expressed as a number between 0 and 1, (or 0 and 100%). In theory, a methylation beta-value of zero indicates that all copies of the CpG site are unmethylated (no methylated molecules were measured) and a methylation beta-value of one indicates that all copies of the CpG site were methylated.

In some embodiments, analysing DNA comprises determining the methylation M-value of the age associated CpG sites. As used herein, the “M-value” is calculated as the log 2 ratio of the intensities of methylated probe versus unmethylated probe. In theory, a M-value of zero indicates that the CpG site is approximately half-methylated, assuming, for example, that the intensity data has been properly normalized by Illumina GenomeStudio or some other external normalization algorithm. Positive M-values indicate that that more CpG sites are methylated than unmethylated, while negative M-values mean that less CpG sites are methylated than unmethylated.

### Determining Methylation Status

In the methods described herein, the presence of methylated cytosine at an age-associated CpG site can be measured using techniques suitable for the analysis of such sites. Suitable techniques are known to the person skilled in the art and allow for the determination of the methylation status of one or more CpG sites within a sample. In addition, these techniques may be used for absolute or relative quantification of methylated cytosine at CpG sites. Non limiting examples of techniques suitable for the identification of methylated cytosine at CpG sites include molecular break light assay for DNA adenine methyltransferase activity, methylation-specific polymerase chain reaction (PCR), whole genome bisulfite sequencing, the HpaII tiny fragment enrichment by ligation-mediated PCR (HELP) assay, methyl sensitive southern blotting, ChIP-on-chip assay, restriction landmark genomic scanning, methylated DNA immunoprecipitation (MeDIP), sequencing of bisulfite treated DNA (e.g. reduced representation bisulfite sequencing (RRBS) and whole genome bisulfite sequencing (WGBS)). Suitable methods are also described in WO2015/048665.

In some embodiments, suitable methods comprise two steps. The first step is a methylation specific reaction or separation, such as (i) bisulfite treatment, (ii) methylation specific binding, or (iii) methylation specific restriction enzymes. In some embodiments, the methylation specific reaction is bisulfite treatment. The second step involves (i) amplification and detection, or (ii) direct detection, by a variety of methods such as (a) PCR (sequence-specific amplification), (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis. In some embodiments, the second step comprises PCR and DNA sequencing. In some embodiments, analysis of DNA obtained from fish or a reptile can be performed in accordance with the Examples described herein.

One technique suitable for use in the method of estimating age described herein comprises treatment of DNA from the biological sample with bisulfite reagent to convert unmethylated cytosines of CpG sites to uracil. In these embodiments, discrimination of methylated cytosines from non-methylated cytosines is possible because uracil base pairs with adenine (thus behaving like thymine), whereas 5-methylcytosine base pairs with guanine (thus behaving like cytosine). After PCR and DNA sequencing, the conversion of unmethylated cytosine to uracil is observed as a C to T sequence change. The term “bisulfite reagent” refers to a reagent comprising bisulfite, disulfite, hydrogen sulfite, or combinations thereof. Methods of said treatment are described in the art (e.g., WO 2005/038051 and WO 2013/116375). In some embodiments, the bisulfite reaction comprises treatment with sodium bisulfite.

In some embodiments, bisulfite treatment is conducted in the presence of denaturing solvents such as but not limited to n-alkylenglycol or diethylene glycol dimethyl ether (DME), or in the presence of dioxane or dioxane derivatives. In some embodiments the denaturing solvents are used in concentrations between 1% and 35% (v/v). In some embodiments, heat denaturation is used. In some embodiments, the sample is heated to a temperature sufficient to denature the DNA. For example, in some embodiments the sample being treated with bisulfite reagent is incubated in the presence of bisulfite reagent at 98° C. and then incubated at 64° C. In some embodiments, the sample is incubated in the presence of bisulfite reagent to 98° C. for 10 minutes, the temperature is reduced to 64° C. and the sample incubated at 64° C. for a further 2.5 h. In some embodiments, the bisulfite reaction is carried out in the presence of scavengers such as but not limited to chromane derivatives, e.g., 6-hydroxy-2,5,7,8-tetramethylchromane 2-carboxylic acid or trihydroxybenzone acid and derivatives thereof, e.g. Gallic acid (see: WO 2005/038051). In some embodiments, the DNA is bisulfite converted using the EZ DNA Methylation Gold Kit (Zymo Research, California, USA), for example, in accordance with the manufacture's protocol. In some embodiments, the DNA is treated with sodium metabisulfite in accordance with the protocol described in Clark et al. (2006). In some embodiments, the bisulfite-treated DNA is purified prior to the quantification. Purification may be conducted by any means known in the art, such as but not limited to ultrafiltration, e.g., by means of Microcon™ columns (Millipore™).

In some embodiments, the level of methylated cytosine at an age-associated CpG site is determined using a polymerase chain reaction (PCR). In some embodiments, the PCR is performed in multiplex. In some embodiments, the nucleic acids are amplified by PCR amplification using methodologies known to a person skilled in the art. In some embodiments, fragments of the treated DNA comprising the CpG site of interest are amplified using sets of primer oligonucleotides (e.g., as listed in Table 4) and an amplification enzyme. The amplification of several DNA segments can be carried out simultaneously in one reaction vessel. Typically, the amplification is carried out using a polymerase chain reaction (PCR). PCR produces an amplified target which can then be analysed for the presence or absence of methylated cytosine using DNA sequencing (e.g., massively parallel or Next Generation sequencing).

In a preferred embodiment, genomic DNA is reacted with sodium bisulfite to convert unmethylated cytosine to uracil while leaving 5-methylcytosine unchanged. The targeted age-associated CpG sites are amplified by PCR (e.g. multiplex PCR), and the resulting product is optionally isolated and used as a template for DNA sequencing. In some embodiments, the amplicons are barcoded prior to DNA sequencing, for example using MiSeq adaptors and barcodes from Fluidigm (San Francisco, USA). In this embodiment, the method detects bisulfite introduced methylation dependent C to T sequence changes. An example of multiplex bisulfite PCR resequencing is described in Korbie et al. (2015). While other techniques can be used for the analysis of methylated cytosine at age-associated CpG sites in the methods described herein, the use of PCR (e.g. multiplex PCR) followed by DNA sequencing advantageously reduces the burden of resources, computational time and/or cost involved in performing the method (c.f. using RRBS as a method to estimate age). Using PCR followed by DNA sequencing also provides a more practical and/or cost-effective method. The present inventors have also found that the use of multiplex PCR followed by DNA sequencing provides improved sensitivity relative to other techniques, such as methylation sensitive PCR.

### Primers

As will be appreciated, PCR (including multiplex PCR) uses primer pairs configured to amplify a region of the DNA comprising the age-associated CpG site. In some embodiments, the multiplex PCR uses two or more primer pairs configured to amplify a region of the DNA comprising the age-associated CpG sites. In some embodiments, the region of the DNA comprising the age-associated CpG site amplified (i.e. the amplicon) is at least 50 bp, at least 80 bp, at least 100 bp, at least 110 bp, at least 120 bp, at least 130 bp, at least 140 bp or at least 150 bp. In some embodiments, the amplicon is less than 500 bp, less than 400 bp, less than 300 bp, less than 260 bp, less the 240 bp, less than 220 bp, less than 200 bp, less than 190 bp, less than 180 bp, less than 170 bp, less than 160 bp, or less than 150 bp. In some embodiments, the amplicon is between 100 bp and 160 bp. In some embodiments, the amplicon is between 130 bp and 150 bp.

In some embodiments, at least one of the primers hybridizes to a region of the DNA within 200, 180, 160, 140, 120, 100, 90, 80, 70, 60, 50, 40, 30 or 20 base-pairs of the age associated CpG site. In some embodiments, at least one of the primers hybridizes to a region of the DNA within 100 or 50 or 20 base-pairs of the age associated CpG site. In at least some embodiments, at least one of the primers is selected from the forward and reverse primers listed in Table 4; and/or can be used to amplify the same CpG site as at least one of the primers is selected from the forward and reverse primers listed in Table 4. Primers that can be used to amplify the same CpG site as the primers listed in Table 4 refers to primers which are not identical in sequence to the primers listed in Table 4 but, when used in PCR (e.g. multiplex PCR), will amplify a region of DNA that includes the same CpG site as listed in Table 4. In some embodiments, at least one of the primers hybridizes to a region of the DNA within 100 or 50 or 20 base-pairs of a primer listed in Table 4 such that the primer is able to be used instead of at least one of the primers listed in Table 4. In some embodiments, one or more or all of the primers pairs provided in Table 4 are used.

The present application also provides use of two or more primer pairs as described herein for amplifying age-associated CpG sites. In some embodiments, the age-associated CpG sites are listed in Tables 1, 2, 3, 7, 8, 9, 12, 16, 19 or 20 or a homolog of one or more thereof. In some embodiments, the age-associated CpG sites are listed in Tables 1, 2 or 3 or a homolog of one or more thereof. In some embodiments, the age-associated CpG sites are listed in Table 3 or a homolog of one or more thereof. In some embodiments, the age-associated CpG sites are listed in Table 7 or a homolog of one or more thereof. In some embodiments, the age-associated CpG sites are listed in Table 8 or Table 9 or a homolog of one or more thereof. In some embodiments, the age-associated CpG sites are listed in Table 8 or a homolog of one or more thereof. In some embodiments, the age-associated CpG sites are listed in Table 12 or a homolog of one or more thereof. In some embodiments, the age-associated CpG sites are listed in Table 16 or a homolog of one or more thereof. In some embodiments, the age-associated CpG sites are listed in Table 19 or a homolog of one or more thereof. In some embodiments, the age-associated CpG sites are listed in Table 20 or a homolog of one or more thereof. In some embodiments, the use comprises two or more primer pairs listed Table 4, and/or primers which can be used to amplify the same CpG site as the primers in Table 4. In some embodiments, the use comprises 2, 3, 4, 5, 6, 7, 8, 9, 10, 11, 12, 13, 14, 15, 16, 17, 18, 19, 20, 21, 22, 23, 24, 25 or 26 of the primer pairs listed Table 4 and/or primers which can be used to amplify the same CpG site as the primers in Table 4. In some embodiments, the use comprises all of the primer pairs listed in Table 4 and/or primers which can be used to amplify the same CpG site as the primers in Table 4.

### Estimating the Age of the Fish or Reptile

The methods of estimating age described herein comprise estimating the age of the fish or reptile based on levels of methylated cytosine at the age-associated CpG sites. As used herein, the term “estimating the age” (and variations thereof) refers to roughly calculating or judging the age (e.g. chronological age) of a subject, for example a fish or reptile.

In some embodiments, the estimation step comprises comparing the levels of methylated cytosine at the age-associated CpG sites to an age correlated reference population. For example, the methods may comprise comparing the level of methylated cytosine at age-associated CpG sites of the fish or reptile being tested with the level of methylated cytosine of the same age-associated CpG sites of an age correlated reference population.

TABLE 4
Example primers for amplifying age-associated CpG sites. The weight is also referred to as coefficient.

| chr | position | strand | Weight | Forward | Reverse | Pool |
|---|---|---|---|---|---|---|
| Intercept | NA | NA | 1.356556 | | | |
| chr12 | 35432443 | + | 0.172804 | gacatggttctacaTGAGTGTTTGTTTGGTtAAGtAT | cagagacttggtctCAaaACAaTTCCTCCACCC [SEQ ID | 2 |
| | | | | [SEQ ID NO: 1] | NO: 2] | |
| chr13 | 31180246 | + | 0.037482 | gacatggttctacaAaCCCcNaAaAACCACTAC [SEQ ID | cagagacttggtctAAtAAGAtAGtTGAAATttTtAAGGT | 1 |
| | | | | NO: 3] | GTtTA [SEQ ID NO: 4] | |
| chr13 | 38582448 | + | 0.12808 | cagagacttggtctTTACATCTaAATAaaTaTTTCCCTTTa | gacatggttctacattttTGtATTGTGAGGAGTTtATAA | 1 |
| | | | | TaAT [SEQ ID NO: 5] | [SEQ ID NO: 6] | |
| chr14 | 45387151 | + | 0.02251 | gacatggttctacaGATTGAGGtAGTTtTGAAGAtAA | cagagacttggtctTCCTTAAAaCATAACCATTaTTTCT | 1 |
| | | | | [SEQ ID NO: 7] | [SEQ ID NO: 8] | |
| chr17 | 52836692 | + | −0.29603 | gacatggttctacaTcNaATCACAAATCTCCAATC [SEQ | cagagacttggtcttAGtAGATGtNgtTTTAGATtAG | 2 |
| | | | | ID NO: 9] | [SEQ ID NO: 10] | |
| chr18 | 38107080 | + | −0.06134 | gacatggttctacaNgGTtTGTGTATGTGAAAGTG [SEQ | cagagacttggtctCCACCTCAAATCATTCTCC [SEQ ID | 2 |
| | | | | ID NO: 11] | NO: 12] | |
| chr18 | 50792250 | + | 0.82028 | cagagacttggtctTaAACATCTCCTaaATCTCTaCA | gacatggttctacaGtAtTGAATATtAAAGtTGAATGTG | 2 |
| | | | | [SEQ ID NO: 13] | [SEQ ID NO: 14] | |
| chr19 | 20077224 | + | 2.50964 | gacatggttctacaAacNTACTTTACTaTCTCACC [SEQ | cagagacttggtctGtTGtNgGtTtAAAtTTTAAtAGG | 1 |
| | | | | ID NO: 15] | [SEQ ID NO: 16] | |
| chr1 | 23386154 | + | 0.199901 | gacatggttctacatATGTtttTGTGGGTGGAGTT [SEQ | cagagacttggtctCcNCCACCATCTTAACCA [SEQ ID | 1 |
| | | | | ID NO: 17] | NO: 18] | |
| chr1 | 43259461 | + | 0.61429 | gacatggttctacaATAGtTGTAttAGTGTTTGTGTG | cagagacttggtctCCCTTCCTaCCCCCTC [SEQ ID | 2 |
| | | | | [SEQ ID NO: 19] | NO: 20] | |
| chr20 | 16578711 | 1 | 0.108758 | gacatggttctacacNaCCAaaTaaAaCAaAaACCC [SEQ | cagagacttggtctAAGGAGAtANgtTGttttTGAAG | 1 |
| | | | | ID NO: 21] | [SEQ ID NO: 22] | |
| chr20 | 21624045 | + | 0.157608 | cagagacttggtctCTCTaACCCCTaCCTCCC [SEQ ID | gacatggttctacagtNgGttTAtAAttTGAtATGTTAA | 2 |
| | | | | NO: 23] | [SEQ ID NO: 24] | |
| chr20 | 26523373 | + | 0.936519 | gacatggttctacaAATTCCAaCTCAAATCTTCTTCT | cagagacttggtctAAAANgTGTAAATGAGAGAGAAA | 2 |
| | | | | [SEQ ID NO: 25] | [SEQ ID NO: 26] | |
| chr20 | 28928268 | + | 1.371212 | cagagacttggtctTATTGtTTtAAGTGTGtAAtTTGTG | gacatggttctacaTATcNTCAaCAATAATACTaCAATT | 1 |
| | | | | [SEQ ID NO: 27] | [SEQ ID NO: 28] | |
| chr21 | 23231786 | + | −3.60E−03 | gacatggttctacaTTTACCcNaTITTATAAATaCCC | cagagacttggtctGAtTAGATTGTtAGAtATTTAGTATG | 2 |
| | | | | [SEQ ID NO: 29] | [SEQ ID NO: 30] | |
| chr21 | 25150743 | + | −0.70924 | gacatggttctacatNgTtAGATTTGGAGttAttTATG | cagagacttggtctTAAACCCAAACCTCCTCCC [SEQ ID | 2 |
| | | | | [SEQ ID NO: 31] | NO: 32] | |
| chr24 | 19868851 | + | 0.187006 | cagagacttggtctGtTtTTttTAtATGtTATGAAATTTtAG | gacatggttctacaCCCCTAACATCTATaTCTACA [SEQ | 2 |
| | | | | AtATG [SEQ ID NO: 33] | ID NO: 34] | |
| chr25 | 14631230 | + | −0.18347 | gacatggttctacaTTATtAGAtAGTGGTAAATAAAGGT | cagagacttggtctCAaATTaATCAAaCTaTCAaCACC | 2 |
| | | | | [SEQ ID NO: 35] | [SEQ ID NO: 36] | |
| chr25 | 16313450 | + | 0.5212 | cagagacttggtctGTGTTTGGAAGAATAGAGAGG | gacatggttctacaCTaTaTAAATTCCCTTCATaTCAAT | 1 |
| | | | | [SEQ ID NO: 37] | [SEQ ID NO: 38] | |
| chr25 | 36872756 | + | −0.18515 | gacatggttctacagAGtAGAGtTGAGGATTAAtAG | cagagacttggtctCTCCTaCACTCATCAaATCAA [SEQ | 1 |
| | | | | [SEQ ID NO: 39] | ID NO: 40] | |
| chr25 | 6461988 | + | −0.74604 | cagagacttggtctAAAAGTtAAAGtAGAtAGGGAGT | gacatggttctacaCCTTTaCTCTTTaaCTTCCCA [SEQ | 1 |
| | | | | [SEQ ID NO: 41] | ID NO: 42] | |
| chr2 | 8207957 | + | 2.467118 | cagagacttggtctCAaaaCcNaTaACATTCTaCATC | gacatggttctacaANgtAGAtTTGtAAAGTGAATAAAA | 1 |
| | | | | [SEQ ID NO: 43] | [SEQ ID NO: 44] | |
| chr3 | 23616782 | + | 0.206688 | cagagacttggtctTTATaTTTTATTTCATTCCCACCC | gacatggttctacaAtAGGTATNgGTTGAAGTGAA [SEQ | 2 |
| | | | | [SEQ ID NO: 45] | ID NO: 46] | |
| chr4 | 17690807 | + | −0.96089 | cagagacttggtctGGtTAAAtATGTGTTTTTGTGTG | gacatggttctacaaTTTaACCcNaAaCTaCTCAaTT [SEQ | 2 |
| | | | | [SEQ ID NO: 47] | ID NO: 48] | |
| chr4 | 18675145 | + | 8.24E−03 | cagagacttggtctATTTCATCTaCAaTaACCACATAC | gacatggttctacaTTtAAAAtAGAGGTGTGTtTGAAAA | 1 |
| | | | | [SEQ ID NO: 49] | [SEQ ID NO: 50] | |
| chr5 | 51679905 | + | −0.10148 | cagagacttggtctttAAATGAAGttATGGtTGTGTG | gacatggttctacaaAaCAaTTCTaACACCTaTCTATAT | 2 |
| | | | | [SEQ ID NO: 51] | [SEQ ID NO: 52] | |

The term “age correlated reference population” refers to a population of fish or reptiles having a known date of conception or birth (i.e., a chronological age).

As used herein, “chronological age” is the actual age of the fish or reptile. For fish or reptiles, chronological age may be based on the age calculated from the moment of conception or based on the age calculated from the time and date of birth. An age correlated reference population comprises fish or reptiles of varying age (e.g., birth, 1 week, 2 weeks, 1 month, 1 year, 2 years etc. until natural death). The level of methylated cytosine at age-associated CpG sites from an age correlated reference population may be analysed using general methodology known to the person skilled in the art, for example, using reduced representation bisulfite sequencing or whole genome sequencing.

In some embodiments, estimating the age of the fish or reptile comprises comparing the methylation profile of the fish or reptile being tested to the methylation profile of an age correlated reference population determined using the same age-associated CpG sites. As used herein, the term “methylation profile” refers to data representing the methylation status of one or more CpG site within a subject's genomic DNA. The profile may indicate the methylation status of every age-associated CpG site in a subject or may indicate the methylation status of a subset the age-associated CpG sites, for example the CpG sites listed in Tables 1, 2, 3, 7, 8, 9, 12 or 15. In some embodiments, the methylation profile is the raw summed methylation beta values for the sample. Raw summed methylation beta values may be calculated by multiplying the coefficient calculated for the age-associated CpG site (for example, the coefficient value provided in Tables 2, 3, 8 or 9) by the corresponding methylation beta value and then adding up all the values with the intercept value (for example, the intercept value provide in Tables 2, 3, 8 or 9). In some embodiments, the methylation profile is compared to a standard methylation profile comprising a methylation profile from a known type of sample (e.g., age correlated reference population). In some embodiments, methylation profiles are generated using the methods described herein.

In some embodiments, the method comprises use of a statistical method to compare the level of methylated cytosine at age-associated CpG sites from the fish or reptile being tested with the level of methylated cytosine of the same age-associated CpG sites from an age correlated reference population. Any suitable statistical comparison methodology known to the person skilled in the art can be used to relate the methylation levels to age.

Examples of suitable statistical methods include but are not limited to multivariate regression method, linear regression analysis, tabular method or graphical method. In some embodiments, the statistical method comprises Elastic Net, Lasso regression method, ridge regression method, least-squares fit, binomial test, Shapiro-Wilk test, Grubb's statistics, Benjamini-Hochberg FDR, variance analysis, entropy statistics, and/or Shannon entropy. In some embodiments, the statistical method comprises use of a linear regression model or an elastic-net generalised linear model. In some embodiments, the estimating comprises use of a linear regression model or an elastic-net generalized linear model as implemented in the GLMNET package (Friedman et al., 2010). In some embodiments, the comparing step comprises use of an elastic-net generalized linear model. In a further embodiment, the comparing step comprises use of an elastic-net generalized linear model as implemented in the GLMNET package (Friedman et al., 2010).

In some embodiments, a linear regression model may be used to estimate age based on a weighted average of the level of methylated cytosine at age-associated CpG sites plus an optional offset. In some embodiments, the chronological age is regressed on the level of methylated cytosine at the CpG sites. In other embodiments, the chronological age is transformed before being regressed on the level of methylated cytosine at the CpG sites. Transformation may lead to an age predictor that is substantially more accurate (in relation to error) and/or that requires substantially fewer CpG sites than one without the transformation. In some embodiments, a transformed version of chronological age can be regressed on the CpG sites using a linear regression model. In some embodiments, the age is transformed using log or natural log before using the linear regression model.

In some embodiments, a reference data set is collected (e.g. of a age correlated reference population which includes a number of fish or reptiles of varying and known ages) using specific technology platform(s) and tissue(s) and an elastic-net generalized linear model is fit to the reference data set to estimate the coefficients (also referred to herein as “weights”) which can be used in the linear regression model. The resultant model can then be used for estimating the age of fish or reptiles. As would be appreciated by the person skilled in the art coefficient values in various models can also reflect the specific technique that is used to measure the methylation levels. For example, for beta values measured as exemplified herein there can be one set of coefficients, while for other methylation measures (e.g. using sequencing technology) there can be another set of coefficients etc

In some embodiments, the statistical method comprises (a) identifying a weight for each age associated CpG site (e.g. from Table 4); (b) multiplying each of the weights with its corresponding age associated CpG methylation level (e.g. beta value) to output a value for each age-associated CpG site; (c) finding the sum of values of (b); (d) transforming the summed values of (c) to the natural log of age in weeks; and (e) calculating the natural exponentiation of (d), wherein the exponentiation is the estimated age of the subject.

In some embodiments, the methods described herein can be used to estimate the age of a fish or reptile across the entire lifespan of the fish or reptile. The methods for estimating the age of a fish or reptile described herein can be used to estimate the age of a sub-population of fish or reptiles. In some embodiments, the methods can be used to estimate the age of younger fish or reptiles. In some embodiments, the methods can be used to estimate the age of a fish or reptile aged about 1 year or less, 2 years or less, 3 years or less, 4 years or less, 5 years or less, 6 years or less, 7 years or less, 8 years or less, 9 years or less, 10 years of less, 15 years or less, 20 years or less or aged about 30 years or less. In some embodiments, the methods can be used to estimate the age of fish aged between 1 to 10 years, 2 to 10, 3 to 10 years, 4 to 10 years or 5 to 10 years. In some embodiments, the methods can be used to estimate the age of fish aged 1 to 5 years. In some embodiments, the methods can be used to estimate the age of fish with an estimated age of greater than 30 years, for example 30 to 50 years. Marine turtles often live between 30 and 90 years, with some living as long as 100 or 150 years. In some embodiments, the methods can be used to estimate the age of a reptile aged about 10 years or less, 20 years or less, 30 years or less, 40 years or less, 50 years or less, 60 years or less, 70 years or less, 80 years or less, 90 years or less, 100 years or less, or 150 years or less. In some embodiments, the methods can be used to estimate the age of a reptile aged between 1 and 90 years, 1 and 50 years, 1 and 40 years, 1 and 30 years, 1 and 20 years, 10 and 90 years, 10 and 50 years or 10 and 30 years.

The methods for estimating the age of a fish or reptile described herein can be used to aid the study of the development of a fish or reptile. They may be used by fisheries for the management of fish or reptile populations and/or the management of over-fishing. The methods provided herein provide one or more advantages over techniques commonly used in the art, for example the use of otoliths to estimate age in fish. In some embodiments, these advantages include increased sensitivity, accuracy and/or reproducibility; reduced cost; and/or decreased invasiveness; and/or flexibility in the choice of biological sample used to estimate age. The methods can be performed without culling the fish or reptile which is important for sustainability reasons. The methods provided herein are also inexpensive compared to other techniques, such as bomb radiocarbon. The methods provided herein may also avoid reader bias which may occur with using otoliths for estimating age. By being both inexpensive and non-lethal epigenetic clocks have implications for wildlife management. For example, in threatened species it may be impossible to determine an age structure of a population. For example, natural resource management of commercial fishing of wild populations is controlled by calculations of total allowable catch and total allowable effort (including, number of licenses and method of fishing). Without an age structure, population growth, risk of extinction, and other population dynamics cannot be accurately defined (Caughley, 1977b).

### Correlation Coefficients, MAE and Percentage Error of Oldest Individual

The methods for estimating age described herein may be used to accurately estimate the age of a fish or reptile. The accuracy of the methods can be measured by statistical measures, such as correlation coefficients, mean average error rates or percentage error of oldest individual in the study. In some embodiments, the accuracy of the method is measured using the Pearson correlation. In some embodiments, the correlation between chronological age and estimated age is at least 70% (i.e. at least 0.7). In some embodiments, the correlation between chronological age and estimated age is at least 75%, at least 80%, at least 85%, at least 90%, least 92%, or at least 95%. In some embodiments, the correlation between chronological age and estimated age is at least 90%. In some embodiments, the correlation between chronological age and estimated age is at least 95%.

In some embodiments, the accuracy of the age estimate is measured using the percentage error of oldest individual in the study. In some embodiments, the percentage error of oldest individual in the study is less than 10%. In some embodiments, the percentage error of oldest individual in the study is less than 9%, less than 8%, less than 7%, less than 6%, less than 5% or less than 4.5%. In some embodiments, the percentage error of oldest individual in the study is less than 5%. In some embodiments, the percentage error of oldest individual in the study is 5%.

In some embodiments, the accuracy of the age estimate is measured using the “mean absolute error” or MAE. The MAE can be determined using methods known to the person skilled in the art. As would be understand by the person skilled in the art an acceptable MAE depends on the average lifespan on the fish or reptile. For fish having a lifespan that is measured in years (for example, a zebrafish which has a lifespan in captivity of 2-3 years, and up to 5-6 years or an Atlantic Salmon which has an average life expectancy of 3-8 years), the MAE is preferably measured in weeks. In some embodiments, the MAE is less than 15 weeks, 12 weeks, 10 weeks, 9 weeks, 8 weeks, 7 weeks, 6 weeks, 5 weeks, 4 weeks, or 3 weeks. In some embodiments, the MAE is less than 5 weeks. In some embodiments, the MAE is less than 3.5 weeks. For fish having a lifespan that is measured in decades (for example, a blue fin tuna which has a life expectancy of 15-30 years, and up to 40 years), the MAE is preferably measured in months or years. In some embodiments, the MAE is less than 24 months, less than 18 months, less than 12 months or less than 8 months.

### Method for Identifying Age-Associated CpG Sites of a Fish or Reptile

The present application also provides a method for identifying age-associated CpG sites for a species of fish or reptile. The method comprises analysing DNA obtained from the species of fish or reptile of different chronological ages for the presence of methylated cytosine at CpG sites. It will be appreciated that any technique suitable for the identification of methylated cytosine at CpG sites known to the person skilled in the art may be used. Examples include, but are not limited to, molecular break light assay for DNA adenine methyltransferase activity, methylation-specific polymerase chain reaction (PCR), whole genome bisulfite sequencing, the Hpall tiny fragment enrichment by ligation-mediated PCR (HELP) assay, methyl sensitive southern blotting, ChIP-on-chip assay, restriction landmark genomic scanning, methylated DNA immunoprecipitation (MeDIP), sequencing of bisulfite treated DNA (e.g. reduced representation bisulfite sequencing (RRBS) and whole genome bisulfite sequencing (WGBS)). Suitable methods are also described in WO2015/048665.

In some embodiments, the analysing step comprises reduced representation bisulfite sequencing. For example, the analysis comprises treatment of genomic DNA from a biological sample obtained from fish or reptile of known age with a bisulfite reagent to convert unmethylated cytosine of CpG sites to uracil. In some embodiments, the genomic DNA is fragmented by enzymatic digestion (such as with MspI) prior to bisulfite treatment. In some embodiments, the fragmented DNA is enriched for CpG islands using known techniques prior to bisulfite treatment. In some embodiments, sequence alignment and DNA methylation level calling is performed using any suitable alignment tool. Non-limiting examples include, Bismark (Krueger and Andrews, 2011), BSMAP/RRBSMAP (Bock et al., 2012) or BS-Seeker2 (Guo et al., 2013). In some examples, sequence alignment and methylation calling is performed using BS-Seeker2. In some embodiment, the analysis step further comprises measurement of the mean methylation level or beta value of each identified CpG site.

Following identification of methylated CpG sites, a statistical algorithm is used to identify age-associated CpG sites. It will be appreciated that any suitable statistical algorithm may be used to identify age-associated CpG sites. In some embodiments, the age of the sample may be subject to a transformation function (such as log or natural log) to enable the use of a linear model. In some embodiments, the statistical algorithm is elastic net regression model. For example, samples of known age may be randomly assigned to either a training or a testing data set and age-associated CpG sites are identified using an elastic net regression model to regress the known age of the DNA samples over all CpG site methylation in the training data set. In some embodiments, the elastic net regression model may be implemented in the GLMNET R package (Friedman et al., 2010). In some embodiments, the age-associated CpG sites are the CpG sites that have a non-zero weight after an elastic net regression model is used to regress the known age of the DNA samples over all CpG site methylation in the training data set. In some embodiments, the performance of the model in the training and testing data set may be assessed, for example, using Pearson correlations between the chronological and predicted age and the MAE rates. The result of this step is the identification of one or more age-associated CpG sites that are considered suitable to use to estimate the age of a fish or reptile.

The age-associated CpG sites identified using the methods described herein may then be used to identify or classify a test DNA sample from a test animal subject, i.e. to determine the age of the animal subject using the methods described herein.

In another embodiment, there is also provided a method of identifying age-associated CpG site for a second species of fish. The method of identifying age-associated CpG sites for a second species of fish described herein comprises (i) analysing DNA of the second fish species for a candidate age-associated CpG site selected from the age-associated CpG sites identified for a first species of fish. In some embodiments, the first species of fish is zebrafish. In some embodiments, the age-associated CpG sites are one or more of the CpG sites listed in Table 1, Table 2 or Table 3. In some embodiments, the first species of fish is school shark. In some embodiments, the age-associated CpG sites are one or more of the CpG sites listed in Table 8 or Table 9. The method further comprises (ii) analysing the methylation patterns of a candidate age-associated CpG site identified in (i) in different ages of the second species of fish to determine if it is an age-associated CpG site in that second fish species. In some embodiments, the second species of fish is a fish described herein. In some embodiments, the second species of fish is a bony fish. In some embodiments, the second species of fish is an Australian lungfish. In some embodiments, the second species of fish is a cod, for example a Murray cod or a Mary River cod. In some embodiments, the second species of fish is Atlantic Salmon. In some embodiments, the second species of fish is a member of the subclass Elasmobranchii. In some embodiments, the second species of fish is a shark or ray.

In another embodiment, there is also provided a method of identifying age-associated CpG site for a second species of reptile (for example a second species of marine turtle). The method of identifying age-associated CpG sites for a second species of reptile described herein comprises (i) analysing DNA of the second reptile species for a candidate age-associated CpG site corresponding to an age-associated CpG site identified for a first species of reptile; and (ii) analysing the methylation patterns of a candidate age-associated CpG site identified in (i) in different ages of the second species of reptile to determine if it is an age-associated CpG site in that second reptile species. In some embodiments, step (i) comprises a pairwise analysis of the DNA of the first reptile species with the DNA of the second reptile species. In some embodiments, the first reptile species is a marine turtle. In some embodiments, the first reptile species is a green sea turtle. In some embodiments, step (i) comprises analysing DNA of the second reptile species for a candidate age-associated CpG site corresponding to an age-associated CpG site listed in Table 19 or 20. In some embodiments, step (i) comprises analysing DNA of the second reptile species for a candidate age-associated CpG site corresponding to an age-associated CpG site listed in Table 20. In some embodiments, the second reptile species is a marine turtle, for example a marine turtle selected from the group consisting of Flatback turtle, Hawksbill turtle, Leatherback turtle, Loggerhead turtle and Olive Ridley turtle. In some embodiments, the first reptile is a green sea turtle and the second reptile species is a marine turtle is selected from the group consisting of Flatback turtle, Hawksbill turtle, Leatherback turtle, Loggerhead turtle and Olive Ridley turtle.

Using zebrafish as an example, a person skilled in the art will be able to identify a methylation site of another species that corresponds to an age-associated CpG site identified for Zebrafish, for example, a CpG site listed in Table 1, Table 2 or Table 3. In some embodiments, the age-associated CpG sites identified for Zebrafish are listed in Table 1. In some embodiments, step (i) comprises a pairwise analysis of the DNA with zebrafish DNA. In some embodiments, step (i) comprises a pairwise analysis of RNA (for example, RNA sequence data) with zebrafish DNA. For example, prediction software, such as ClustalW (Thompson et al., 1994; available at www.genome.jp/tools-bin/clustalw), LASTZ (Harris 2007; available at www.bx.psu.edu/miller_lab/dist/README.lastz-1.02.00/README.lastz-1.02.00a.html#intro) or HISAT2 (Kim et al., 2015), may be used to align the sequences of pairs of species. In some embodiments, genome pairwise alignment is performed against a zebrafish reference genome, such as danRer10 (illumine iGenomes). In some embodiments, candidate age-associated CpG are identified using LASTZ v1.04.00 with the following conditions: [multiple]--notransition --step=20 -nogapped (Harris 2007). In some embodiments, candidate age-associated CpG are identified using HISAT2 v2.1.0 with default parameters (Kim et al., 2015). In some embodiments, homologous CpG sites can be identified, e.g., by applying the Perl module Bio::AlignIO. In some embodiments, suitable software, such as bedtools (available at bedtools.readthedocs.io/en/latest/) may be used to identify DNA or RNA sequences that overlap with the age-associated CpG sites identified for the reference genome. In some embodiments, potential error due to misalignment may be removed, by further filtering the sites by requiring that the two flanking nucleotides (immediately upstream and downstream of each focal CpG) also are identical between the pair of species. In some embodiments, genomic context is also considered, for example, the CpG content of the surrounding nucleotides, presence within a CpG island of high CpG density, and/or location within promoters, first exons, first introns, internal exons, internal introns or last exons. In some embodiments, candidate age-associated CpG sites include CpG sites with a significant Pearson correlation with age (p<0.05) in zebrafish and which are conserved as identified by genome pairwise alignment with the zebrafish genome. As would be understood by the person skilled in the art, typically a p-value of less than 0.05 indicates that the correlation between variable is significant. In some embodiments, RNA-seq alignments that overlap with age associated CpG sites identified for the reference genome are targeted for primer design. In some embodiments, DNA sequences that are conserved between the candidate genome and the reference genome and which contain methylation-age associated CpG sites are targeted for primer design. Primers can be designed by the person skilled in the art, for example, using Primersuite (www.primer-suite.com/ (Lu et al., 2017)).

The method of identifying age-associated CpG sites for a species of fish described herein comprises (ii) analysing the methylation patterns of a candidate age-associated CpG site identified in (i) in different ages of the species of fish to determine if it is an age-associated CpG site in that fish species. In some embodiments, the step (ii) analysis comprises determining if the level of methylated cytosine at the candidate age-associated CpG site changes (e.g. increases or decreases) as a fish ages. This can be analysed in a single fish over time or preferably using an age correlated reference population comprising fish of varying age (e.g., birth, 1 week, 2 weeks, 1 month, 1 year, 2 years etc. until natural death). It will be appreciated that the level of methylated cytosine at the candidate age-associated CpG sites may be analysed using general methodology known to the person skilled in the art, including those described herein. For example, PCR followed by DNA sequencing may be used. The PCR may be performed in multiplex. In some embodiments, the DNA is bisulfite treated prior to PCR.

In some embodiments, the step (ii) analysis comprises use of a statistical method to determine if there is a relationship between the level of methylated cytosine at one or more candidate age-associated CpG sites and the age of the fish. Any suitable statistical comparison methodology known to the person skilled in the art can be used to relate the methylation levels to age. Examples of suitable statistical methods include, but are not limited to, multivariate regression method, linear regression analysis, tabular method or graphical method. In some embodiments, the statistical method comprises the elastic-net generalised linear model. In some embodiments, the statistical method comprises use of an elastic-net generalized linear model as implemented in the GLMNET package (Friedman et al., 2010). The result of this step is the identification of one or more confirmed age-associated CpG sites that are considered suitable to use to estimate the age of a fish. These confirmed age-associated CpG sites may then be used in the methods described herein, for example, to estimate the age of a fish.

### Fish or Reptile

The methods described herein can be applied to fish or reptiles. In some embodiments, the DNA is obtained from a fish. Fish include, but are not limited to, jawless fish (Agnatha), cartilaginous fish (Chondrichthyes, which includes the sub class Elasmobranchii (sharks and rays) and the subclass Holocephali (chimaeras), and bony fish (Osteichthyes, which includes the subclass Actinopterygii (ray finned fish) and the subclass Sarcopterygii (fleshy finned fish)). In some embodiments, the fish is a cartilaginous fish or a bony fish. In some embodiments, the fish is a cartilaginous fish. In some embodiments, the fish is a bony fish.

In some embodiments, the fish is a member of the class Chondrichthyes. In some embodiments, the fish is a member of the subclass Elasmobranchii. In some embodiments, the fish is a shark, ray, skate, or sawfish. In some embodiments, the fish is a shark, ray or skate. In some embodiments, the fish is a shark or ray. In some embodiments, the fish is a shark. Sharks include, but are not limited to, ground sharks, bull head sharks, mackerel sharks, carpet sharks, frilled and cow sharks, sawsharks, dogfish sharks and angel sharks. In some embodiments, the shark is a member of the family Triakidae. In some embodiments, the shark is a school shark (Galeorhinus galeus). School shark are also referred to as snapper shark, eastern school shark, soupfin shark or tope.

In some embodiments, the fish is a member of the class Actinopterygii. In some embodiments, the fish is a member of the order Cypriniformes, Percoidei, Ceratodontiformes, Lepidosireniformes, Polypteriformes, Amiiformes, Lepisosteiformes, Clupeiformes, Gonorynchiformes, Esociformes, Osteoglossiformes, Characiformes, Gymnotiformes, Siluriformes, Anguilliformes, Beloniformes, Gadiformes, Gasterosteiformes, Cyprinodontiformes, Percopsiformes, Atheriniformes, Synbranchiformes, Gobioidei, Stromateoidei, Anabantoidei, Other Perciformes, Scorpaeniformes, Pisces Miscellanea, Acipenseriformes, Salmoniformes, Petromyzontiformes, Pleuronectiformes, Myxiniformes, Elopiformes, Albuliformes, Aulopiformes, Syngnathiformes, Ophidiiformes, Beryciformes, Mugiliformes, Zoarcoidei, Trachinoidei, Acanthuroidei, Tetraodontiformes, Gobiesociformes, Batrachoidiformes, Lophiiformes, Coelacanthiformes, Stomiiformes, Myctophiformes, Saccopharyngiformes, Notacanthiformes, Cetomimiformes, Zeiformes, Scombroidei, Lampriformes, Heterodontiformes, Hexanchiformes, Lamniformes, Orectolobiformes, Carcharhiniformes, Squaliformes, Rajiformes, Torpediniformes, or Chimaeriformes. In some embodiments, the fish is a member of the family Catostomidae, Cyprinidae, Gyrinocheilidae, Cobitidae, Psilorhynchidae, Balitoridae, Cichlidae, Ceratodontidae, Lepidosirenidae, Polypteridae, Protopteridae, Amiidae, Lepisosteidae, Sundasalangidae, Clupeidae, Engraulidae, Denticipitidae, Kneriidae, Phractolaemidae, Umbridae, Esocidae, Osteoglossidae, Notopteridae, Hiodontidae, Pantodontidae, Mormyridae, Gymnarchidae, Characidae, Gasteropelecidae, Ctenoluciidae, Anostomidae, Hemiodontidae, Citharinidae, Erythrinidae, Hepsetidae, Lebiasinidae, Curimatidae, Alestidae, Cynodontidae, Acestrorhynchidae, Distichodontidae, Rhamphichthyidae, Gymnotidae, Electrophoridae, Apteronotidae, Hypopomidae, Sternopygidae, Diplomystidae, Doradidae, Auchenipteridae, Ageneiosidae, Plotosidae, Siluridae, Bagridae, Ictaluridae, Amblycipitidae, Akysidae, Sisoridae, Amphiliidae, Chacidae, Schilbeidae, Clariidae, Olyridae, Malapteruridae, Pimelodidae, Helogeneidae, Trichomycteridae, Callichthyidae, Loricariidae, Cranoglanididae, Pangasiidae, Heteropneustidae, Mochokidae, Aspredinidae, Cetopsidae, Astroblepidae, Parakysidae, Ophichthidae, Belonidae, Adrianichthyidae, Gadidae, Indostomidae, Cyprinodontidae, Goodeidae, Anablepidae, Poeciliidae, Aplocheilidae, Profundulidae, Fundulidae, Valenciidae, Percopsidae, Aphredoderidae, Amblyopsidae, Atherinidae, Bedotiidae, Melanotaeniidae, Pseudomugilidae, Synbranchidae, Mastacembelidae, Chaudhuriidae, Centropomidae, Terapontidae, Moronidae, Percichthyidae, Centrarchidae, Percidae, Sciaenidae, Toxotidae, Nandidae, Coiidae, Eleotridae, Gobiidae, Rhyacichthyidae, Odontobutidae, Anabantidae, Osphronemidae, Belontiidae, Helostomatidae, Amarsipidae, Luciocephalidae, Tripterygiidae, Kurtidae, Channidae, Elassomatidae, Cottidae, Cottocomephoridae, Comephoridae, Abyssocottidae, Acipenseridae, Polyodontidae, Anguillidae, Salmonidae, Thymallidae, Plecoglossidae, Osmeridae, Salangidae, Retropinnidae, Coregonidae, Lepidogalaxiidae, Galaxiidae, Pristigasteridae, Petromyzontidae, Mordaciidae, Geotriidae, Chanidae, Gasterosteidae, Bothidae, Pleuronectidae, Soleidae, Cynoglossidae, Scophthalmidae, Citharidae, Psettodidae, Paralichthyidae, Achiridae, Achiropsettidae, Samaridae, Muraenolepididae, Moridae, Bregmacerotidae, Merlucciidae, Macrouridae, Melanonidae, Euclichthyidae, Myxinidae, Gonorynchidae, Elopidae, Megalopidae, Albulidae, Aulopidae, Alepisauridae, Anotopteridae, Pseudotrichonotidae, Synodontidae, Ariidae, Muraenidae, Heterenchelyidae, Moringuidae, Chlopsidae, Aulorhynchidae, Pegasidae, Hypoptychidae, Aulostomidae, Fistulariidae, Centriscidae, Solenostomidae, Syngnathidae, Carapidae, Bythitidae, Holocentridae, Mugilidae, Caesionidae, Serranidae, Glaucosomatidae, Polyprionidae, Plesiopidae, Kuhliidae, Priacanthidae, Apogonidae, Sillaginidae, Malacanthidae, Pseudochromidae, Nematistiidae, Banjosidae, Menidae, Arripidae, Inermiidae, Lutjanidae, Nemipteridae, Leiognathidae, Haemulidae, Lethrinidae, Sparidae, Centracanthidae, Mullidae, Dichistiidae, Monodactylidae, Gerreidae, Kyphosidae, Pempheridae, Lateolabracidae, Drepaneidae, Chaetodontidae, Enoplosidae, Oplegnathidae, Embiotocidae, Pomacentridae, Labridae, Odacidae, Scaridae, Pomacanthidae, Cirrhitidae, Chironemidae, Aplodactylidae, Opistognathidae, Grammatidae, Polynemidae, Notograptidae, Parascorpididae, Centrogeniidae, Dinolestidae, Callanthiidae, Dinopercidae, Bovichtidae, Nototheniidae, Ambassidae, Leptobramidae, Bathymasteridae, Stichaeidae, Pholidae, Ptilichthyidae, Zoarcidae, Scytalinidae, Cryptacanthodidae, Ammodytidae, Percophidae, Pinguipedidae, Trichonotidae, Creediidae, Trachinidae, Leptoscopidae, Kraemeriidae, Microdesmidae, Xenisthmidae, Acanthuridae, Ephippidae, Scatophagidae, Siganidae, Luvaridae, Zanclidae, Pholidichthyidae, Dactyloscopidae, Clinidae, Blenniidae, Schindleriidae, Callionymidae, Labrisomidae, Chaenopsidae, Caracanthidae, Aploactinidae, Synanceiidae, Pataecidae, Hexagrammidae, Platycephalidae, Normanichthyidae, Agonidae, Tetrarogidae, Dactylopteridae, Gnathanacanthidae, Apistidae, Zaniolepididae, Hemitripteridae, Ostraciidae, Tetraodontidae, Diodontidae, Triacanthidae, Triodontidae, Monacanthidae, Balistidae, Gobiesocidae, Batrachoididae, Antennariidae, Brachionichthyidae, Tetrabrachiidae, Latimeriidae, Argentinidae, Bathylagidae, Microstomatidae, Opisthoproctidae, Alepocephalidae, Platytroctidae, Leptochilichthyidae, Gonostomatidae, Stemoptychidae, Stomiidae, Phosichthyidae, Giganturidae, Scopelarchidae, Evermannellidae, Omosudidae, Paralepididae, Chlorophthalmidae, Notosudidae, Ipnopidae, Neoscopelidae, Myctophidae, Saccopharyngidae, Eurypharyngidae, Monognathidae, Cyematidae, Derichthyidae, Myrocongridae, Muraenesocidae, Nettastomatidae, Congridae, Synaphobranchidae, Nemichthyidae, Colocongridae, Serrivomeridae, Halosauridae, Notacanthidae, Macroramphosidae, Ophidiidae, Aphyonidae, Parabrotulidae, Rondeletiidae, Barbourisiidae, Cetomimidae, Polymixiidae, Berycidae, Diretmidae, Trachichthyidae, Monocentridae, Anomalopidae, Gibberichthyidae, Melamphaidae, Anoplogasteridae, Stephanoberycidae, Hispidoberycidae, Zeidae, Grammicolepididae, Caproidae, Oreosomatidae, Parazenidae, Macrurocyttidae, Acropomatidae, Branchiostegidae, Scombropidae, Emmelichthyidae, Lobotidae, Howellidae, Bathyclupeidae, Caristiidae, Pentacerotidae, Cepolidae, Cheilodactylidae, Latridae, Ostracoberycidae, Symphysanodontidae, Artedidraconidae, Bathydraconidae, Channichthyidae, Epigonidae, Harpagiferidae, Anarhichadidae, Zaproridae, Champsodontidae, Chiasmodontidae, Uranoscopidae, Trichodontidae, Gempylidae, Trichiuridae, Ariommatidae, Centrolophidae, Icosteidae, Draconettidae, Scombrolabracidae, Scorpaenidae, Triglidae, Anoplopomatidae, Hoplichthyidae, Congiopodidae, Psychrolutidae, Cyclopteridae, Peristediidae, Liparidae, Ereuniidae, Bembridae, Bathylutichthyidae, Triacanthodidae, Lophiidae, Chaunacidae, Ogcocephalidae, Caulophrynidae, Melanocetidae, Diceratiidae, Himantolophidae, Oneirodidae, Gigantactinidae, Neoceratiidae, Ceratiidae, Linophrynidae, Lophichthyidae, Centrophrynidae, Chirocentridae, Scombridae, Istiophoridae, Xiphiidae, Scomberesocidae, Hemiramphidae, Exocoetidae, Lampridae, Veliferidae, Lophotidae, Trachipteridae, Regalecidae, Stylephoridae, Ateleopodidae, Mirapinnidae, Megalomycteridae, Radiicephalidae, Phallostethidae, Notocheiridae, Telmatherinidae, Dentatherinidae, Lactariidae, Pomatomidae, Rachycentridae, Carangidae, Bramidae, Coryphaenidae, Echeneidae, Tetragonuridae, Stromateidae, Nomeidae, Sphyraenidae, Molidae, Heterodontidae, Chlamydoselachidae, Hexanchidae, Cetorhinidae, Odontaspididae, Mitsukurinidae, Pseudocarchariidae, Megachasmidae, Alopiidae, Lamnidae, Stegostomatidae, Orectolobidae, Ginglymostomatidae, Hemiscylliidae, Rhincodontidae, Brachaeluridae, Parascylliidae, Scyliorhinidae, Carcharhinidae, Sphyrnidae, Triakidae, Pseudotriakidae, Hemigaleidae, Leptochariidae, Proscylliidae, Squalidae, Pristiophoridae, Squatinidae, Oxynotidae, Echinorhinidae, Rhinobatidae, Pristidae, Rajidae, Dasyatidae, Potamotrygonidae, Myliobatidae, Mobulidae, Gymnuridae, Hexatrygonidae, Urolophidae, Anacanthobatidae, Plesiobatidae, Torpedinidae, Narcinidae, Chimaeridae, Rhinochimaeridae, or Callorhinchidae. Non-limiting examples of fish may be found in the ASFIC list of Species published by the Food and Agriculture Organization of the United Nations (available online at www.fao.org/fishery/collection/asfis/en).

In some embodiments, the fish is a member of the class Actinopterygii. In some embodiments, the fish is a member of the infraclass Teleostei. In some embodiments, the fish is a Grouper, Tuna (e.g. Skipjack tuna, Blue fin tuna, yellow fin tuna, bigeye tuna), Cobia, Sturgeon, Mahi-mahi, Bonito (e.g. Atlantic bonito, Australian Bonito) Dhufish, Murray cod, Barramundi, Herring (e.g. Atlantic Herring and Pacific Herring), Tra catfish, Mekong giant catfish, Cod (e.g. Pacific cod), pilchard, Pollock, Turbot, Hake, Anchovy, Haddock, Black carp, Grass carp, Eels, Koi Carp, Giant gourami, zebrafish, Mackerel, Australian lungfish, Mary river cod or Salmon (e.g. Atlantic salmon, pink salmon) or trout (e.g. Rainbow trout). In some embodiments, the fish is a Grouper (e.g. Epinephelus spp.), Blue fin tuna (e.g. Thunnus thynnus, T. orientalis, T. maccoyii), Yellow fin tuna (e.g. T. albacares), Cobia (e.g. Rachycentron canadum), Sturgeon (e.g. Acipenser spp. such as A. sturio), Mahi-mahi (Coryphaena hippurus), Dhufish (e.g. Glaucosoma hebraicum), Murray cod (e.g. Maccullochella peeli), Barramundi (e.g. Lates calcarifer), Tra catfish (Pangasianodon hypophthalmus), Mekong giant catfish (Pangasius gigas), Cod (e.g. Gadus spp. such as Gadus morhua), Turbot (Scophthalmus maximus), Black carp (Mylopharyngodon piceus), Grass carp (Ctenopharyngodon idellus), Eels, Koi Carp (e.g. Cyprinus rubrofuscus), Giant gourami (Osphronemus goramy), zebrafish (Danio rerio), Australian lungfish (Neoceratodus forsteri), Mary river cod (Maccullochella mariensis), Salmon, (e.g. Salmo spp., Oncorhynchus spp.) or trout. In some embodiments, the fish is zebrafish, yellow fin tuna, skipjack tuna, Atlantic cod, Atlantic herring, Alaska pollock, Australian lungfish, Mary River Cod or Atlantic Salmon. In some embodiments, the fish is zebrafish, Australian lungfish, Mary River Cod or Atlantic Salmon. In some embodiments, the fish is zebrafish. In some embodiments, the fish is an Atlantic salmon. In some embodiments, the fish is Blue fin tuna. In some embodiments, the fish is not European sea bass (Dicentrarchus labrax).

In some embodiments, the DNA is obtained from a reptile. In some embodiments, the reptile is a member of the class Reptilia. In some embodiments, the reptile is a turtle, crocodilian, snake, amphisbaenian, lizard or tuatara. In some embodiments, the reptile is a caiman, alligator or crocodile. In some embodiments, the reptile is a turtle. In some embodiments, the turtle is a marine turtle (also referred to as a sea turtle). Examples of marine turtles include the green sea turtle, loggerhead sea turtle, Kemp's ridley sea turtle, olive ridley sea turtle, hawksbill sea turtle, flatback sea turtle, and leatherback sea turtle.

### Biological Sample

In some embodiments, the methods described herein further comprise obtaining a biological sample comprising the DNA from the fish or reptile. Any biological sample which comprises DNA from a fish or reptile, can be used in the methods described herein. Examples of biological samples include, but are not limited to, blood, plasma, serum, or tissue biopsy. In some embodiments, the sample is obtained from tissues that can be accessed without sacrificing the fish or reptile. Examples of tissue biopsies that can be used include, but are not limited to, from muscle, head, neck, fin, or skin. In some embodiments, the sample is a tissue biopsy obtained from head, neck, fin, or skin. In some embodiments, the sample is not obtained from muscle. In some embodiments, the biological sample is a skin tissue biopsy. In some embodiments, the biological sample is from the fin of a fish. In some embodiments, the biological sample is from the caudal fin of a fish. In some embodiments, the biological sample comprises, or is, blood or a fraction thereof. Preferably, the biological sample is obtained by non-lethal means. Advantageously, in some embodiments, it is thought that age-associated CpG sites identified using the methods described herein can be used to estimate the age of a fish or reptile based on a biological sample obtained from different tissue types. In other words, it is thought that the methods are “tissue agnostic” in that they may be used to estimate the age of a fish or reptile irrespective of the biological sample.

The sample may be stored prior to processing. In some embodiments, the sample is stored in a storage reagent, for example, RNAlater (Thermo Fisher) or 70% ethanol.

Typically, the biological sample will be obtained from a fish or reptile with most of the DNA within intact cells. In these circumstances, it is preferred that the sample is at least partially processed to liberate the DNA from the cells. Techniques for processing samples to isolate DNA are known in the art and include, but are not limited to, phenol/chloroform extraction (Green and Sambrook 2012), QIAampR™ Tissue Kit (Qiagen, Chatsworth, Calif), DNeasy Blood & Tissue Kit (Qiagen, Chatsworth, Calif), WizardR™ Genomic DNA purification kit (Promega, Madison, Wis.), the A.S.A.P.™ Genomic DNA isolation kit (Boehringer Mannheim, Indianapolis, Ind.) and the Easy-DNA™ Kit (Invitrogen). Typically, samples are processed in accordance with the manufacturer's instructions. Before DNA extraction, the sample may also be processed to decrease the concentration of one or more sources of non-target DNA.

### Kits

The present application further provides kits for estimating the age of a fish or reptile. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Kit also includes delivery systems comprising two or more separate containers that each contain a subportion of the total kit components. The containers may be delivered to the intended recipient together or separately. For example, a first container may contain an enzyme for use in an assay, while a second container contains oligonucleotides.

The kits may further contain reagents for analysing the methylation profile of the DNA obtained from the fish or reptile, optionally together with instructional material. Reagents for detection of methylation include, e.g., sodium bisulfite, nucleic acids including primers and oligonucleotides designed to amplify an amplicon containing an age-associated CpG site, buffering agents, thermostable DNA polymerase, dNTPs restriction enzymes and/or the like. In some instances, the kit comprises a plurality of primers or probes to detect or measure the methylation status/levels of one or more samples. In some embodiments, the kit comprises a set of primers for detecting the age-associated CpG sites defined herein, for example, those listed in Table 1, 2, 3, 7, 8 or 9 or a homolog of one or more thereof. In some embodiments, the kit comprises a set of primers for detecting the age-associated CpG sites defined herein, for example, those listed in Table 1, 2, 3, 7, 8, 9, 12, 16, 19 or 20 or a homolog of one or more thereof. In some embodiments, the kit comprises a set of primers for detecting the age-associated CpG sites defined herein, for example, those listed in Table 1, Table 2 or Table 3 or a homolog of one or more thereof. In some embodiments, the kit comprises one or more of the primer pairs listed in Table 4. In some embodiments, the kit comprises 2, 3, 4, 5, 6, 7, 8, 9, 10, 11, 12, 13, 14, 15, 16, 17, 18, 19, 20, 21, 22, 23, 24, 25 or 26 of the primer pairs listed Table 4 or any primer pair that is capable of amplify the age-associated CpG sites listed in Table 1, Table 2 or Table 3 or a homolog of one or more thereof. In some embodiments, the kit comprises one or more all of the primer pairs listed in Table 4. In some embodiments, the kit comprises a set of primers for detecting the age-associated CpG sites defined herein, for example, those listed in Table 7, Table 8, Table 9, Table 12, Table 16, Table 19 or Table 20 or a homolog of one or more thereof. In some embodiments, the kit comprises one or more of the primer pairs listed in Table 11. In some embodiments, the kit comprises one or more of the primer pairs listed in Table 15.

In some embodiments, the kit includes a packaging material. In some embodiments, the packaging material maintains sterility of the kit components, and is made of material commonly used for such purposes (e.g., paper, corrugated fibre, glass, plastic, foil, ampules, etc.). Other materials useful in the performance of the assays are included in the kits, including test tubes, transfer pipettes, and the like. In some cases, the kits also include written instructions for the use of one or more of these reagents in any of the assays described herein.

### Computer Readable Medium

The present application further provides a computer-readable medium for estimating the age of a fish or reptile. The present application also provides a computer-readable medium which comprises a training data set comprising one or more or all of the CpG defined herein or a homolog thereof. In some embodiments, there is provided a computer-readable medium which comprises a training data set comprising one or more or all of the CpG sites listed in Tables 1, 2, 3, 7, 8, 9, 12, 16, 19 or 20 or a homolog of one or more thereof.

In some embodiments, there is provided a computer-readable medium which comprises a training data set comprising one or more or all of the CpG sites listed in Tables 1, 2 or 3 or a homolog of one or more thereof. For example, in some embodiments the training data set comprises any of the CpG sites listed in Table 1 or at least 5, 10, 25, 50, 100, 150, 200, 250, 300, 350, 400, 450, 550, 600, 650, 700, 750, 800, 850, 900, 950, 1000, 1050, 1100, 1150, 1200, 1250, 1300 or all of the 1311 CpG sites listed in Table 1 or a homolog of one or more thereof. For example, in some embodiments the training data set comprises any of the CpG sites listed in Table 2 or at least 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the CpG sites listed in Table 2 or a homolog of one or more thereof. For example, in some embodiments the training data set comprises any of the CpG sites listed in Table 3 or at least 1, 2, 3, 4, 5, 6, 7, 8, 9, 10, 11, 12, 13, 14, 15, 16, 17, 18, 19, 20, 21, 22, 23, 24, 25, or 26 of the CpG sites listed in Table 3 or a homolog of one or more thereof. In a yet further embodiment, the computer-readable medium comprises the training data set comprising all of the 1311 CpG sites listed in Table 1 or a homolog thereof.

In some embodiments, there is provided a computer-readable medium which comprises a training data set comprising one or more or all of the CpG sites listed in Table 7 or a homolog of one or more thereof. For example, in some embodiments the training data set comprises any of the CpG sites listed in Table 7 or at least 5, 10, 15, 20, 25, 30, 35, 40, 45, 48, 50, 60, 70, 80, 90, 100, 110, 120 or 130 or all of the 1311 CpG sites listed in Table 7 or a homolog of one or more thereof.

In some embodiments, there is provided a computer-readable medium which comprises a training data set comprising one or more or all of the CpG sites listed in Table 8 or a homolog of one or more thereof. In some embodiments the training data set comprises any of the CpG sites listed in Table 8 or at least 1, 2, 3, 4, 5, 6, 7, 8, 9, 10, 11, 12, 13, 14, 15, 16, 17, 18, 19, 20, 21, 22, 23, 24, 25, 26, 27, 29, 29 or 30 of the CpG sites listed in Table 8 or a homolog of one or more thereof.

In some embodiments, there is provided a computer-readable medium which comprises a training data set comprising one or more or all of the CpG sites listed in Table 9 or a homolog of one or more thereof. In some embodiments the training data set comprises any of the CpG sites listed in Table 9 or at least 1, 2, 3, 4, 5, 6, 7, 8, 9, 10, 11, 12, 13, 14, 15, 16, 17, 18, 19, 20, 21, 22 or 23 of the CpG sites listed in Table 9 or a homolog of one or more thereof.

In some embodiments, there is provided a computer-readable medium which comprises a training data set comprising one or more or all of the CpG sites listed in Table 12 or a homolog of one or more thereof. In some embodiments the training data set comprises any of the CpG sites listed in Table 12 or at least 1, 2, 3, 4, 5, 6, 7, 8, 9, 10, 11, 12, 13, 14, 15, 16, 17, 18, 19, 20, 21, 22, 23, 24, 25, 26, 27, 29, 30 or 31 of the CpG sites listed in Table 12 or a homolog of one or more thereof.

In some embodiments, there is provided a computer-readable medium which comprises a training data set comprising one or more or all of the CpG sites listed in Table 16 or a homolog of one or more thereof. In some embodiments the training data set comprises any of the CpG sites listed in Table 16 or at least 1, 2, 3, 4, 5, 6, 7, 8, 9, 10, 11, 12, 13, 14, 15, 16, 17, 18, 19, 20, 21, 22, 23, 24, 25 or 26 of the CpG sites listed in Table 16 or a homolog of one or more thereof.

In some embodiments, there is provided a computer-readable medium which comprises a training data set comprising one or more or all of the CpG sites listed in Tables 19 or 20 or a homolog of one or more thereof. For example, in some embodiments the training data set comprises any of the CpG sites listed in Table 19 or at least 5, 10, 15, 20, 25, 30, 35, 40, 45, 50, 60, 70, 80, 90, 100, 110 or all of the 119 CpG sites listed in Table 19 or a homolog of one or more thereof. For example, in some embodiments the training data set comprises any of the CpG sites listed in Table 20 or at least 1, 2, 3, 4, 5, 6, 7, 8, 9, 10, 11, 12, 13, 14, 15, 16, 17, 18, 19, 20, 21, 22, 23, 24, 25, 26, 27, 28 or 29 of the CpG sites listed in Table 20 or a homolog of one or more thereof. In a yet further embodiment, the computer-readable medium comprises the training data set comprising all of the 119 CpG sites listed in Table 20 or a homolog thereof.

In some embodiments, a computer-readable medium refers to any storage device used for storing data accessible by a computer, as well as any other means for providing access to data by a computer. Examples of a storage device-type computer-readable medium include: a magnetic hard disk; a floppy disk; an optical disk, such as a CD-ROM and a DVD; a magnetic tape; a memory chip. Computer-readable physical storage media useful in various embodiments of the disclosure can include any physical computer-readable storage medium, e.g., solid state memory (such as flash memory), magnetic and optical computer-readable storage media and devices, and memory that uses other persistent storage technologies. In some embodiments, a computer readable media is any tangible media that allows computer programs and data to be accessed by a computer. Computer readable media can include volatile and non-volatile, removable and non-removable tangible media implemented in any method or technology capable of storing information such as computer readable instructions, program modules, programs, data, data structures, and database information. In some embodiments of the disclosure, computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store information and which can read by a computer including and any suitable combination of the foregoing. In some embodiments, there is provided a computer that includes the computer-readable medium as defined herein. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein. In some embodiments, computers are connected to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

### EXAMPLES

### Example 1—Materials and Methods

### Zebrafish Ageing Colony

Zebrafish (AB strain) were bred and maintained at the Western Australian Zebrafish Experimental Research Centre (WAZERC). Animal ethics was approved by the University of Western Australia animal ethics committee (RA/3/100/1630). Animals aged between 3 and 18 months were euthanized using rapid cooling. Once deceased all organs and tissues were collected and stored into RNAlater (Thermo Fisher). DNA was extracted using the DNeasy Blood & Tissue Kit (QIAGEN) following the manufacture's protocol.

### Reduced Representation Bisulfite Sequencing

A total of 96 RRBS libraries were prepared as previously described with digestion of the restriction enzyme MspI (Smallwood et al., 2011) at the Australian Genome Research Facility (AGRF) and were sequenced using an Illumina NovaSeq.

### RRBS Sequence Data Analysis

Fastq files were quality checked using FastQC v0.11.8 (www.bioinformatics.babraham.ac.uk/projects/fastqc/). Reads were trimmed using trimmomatic v 0.38 (Bolger et al., 2014) with the following options: SE -phred33 ILLUMINACLIP:TruSeq3-SE:2:30:10 LEADING:3 TRAILING:3 SLIDINGWINDOW:4:15 MINLEN:36. Trimmed reads were aligned to the zebrafish genome (danRer10) using BS-Seeker2 v 2.0.3 default settings (Guo et al., 2013) and bowtie2 v2.3.4 (Langmead and Salzberg, 2012). Methylation calling was performed using BS-Seeker2 call methylation module with default settings. CpG sites were filtered out of the analysis if they had a mean coverage of <2 reads or >100 reads.

Predicting Age from CpG Methylation

In order to predict age from CpG methylation samples were randomly assigned to either a training (67 samples) or a testing data set (29 samples) using the createDataPartition function in the caret R package (Kuhn et al., 2008). Age was transformed to natural log to fit a linear model. Using an elastic net regression model, the age of the zebrafish was regressed over CpG site methylation (all sites included initially) in the training data set. The glmnet function in the glmnet R package (Friedman et al., 2010) was set to a 10-fold cross validation with an α-parameter of 0.5, which returned a minimum k-value based on the training data of 0.02599415. These parameters identified 29 CpG sites (Table 2) that could be used estimate the age of zebrafish. The performance of the model in the training and testing data set were assessed using Pearson correlations between the chronological and predicted age and the MAE rates.

### Principle Component Analysis and Gene Ontology

A PCA was used as a form of unsupervised clustering to visualise the age associated CpG sites in terms of separating samples by age. PCA was performed using FactoMineR (Lê et al., 2008). Gene ontology (GO) enrichment was performed using the 2018 terms in in the R package Enrichr (Kuleshov et al., 2016). All analyses were performed in R using version 3.5.1.

### DNA Bisulfite Conversion

DNA was bisulfite converted using the EZ DNA Methylation Gold Kit (Zymo Research) in accordance with the manufacturer's instructions. DNA was also bisulfite converted using the protocol as previously described (Clark et al., 2006).

### Multiplex PCR

A total of 96 independent zebrafish caudal fin tissue, which was not used for the initial RRBS ranging from 10.9-78.1 weeks was used for the multiplex PCR assay. For each age-associated CpG site, primers were designed to amplify a 140 bp amplicon with the site of interest (Tables 4 and 5). Primers were designed using Primersuite (Lu et al., 2017) and were divided into two PCR reaction pools prior to barcoding (Table 4). Samples were run in triplicate to determine reproducibility of the method. The final 50 μL PCR reaction contained 1× Green GoTaq Flexi Buffer (Promega), 0.025 U/μL of GoTaq Hot Start Polymerase (Promega), 4.5 mM MgCl₂ (Promega), 0.5× Combinatorial Enhancer Solution (CES) (Refer to Ralser et al., 2006), 200 μM of each dNTP (Fisher Biotec), 15 mM Tetramethylammonium chloride (TMAC) (Sigma-aldrich), 200 nM forward primer, 200 nM reverse primer and 2 ng/μL bisulfite treated DNA. Cycling conditions were 94° C./5 mins; 12 cycles of 95° C./20 seconds and 60° C./60 seconds; 12 cycles of 94° C./20 seconds and 65° C./90 seconds; 65° C./3 mins; 10° C./hold. An Eppendorf ProS 384 thermocycler was used for amplification.

TABLE 5
Amplicons amplified for example age-associated CpG sites. The genomic coordinates are from the Zebrafish genome version
danRer10.

| CpG site | | | Amplicon | |
|---|---|---|---|---|
| chr | position | strand | Size (bp) | Amplicon Sequence |
| chr12 | 35432443 | + | 146 | TGAGTGTTTGTTTGGTCAAGCATCGGCTCTCTCTCTCCCTCGTGCACGCGCAGATATCTGCACAGCTGTGACCC |
| | | | | ACATAGCGGACAAGACAGGTGCTGCTGCTCGGCCGGGCAGGTGGTCATCCTGTGGGTGGAGGAACTGTCCTG |
| | | | | [SEQ ID NO: 53] |
| chr13 | 31180246 | + | 143 | AGCCCCGGAGAACCACTACAGGTTACTTTCCAGGCCTTCTTCAACAAATGAGCTGTTGCCGGCGGCTGGTTTCC |
| | | | | CTGTGAGCCCTCTGAATAATTATGTGTACCGCCGGCTCTAGACACCTTGAGGATTTCAGCTGTCTTGTT [SEQ |
| | | | | ID NO: 54] |
| chr13 | 38582448 | + | 135 | TTACATCTGAATAGGTGTTTCCCTTTGTGATGTCGCCGCTTTTGTTAAGTGGGGAAGGCGCGAAGCCGGAGACA |
| | | | | ATGGCGTTCGTGTTTGGTGCGGAAAAGCGGCTCTGTTTATGAACTCCTCACAATGCAGGGG [SEQ ID |
| | | | | NO: 55] |
| chr14 | 45387151 | + | 127 | gattgaggcagttctgaagacaaaagggggtccaacacggtactcataaggtgtacctaataaagtggccggtg |
| | | | | agtgTAAATGGGGAAAAAACGAAGCGCTAGAAACAATGGTTATGCTTTAAGGA [SEQ ID NO: 56] |
| chr17 | 52836692 | + | 133 | TCGGATCACAAATCTCCAATCATTTCTGAAAGTCGAGACCGCATTAATTTATTCACACGAAACACACACTTGTT |
| | | | | TTCCACACACATACAGCATTTAATGCCGGGAACACGCTGATCTAAAGCGGCATCTGCtg [SEQ ID NO: 57] |
| chr18 | 38107080 | + | 149 | CGGTCTGTGTATGTGAAAGTGCGAGGCACTCCGGCTTCAAATAGCAGGGACAGGGAGACGGACGGATTTATTAA |
| | | | | TGGCCAGGAACCAGGGTGCAGGGGGGGGGGGGATCCGAGATCCGGAGAGGAAGACTGGAGAATGATTTGAGGTG |
| | | | | G [SEQ ID NO: 58] |
| chr18 | 50792250 | + | 136 | TGAACATCTCCTGGATCTCTGCATCTCTGCTTGCTGTCTGCTTTGAGAGTAAATATATGATATAAAGGTGCTGT |
| | | | | AGAGGAGCCGTGCCGGCCTGTAGAGCGAGCGCCGGTGCACATTCAGCTTTGATATTCAGTGC [SEQ ID |
| | | | | NO: 59] |
| chr19 | 20077224 | + | 130 | AGCGTACTTTACTGTCTCACCACCGCTGCCGCTCCGGCGTGAGGGCACACTCCCACACACATGCGCGTGTATGT |
| | | | | TAAGTCGCTCTTGATGTGGTCATATTTATGTTCCTGTTAAAGTTTGAGCCGGCAGC [SEQ ID NO: 60] |
| chr1 | 23386154 | + | 134 | CATGTCCCTGTGGGTGGAGTTCATCACCGCCTCCGGTTATCTATCGGCACGAAAGATTCGCTCCCGCTTTCAGA |
| | | | | CGCTGGTTGCCCAGGCCGTGGATAAATGCAGCTACCGGGACGTGGTTAAGATGGTGGCGG [SEQ ID |
| | | | | NO: 61] |
| chr1 | 43259461 | + | 141 | ATAGCTGTACCAGTGTTTGTGTGTGTGTACCGGAGGAAGGTTTTGTGCTGGTATGCCGTTGTTGGGACCAAGAC |
| | | | | CCGGCACAGATGGGTCCTGCTGCAGTGGAGGGATCTGAGCGGGGTTAATCTGAGGGGGCAGGAAGGG [SEQ |
| | | | | ID NO: 62] |
| chr20 | 16578711 | − | 123 | CGGCCAGGTGGAGCAGAGACcctgccgcctccttcatctcctcgtcctcctcctcttcctcctcctcttcTTTG |
| | | | | ACCAGGATGTTTTCGGCGCGCCTCTTCTTCAGGGGCAGCGTGTCTCCTT [SEQ ID NO: 63] |
| chr20 | 21624045 | + | 147 | CTCTGACCCCTGCCTCCCCAGGAGCCTTGGCTCTGTCGGAGACGATTCAAATCACAGGGACTGTGGCTATCAAT |
| | | | | CAAACACGGGGACCGGAGCTCAACCGAAGAATATGTCAAGAAGCCATTTTAACATGTCAGGTTGTAGGCCGGC |
| | | | | [SEQ ID NO: 64] |
| chr20 | 26523373 | + | 149 | AATTCCAGCTCAAATCTTCTTCTAAACGAGTGATAACAACCCTAATCCAGTTTGGTCCGGAACCTGCAGACAAC |
| | | | | ACGGAAACTCATCTGGTTAAGCCTGGTATTTTATCTCTGCTAAACTGGATGCTTTCTCTCTCATTTACACGTTT |
| | | | | T [SEQ ID NO: 65] |
| chr20 | 28928268 | + | 129 | TATTGCTTCAAGTGTGCAACTTGTGCGCGGTTCCAAACAGGAAGTGGCGCGCTGGCAACCGGGAAGATATACAT |
| | | | | CACTGCACAGCGCTGATAAGTAAAAACTATAATTGCAGTATTATTGCTGACGATA [SEQ ID NO: 66 |
| chr21 | 23231786 | + | 149 | TTTACCCGGTTTTATAAATGCCCAACAATGCAAAGTTTGCGAAGCAAGAACACTCTGTCGTGCTGCTGATAGGC |
| | | | | CAAACTCTGTTCCAACGCCCGGGGAGAACTTTAAATAAACAATTGTCTTCATACTAAATGTCTGACAATCTAGT |
| | | | | C [SEQ ID NO: 67] |
| chr21 | 25150743 | + | 122 | CCGTCAGATTTGGAGCCACCTATGGAGGCAACCGTCTGTTCTCTGGAGCCCGGAGTGGTGGAGGCGCCAGCTCG |
| | | | | GCTCTGTCCCGTTCACTCGGCCTGGCAAGGGGAGGAGGTTTGGGTTTA [SEQ ID NO: 68] |
| chr24 | 19868851 | + | 148 | GCTCTTCCTACATGCTATGAAATTTCAGACATGTGCGGGCATTGAAAGGAGTCAAAGGCAATACCCAGAACAAA |
| | | | | TGTGTTGATAGAGATCCCGGATATTGTGGTGCCAACAACAAAACGGAGGGCAATGTAGACATAGATGTTAGGGG |
| | | | | [SEQ ID NO: 69] |
| chr25 | 14631230 | + | 129 | TTATCAGACAGTGGTAAATAAAGGTCTGGCCCGGGTTACCGCAGGCTGTCAGCAGGCCCGTCCCGGAGGGGAAA |
| | | | | TAAAACTCTTATTAACATGCTTCTGCTCATTGGTGCTGACAGCTTGATCAATCTG [SEQ ID NO: 70] |
| chr25 | 16313450 | + | 152 | GTGTTTGGAAGAATAGAGAGGCCTAGGTCTGGGTTAGAGGAGATTACACTGCAGGCACGTCGGGTGAAGACTGG |
| | | | | CTGGAGAAACTGCACACCGGCTCACTGTCACCATATTGTCCTTAAAGCAATAGATTGACATGAAGGGAATTTAC |
| | | | | ACAG [SEQ ID NO: 71] |
| chr25 | 36872756 | + | 153 | GAGCAGAGCTGAGGATTAACAGCAGTCTCTGACCTACAGCTGCGTTTCTGAGCAGCACCGCGGAGTCCAGAGCC |
| | | | | ATGAGATGATGGAGATCCTGACCTGAAGTGAGTCTGAGTGTGAATGAGCGGATCCGGATTGATCTGATGAGTGC |
| | | | | AGGAG [SEQ ID NO: 72] |
| chr25 | 6461988 | + | 146 | AAAAGTCAAAGCAGACAGGGAGTGGGTTTTTATGAACATGCATTTCTGAGCGCCGATGAAATTTTGGTCCTGGA |
| | | | | CAGATGGATGAATATCTGCCGGGAGATGGCGAACATGAAGAGAGTCAGAAATGGGAAGCCAAAGAGCAAAGG |
| | | | | [SEQ ID NO: 73] |
| chr2 | 8207957 | + | 137 | CAGGGCCGGTGACATTCTGCATCCCAGGCGCTCGCTGTTCTTTAAACACACTCCGATCAGTGGAGCAAAACTGT |
| | | | | GTGAGCCGGCTTCCGCGAATTAACCGCACATGCCAATGTTTTATTCACTTTGCAAGTCTGCGT [SEQ ID |
| | | | | NO: 74] |
| chr3 | 23616782 | + | 150 | TTATGTTTTATTTCATTCCCACCCCAGCGGAGAGCAGCGGTGGTGAGAAAAGCCCTCCGGGTTCTGCAGCTTCC |
| | | | | AAGAGAGCACGCACCGCTTACACCAGCGCACAGCTGGTGGAGCTCGAGAAGGAGTTTCACTTCAACCGATACCT |
| | | | | GT [SEQ ID NO: 75] |
| chr4 | 17690807 | + | 130 | GGCTAAACATGTGTTTTTGTGTGGCAGACGACTGATAAAGAGGCTCCGGGATTGGTCCGCATGCACACTCTGGC |
| | | | | CTACTTGAGCGGCTTTCCAGCATCGCTGAAGGAAACTGAGCAGCTCCGGGTCAAAC [SEQ ID NO: 76] |
| chr4 | 18675145 | + | 139 | ATTTCATCTGCAGTGACCACATACACACACACACTTTGCATCCACGGCCTACCTGGGAATCCGCCTGGTAGAGA |
| | | | | GATAATTACCGGATCACAATCCCCATCTCCTTTTTTTCGATTTTCAGACACACCTCTGTTTTGAA [SEQ ID |
| | | | | NO: 77] |
| chr5 | 51679905 | + | 149 | CCAAATGAAGCCATGGCTGTGTGATTGAGGTTTATAGTGGGAGGTCAGCAGTCTGGGCTCAGGAGTCCTCCGGG |
| | | | | CACCATAAATCACCACAGGCCAATAAACACAGACAGCAGAATTTCTCAGTATATAGACAGGTGTCAGAACTGCT |
| | | | | C [SEQ ID NO: 78] |

### Barcoding and DNA Sequencing

Oligonucleotides with attached MiSeq adaptors and barcodes were used for the barcoding reaction (Fluidigm PN100-4876). Barcoding was performed using 1× Green GoTaq Flexi Buffer, 0.05 U/μL of GoTaq Hot Start Polymerase, 4.5 mM MgCl₂, 200 μM of each dNTP, 25 μL of the pooled template after Sera-Mag Magnetic SpeedBeads (GE Healthcare Life Sciences) clean up. Cycling conditions for barcoding were as follows 94° C./5 mins; 9 cycles of 97° C./15 seconds, 60° C./30 seconds and 72° C./2 mins; 72° C./2 mins; 6° C./5 mins. Barcoding was performed using an Eppendorf ProS 96 or 384 thermocycler. Sequencing was performed on an Illumina Miseq using the MiSeq Reagent Kit v2 (300 cycle; PN MS-102-2002).

### Sequencing Data Analysis

Sequencing data was hard clipped by 15 bp at both 5′ and 3′ ends to remove adaptor sequences by SeqKit v 1.2 (Shen et al., 2016). Reads were aligned to a reduced representation of the genome focusing on a 500 bp upstream and downstream of the zebrafish age-associated sites. Reads were aligned using Bismark v 0.20.0 with the following options: --bowtie2 -N 1 -L 15 --bam -p 2 --score L,−0.6,−0.6 --non_directional and methylation calling was performed using bismark_methylation_extractor (Krueger and Andrews 2011). The methylation beta values were calculated using the bismark_methylation_extractor and calculating the percentage of reads that were methylated.

### Methylation Sensitive PCR

msPCR primers were designed using MethPrimer v2.0 (Li and Dahiya, 2002) which produces two pairs of primers for when the DNA is methylated and unmethylated (Table 6). msPCR was optimised using the protocol detailed previously (Huang et al., 2013) with the final cycling conditions: Initialisation step 95° C./15 mins, denaturation step 95° C./30 seconds, annealing 55° C./40 seconds and extension 72° C./40 seconds, for 40 cycles. msPCR was performed using an AllTaq Mastermix (Qiagen) with 1×SYBR Green (Thermo Fisher) in a Bio-Rad CFX96. The ΔCt values for each primer pair was used as a quantitative method for methylation. A leave-one-out cross validation approach was used to determine the level of precision for using msPCR to estimate age (Kuhn, 2008; Picard and Cook, 1984).

TABLE 6
Primers used in msPCR assay exemplified herein.

| CpG site | | | Methylated | |
|---|---|---|---|---|
| chr | position | strand | Forward | Reverse |
| chr12 | 21540399 | + | ATATATATAAACGGATGGTTTCGG [SEQ ID NO: 79] | TTATATAAAACTAAACGAACCTAACG [SEQ ID NO: 80] |
| chr12 | 35432443 | + | GGATAAGATAGGTGTTGTTGTTCG [SEQ ID NO: 81] | GTATAACCTCTTTCTATCATCCCG [SEQ ID NO: 82] |
| chr13 | 31180246 | + | TTGTGAGTTAATAAAGAAAAGAATAGAC [SEQ ID NO: 83] | AAATCCTCAAAATATCTAAAACCGAC [SEQ ID NO: 84] |
| chr13 | 38582448 | + | GAGAAGAAATGAAGATGATTACG [SEQ ID NO: 85] | ACCTATAACTACGTAAAAACAACGCA [SEQ ID NO: 86] |
| chr14 | 38455793 | − | TGAGTTATTATGGTAAGAAGAGTGC [SEQ ID NO: 87] | TATATTACAAAAACTAATTTCGCA [SEQ ID NO: 88] |
| chr14 | 45387151 | + | GATAAAAGGGGGTTTAATACGGT [SEQ ID NO: 89] | ATAAAATACCTAAAACAAATTAATCG [SEQ ID NO: 90] |
| chr17 | 52836692 | + | GCGAATATATAAAAGTAGAAGAAACGC [SEQ ID NO: 91] | TACCGCTTTAAATCAACGTAT [SEQ ID NO: 92] |
| chr18 | 38107080 | + | TAGATAGATGTAACGTTGCGAG [SEQ ID NO: 93] | CTTAATCTCACAATATAAAACGATAAACG [SEQ ID NO: 94] |
| chr18 | 50792250 | + | AGGTGTTGTAGAGGAGTCGTGTC [SEQ ID NO: 95] | TAATTCTCTATACTCTAAAACCCGA [SEQ ID NO: 96 |
| chr19 | 20077224 | + | AGATTTGTAAAAGTGTTGGTGC [SEQ ID NO: 97] | GCGCATATATATAAAAATATACCCTCAC [SEQ ID NO: 98] |
| chr1 | 23386154 | + | GCGGTGTTTAAGTTTAGCGAC [SEQ ID NO: 99] | GAATACGCAATTTCACTTCGC [SEQ ID NO: 100] |
| chr1 | 43259461 | + | GGGTTTTAATGAGGAAGACGATT [SEQ ID NO: 101] | CAAAACCCATCTATACCGAAT [SEQ ID NO: 102] |
| chr20 | 16578711 | − | TTGAATAGAAGTATTTAGATTTGCG [SEQ ID NO: 103] | CTCTACTCCACCTAACCGACG [SEQ ID NO: 104] |
| chr20 | 21624045 | + | TGGTTATTAATTAAATACGGGG [SEQ ID NO: 105] | AAATCTTACGAAACGTATCTCGCT [SEQ ID NO: 106] |
| chr20 | 26523373 | + | AGTAGGATGATTAAAGAATGTTAGCGA [SEQ ID NO: 107] | AACTTAACCAAATAAATTTCCGTAT [SEQ ID NO: 108] |
| chr20 | 28928268 | + | AGTTGTATATATAATAAAATAAAGACGTT [SEQ ID NO: 109] | CACAATAATATAAAAACAATAATTATACCG [SEQ ID NO: 110] |
| chr21 | 23231786 | + | ATAGAAGCGGAGTTATTAAGCGAA [SEQ ID NO: 111] | AAACTTATAAAACCAATACTCGAAA [SEQ ID NO: 112] |
| chr21 | 25150743 | + | ATGATAGAGTTAAGTTTGCGGAT [SEQ ID NO: 113] | CGAATAAACGAAACAAAACCG [SEQ ID NO: 114] |
| chr24 | 19868851 | + | TATGAAATTTTAGATATGTGCGGG [SEQ ID NO: 115] | ATCTATATCTACATTACCCTCCGTT [SEQ ID NO: 116] |
| chr24 | 4215673 | + | GATTGATCGGTAAATCGAGA [SEQ ID NO: 117] | TCCAAACAAACACTCCTAACGAT [SEQ ID NO: 180] |
| chr25 | 14631230 | + | TAGTGGTAAATAAAGGTTTGGTTCG [SEQ ID NO: 119] | TTTCAACCTCCATCAAAACG [SEQ ID NO: 120] |
| chr25 | 16313450 | + | AGAGGAGATTATATTGTAGGTACGTCG [SEQ ID NO: 121] | TATATAAACAATCTAAACTACACGACC [SEQ ID NO: 122] |
| chr25 | 36872756 | + | AGTTTGAGTGTGAATGAGCG [SEQ ID NO: 123] | AACTCTCGAACGAAACCGTC [SEQ ID NO: 124] |
| chr25 | 6461988 | + | GGTAATGGTTTAAATATGTGGTTCG [SEQ ID NO: 125] | ACGTTAAATTAAATCAACACGTTA [SEQ ID NO: 126] |
| chr2 | 8207957 | + | TGCGTATCGTAGGGATGTTC [SEQ ID NO: 127] | ATATACGATTAATTCGCGAAAACC [SEQ ID NO: 128] |
| chr3 | 23616782 | + | GTTTTATTAGTGGGAACGATG [SEQ ID NO: 129] | CGATTAAAATAAAACTCCTTCTCG [SEQ ID NO: 130] |
| chr4 | 17690807 | + | GTTTTATTAGTGGGAACGATG [SEQ ID NO: 131] | CGATTAAAATAAAACTCCTTCTCG [SEQ ID NO: 132] |
| chr4 | 18675145 | + | AATCGACGAGTGAGACGGTT [SEQ ID NO: 133] | AAACAAAAATATATCTAAAAATCGAAA [SEQ ID NO: 134] |
| chr5 | 51679905 | + | AAAAGGTTGTTGAGGTTGATACG [SEQ ID NO: 135] | TTAACCTTAAACCTTATACCGAAA [SEQ ID NO: 136] |
| CpG site | | | Unmethylated | |
| chr12 | 21540399 | + | ATATAAATGGATGGTTTTGGGA [SEQ ID NO: 137] | TTATATAAAACTAAACAAACCTAACATTC [SEQ ID NO: 138] |
| chr12 | 35432443 | + | ATAAGATAGGTGTTGTTGTTTGG [SEQ ID NO: 139] | TCATATAACCTCTTTCTATCATCCCA [SEQ ID NO: 140] |
| chr13 | 31180246 | + | TGTGAGTTAATAAAGAAAAGAATAGAT [SEQ ID NO: 141] | AAAATCCTCAAAATATCTAAAACCAAC [SEQ ID NO: 142] |
| chr13 | 38582448 | + | GAGAAGAAATGAAGATGATTATG [SEQ ID NO: 143] | CTACCTATAACTACATAAAAACAACACAAC [SEQ ID NO: 144] |
| chr14 | 38455793 | - | GAGTTATTATGGTAAGAAGAGTGTG [SEQ ID NO: 145] | TATATTACAAAAACTAATTTCACAA [SEQ ID NO: 146] |
| chr14 | 45387151 | + | AAGATAAAAGGGGGTTTAATATGGTA [SEQ ID NO: 147] | AAATACCTAAAACAAATTAATCATTC [SEQ ID NO: 148] |
| chr17 | 52836692 | + | GTGAATATATAAAAGTAGAAGAAATGTGTA [SEQ ID NO: 149] | AATACCACTTTAAATCAACATATTCCC [SEQ ID NO: 150] |
| chr18 | 38107080 | + | GGTAGATAGATGTAATGTTGTGAG [SEQ ID NO: 151] | TCACAATATAAAACAATAAACAAAA [SEQ ID NO: 152] |
| chr18 | 50792250 | + | GTAGAGGAGTTGTGTTGGTT [SEQ ID NO: 153] | AATTCTCTATACTCTAAAACCCAAT [SEQ ID NO: 154] |
| chr19 | 20077224 | + | TGTAAAAGTGTTGGTGTGTG [SEQ ID NO: 155] | ACACACATATATATAAAAATATACCCTCAC [SEQ ID NO: 156] |
| chr1 | 23386154 | + | GTGGTGTTTAAGTTTAGTGATGG [SEQ ID NO: 157] | CCAAATACACAATTTCACTTCACTC [SEQ ID NO: 158] |
| chr1 | 43259461 | + | GGGGTTTTAATGAGGAAGATGAT [SEQ ID NO: 159] | AACAAAACCCATCTATACCAAAT [SEQ ID NO: 160] |
| chr20 | 16578711 | − | TGAATAGAAGTATTTAGATTTGTG [SEQ ID NO: 161] | CTCTACTCCACCTAACCAACAT [SEQ ID NO: 162] |
| chr20 | 21624045 | + | TGTGGTTATTAATTAAATATGGGG [SEQ ID NO: 163] | CTTTCAAATCTTACAAAACATATCTCACTA [SEQ ID NO: 164] |
| chr20 | 26523373 | + | GAAGTAGGATGATTAAAGAATGTTAGTGAG [SEQ ID NO: 165] | AAACTTAACCAAATAAATTTCCAT [SEQ ID NO: 166] |
| chr20 | 28928268 | + | TTAAATAGGAAGTGGTGTGTTG [SEQ ID NO: 167] | AAACTATTAATATACAAATCCACAA [SEQ ID NO: 168] |
| chr21 | 23231786 | + | TGGATAGAAGTGGAGTTATTAAGTGAA [SEQ ID NO: 169] | AAAACTTATAAAACCAATACTCAAAA [SEQ ID NO: 170] |
| chr21 | 25150743 | + | AAAATGATAGAGTTAAGTTTGTGGAT [SEQ ID NO: 171] | ACCAAATAAACAAAACAAAACCAAA [SEQ ID NO: 172] |
| chr24 | 19868851 | + | TATGAAATTTTAGATATGTGTGGG [SEQ ID NO: 173] | AACATCTATATCTACATTACCCTCCATT [SEQ ID NO: 174] |
| chr24 | 4215673 | + | GAAATGTAGATTGATTGGTAAATTGAG [SEQ ID NO: 175] | CAAACAAACACTCCTAACAATC [SEQ ID NO: 176] |
| chr25 | 14631230 | + | AATAAAGGTTTGGTTTGGGT [SEQ ID NO: 177] | TCAACCTCCATCAAAACATCC [SEQ ID NO: 178] |
| chr25 | 16313450 | + | GGAGATTATATTGTAGGTATGTTGGG [SEQ ID NO: 179] | CTATATAAACAATCTAAACTACACAACC [SEQ ID NO: 180] |
| chr25 | 36872756 | + | GTTTGAGTGTGAATGAGTGGAT [SEQ ID NO: 181] | AAACTAAAACTCTCAAACAAAACCATC [SEQ ID NO: 182] |
| chr25 | 6461988 | + | AATGGTTTAAATATGTGGTTTGG [SEQ ID NO: 183] | CACATTAAATTAAATCAACACATTA [SEQ ID NO: 184] |
| chr2 | 8207957 | + | GTGTATTGTAGGGATGTTTGTAG [SEQ ID NO: 185] | AAAACATTAACATATACAATTAATTCACAA [SEQ ID NO: 186] |
| chr3 | 23616782 | + | ATTTTAGTGGAGAGTAGTGGTG [SEQ ID NO: 187] | CAATTAAAATAAAACTCCTTCTCAAAC [SEQ ID NO: 188] |
| chr4 | 17690807 | + | GTGTTTTTGTGTGGTAGATGA [SEQ ID NO: 189] | AAAACTACTCAATTTCCTTCAACAATA [SEQ ID NO: 190] |
| chr4 | 18675145 | + | GTTATGAATTGATGAGTGAGATGGTT [SEQ ID NO: 191] | AAAACAAAAATATATCTAAAAATCAAAA [SEQ ID NO: 192] |
| chr5 | 51679905 | + | TAAAAGGTTGTTGAGGTTGATATGTA [SEQ ID NO: 193] | CTTAACCTTAAACCTTATACCAAAA [SEQ ID NO: 194] |

### Example 2—Age Estimation

Age Estimation from Bisulfite Sequencing

RRBS data was used to generate a model to estimate age in Zebrafish. On average, 45.1 million reads per RRBS library was aligned to the zebrafish genome with an alignment rate of 87.4%. This resulted in a total of 524,038 CpG sites with adequate coverage in at least 90% of all samples. Of these sites, 60.9% were found to be within gene bodies such as exons. Global methylation was found to be on average 79.5% similar to what has been observed in other zebrafish tissues (Falisse et al., 2018; Ortega-Recalde et al., 2019; Adam et al., 2019). No correlation was found between global methylation and age (r=0.030, p-value=0.77). However, methylation at 1,311 CpG sites was found to significantly correlate (p-value<0.05) with increasing age (Table 1). This suggests specific CpG sites are associated with ageing but not global methylation.

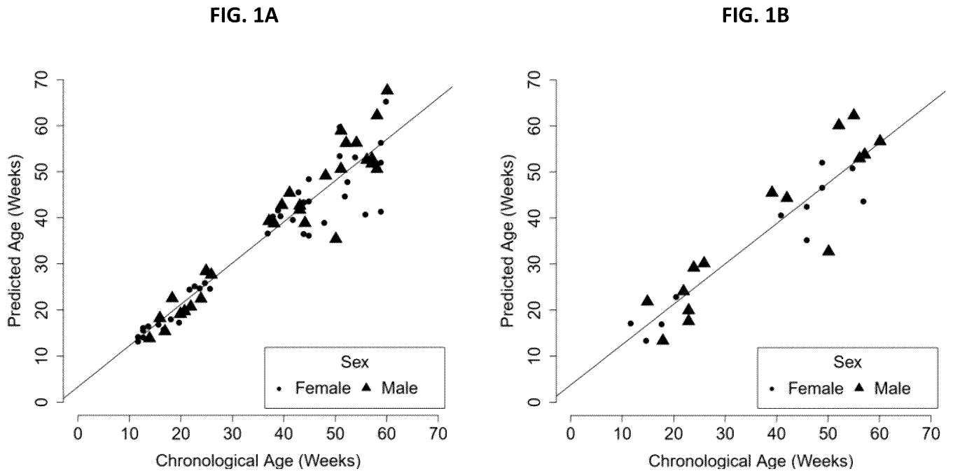

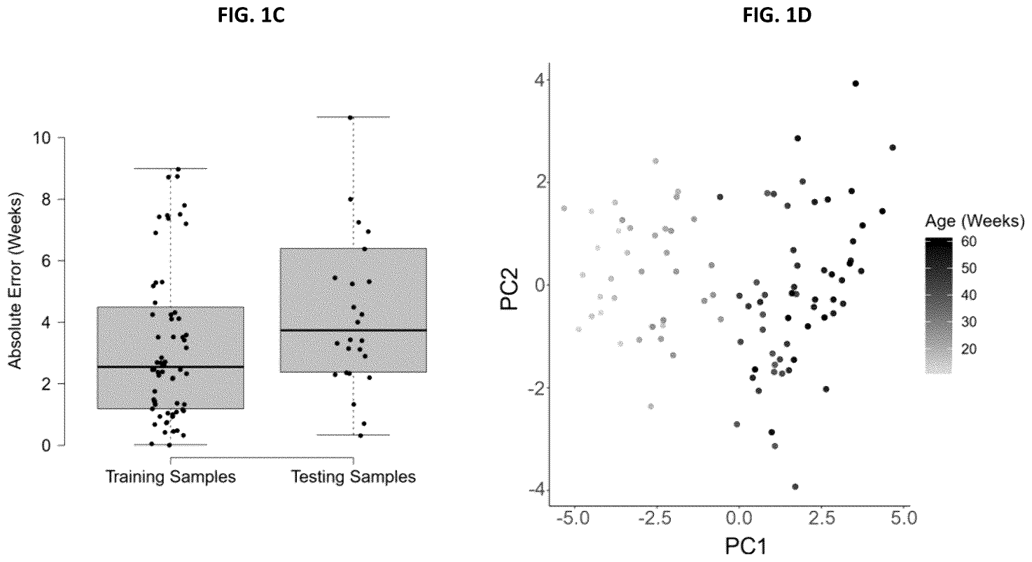

In order to predict age from CpG methylation samples were randomly assigned to either a training or a testing data set. Age was transformed to natural log to fit a linear model. Using an elastic net regression model, the age of the zebrafish was regressed over CpG site methylation in the training data set. This identified 29 CpG sites (Table 2) that could be used estimate the age of zebrafish. A high correlation (cor=0.95, p-value<2.20×10⁻¹⁶) between the chronological and known age of the zebrafish was observed (FIG. 1a). In addition, a high correlation (cor=0.92, p-value=9.56×10⁻¹¹) in the testing data set was also observed (FIG. 1b). A median absolute error (MAE) rate of 3.7 weeks was observed in the testing data set (FIG. 1c) and no statistical difference was observed between the absolute error rate between the training and testing data sets (p-value=0.14, t-test). The similar performance rate between the training and testing data sets suggests a low possibility of overfitting.

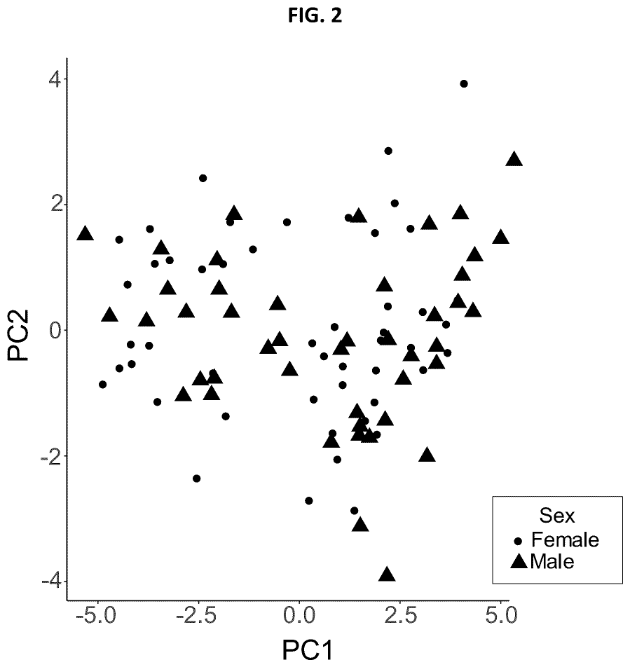

A principle component analysis (PCA) was used to visualise the separation of samples by age using the methylation levels of the 29 CpG sites (FIG. 1d). This unsupervised clustering shows separation of the samples solely on increasing age, suggesting the 29 CpG sites are suitable candidates to estimate age. No significant GO enrichment was found using the 29 CpG sites. Samples were not found to separate by sex which was the only other phenotypic difference between individuals (FIG. 2).

### Epigenetic Drift

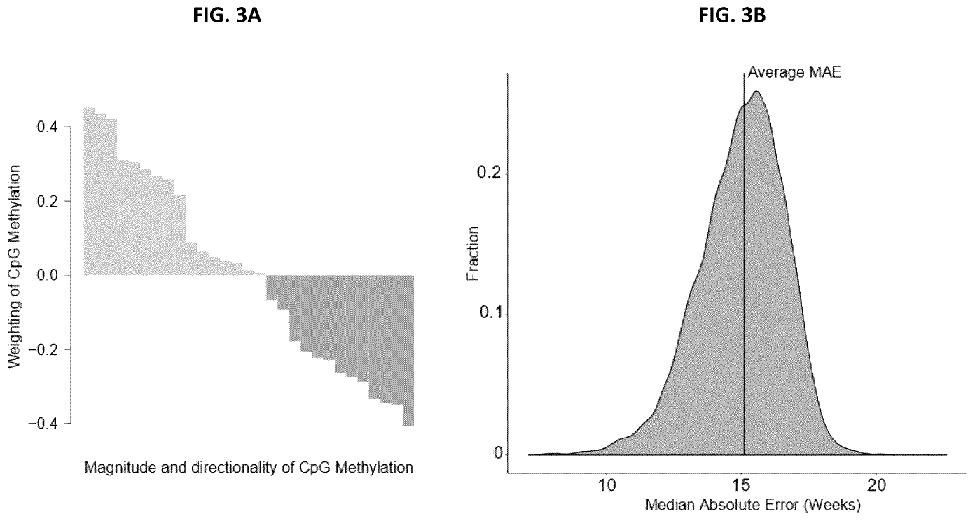

The elastic net regression model identified 29 age-associated CpG sites that can be used to estimate age. However, these sites differ in terms of importance. Each CpG site has a different weight (FIG. 3a), but collectively could be used to estimate age. This demonstrates that despite each CpG site having a different level of age-association, they can be used collectively in a method to estimate age of a fish. To assess the level of age-association in other age-associated CpG sites we used a ridge model (α-parameter=0 in glmnet) and randomly selected 29 CpG sites out of the possible 524,038 CpG sites. This was repeated 10,000 times and produced an average MAE of 15.1 weeks (FIG. 3b). This analysis demonstrates that any of the CpG sites identified have some level of age-association, however others (for example, those listed in Tables 1, 2 or 3) are more associated with age than others.

### Methylation Sensitive PCR

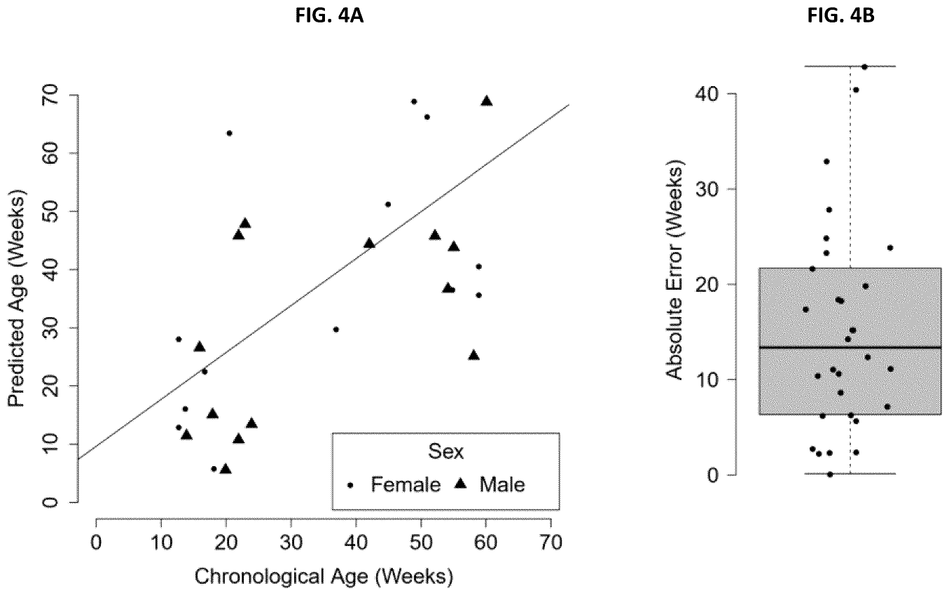

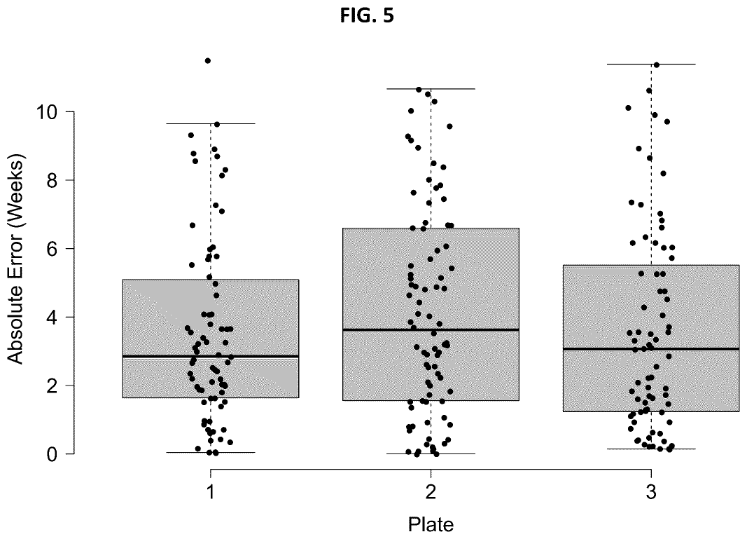

To reduce the burden of resources, computational time and/or cost that is involved in using RRBS as a method to estimate age, the Inventors set out to determine a more practical and cost-effective method. Methylation sensitive PCR (msPCR) has previously been used as an alternative method to assay methylation of CpG sites (Herman et al., 1996). Despite a significant correlation between the chronological and predicted age (cor=0.62, p-value=0.00028) the MAE rate increased 261% from what was found in RRBS to 13.4 weeks (FIG. 4). This suggests msPCR is not as sensitive as RRBS for detecting the minute changes in methylation used for age-estimation.

### Multiplex PCR Followed by Sequencing

Multiplex PCR followed by sequencing was also investigated as an alternative to RRBS for measuring the level of methylated cytosine at multiple CpG sites. For each CpG site, primers were designed to amplify a 140 bp amplicon containing an age-associated CpG site. Three primer pairs were unable to be optimised as part of the overall multiplex PCR assay and were removed from the analysis. The remaining 26 CpG sites were remodelled using the RRBS methylation data by applying the ridge model component in the glmnet function (α-parameter=0) resulting in alternative weights for each site (Table 3). A generalised linear model was applied to the raw prediction values from the elastic net regression model (sum of the coefficient weights multiplied by the DNA methylation beta values). The final model to estimate age in zebrafish is:

ln(age)=1.008x

where x is the sum of the methylation beta values multiplied by their weights listed in Table 3 for each sample.

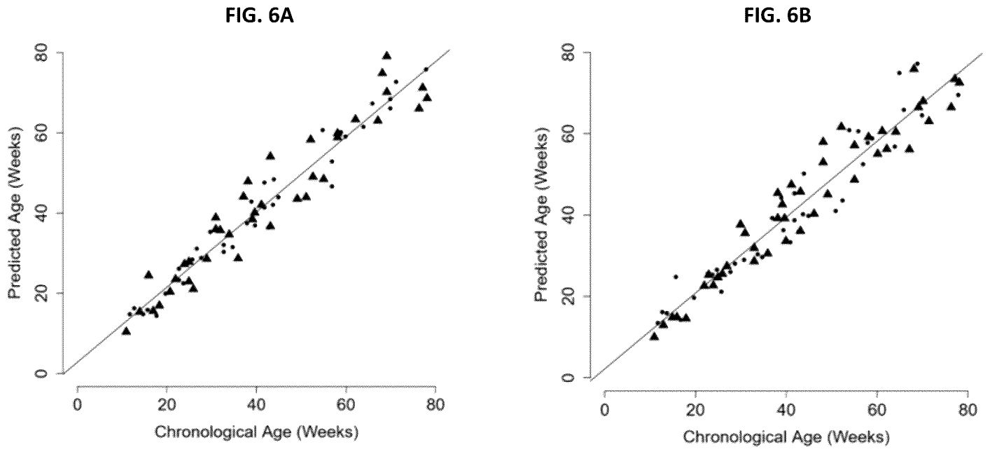

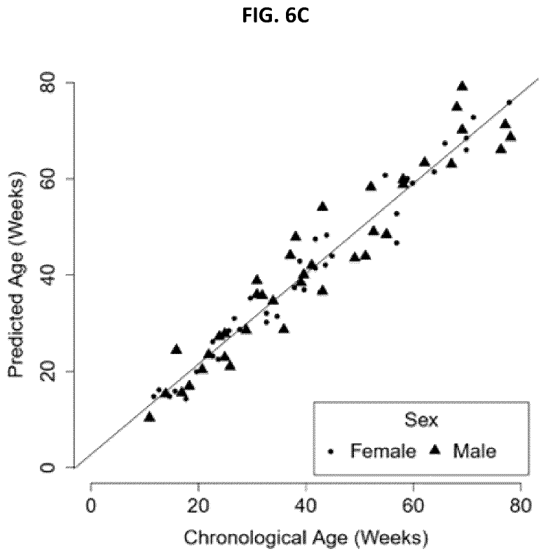

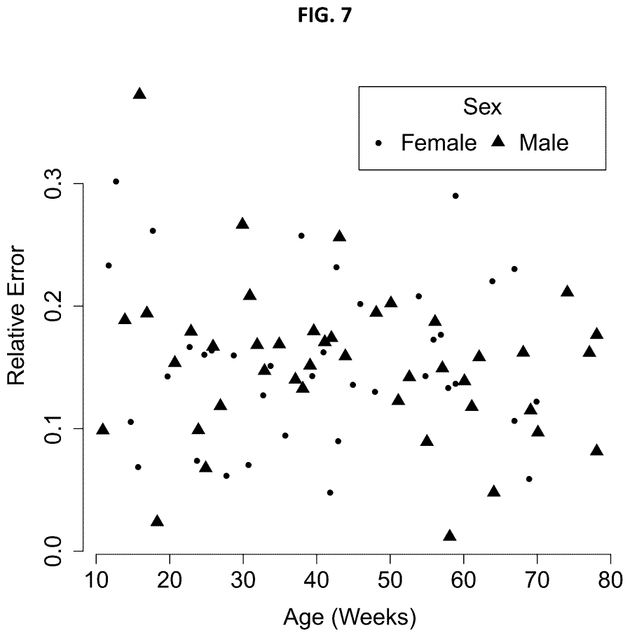

The final model was used to estimate the age of the zebrafish from the methylation beta value determined using multiplex PCR followed by DNA sequencing. The estimated age was compared to the calculated age to assess the accuracy of the model. A high average correlation across the replicates between the chronological and predicted age (cor=0.97) and a low average MAE of 3.18 weeks (FIG. 5 and FIG. 6) was observed. In addition, no statistically significant difference was found between the absolute error rates between replicates (p-value=0.366, ANOVA), suggesting the method was highly reproducible. In addition, no statistically significant difference was found between the absolute error rate in the RRBS testing data set and the multiplex PCR samples (p-value=0.23, t-test). This suggests RRBS and multiplex PCR return similar sensitivities in methylation values given the similar absolute error rates. Moreover, no significant difference was found in the absolute error rate compared to the age of the zebrafish. Therefore, both RRBS and multiplex PCR are suitable for use in methods of estimating age. Multiplex PCR provides a cost effective method to measure methylation from multiple sites and estimate age.

### Example 3—Age Estimation for Atlantic Salmon

### DNA Extraction and Bisulfite Treatment

Atlantic salmon fin clip samples and associated age information were obtained from a Tasmanian based salmon fish farm. Approximately 15 mg of tissue was used for DNA extraction. DNA was extracted using the DNeasy Blood & Tissue Kit (QIAGEN) as instructed in the manufacture's protocol. Extracted DNA was bisulfite converted using the protocol as previously described (Clark et al., 2006).

### Identification of Conserved Age Associated CpG Sites

The genome of Atlantic salmon was analysed for candidate age-associated CpG sites corresponding to an age-associated CpG site of Zebrafish listed in Table 1. Genome pairwise alignment was performed between the zebrafish reference genome danRer10 (Illumina iGenomes) and the Atlantic Salmon genome (ICSASG_v2). CpG sites conserved between zebrafish and Atlantic salmon were identified using LASTZ v1.04.00 with the following conditions: [multiple] --notransition --step=20 -nogapped (Harris 2007). A total of 1,311 CpG sites were analysed to determine if they are conserved between the two species. Genome pairwise alignment identified a total of 131 CpG sites that are both age-associated in zebrafish and conserved between zebrafish and Atlantic Salmon. These candidate age-associated CpG sites are listed in Table 7. The candidate age-associated CpG sites in Atlantic salmon listed in Table 7 were used to develop a DNA age estimator.

TABLE 7
Candidate age-associated CpG sites from Atlantic Salmon. Genomic
locations are from the Atlantic Salmon genome (ICSASG_v2).

| Zebrafish Coordinate | | | Atlantic Salmon Coordinate | | | Age Association in Zebrafish | |
|---|---|---|---|---|---|---|---|
| chr | position | strand | contig | position | strand | correlation | p-value |
| chr15 | 17357026 | + | NC_027303.1 | 50918378 | − | 0.968883543 | 3.95E−06 |
| chr15 | 47234443 | + | NC_027308.1 | 130985161 | + | 0.376341991 | 0.000200895 |
| chr21 | 32529874 | + | NC_027303.1 | 31851318 | + | −0.38997311 | 0.000846332 |
| chr7 | 8087597 | + | NC_027309.1 | 50163068 | − | −0.449553599 | 0.001193076 |
| chr13 | 24280410 | + | NC_027301.1 | 42036470 | + | 0.651541353 | 0.001375676 |
| chr4 | 32478295 | + | NC_027302.1 | 34712218 | + | −0.326039135 | 0.001426135 |
| chr3 | 23093561 | + | NC_027305.1 | 41492200 | + | −0.320944435 | 0.001813068 |
| chr14 | 30746839 | + | NC_027308.1 | 78024899 | + | −0.318528116 | 0.002090037 |
| chr10 | 37126370 | + | NC_027308.1 | 120663254 | + | −0.413205802 | 0.002103962 |
| chr7 | 38385417 | + | NC_027309.1 | 103032401 | − | 0.315386696 | 0.002462809 |
| chr2 | 1162082 | + | NC_027309.1 | 25717750 | − | 0.322729261 | 0.002591851 |
| chr16 | 2641633 | + | NC_027301.1 | 17141560 | + | 0.494717886 | 0.002936565 |
| chr11 | 12237381 | + | NC_027305.1 | 70893269 | + | −0.302626264 | 0.003194052 |
| chr21 | 6748808 | + | NC_027300.1 | 140074703 | + | 0.344282561 | 0.003518393 |
| chr7 | 47984196 | + | NC_027309.1 | 63172783 | − | 0.483979141 | 0.00373004 |
| chr6 | 10299607 | + | NC_027301.1 | 43618872 | + | 0.339731076 | 0.003748841 |
| chr16 | 68735 | + | NC_027301.1 | 331670 | + | −0.374723588 | 0.003754416 |
| chr17 | 41476353 | + | NC_027305.1 | 51767458 | − | 0.294307638 | 0.003985601 |
| chr17 | 732992 | + | NC_027300.1 | 82774975 | + | −0.293623853 | 0.004282587 |
| chr2 | 8193411 | + | NC_027302.1 | 36817896 | − | −0.559546153 | 0.004469557 |
| chr7 | 25869438 | + | NC_027306.1 | 23003313 | + | −0.653153332 | 0.004469947 |
| chr18 | 22764335 | + | NC_027309.1 | 79692472 | − | −0.315222177 | 0.005544174 |
| chr16 | 49140690 | + | NC_027301.1 | 25550820 | + | −0.299461199 | 0.005652394 |
| chr1 | 43745060 | + | NC_027304.1 | 30076702 | − | −0.737842424 | 0.006155412 |
| chr13 | 48241878 | + | NC_027300.1 | 85360242 | − | −0.280767225 | 0.006409426 |
| chr12 | 9743929 | + | NC_027300.1 | 89493581 | + | 0.28457814 | 0.006558698 |
| chr1 | 14453599 | + | NC_027307.1 | 26002570 | + | 0.324555128 | 0.006928768 |
| chr4 | 1480122 | + | NC_027309.1 | 57660635 | + | 0.583229701 | 0.006950113 |
| chr6 | 58779489 | + | NC_027302.1 | 42502551 | + | −0.704059589 | 0.00722749 |
| chr20 | 9312922 | + | NC_027305.1 | 76677672 | − | 0.511607687 | 0.00755174 |
| chr20 | 5257938 | + | NC_027308.1 | 43253542 | − | −0.276447589 | 0.007640966 |
| chr22 | 33941568 | + | NC_027306.1 | 35711055 | − | 0.315123785 | 0.007881391 |
| chr10 | 134655 | + | NC_027300.1 | 97661352 | + | 0.290629885 | 0.008486164 |
| chr3 | 19106044 | + | NC_027302.1 | 68905377 | + | 0.304481599 | 0.008816336 |
| chr22 | 23236563 | + | NC_027300.1 | 146922099 | − | −0.279892457 | 0.009477059 |
| chr4 | 65086486 | + | NC_027300.1 | 73738349 | − | −0.279777318 | 0.009952581 |
| chr17 | 38466634 | + | NC_027300.1 | 40884662 | + | 0.533961576 | 0.010478507 |
| chr20 | 23433532 | + | NC_027309.1 | 30492621 | − | 0.569976673 | 0.010839258 |
| chr4 | 3336579 | + | NC_027306.1 | 35582104 | + | −0.304287865 | 0.011021446 |
| chr16 | 33254083 | + | NC_027304.1 | 52203877 | − | −0.261662759 | 0.011290967 |
| chr25 | 16343105 | + | NC_027309.1 | 99182497 | − | −0.294686723 | 0.011379692 |
| chr25 | 974579 | + | NC_027308.1 | 61391706 | − | −0.26682999 | 0.011481545 |
| chr14 | 2345331 | + | NC_027304.1 | 35371127 | − | −0.277640936 | 0.011556359 |
| chr14 | 37251768 | + | NC_027304.1 | 16955205 | + | −0.272669732 | 0.011579568 |
| chr22 | 17546119 | + | NC_027300.1 | 129472787 | + | −0.265923754 | 0.011776143 |
| chr3 | 5695875 | + | NC_027302.1 | 64012306 | + | −0.336749413 | 0.011939231 |
| chr17 | 8468762 | + | NC_027300.1 | 63287595 | − | −0.287830561 | 0.012277931 |
| chr10 | 24757446 | + | NC_027308.1 | 125230978 | + | −0.626229227 | 0.012501482 |
| chr5 | 58167285 | + | NC_027308.1 | 98147779 | − | −0.258860937 | 0.013755349 |
| chr1 | 30545540 | + | NC_027300.1 | 58014749 | − | 0.615810869 | 0.014518716 |
| chr2 | 6379882 | + | NC_027302.1 | 14618842 | + | 0.383121654 | 0.014681063 |
| chr1 | 13619475 | + | NC_027300.1 | 102870971 | + | 0.335962041 | 0.015940056 |
| chr10 | 15845129 | + | NC_027310.1 | 15681149 | + | 0.698538566 | 0.016795575 |
| chr8 | 18518961 | + | NC_027309.1 | 10588861 | − | −0.514689625 | 0.016969763 |
| chr21 | 253721 | + | NC_027305.1 | 14087297 | + | −0.25217242 | 0.017124471 |
| chr15 | 16387175 | + | NC_027308.1 | 114191088 | + | 0.389406063 | 0.017207018 |
| chr3 | 5187775 | + | NC_027304.1 | 27014317 | − | 0.378942643 | 0.017360353 |
| chr21 | 2202642 | + | NC_027303.1 | 4734518 | + | −0.252571628 | 0.01759258 |
| chr23 | 40383210 | + | NC_027301.1 | 37010921 | − | −0.267607334 | 0.017852581 |
| chr8 | 19637396 | + | NC_027303.1 | 8179977 | − | 0.582674172 | 0.017854389 |
| chr21 | 30233261 | + | NC_027303.1 | 74164827 | − | 0.243609507 | 0.017979343 |
| chr5 | 71613665 | + | NC_027301.1 | 15779838 | + | −0.275802671 | 0.018186775 |
| chr3 | 60744371 | + | NC_027301.1 | 42793379 | + | −0.270204074 | 0.019051356 |
| chr12 | 35912024 | + | NC_027300.1 | 96728640 | + | 0.355175941 | 0.019429321 |
| chr11 | 12330890 | + | NC_027305.1 | 83104817 | − | −0.241663033 | 0.019611561 |
| chr15 | 44116998 | + | NC_027305.1 | 3358345 | + | 0.24184575 | 0.02020184 |
| chr2 | 10263502 | + | NC_027302.1 | 11427598 | + | 0.240921644 | 0.020698918 |
| chr18 | 44411207 | + | NC_027308.1 | 138755862 | − | 0.237935848 | 0.020928141 |
| chr17 | 36699079 | + | NC_027305.1 | 52341142 | − | −0.263660823 | 0.021372928 |
| chr16 | 50254087 | + | NC_027301.1 | 25401320 | + | 0.236983132 | 0.021461906 |
| chr19 | 17057661 | + | NC_027302.1 | 48264399 | − | 0.256582975 | 0.021593954 |
| chr13 | 25722247 | + | NC_027300.1 | 59755239 | − | 0.266313254 | 0.02181931 |
| chr15 | 47298799 | + | NC_027305.1 | 72133159 | + | −0.236117685 | 0.021956841 |
| chr17 | 52770474 | + | NC_027300.1 | 30846145 | − | 0.234801447 | 0.023482635 |
| chr12 | 1483691 | + | NC_027302.1 | 84367807 | − | 0.232995264 | 0.023824509 |
| chr20 | 34183179 | + | NC_027302.1 | 15017906 | − | −0.235259109 | 0.023979469 |
| chr16 | 17296452 | + | NC_027301.1 | 8969234 | − | 0.236297288 | 0.024946375 |
| chr21 | 43005697 | + | NC_027303.1 | 70034578 | − | 0.358331258 | 0.025095104 |
| chr3 | 51652824 | + | NC_027305.1 | 3566898 | + | −0.235104068 | 0.025708942 |
| chr12 | 24783489 | + | NC_027300.1 | 85334246 | + | 0.355141544 | 0.026515353 |
| chr22 | 1387254 | + | NC_027301.1 | 71532758 | + | −0.291940397 | 0.027558298 |
| chr14 | 2168951 | + | NC_027303.1 | 39302544 | + | 0.258920311 | 0.028080551 |
| chr22 | 4204857 | + | NC_027309.1 | 17497966 | − | −0.276704966 | 0.028138036 |
| chr16 | 32288631 | + | NC_027304.1 | 46165457 | + | −0.232686546 | 0.028210811 |
| chr16 | 739507 | + | NC_027301.1 | 828921 | + | 0.60490816 | 0.028501648 |
| chr14 | 2165004 | + | NC_027303.1 | 39302205 | + | −0.257899 | 0.028728631 |
| chr18 | 3143565 | + | NC_027308.1 | 110172382 | + | 0.248702084 | 0.029179739 |
| chr7 | 22128670 | + | NC_027303.1 | 38885318 | + | 0.464736283 | 0.02931927 |
| chr14 | 36379887 | + | NC_027303.1 | 12632470 | + | 0.224938196 | 0.030177794 |
| chr14 | 2149839 | + | NC_027303.1 | 39302544 | + | −0.649021375 | 0.030720732 |
| chr23 | 31628732 | + | NC_027305.1 | 85272598 | − | −0.227815322 | 0.030810031 |
| chr5 | 35671018 | + | NC_027303.1 | 38675341 | + | −0.226740018 | 0.031630451 |
| chr16 | 7277579 | + | NC_027304.1 | 57998480 | + | 0.283794565 | 0.032408736 |
| chr12 | 47334248 | + | NC_027308.1 | 20375348 | + | −0.282761301 | 0.033071956 |
| chr2 | 4367487 | + | NC_027302.1 | 33540186 | + | 0.417926492 | 0.033624776 |
| chr14 | 2129904 | + | NC_027303.1 | 39448732 | + | 0.56786301 | 0.034145023 |
| chr19 | 1627762 | + | NC_027301.1 | 2833473 | + | 0.220911746 | 0.034331248 |
| chr7 | 24537570 | + | NC_027309.1 | 697280 | − | −0.220681647 | 0.034523872 |
| chr3 | 18683277 | + | NC_027301.1 | 62508156 | + | −0.260623798 | 0.034554699 |
| chr21 | 30233442 | + | NC_027303.1 | 74165008 | − | −0.23786054 | 0.03478519 |
| chr22 | 37962037 | + | NC_027300.1 | 71304745 | + | −0.2579366 | 0.035085357 |
| chr15 | 30555023 | + | NC_027303.1 | 42335448 | − | −0.217453205 | 0.035261609 |
| chr10 | 21822975 | + | NC_027308.1 | 66909646 | + | −0.304556204 | 0.035318077 |
| chr19 | 11152824 | + | NC_027300.1 | 141104293 | + | 0.231294023 | 0.035391497 |
| chr15 | 34884834 | + | NC_027301.1 | 26588161 | + | 0.220650058 | 0.035575294 |
| chr2 | 7266340 | + | NC_027302.1 | 8733681 | + | −0.413397141 | 0.03579974 |
| chr2 | 35878840 | + | NC_027302.1 | 21240140 | − | 0.216264373 | 0.036300079 |
| chr12 | 9563630 | + | NC_027300.1 | 3295402 | + | 0.233643631 | 0.036992577 |
| chr13 | 42191818 | + | NC_027300.1 | 50071712 | + | −0.436947153 | 0.037089306 |
| chr20 | 35156150 | + | NC_027305.1 | 72678812 | + | 0.244226181 | 0.037315157 |
| chr13 | 11870444 | + | NC_027300.1 | 47441710 | + | 0.298324398 | 0.037339535 |
| chr10 | 35045356 | + | NC_027310.1 | 87433575 | + | −0.267040925 | 0.037483956 |
| chr18 | 15010635 | + | NC_027309.1 | 95197445 | − | 0.216624727 | 0.03916397 |
| chr7 | 5326746 | + | NC_027306.1 | 25736220 | + | 0.277897952 | 0.039950008 |
| chr20 | 39683166 | + | NC_027305.1 | 56327093 | + | −0.222746781 | 0.041692005 |
| chr4 | 22089381 | + | NC_027306.1 | 44020379 | + | 0.213532365 | 0.042121872 |
| chr7 | 38477940 | + | NC_027302.1 | 53759243 | + | −0.215816987 | 0.0422311 |
| chr15 | 16216506 | + | NC_027308.1 | 114101382 | + | −0.212245007 | 0.042239574 |
| chr17 | 592752 | + | NC_027308.1 | 18987595 | − | 0.209670125 | 0.04253656 |
| chr2 | 266670 | + | NC_027302.1 | 29105824 | + | 0.262729598 | 0.042553644 |
| chr16 | 12825400 | + | NC_027301.1 | 11051947 | + | −0.209503187 | 0.042705337 |
| chr18 | 15132113 | + | NC_027306.1 | 29689351 | − | −0.310335448 | 0.042831396 |
| chr14 | 2382080 | + | NC_027304.1 | 35453027 | + | −0.371560452 | 0.043209994 |
| chr18 | 23659524 | + | NC_027309.1 | 81460242 | + | −0.210801873 | 0.043693288 |
| chr4 | 5329774 | + | NC_027306.1 | 52831544 | + | 0.234874161 | 0.043973875 |
| chr19 | 48124943 | + | NC_027301.1 | 27743870 | − | 0.220865587 | 0.044800984 |
| chr15 | 681621 | + | NC_027301.1 | 71676428 | − | −0.219849236 | 0.047187929 |
| chr18 | 5599371 | + | NC_027300.1 | 147561591 | − | 0.363631124 | 0.048237531 |
| chr2 | 19078782 | + | NC_027300.1 | 152259762 | − | −0.213330518 | 0.048591067 |
| chr24 | 38867550 | + | NC_027301.1 | 48432202 | + | 0.257855482 | 0.048640423 |
| chr17 | 43986249 | + | NC_027300.1 | 25559015 | − | −0.205899699 | 0.048944936 |

T
1
1

### Primer Design and Single-Plex Testing

Primers were designed using Primersuite (Lu et al., 2017) and were designed for one PCR reaction pool. Initially, the top 60 age associated and conserved CpG sites were targeted for primer design. A total of 48 primer pairs were successfully designed for one multiplex PCR reaction pool.

Each individual primer pair was tested individually using the GoTaq Hot Start Polymerase (Promega) using the manufacture's cycling conditions: 95° C., 2 min; 35 cycles (95° C., 1 min; 65° C., 1 min; 72° C., 30 s); 72° C., 5 min; 10° C. hold. Gel electrophoresis with sodium borate buffer using a 1.5% agarose gel was used to visualise PCR products. All primer pairs produced a single amplicon and were used as part of the multiplex PCR (data not shown).

### Multiplex PCR

The final multiplex PCR reaction consisted of 1× Green GoTaq Flexi Buffer (Promega), 0.025 U/μL of GoTaq Hot Start Polymerase (Promega), 4.5 mM MgCl2 (Promega), 0.5× Combinatorial Enhancer Solution (CES) (Refer to Ralser et al., 2006), 200 μM of each dNTP (Fisher Biotec), 15 mM TMAC (Sigma-Aldrich), 200 nM forward primer, 200 nM reverse primer and 2 ng/μL bisulfite treated DNA. Cycling conditions were 94° C./5 mins; 12 cycles of 95° C./20 seconds and 60° C./60 seconds; 16 cycles of 94° C./20 seconds and 65° C./90 seconds; 65° C./3 mins; 10° C./hold. An Eppendorf ProS 384 thermocycler was used for amplification.

### Barcoding

The barcoding reaction was performed as described in Example 1 with the following modifications. The reaction mixture contained 30 μL of the pooled template after Sera-Mag Magnetic SpeedBeads (GE Healthcare Life Sciences) clean up. The cycling conditions included 12 cycles of 97° C./15 seconds, 60° C./30 seconds and 72° C./2 mins; 72° C./2 mins; 6° C./5 mins.

### Data Analysis

SeqKit v 1.2 was used to hard clip the reads by 15 bp at both 5′ and 3′ ends to remove adaptor sequences (Shen et al., 2016). Reads were aligned to a reduced representation of each species closest relative genome. Salmon fish reads were aligned to the zebrafish genome. Bismark v 0.20.0 was used to align reads with the following parameters: --bowtie2 -N 1 -L 15 --bam -p 2 --score L,-0.6,-0.6 --non_directional. Methylation calling was performed using bismark_methylation_extractor function with default parameters (Krueger and Andrews 2011).

Predicting Age from CpG Methylation

In order to predict age from CpG methylation samples are randomly assigned to either a training or a testing data set using the createDataPartition function in the caret R package (Kuhn et al., 2008). Age will be transformed to natural log to fit a linear model. An elastic net regression model will be used to regress the age of the Atlantic salmon over the CpG site methylation in the training data set for the sites identified in Table 7. The glmnet function in the glmnet R package (Friedman et al., 2010) will be set to a 10-fold cross validation with an α-parameter of 0.5, which returned a minimum k-value based on the training data. The performance of the model in the training and testing data set will be assessed using Pearson correlations between the chronological and predicted age and the MAE rates. The model will be used to estimate the age of Atlantic salmon based on the methylation beta value.

### Example 4—Age Estimation for Southern Bluefin Tuna

Since 2003, the Commission for the Conservation of Southern Bluefin Tuna (CCSBT) agreed that all Southern Bluefin Tuna fisheries should collect and analyse hardparts (otoliths) to characterise the age distribution of their catch. However, collecting large numbers of otolthis can be difficult and time consuming, particularly as Sashimi-grade fish are very valuable and often frozen soon after capture. The successful development of a rapid epigenetic age estimation method for Southern Bluefin Tuna would substantially improve our ability to get representative age data for all fisheries, as it would only require the collection of a tissue sample, not the extraction of otoliths, which requires much less time and expertise. It would also provide the basis for age estimation of live fish released as part of tagging programs.

A population of Southern Bluefin Tuna with high confidence age estimates with an approximately equal male:female ratio will be selected. Approximately 15 mg of tissue (for example, a fin clip tissue sample) will be used for DNA extraction. DNA will be extracted using the DNeasy Blood & Tissue Kit (QIAGEN) as instructed in the manufacture's protocol. Extracted DNA will bisulfite converted using the protocol as previously described (Clark et al., 2006) or using the EZ DNA Methylation Gold Kit (Zymo Research) in accordance with the manufacturer's instructions. The genome of Southern Bluefin Tuna will be analysed by pairwise alignment with the zebrafish reference genome danRer10 (Illumina iGenomes) to identify candidate age-associated CpG sites corresponding to an age-associated CpG site of Zebrafish listed in Table 1. The candidate Age-associated CpG sites identified for Southern Bluefin Tuna will be used to develop a DNA age estimator. Multiplex PCR and DNA sequencing will be performed for candidate age associated sites. The performance of the DNA age estimator will be assessed by the correlation and the absolute error rate between the age from otoliths and the estimated age from DNA.

### Example 5—Age Estimation for School Shark

### DNA Extraction and Bisulfite Treatment

DNA was extracted from shark fin tissue (approx. 15 mg) using the DNeasy Blood & Tissue Kit (QIAGEN) as instructed in the manufacture's protocol. Extracted DNA was bisulfite converted using the protocol as previously described (Clark et al., 2006).

### RRBS and Data Analysis

A total of 96 RRBS libraries were prepared as described in Example 1 and sequenced using an Illumina NovaSeq. The RRBS data was analysed as described in Example 1 with trimmed reads aligned to a reference shark genome using BS-Seeker2 v 2.0.3 default settings (Guo et al., 2013) and bowtie2 v2.3.4 (Langmead and Salzberg, 2012). The trimmed reads were aligned to either a reference genome from great white shark (Carcharodon carcharias) or a reference genome from whale shark (Rhincodon typus) (ASM164234v2).

### Identification of Age-Associated CpG Sites and Model for Estimating Age

In order to predict age from CpG methylation samples were randomly assigned to either a training or a testing data set using the createDataPartition function in the caret R package (Kuhn et al., 2008). Age was transformed to natural log to fit a linear model and using an elastic net regression model, the age of the sharks (2-10 years) was regressed over all CpG site methylation obtained from RBBS in the training data set. The glmnet function in the glmnet R package (Friedman et al., 2010) was used to identify the minimum CpG sites required to estimate the age of school sharks. This identified 30 CpG sites (Table 8) defined by reference to a great white shark reference genome that could be used estimate the age of school sharks and 23 CpG sites (Table 9) defined by reference to a whale shark reference genome (ASM164234v2) that could be used estimate the age of school sharks.

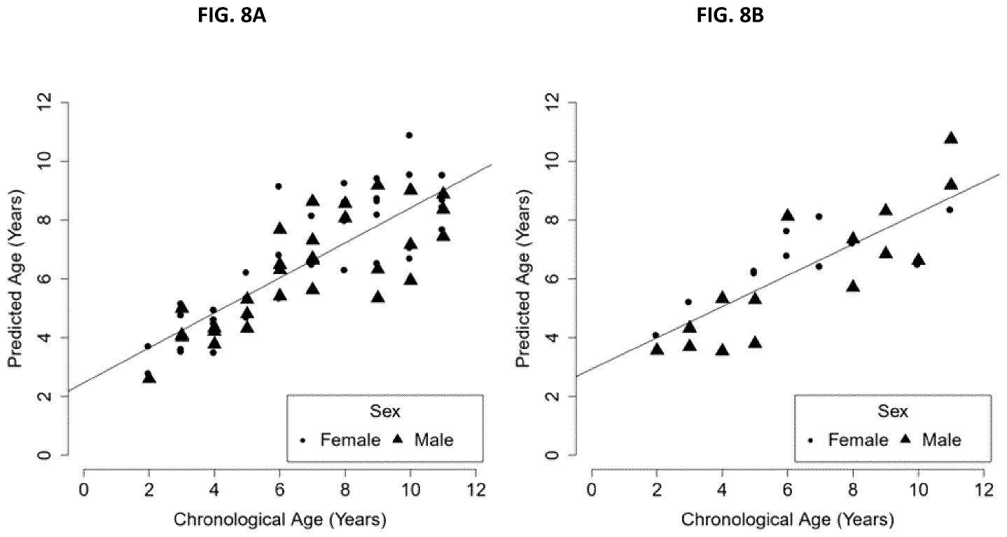

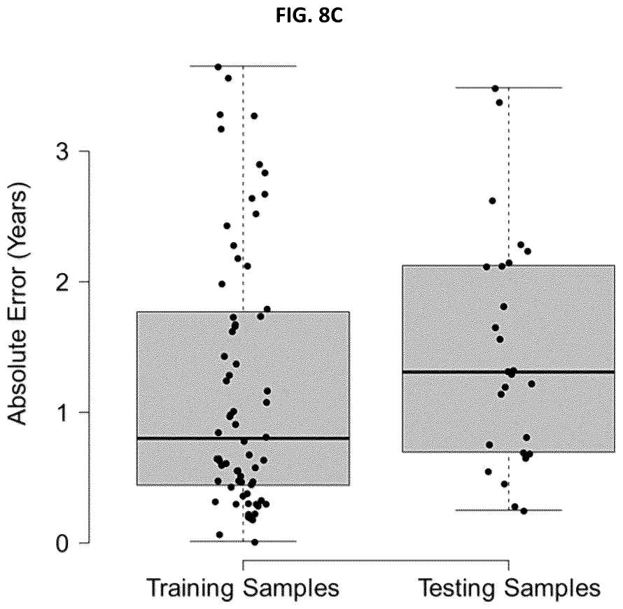

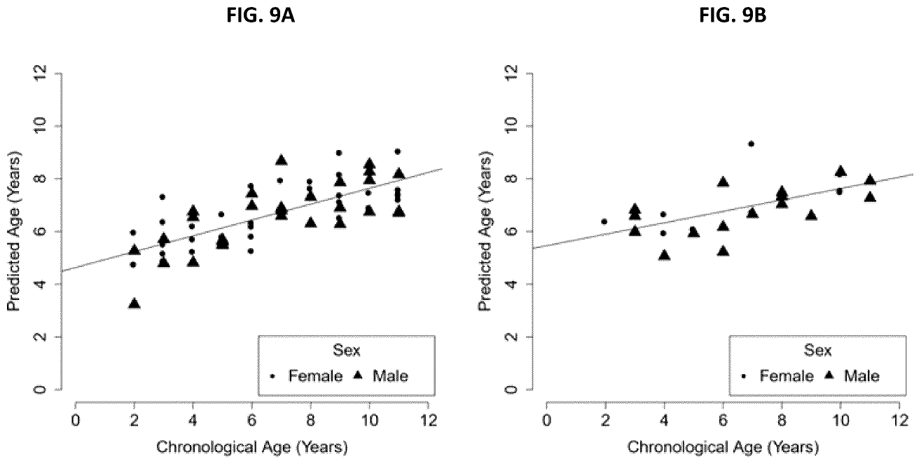

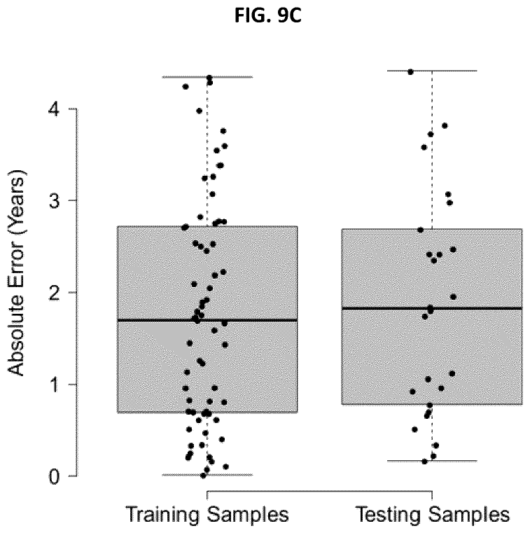

The performance of the model in the training and testing data set was assessed using Pearson correlations between the chronological and predicted age and the MAE rates (FIGS. 8 and 9). The CpG sites required to estimate the age of school sharks was used to generate a generalized linear model based on the raw prediction values from the elastic net regression model.

### Multiplex PCR

Primers for amplifying the age associated CpG sites in the final RRBS model and for one PCR reaction pool will be designed using Primersuite (Lu et al., 2017). Each individual primer pair will be tested individually using the GoTaq Hot Start Polymerase (Promega) and the manufacture's cycling conditions: 95° C., 2 min; 35 cycles (95° C., 1 min; 65° C., 1 min; 72° C., 30 s); 72° C., 5 min; 10° C. hold. Gel electrophoresis with sodium borate buffer using a 1.5% agarose gel will be used to visualise PCR products. Primer pairs which produce a single amplicon will be used for multiplex PCR.

The final multiplex PCR reaction will consisted of 1× Green GoTaq Flexi Buffer (Promega), 0.025 U/μL of GoTaq Hot Start Polymerase (Promega), 4.5 mM MgCl₂ (Promega), 0.5× Combinatorial Enhancer Solution (CES) (Refer to Ralser et al., 2006), 200 μM of each dNTP (Fisher Biotec), 15 mM Tetramethylammonium chloride (TMAC) (Sigma-aldrich), 200 nM forward primer, 200 nM reverse primer and 2 ng/μL bisulfite treated DNA. Initial cycling conditions will be 94° C./5 mins; 12 cycles of 95° C./20 seconds and 60° C./60 seconds; 16 cycles of 94° C./20 seconds and 65° C./90 seconds; 65° C./3 mins; 10° C./hold, although this can be optimised.

### Barcoding and Sequencing

Oligonucleotides with attached MiSeq adaptors and barcodes will be used for the barcoding reaction as described in Example 1. Sequencing will be performed as described in Example 1.

TABLE 8
Age associated CpG site location predictive of age in school sharks. Genomic locations are from a great white shark
(Carcharodon carcharias) reference genome (v. sCarCar2). The intercept is −4.45038. The coefficient is also referred to as weight.

| | | | Association with age | | | 300 bp amplicon comprising CpG site. |
|---|---|---|---|---|---|---|
| CpG site | | | | | p- | The CpG site of interest is in the middle of the 300 bp |
| scaffold | position | strand | Coefficient | Correlation | value | amplicon that can be used to design primers for multiplex PCR. |
| scaffold_ | 37483 | + | 0.869918 | 0.169757 | 0.101896 | ACAGATACCCTGGAGTGAGTTACAGACTGGAATCTAATCGAGGTGTTTGGGTTGGTTTATA |
| 1016 | | | | | | TATAGAATAACAGATAACCGGGAGTGAGTTACAGACTGGAATCTAATCGAGGGTGTAGGGG |
| | | | | | | TGGTTTATATATAGAATAACAGATACCCGGGAGTGAGTTACAGACTGGAATCTAATCGAGG |
| | | | | | | GGTTCGGGGTGGTTTATATATAGAATAACAGATACCCGCCAGTGAGTCACAGGCTGGAATC |
| | | | | | | TAATCGAGGTGTTCGGGGTGGTTTATATATAGAGTAACAGATACCCTGGAGTGAGT (SEQ |
| | | | | | | ID NO: 195) |
| scaffold_ | 235139 | + | 1.341429 | 0.263447 | 0.010302 | ACAGATACCCGGGAGTGAGTTACAGACTGGAATCTAATCGAGGGGTTCGGGGGGGTTTATA |
| 137 | | | | | | TATAGAATAACAGATACCCGGGAGTGAGTTACAGACTGGAATCTAATCGAGGGGTTCGGGG |
| | | | | | | TGGTTTATATATAGAATAACAGATACCCGGGAGTGAGTTACAGGCTGGAATCTAATCGAGG |
| | | | | | | GGTTCAGGGTGGGTTTACATATAGAATAACAGATACCCGAAAGTGAGTTACAGACTGGAAT |
| | | | | | | CTTATCAAGGGGTTCGGGGGGGTTTATATATGTAATAACAGATATCCGGGAGTGAG (SEQ |
| | | | | | | ID NO: 196) |
| scaffold_ | 518192 | + | 2.370049 | 0.194555 | 0.060238 | ACTCACTCCCGGGTACCTGTTATTCTATATATAAACCCCCCCCCGAACCCCTCGATTACAT |
| 137 | | | | | | TCCAGTCTGTAACTTCACTCCCGGGTATCTGTTATTCTATATGTAAACCACCCCTAACCCT |
| | | | | | | CGATTAGTTTCCAGTCTGTAACTCACTCGCGGGTATCTGTTATTCTATATATAAACCACCC |
| | | | | | | CGAACCCCTCGATTAGATTCCAGTCTGTAACTCACTCCTGGGTATCTGTTATTCTGTATAT |
| | | | | | | AAACCGCCCCGAACCCCTCGAtTAGATTCCAGTCTGTAACTCACTCCCGGGTATCT (SEQ |
| | | | | | | ID NO: 197) |
| scaffold_ | 24527306 | + | −0.56649 | −0.23605 | 0.021998 | AAATAGGTGGGAAAAGAAAAATCTATATAAATTATTGGGAAAAACAAGGAGGGGGAAGAAA |
| 17 | | | | | | CAAAAAGTGGGTGGGGACGAAGGAGAGAGTTCAAGATCTAAAATTGTTGAACTCAGTATTC |
| | | | | | | AGTCCGGAAGGCTGTAAAGTGCTTAGTCGGAAGATGAGATGCTGTTCCTCCAGTTTGTGTT |
| | | | | | | GAGCTTCACTGGAACAATGCAGCAAGCCAAGGGTAGACATGTGGGCATGGGAGCAGGGTGG |
| | | | | | | AGTGTTGAAATGGCAAGCGAGAGGGAGGTCTGGGTAATGCTTACGGACAGACCGAA (SEQ |
| | | | | | | ID NO: 198) |
| scaffold_ | 896154 | + | −0.40008 | −0.18704 | 0.071049 | ACCTTATCCACCTGCGTTGCCACTTTCAGTGACCTGTGGACCTGTACACCCAGATCCCTCT |
| 182 | | | | | | GCCTGTCGATGCACTTAAGGGTTCTGCCATTTACTGTATAATTCCTGCCTGTATTAGACCT |
| | | | | | | TCCAAAATGCATTACCTCGCATTTGTCCGGATTAAACTCCATCTGCCATTTCTGCGCCCAA |
| | | | | | | GTCTCCAACCAATCTATATCCCGTTGTATCCTTTGACAATCCTCTTCACCATCTGCAACTC |
| | | | | | | CTCCAACCTTAGTGTTGTCTGCAAACTTACTAATTAATCCAGTTACATTTTCCTCC (SEQ |
| | | | | | | ID NO: 199) |
| scaffold_ | 317376 | + | −0.81931 | −0.17161 | 0.098156 | TCCCCATTTACACACACCGGGCAGTCTGTTCCCTATCGGAGGGGGATTAACCCTCTCACCC |
| 198 | | | | | | ACCTCCCCCCATTTACACACACCGGGCAGTCTGTTCCCTATCGGAGGGGGATTAACCCTCT |
| | | | | | | CACCCACCTCTCCCCATTTACACACACCGGGCAGTCTGTTCCCCTATCGGAGGGGGATTAA |
| | | | | | | CCCTCTCACCCACCTCTCCCCATTTACACACACCGGGCAGTCTGTTCCCTATCGGAGGGGG |
| | | | | | | ATTAACCCTCTCACCCACCTGTCCACATTTACACACATCGGGCAACCTTTTCCCTA (SEQ |
| | | | | | | ID NO 200) |
| scaffold_ | 120463762 | + | 6.742987 | 0.182218 | 0.078785 | TTTTAGTAAAACACAAATTTAATAATGGGGGCAACGTGGTGGTTTGGTGCTGCTGCCTCAC |
| 2 | | | | | | AGCTCTATGGACCTGGGTTCGATCTTGGCCTCAGGTGCCTGTCTGCTTGGAGTTTGTACGT |
| | | | | | | TTCCCCCATGTCTGCGTGGGTCTCCCTCGGGTGCTTTGGTTTCTTCCCACTGCCCAAAGAC |
| | | | | | | ATGCTGGTTAGGTGTATTGACTAAGGTAAATTGTCCCCCAGTGTGTGTATGTCTACATGTG |
| | | | | | | AGAGTGTGCCCTGTGATGGATTGGTTTCCCATCCTTGATGTGTCCTGCTTAATGCC (SEQ |
| | | | | | | ID NO: 201) |
| scaffold_ | 200259206 | + | −0.32431 | −0.18881 | 0.068371 | AAATGCTATTGGTTTCTGTGGGGGTAGTACGGTTGCGCAGTAGCTAGCACTGCTGCCTTGC |
| 2 | | | | | | AGTTGCAAGGACCCAGGCTTGATTCTGACCTTGGATGTCTGTCTGCGTGGAGTTTGCACGT |
| | | | | | | TCCCCGTGTGTCCACGTGGGTTTCTGCCGGGTGCTTCAGTTTCCTCCCACCATCCAAAGAC |
| | | | | | | GTGCTGGTTAGATGGATTGGCTACGCCTATATTGCCCCTTAGAGTGTGCTTATGTGCATCT |
| | | | | | | GAGTGTGTGCCCTGTGATAGGCTGGCATCCCATCCTGGGTGTAATGGCCATTGCCT (SEQ |
| | | | | | | ID NO: 202) |
| scaffold_ | 270371 | + | −0.77662 | −0.24287 | 0.018344 | ATAAATAGAATAACAGATACCCAGGAGAGACTTACAGACTGGAATATAATTAAGGGTGTCA |
| 222 | | | | | | GGGTGGTTTATATAGAGAATTAGAGATAGCTACAGAGTGGAATCTAATCGAGTGTTTCGCA |
| | | | | | | GTGTTTATAAATGGAATAACAGATACACGGGAGTGAGTTACAGACTGGAATCTGATCGAGG |
| | | | | | | GGTTCGGGGTGGTTTATATATAGAATAACAGATATCCTGGCGTGAGTTACAAACAGGAATT |
| | | | | | | TTATCGAGGTTTTCAGGGGCTTTATATATAAAATAACAGATACCCGGGAGTGAGTT (SEQ |
| | | | | | | ID NO: 203) |
| scaffold_ | 19279262 | + | −0.23354 | −0.17431 | 0.092904 | CCACCATTGGAACATCCTTCCTGCATCTACCCTGTCTAGTCCTGTTAGAATTTTATAGGTT |
| 26 | | | | | | TCTATGAGCTCTCTCCTCTTTCTTCTGAACTCCAGTGAATATAATCCTAACCAACTCAATC |
| | | | | | | TCTTCTCATATGTCAGTCCCACCATCCCGGAATCAGTCTGGTAAACCTTCGCTGCACTCCC |
| | | | | | | TCTATAGCAAGAACATCCTTCCTCAGATAAGGAGACCAAAACTGCACATAATATTCCAGGT |
| | | | | | | GTGGCCTCACCAAGGCCCTGTATAATTGCAGCAAGACATCCCTGCTCCTGTACTCG (SEQ |
| | | | | | | ID NO: 204) |
| scaffold_ | 29408365 | + | −0.64823 | −0.17143 | 0.098514 | GCACACTAAGGGGAAATATACTGTAGCTAATCCAACTAACCAGCACGTCTTTGGTCAGTGG |
| 31 | | | | | | AAGGAAACTGGAGCACCTGACAGAAACCCAAGCAAACACAAGCCGAACGTGTGATCTCCAC |
| | | | | | | ACAGACATCCGAGGTCAGAATTGAACCCGGGTCCCTGGAGCTGTGAGGCAGCAGCACTAAC |
| | | | | | | CACTGTGCCACCATGCCGCCCAAAATTTATAAACACTAATAAATAGATTTACTAAAATTAG |
| | | | | | | AATGTTAAAGTTAATTTTATTGCAGAGTTGATATTCTCCTTAATGAATTGTTATAT (SEQ |
| | | | | | | OD NO: 205) |
| scaffold_ | 29021 | + | 0.187594 | 0.169921 | 0.101562 | ACAGGGATGGGGAGCAGGAACCCGGGCTGATTCACACCCCCTCCCTAACCCAGGGGTCAGT |
| 348 | | | | | | GGACAGGGATGGGGAGCAGGAACCCGGGCTGATTCACACCCCCCTCCCTAACCCAGGGGTC |
| | | | | | | AGTGGACAGGGATGGGGAGCAGGAACCCGGGCTGATTCACcCCCCCTCCCTAACCCAGGGG |
| | | | | | | TCAGTGGACAGGacTGGGAGCAGGAACCCGGGCTGATTCACACCCTCCCTAACCCAGGGG |
| | | | | | | TCAGTGGACAGGGATGGGGAGCAGGAACCCGGGCTGATTCACACCCCCCTCCCTAA (SEQ |
| | | | | | | ID NO: 206) |
| scaffold_ | 296750 | − | −0.4645 | −0.23708 | 0.021409 | CACCCGGCCCGGACACGGAAAGGATTGACAGATTGATAGCTCTTTCTCGATTCTGTGGGTG |
| 353 | | | | | | GTGGTGCATGGCCGTTCTTAGTTGGTGGAGCGATTTGTCTGGTTAATTCCGATAACGAACG |
| | | | | | | AGACTCCTCCATGCTAAATAGTTACGCGACCCCCGAGCGGTCCGCGTCCAACTTCTTAGAG |
| | | | | | | GGACAAGTGGCGTACAGCCACACGAGATTGAGCAATAACAGGTCTGTGATGCCCTTAGATG |
| | | | | | | TCCGGGGCTGCACGCGCGCTACACTGAATGGATCAGCGTGTGTCTACCCTTCGCCG (SEQ |
| | | | | | | ID NO: 207) |
| scaffold_ | 298628 | − | 0.31245 | 0.186651 | 00.01656 | TTTTATGGCGTGCCTGGGCACGCCGGGGCCGCGCCTTCGGGATGGGGCTTCCGGCAGATGT |
| 353 | | | | | | CGGCGAGCGTGGGGTGCGGTCCGTGCGCGGCTTCCTCGCGGGAGGATCCGACCGAAAGCTC |
| | | | | | | TGTACAACTCTTAGCGGTGGATCACTCGGCTCGTGCGTCGATGAAGAACGCAGCTAGCTGC |
| | | | | | | GAGAATTAATGTGAATTGCAGGACACATTGATCATCGACACTTTGAACGCACTTTGCGGCC |
| | | | | | | CCGGGTTCCTCCCGGGGCTACGCCTGTCTGAGGGTCGCTTGACGATCAATCGCACT (SEQ |
| | | | | | | ID NO: 208) |
| scaffold_ | 325748 | − | −0.14995 | −0.18034 | 0.081964 | CACCCGGCCCGGACACGGAAAGGATTGACAGATTGATAGCTCTTTCTCGATTCTGTGGGTG |
| 353 | | | | | | GTGGTGCATGGCCGTTCTTAGTTGGTGGAGCGATTTGTCTGGTTAATTCCGATAACGAACG |
| | | | | | | AGACTCCTCCATGCTAAATAGTTACGCGACCCCCGAGCGGTCCGCGTCCAACTTCTTAGAG |
| | | | | | | GGACAAGTGGCGTACAGCCACACGAGATTGAGCAATAACAGGTCTGTGATGCCCTTAGATG |
| | | | | | | TCCGGGGCTGCACGCGCGCTACACTGAATGGATCAGCGTGTGTCTACCCTTCGCCG (SEQ |
| | | | | | | ID NO: 209) |
| scaffold_ | 135081937 | + | 1.285505 | 0.248236 | 0.015847 | TCCTTTTGTCTTCTTAGTCTCATGCACTGCCCCTTTCATGAGCTACAAGGACTACCCCCTC |
| 4 | | | | | | CCCCTCCCTGGCTCCAGTTGTTGACTACACCCATGTTTTGACACCGTGCTGCAGTTTCCAC |
| | | | | | | ACTTGCTCCCTCACCCTAATCTCTCTCCGGCGTGCTCACTCTCTCTCTTTCTCTTCTCTTC |
| | | | | | | TCTCTCTCTCTTTCTCTCTCTCTTTCGCTCTCTCAGACATTCTTCTGTTCTCTCTTTTTAA |
| | | | | | | GCCATCTGCTCTCTCATTACTTACTACTTTCTTGCACTCCCTTTGTCTCCCTTACA (SEQ |
| | | | | | | ID NO: 210) |
| scaffold_ | 316932 | + | 0.94837 | 0.239066 | 0.02031 | TATTTACAGGGGAGTCCCTGGGGAGTGTGTCAGTATTTACAGGGGGTCCCCGGGGAGTGTG |
| 462 | | | | | | TCAGTATTTACAGGGGGGTCCCCAGGGAGTGTGTCAGTATTTACAGGGGTGTCCCCGGGGG |
| | | | | | | AGTGTGTCAGTATTTACAGGGGGAATCCGGGGGGAGTGTGTCAGTATTTACAGGGGGGGGT |
| | | | | | | CCCCGGGGAGTGTGTCAGTATTTACAGGGGTCCCCGGGGAGTGTGTCAGTATTTACAGGGG |
| | | | | | | GGTCTCCGGGGAGTGAGTCAGTATTTACAGGGGGTCCCCAGGGAGTGAGTCAGTAT (SEQ |
| | | | | | | ID NO: 211) |
| scaffold_ | 110576 | + | 0.627291 | 0.213323 | 0.03898 | CACGGTGCTCCGAGTGTGGTCTAACCGAGGGGGATACGGAGACCGGAACTGTGCACGGTGC |
| 472 | | | | | | TCCGAGTGTGGTCTAACCGAGGGGGATACGGAGACCGGAACTGTGCACGGTGCTCCGAGTG |
| | | | | | | TGGTCTAACCGAGGGGGATACGGAGACCGGAACTGTGCACGGCGCTCCGAGTGTGGGTCTA |
| | | | | | | ACCGAGGGATATGGAGACCGGAACTGTGCACGGTGCTCCGAGTGTGGGCTAACCCAGGGGG |
| | | | | | | ATACGGAGACTGGGACTGTGCACGGTGCTCCGAGTGTGGTCTAACCGAGGGGGATA (SEQ |
| | | | | | | ID NO: 212) |
| scaffold_ | 431059 | + | −0.27269 | −0.20622 | 0.046135 | CGGTCTCCTATCCTCGGTTAGACCACACTCGGAGCACCGTGCACAGTTCCGGTCTCCGTAT |
| 488 | | | | | | CCCTCGATTGACCACACTCGGAGCACCGTGCACAGTTCCGGTCTCCTATCCCTCGGTTAGA |
| | | | | | | CCACACTCGGAGCACCGTGCACAGTTCCGGTCTCCCTATCCCTCGGTTAGACCCACTCGGA |
| | | | | | | GCACCGTGCACAGTTCCGGTCTCCGTATCCCCTCGGTTAGACCACACTCGGAGCACCGTGC |
| | | | | | | ACAGTTCGGTCTCCGTATCCCTCGATTAGACCACACTCGGAGCACCGTGCACAGTT (SEQ |
| | | | | | | ID NO: 213) |
| scaffold_ | 125160496 | + | −0.32433 | −0.23409 | 0.023156 | TGCTGTAGAGGCTGCTATGGGAAGTGTCAATGCTGCTGCTATCTCAAGGCGGCATGTAGAT |
| 6 | | | | | | GGTGTAGCTTAAAAAATACCATAACTTAGTTATTATCTCAGGGTCTGGTCAATTGTTCTCA |
| | | | | | | ATTTTAAATTTCTCACCCTGGGCAGCACGGTGACACAGTGGTTAGCACTGCTGCCTCAGCT |
| | | | | | | CCAGGGACCCGGGTTCAATCCTGACCTCTGGTCTCTGTCTGTATTTAGAGTCTCCATGTCT |
| | | | | | | GCGTGGGCTTCTGCCAGGTGCTCCGATTCTCTTTTCCACCCCCCACCATCCAAAGA (SEQ |
| | | | | | | ID NO: 214) |
| scaffold_ | 35876984 | + | 0.645001 | 0.300857 | 0.003214 | TTGTAGGACAATTTTACCGTAGCCGATCCACCTAACCAGTATGTCTTTGGATGGTGGGAGG |
| 6 | | | | | | AAACCGGAGCACCCAGTGGAAACTCACGCAGACACGGGGAGAATGTGCAATCTCCACATAG |
| | | | | | | ACAGACACCTGAGGTCAAGATCAAACCCGGGTGCCTGGAGCTGTGAGGCAGCAGCACTAAC |
| | | | | | | CACTCCGCCACCGTGCTGCCTCAAGAATCAATTTATTGCAATTATGGAAGAACTTGTAATG |
| | | | | | | TGCAATGGATTTAACATTTTGTCTGAAATCAAGAAGCGAGATGGTATTAATGATGG (SEQ |
| | | | | | | OD NO: 215) |
| scaffold_ | 4244906 | + | −1.00572 | −0.23164 | 0.024673 | Gtcagtatttacagggggtccccggggagtgtgtcagtatttacagggggaccaggggagt |
| 60 | | | | | | gtgtcagtatttacagggaattacttggtagtgtgtcagtatttacagtgggtcccggggg |
| | | | | | | agtgtgtcagtatttacagggggtccccggggagtgtgtcagtatttacagggggtccccg |
| | | | | | | gggagtgtgtcagtatttacagggggtaccgaggagtgtgtcagtatttacagggggtccc |
| | | | | | | cggggagtgtgtcagtatttacagggggtcccggggagtgtgtcagtatttacagg (SEQ |
| | | | | | | ID NO: 216) |
| scaffold_ | 4307183 | + | −0.09634 | −0.16969 | 0.102043 | TCAGTATTTACAGTGTTTCCCCGGTGAGTGTGTCAGTATTTACAGTGTTTCCCCGGGGAGT |
| 60 | | | | | | GTGTCAGTATTTACAGGGGGTCCCCGGGCAGTGTGTCAGTATTTACAGTGTTTCCCCGGTG |
| | | | | | | AGTGTGTCAGTATTTACAGTGTTTCCCCGGGGAGTGTGTCAGTATTTACAGTGTTTCCCCG |
| | | | | | | GTGAGTGTGTCAGTATTTACAGTGTTTCCCCGGGGAGTGTGTCAGTATTTACAGGGGGTCC |
| | | | | | | CCGGGGAGTGTGTCAGTATTTACAGGGGAGTCCCTGGGGAGTGTGTCAGTATTTAC (SEQ |
| | | | | | | ID NO: 217) |
| scaffold_ | 13970134 | + | −0.63662 | −0.25905 | 0.011696 | CATGAGGACCCTACAAAGAGGTTCTGGGAACTGGTCATAGTTGCGACCACACTAAAAAATT |
| 7 | | | | | | GTACTGTACAGATAGGTAGGTAGAAAGGTGGGCAGGTAGGTGGGTAGATAGGAAGGTAGAT |
| | | | | | | AGATAAGTAGGTAGGCAGCCAGATAGGCGGATAGATAGGTAAGCAGGTAGGCAGGCAGGTT |
| | | | | | | GATGGGTAGAGAGGCAGCGAGGTAGTTTTATAGGCAGGGTGGGCAGGTGGGCAGGTAGGTG |
| | | | | | | GGTAGGTAGGTAGGTAGGTAGATAAGTAGGCAGGTTGGTAGGCAGGCAAGTAGGCA (SEQ |
| | | | | | | ID NO: 218) |
| scaffold_ | 41259033 | + | −0.7362 | −0.16939 | 0.102643 | CAAGATGTTAACTCTTCTTGTGATACATTTCCACAAGTGGTGCCAACTCCACAGCACTTTG |
| 7 | | | | | | CCTAAGTGGCCATTCTTCATTTCTGAGACTGACACTAAGAGTTGACAAATTATTGAATTGT |
| | | | | | | GAAAGGTGATACAACTGAGTCCTATCCCGGGTTCACTCTCTGTCCACATATCGATACTTTC |
| | | | | | | AAGCAAGATTCACAAATTAGTGAGCAGGAACTGGGACCATGACTGAGATCTAGCTTGTCAA |
| | | | | | | AAGTACTTAGACCAACTTTTGGGCCTGAGTGAGATCAGTTGGTTCAGTGCAGGCCA (SEQ |
| | | | | | | ID NO: 219) |
| scaffold_ | 62564082 | + | −0.08965 | −0.20863 | 0.043594 | GAGATAATTGATGAACAAATCAAAGTTAGAGATGATGAAGCCTCTGGCCCTTTCAGATTGC |
| 7 | | | | | | TTCCGGCAATTTAAAAGAAGTTGATGGCAACTTCTGTGTGGAGTTTGCATATTCTCCCCAT |
| | | | | | | GTCTGCGTGGGTTTCTGCCAGGTGCTTCGGTTTCCTCCCACCATCCAAAGACGTGCTGGTT |
| | | | | | | AGGTGGATTGGCTACGATAAATTGTCTCCTAGTGTGTGCGTGTCTGCGTGTGTATGTGTGA |
| | | | | | | GTATGTGCCCTGTGATGGACTGATGTCCTGTCTTGGGTGTACCCTGCCTAGCACCC (SEQ |
| | | | | | | ID NO: 220) |
| scaffold_ | 1636216 | + | 0.454434 | 0.201792 | 0.05113 | GAGGGCAGAGATGGACAAGATGGGCTCATGGTTCACGAATTACAAAAAACTCAAACACAAG |
| 89 | | | | | | CATGAAATAACAGATACCGGGGAGTGAGTTACAGACTGGAATCTAATCGAAAGGTTCGGGG |
| | | | | | | TGGTTTATATATAGAATTACAGATACCCGGGAATGAGTTACAGACTGGAATCTGATCGAGG |
| | | | | | | GGTTCGGGGTGGTTTATATATAGAATAACAGATACCCGGGAGTGAGTTACAGACTGGAATC |
| | | | | | | TAATTGAAGGGTTTAGGGGTGGGGTTTAAACGTAGAATAGCATGATAACAACTGAG (SEQ |
| | | | | | | ID NO: 221) |
| scaffold_ | 70262 | + | −0.55927 | −0.17945 | 0.083516 | TGTGACACCCGGTCCCAGAGGGGGAAAGGACAGTGTATCTAAAGCCCTGTGTACCTGGTCC |
| 893 | | | | | | CAGAGGGGGAAAGGACAGTGTATCTAACGCACTGTGACACCTGGTCCGAGAGGGGGAAAGG |
| | | | | | | ACAGTGTATCTAACGCACTGTGACACCCGGTCCCAGAGGGGGAAAGGACAGTGTATCGAAC |
| | | | | | | GCACTGTGACACCCGGTCCCAGAGGGGGAAAGGACAGTGTATCTAACGCAATGTTACAACC |
| | | | | | | GGTCCCAGTGGGGGAAAGGACAGTGTATCTAACGCACTGTGACACCCGGTCCCAGA (SEQ |
| | | | | | | ID NO: 222) |
| scaffold_ | 93698258 | + | −0.48441 | −0.20777 | 0.044486 | TGCCTTTTTTCCAGCGAGTAGAAGAAGGGGGAGCCGCGGTCCATCTCCTTGAGGAACCGGA |
| 9 | | | | | | TCCGCGACCTCACGAACGCGCCCCGAGACCCGACCAGTTGCAGGTCCCGCAGCGCGCCCTT |
| | | | | | | CTTCTCATCGTACACCGACCGCAGGGCCGGGTCCGCGTCGGGCTGACCGAGACGTGCCTCC |
| | | | | | | AGGTCGAGCACCTCCTTCTCCAACTCCTCGACCCTGGATTTGCGTCGCTTTGTCGACCCCC |
| | | | | | | TCACGTACTCCTGACAGAAAACTCGGACGTGAGTCTTGCCCACGTCCCACCAGAGC (SEQ |
| | | | | | | ID NO: 223) |
| scaffold_ | 3488581 | + | 1.125919 | 0.197831 | 0.055964 | cccggaatgtgtcagtatttacaggggtccccgggagtgtgtcagtatttacaggggtccc |
| 90 | | | | | | gaagtgagtcagtatttacagggtttccccgggagtgtgtcagtatttacagggtccccgg |
| | | | | | | gagtgtgtcagtattacagggggtccccggggggagtgtgtcagtatttacagggggtccc |
| | | | | | | cgggagtgtgtcagtatttacagggtgtcccgggaggtgtcagtatttacagggggtcccg |
| | | | | | | ggagtgtgtcagtatttacagggggtccccgggagtgtcagtatttacaggggtcc |

TABLE 9
Age associated CpG site location predictive of age in school sharks. Genomic locations
are from a whale shark (Rhincodon typus) reference genome (ASM164234v2). The
intercept is 4.243827. The weight is also referred to as coefficient.

| CpG site | | | Association with age | | |
|---|---|---|---|---|---|
| scaffold | position | strand | Weight | Correlation | p-value |
| NW_018028146.1 | 122365 | + | 1.564966 | 0.067221 | 0.522036 |
| NW_018028307.1 | 149819 | + | −0.01501 | −0.19421 | 0.062127 |
| NW_018031832.1 | 288010 | + | 0.062081 | 0.064188 | 0.541023 |
| NW_018033035.1 | 68874 | + | 0.533872 | 0.151402 | 0.147426 |
| NW_018037231.1 | 8655 | + | −0.60951 | −0.16467 | 0.114722 |
| NW_018037876.1 | 86765 | + | 0.585141 | 0.190717 | 0.067073 |
| NW_018038722.1 | 28181 | + | −0.17484 | −0.13458 | 0.198389 |
| NW_018040670.1 | 64912 | + | 1.626296 | 0.153977 | 0.140584 |
| NW_018041289.1 | 23746 | + | −0.17023 | −0.17003 | 0.103215 |
| NW_018046236.1 | 52414 | + | 0.616872 | 0.112318 | 0.283763 |
| NW_018048825.1 | 11834 | + | −0.27711 | −0.2178 | 0.035972 |
| NW_018049359.1 | 30850 | + | −0.62566 | −0.2105 | 0.042836 |
| NW_018049493.1 | 5425 | + | −1.16641 | −0.20884 | 0.044538 |
| NW_018049493.1 | 6271 | + | 1.090685 | 0.176491 | 0.090586 |
| NW_018051531.1 | 12502 | + | 1.339225 | 0.262424 | 0.011047 |
| NW_018052368.1 | 7555 | + | 0.267859 | 0.120371 | 0.250432 |
| NW_018053486.1 | 576 | − | −1.96658 | −0.34179 | 0.000799 |
| NW_018056678.1 | 605623 | + | −0.14132 | −0.2016 | 0.052642 |
| NW_018057511.1 | 33747 | + | −1.15988 | −0.18087 | 0.082739 |
| NW_018060849.1 | 2865 | + | −0.21061 | −0.05346 | 0.610779 |
| NW_018063713.1 | 27779 | + | −0.17015 | −0.16955 | 0.104206 |
| NW_018069220.1 | 73762 | + | −1.87249 | −0.21674 | 0.036913 |
| NW_018069264.1 | 360954 | + | −0.49692 | −0.20591 | 0.047683 |

### Data Analysis and Age Estimation

SeqKit v 1.2 will be used to hard clip the reads by 15 bp at both 5′ and 3′ ends to remove adaptor sequences (Shen et al., 2016). Clipped reads will be aligned to a reference school shark genome. A reference genome may be the genome of a close relative. Bismark v 0.20.0 will be used to align reads as described in Example 1. Methylation calling will be performed as described in Example 1. The generalized linear model developed above will be used to estimate the age of school sharks based on the methylation beta value.

### Example 7—Age Estimation for Australian Lungfish (Neoceratodus forsteri)

The age of Australian lungfish (Neoceratodus forsteri) cannot be estimated using otoliths as growth annual increments are not visible (Gauldie et al., 1986). It is also undesirable to use a lethal methodology as the Australian lungfish is considered threatened under the Australian Environment Protection and Biodiversity Conservation Act, 1999 (“Threatened Species Scientific Committee. Commonwealth Listing Advice on Neoceratodus forsteri (Australian Lungfish),” 2003). Bomb radiocarbon techniques have been used previously to estimate age in Australian Lungfish (Fallon et al., 2019). Although bomb radiocarbon is an effective method to determine age it can be expensive making it difficult to estimate age for large populations. In this example, the inventors have used the Zebrafish age-associated sites identified in Examples 1 and 2 to develop an epigenetic clock for the Australian lungfish (Neoceratodus forsteri). This study demonstrates age associated CpG methylation at sites in one fish species can be predictive of age in other species.

### Animal Ethics and Tissue Collection

Australian lungfish samples were collected from the Brisbane, Burnett, and Mary rivers in south east Queensland, Australia. Collection of fin tissue was approved under General Fisheries Permits 174232 and 140615 and approved by Australian Ethics Committee protocol numbers CA2011/10/551 and ENV/17/14/AEC. A mix of known age and age determined by bomb radiocarbon dating Australian lungfish samples were used in this study (Table 10) (Fallon et al., 2015; James et al., 2010). The Australian lungfish samples were used from previous research projects and mortalities of captive-raised and CITES-registered fish including public aquarium and private aquarium collections (Fallon et al., 2019). An additional sample was provided from a euthanized captive Australian lungfish maintained by the Shedd Aquarium, Chicago, USA.

TABLE 10
Total number of samples and age ranges
used for Australian lungfish.

| Species | Total Samples | Age Range (Years) |
|---|---|---|
| Australian | 141 | bomb radiocarbon |
| lungfish | (102 bomb | age: 2-77 |
| (Neoceratodus | radiocarbon age and | known age: 0.1-14 |
| forsteri) | 39 known age) | |

### DNA Extraction and Bisulfite Treatment

DNA was extracted using the DNeasy Blood & Tissue Kit (QIAGEN) as instructed in the manufacture's protocol. Extracted DNA was bisulfite converted using the protocol as previously described (Clark et al., 2006).

### Identification of Age-Associated CpG Sites and Primer Design

Multiplex PCR was used to develop an assay for age estimation using sites known to be age associated in zebrafish. Primers were designed targeting CpG sites with methylation levels that are both known to significantly correlate with age in zebrafish and are conserved between species. At the time when this study was conducted a reference genome for the Australian lungfish was unavailable. Instead a publicly available RNA-seq data (BioProject accession ID: PRJNA282925) was used as a substitute for genomic data (Biscotti et al., 2016). HISAT2 v2.1.0 with default parameters was used to align the RNA-seq data to the zebrafish reference genome (danRer10, Illumina iGenomes) (Kim et al., 2015). RNA-seq alignments that overlapped with age associated CpG sites identified by bedtools v2.25.0 were targeted for primer design (primers shown in Table 11). Primer pairs were designed using Primersuite and for one PCR reaction pool (Lu et al., 2017).

### Singleplex and Multiplex PCR

Each primer pair were tested individually using the GoTaq Hot Start Polymerase (Promega) as instructed using the manufacture's cycling conditions. Gel electrophoresis with sodium borate buffer using a 1.5% agarose gel was used to visualise PCR products. Primer pairs that produced one product at the predicted size were used together as a multiplex PCR reaction.

The final multiplex PCR reaction consisted of 1× Green GoTaq Flexi Buffer (Promega), 0.025 U/μL of GoTaq Hot Start Polymerase (Promega), 4.5 mM MgCl₂ (Promega), 0.5× Combinatorial Enhancer Solution (CES) (Refer to Ralser et al., 2006), 200 μM of each dNTP (Fisher Biotec), 15 mM Tetramethylammonium chloride (TMAC) (Sigma-aldrich), 200 nM forward primer, 200 nM reverse primer and 2 ng/μL bisulfite treated DNA. Multiplex PCR cycling conditions were: 94° C., 5 min; 12 cycles (94° C., 20 s; 60° C., 60 s); 16 cycles (94° C., 20 s; 65° C., 90 s); 65° C., 3 min; 4° C. hold. Table 11 contains the full list of primer pairs that were screened as part of developing the multiplex PCR assays.

TABLE 11
Primers used to amplify conserved age associated CpG sites in the Australian lungfish. X = Validated for multiplex PCR.

| X | Forward primer | Reverse primer | gDNA amplicon sequence |
|---|---|---|---|
| Yes | gacatggttctacaTGTtTAt | cagagacttggtctCTaaCcN | TGTCTACTAATGAAGTGTTACCTGTGGGCGCGAGGGTGGTGGGGGCCCTGAGCGGGTCTATC |
| | TAATGAAGTGTTAttTGTG | aAaATaTTCAACTaCA | TCCCCGGGGATGCAGAGCTCGGGTCCGTCGGTAAACCGGCTGCAGTTGAACATCTCCGGCCA |
| | (SEQ ID NO: 225) | (SEQ ID NO: 280) | G (SEQ ID NO: 335) |
| Yes | gacatggttctacaGGNgttT | cagagacttggtctCTTATTc | CTAAAACAGACAATAGTGCAAACAACGCTTTCATTTCGGTGTCCATGTTGTTGTTTACAGTA |
| | AAAAtAGATAATAGTG | NTCAAaCCCCTATC | CTGTCTTCCGGTAGCGTTAAAGCCATGTGTTATAGTCATGTGATAGGGGCTTGACGAATAAG |
| | (SEQ ID NO: 226 | (SEQ ID NO: 281) | GGAAG (SEQ ID NO: 336) |
| Yes | cagagacttggtctCTTATTT | gacatggttctacaGGATGAA | CTTATTTTATCCATCCCCCTCCTCAACTCTCCTTCTGCCCTGTCCGCTGTTCACTTTCACTT |
| | TATCCATCCCCCTC | AGTGAAGAGtAGG | TCTTCCGCGACTCCCCATCCCTCTTTACTTTCGTCTGCAACAACCCCCTGCTCTTCACTTTC |
| | (SEQ ID NO: 227) | (SEQ ID NO: 282) | ATCC (SEQ ID NO: 337) |
| Yes | gacatggttctacaATGGTGt | cagagacttggtctCTATTCN | CCTAAAACAGACAATAGTGCAAACAACGCTCCCATTTCTGTGCCCATGTTATTGTTTACAGT |
| | tTAAAAtAGAtAATAGTG | TCAAaCCCCTATC | GAAGGCTGGTCTGCTGTCTTCCGGTAGCGTTAAAGTCATGTGTTATAGTCATGTGATAGGGG |
| | (SEQ ID NO: 228) | (SEQ ID NO: 283) | CTTGACGAATAGGGAAAG (SEQ ID NO: 338 |
| Yes | cagagacttggtctTCAATCT | gacatggttctacaGGATNgG | GTGCCGGAACATGCCAGATCTGGAACCTCTGAAATCTGATCCGGCACCTCATTTTACCGATC |
| | TAATTTTTTTTATTCCTTTTT | TAAAATGAGGTG | CCCCTTCTAAATCACCCTCCTCCCCCGTCCACTTTCACTTTCTTCTGCGACTCCCCAACCCG |
| | TTTCC (SEQ ID NO: | (SEQ ID NO: 284) | CTGTTGTCCGAGACAACCCCCC (SEQ ID NO: 339) |
| | 229) | | |
| Yes | cagagacttggtctCCCATTC | gacatggttctacaNgTTGGt | CCCATTCTCACAACTCCCACCCAGTCCGTTCACTTTCGTTTGTGACACAGCAACCCTCTGGC |
| | TCACAACTCCC | tAGTGATTGGTG | CTCCCCACCGCTCTTCACTTTCGTCCATGAAAGCCACCACCCCCCGCTCTTCACCTTCGTCT |
| | (SEQ ID NO: 230) | (SEQ ID NO: 285) | ATGACACCAATCACTGGCCAACG (SEQ ID NO: 340) |
| Yes | gacatggttctacaCATTacN | cagagacttggtctGGNgttT | ACACATGGCTTTATCGCTACCGGAAGACAGTACTGTAAACAACAACATGGACACAGAAATGG |
| | TATCCTTCCCTTAT | AAAAtAGAtAATAGTG | GAGCGTTGTTTGCACTATTGTCTGTTTTAGGCGCCATTGTGCAGTTATTCATTTGATACATT |
| | (SEQ ID NO: 231) | (SEQ ID NO: 286) | TAGTAACAACTTGACCTCCTTGTC (SEQ ID NO: 341) |
| Yes | gacatggttctacaaTCAaAa | cagagacttggtctGTGAATG | GTCAGAGTTCTGGTTGCTGGCTTGTCTGGTCAGTGCAGCGCTGCCGACTGAAGCTTCATCCT |
| | TTCTaaTTaCTaaCTTaTCTa | TTTTAGTGTGTGTG | CCCTCCTCTTCTTCATCGCCATGGAGACCTGCCATCATCATAACGCATTAGCAACTACACAC |
| | aTC | (SEQ ID NO: 287) | ACACACTAAAACATTCAC (SEQ ID NO: 342) |
| | (SEQ ID NO: 232) | | |
| Yes | gacatggttctacatTNgtTt | cagagacttggtctACAaATA | CTCGCTCACTCTGAGCATGTGTGCAGATCTGACGGTCAAGTCTTTTTTTTCTTCATTTATTC |
| | AtTtTGAGtATGTGTG | aTATAAAAATCAaaAATTTaA | CCAAATACTTACAGCCATTAAAGATACCACTCTGTCACCACGAAGACCAAGTCAAATTCCTG |
| | (SEQ ID NO: 233) | CTTaaTC | ATTTTTATACTATCTGT (SEQ ID NO: 343) |
| | | (SEQ ID NO: 288) | |
| Yes | cagagacttggtctCCTTATa | gacatggttctacaGTtTGtA | CTCCCTCCTGAAGTGTCTTCATTTCATCTCGTCTTTTCTCAAGCACTTGTTCAATAAATGCT |
| | CTaaAaCTCCCTCC | TTAtTtTGAGTGTGTG | GAAGTTTGAGAGCCGTGTTGAGTCCGTCATTACTGTGTTCAGTGTTTCCTCCACACACTCAG |
| | (SEQ ID NO: 234) | (SEQ ID NO: 289) | AGTAATGCAGACCTGACCATCAAAA (SEQ ID NO: 344) |
| Yes | gacatggttctacaATGANgG | cagagacttggtctCATTTAC | ATGACGGCAACTGTTCATCATAAAATGTGAAAAGTTCTCAAAATTAAACACCCTGGTTAATG |
| | tAAtTGTTtATTATAAAATGT | CCAATTCTCAaTTCC | ATATTAACAATGACTTCATGTGCTCATAAACATTCAAACTCATTAACGTAACATGGGAACTG |
| | GAA | (SEQ ID NO: 290) | AGAATTGGGTAAATG (SEQ ID NO: 345) |
| | (SEQ ID NO: 235) | | |
| Yes | gacatggttctacaACCCCAC | cagagacttggtctGGAANgT | ACCCCACATCTAGTTCTCCTGTATGTCCACACATGTAGAAGACAGACAATAAAGCCGGATTT |
| | ATCTAaTTCTCC | GTtTGTGTGGTG | GACTCACATGATGGTCTGGACGACGCGCGGGTGGCGGCTCATGCCCAGATAGTCATTACTGC |
| | (SEQ ID NO: 236) | (SEQ ID NO: 291) | ACCACACAGACACGTTCC (SEQ ID NO: 346) |
| Yes | gacatggttctacatAAAtTA | cagagacttggtctcNCTATa | CAAACTACCTTCTGCCAGCAGGTGGCGTTATGACTGTGACTCAATATTGTTATGTGGATGTG |
| | ttTTTGttAGtAGGTGG | aAACAaaAAaTCCTTC | TTCAGGAGCGGACTTTTATCAATCATGTGAAGTTTCAGGCAGATCAGATATTGTGTGGTTGA |
| | (SEQ ID NO: 237) | (SEQ ID NO: 292) | GTTAGAAGGACTTCCTGTTCCATAGCG ( (SEQ ID NO: 347) |
| No | gacatggttctacagtTNgAt | cagagacttggtctaTaCAaT | GCTCGACATTTGGTTATTGTTAGTCAACAAACGCCGGATGAACAATGAATGCGATGGCAAAG |
| | ATTTGGTTATTGTTAG | TaTTCATTTaTTACATTTAaT | AAAGCAAGAGAGGTATTTTTGTGGACAGGCGACGAGGTCGAGTTGTTACTAAATGTAACAAA |
| | (SEQ ID NO: 238) | AACAACT | TGAACAACTGCAC |
| | | (SEQ ID NO: 293) | (SEQ ID NO: 348) |
| No | gacatggttctacatAttNgG | cagagacttggtctATCTACA | CACCCGGTGGTCTTAGGATGACAGAGAATAGTGAACAATAATAATTTAAGCTGATAAATATT |
| | TGGTtTTAGGATGA | TACCAATATTaAaTTACAaTC ATA | TTAAAAATCAGAGAAAATCACTGATCTATGCCAAACTTCCTTCTGTCAGCAGGTGGCGGTAT |
| | (SEQ ID NO: 239) | (SEQ ID NO: 294) | GACTGTAACTCAATATTGGTATGTAGAT (SEQ ID NO: 349) |
| No | cagagacttggtctCATTacN | gacatggttctacaGTGtAAA | CATTGCGTATCCTTCCCTTATTCGTCAAGCCCTTATCACATGACTATAACATACGGCTTTAA |
| | TATCCTTCCCTTAT | tAANgtTtTTATTTtTGTG | CGCTACCGGAAGACAGCAGACCAGCCTTCACTGTGAACAACAACACGGGCACAGAAATAAGA |
| | (SEQ ID NO: 240) | (SEQ ID NO: 295) | GCGTTGTTTGCAC (SEQ ID NO: 350) |
| No | cagagacttggtctCTCTaAa | gacatggttctacagNgGATG | CTCTGAGATCTGATTTGGCATTTTACTGATCCTCCTTCAACCCCGTTTACTTTCACTTTCT |
| | ATCTaATTTaaCATTTTACTa | AAAGTGAGtAGG | CCGCGACTCTCCAACCCCCATCACTGCTGTCCGTGACACCCCCGCCCTGCTCACTTTCATCC |
| | ATC | (SEQ ID NO: 296) | GC (SEQ ID NO: 351) |
| | (SEQ ID NO: 241) | | |
| No | gacatggttctacaGTGNgAA | cagagacttggtctCATTacN | GTCCATGTTGTTGTTTACAGTGAAGGCTGGTCTGCTGTCTTCCAGTAGTGTTAAAGCCATGT |
| | tAAtAtTtttAtTTtTGTG | TATCCTTCCCTTAT | GTTATAGTCATGTGATAGGGGCTTGACGAATAAGGGAAGGATACGCAATGACACaaaagccc |
| | (SEQ ID NO: 242) | (SEQ ID NO: 297) | caatcaggtagcg (SEQ ID NO: 352) |
| No | cagagacttggtctTTATtAA | gacatggttctacacNACTCC | AGTGCTTAATTTGTGAATTGCGAGGTCCCGGAACAGATCGGGGTAACGGATCCAGAATATAA |
| | GTTtAAtAGTGtTTAATTTGT | CCAACCCTC | CTCAAGGATGGAGAGGTGTCGCGGTTGAAAGTGAAGAGGGTTGGGGAGTCGCAGAAGAAAGT |
| | GAATT | (SEQ ID NO: 298) | GAA (SEQ ID NO: 353) |
| | (SEQ ID NO: 243) | | |
| No | cagagacttggtctcNAAaTA | gacatggttctacaGTGATGN | CGAAGTACATTATTCTGCTTATACCACAGTTCCCACAGCTAAATCCACTGTTCAGATGTTGT |
| | CATTATTCTaCTTATACC | gTAAGAAAtTAGTG | ATTTTATACATTTGTCAGGTTTTTGTTCTCAAGCTGTTCCTGTGTGCAGCACTAGTTTCTTA |
| | (SEQ ID NO: 244) | (SEQ ID NO: 299) | CGCATCAC (SEQ ID NO: 354) |
| No | gacatggttctacaGGNgttT | cagagacttggtctCCTTATT | AACAGACAATAGTGCAAACAACGCTCCCATTTCTGTGTCCATGTTGTTGTTTACAGTGAAGG |
| | AAAAtAGATAATAGTG | CNTCAAaTTCCTATC | CTGGTCTGCTGTCTTCCGGTAGCGTTAAAGCCATGTGCAGTCATGTGATAGGAACTTGACGA |
| | (SEQ ID NO: 245) | (SEQ ID NO: 300) | ATAAGGGAAGGATAC (SEQ ID NO: 355) |
| No | gacatggttctacaaTCATaT | cagagacttggtctTATAtTG | GTCATGTGACACTCAGTCATGTTACTAATTCGTTTACTTTATTTTCGTTGTGAGTATGATTT |
| | aACACTCAaTCATaTTAC | GAtNgtTTTAAAGAAtAG | TAGTGATGTCGTCCTTACTGTGAGGGTAAGCAGCGTCATCAGCAGGATACTGTTCTTTAAAG |
| | (SEQ ID NO: 246) | (SEQ ID NO: 301) | CGGTCCAGTATA (SEQ ID NO: 356) |
| No | cagagacttggtctNgGATGT | gacatggttctacaATAAAAA | CGGATGTGATGTATTTCAGTGTTTAAGCAGGACCGTGCATGCGAGATAAGAAATTGTAGTTT |
| | GATGTATTTtAGTG | AAAAATATTAaTTTTACTACT | TACTGGTTATTGTTTATATCAGAAAAAGTCTATATTAAAATAAAACATTTCTATTCATAAAT |
| | (SEQ ID NO: 247) | CATTTAT | GAGTAGTAAAACTAATATTTTTTTTTTAT (SEQ ID NO: 357) |
| | | (SEQ ID NO: 302) | |
| No | gacatggttctacaAaTCcNa | cagagacttggtcttATtATt | AGTCCGGATACCAATTAATTTCCTATGACGGTCACTTTTGACCGGGAACACCACAGGTGTAA |
| | ATACCAATTAATTTCC | tAtATTATGtAttTAAATATA | CAAAGTTGATTAAAACACTCAAAATTCAATGAAAGAGATGATAATCACTTCTATATATTTAG |
| | (SEQ ID NO: 248) | TAGAAGTG | GTGCATAATGTGGATGATG (SEQ ID NO: 358) |
| | | (SEQ ID NO: 303) | |
| No | gacatggttctacaATTtATT | cagagacttggtctTTCCATa | ATTCATTATAACTCATTACCAGTGCTTAATTTGTGAATTGCGAGGTCCCGGAACAGATCGGG |
| | ATAAtTtATTAttAGTGtTTA | ACTCCTCAACCC | GTAACAAGGACGTGGACGAAGGACGAAAGGGAAGAGCAGAGGTTTGTCGCGGACAAAAGTAA |
| | ATTTGTGA | (SEQ ID NO: 304) | AGAGGGTTGAGGAGTCATGGAA (SEQ ID NO: 359) |
| | (SEQ ID NO: 249) | | |
| No | cagagacttggtctTCATTTT | gacatggttctacaTTGNgGA | TCATTTTACCAATCCCCCTCTTCAACCGCCCTTCTCCCCTGTCCGCTGTTTACTTTCCCTTT |
| | ACCAATCCCCCTC | tAAAGAATAAAAGTGA | CTTCTGCGACTCCCCAACCCTCTTCACTTTTTTCCACGACAACCTTCCGCTCTTCACTTTTA |
| | (SEQ ID NO: 250) | (SEQ ID NO: 305) | TTCTTTGTCCGCAA (SEQ ID NO: 360) |
| No | gacatggttctacaTcNaAAC | cagagacttggtctTttAtTT | AATAATAAGAGCAAGTGGATTTTTAAAAATCTTTATAAAAAGAACAAACAAAAACATATTCA |
| | TCCACTCAaCTC | GtTtTTATTATTAtAtTGGAT | GCCCCCTTTTTTatatttattatctaaattttatttttttcaatggtttccagatcttattt |
| | (SEQ ID NO: 251) | TTAAAGT | caatctcaataaatatagatttt |
| | | (SEQ ID NO: 306) | (SEQ ID NO: 361) |
| No | gacatggttctacaaaCcNTa | cagagacttggtctATGGTGt | CCCTTATTCGTCAAGGCCCTATTACTTGACTAAAAATAAATTACTTTAATGCTGGAAGACAG |
| | TATCCTTCCCTTAT | tTAAAAtAGAtAATAGTG | ACCAGCCTTCACTGTAAACAACAACACAGACACAGAAATGGGAGCTTGTTTGCACTATTGTC |
| | (SEQ ID NO: 252) | (SEQ ID NO: 307) | TGTTTTAGGCACCATTGTGAATGTATTCA (SEQ ID NO: 362) |
| No | cagagacttggtctcNCTCTa | gacatggttctacaTtATTtN | CGCTCTGCTGCCCCCTTCACGACAGTCCCGTTATCGGTTGTCAGGACAACAGCCCGTTCTGT |
| | CTaCCCCCTTC | gAAtAGtAGAAGTGTG | GCGCGGGCACGTATCACAAAAGACCGCCAAGCGTGTGCCCGAGAACACCAAAAGAGCACGCG |
| | (SEQ ID NO: 253) | (SEQ ID NO: 308) | CACAGACACACTTCTGCTGTTCGGAATGA (SEQ ID NO: 363) |
| No | cagagacttggtctcNTCATT | gacatggttctacaGTGtTGt | GCTTCAACGCTACCGGGAGACAGCACTGTAAACAACAACATGGGCACAGAAAAGGGATCGTT |
| | aTATCCTTCCCTTA | tTAAAAtAGATAATAGTG | GTTTGCACTATTGTCTGTTTTAGGCAGCACAGTTATTCATTTGTTACGTTTAGTAACAACTC |
| | (SEQ ID NO: 254) | (SEQ ID NO: 309) | GTCCTCGTCGTCTGTCCACAAAAA (SEQ ID NO: 364) |
| No | gacatggttctacatAGAtTG | cagagacttggtctacNCTTC | CAGACTGGTACAGACTGATGTGACTTGAGTGGGGTGGGGGTGTTTTATTTCTACATATATAC |
| | GTAtAGAtTGATGTGA | AAaTaaaaaAAAAAaTaaaaa | GCCTTTTTTTAGGTGAGGGAATGGAAGTTTTGAGAGAGTTCCCCCACTTTTTCCCCCACTTG |
| | (SEQ ID NO: 255) | AACTC | AAGCGC (SEQ ID NO: 365) |
| | | (SEQ ID NO: 310) | |
| No | gacatggttctacaCATTacN | cagagacttggtctTGTttAT | CATTGCGTATCCTTCCCTTATTCGTCATCTATACCCCTATAACCACGTACCCCTATCACATG |
| | TATCCTTCCCTTAT | GTTGTTGTTTAtAGTGAA | ACTATAACACATGGCTTTAACGCTACCGGAAGACAGCAGACCAGCCTTCACTGTAAACAACA |
| | (SEQ ID NO: 256) | (SEQ ID NO: 311) | ACATGGACA (SEQ ID NO: 366) |
| No | gacatggttctacaTaTCATT | cagagacttggtctttATTTt | TGTCATTGCGTATCCTTCCCTTATTCGTCAAGCCCATATCACATGACTATAACACATGGCTT |
| | acNTATCCTTCCCT | TGTGTttATGTTGTTGTTTAt | TAATGCTACCGGAAGACAGCAGACCAGCCTTCACTGTAAACAACAACATGGACACAGAAATG |
| | (SEQ ID NO: 257) | AGT | G (SEQ ID NO: 367) |
| | | (SEQ ID NO: 312) | |
| No | gacatggttctacatTGAGTA | cagagacttggtctCATTaAT | CTGAGTAGCTTTCTCAGATGTGGGCATAACTTATGTTCGCTCATTTAATATGTTACGTGTCG |
| | GtTTTtTtAGATGTGG | CAcNaAATACATACCT | CCAGATGGTTTTACTCACTCGTTTCCAGTGGTTTTTGGATTGCCAGGTATGTATTCCGTGAT |
| | (SEQ ID NO: 258) | (SEQ ID NO: 313) | CAATG (SEQ ID NO: 368) |
| No | gacatggttctacatTTTTTN | cagagacttggtctAATaaac | CTTTTTCGTATGTTTTGGCATGTTTGAGGTGTGTGCCGATTTTCTTGCATGTGCGTGATTCG |
| | gTATGTTTTGGtATGTTT | NTaaCAAAATaACTCAAC | TGGATCGGGGGCTTGTCCGGTTAATTTTTCTAGGTGGCGCTGTTGAGTCATTTTGCCACGCC |
| | (SEQ ID NO: 259) | (SEQ ID NO: 314) | CATT (SEQ ID NO: 369) |
| No | gacatggttctacaTaATTAA | cagagacttggtctTtTTAAG | TGATTAAATTCCTCTCCTGAAGAAATCTACATTGCAATATTGAGTCACGGTCATAGCGCCAC |
| | ATTCCTCTCCTaAAaAAATCT | tNgAtATATATATAAAAttTG | CTGCTGACAGAAGGAAGTTTGGCATAAATCTGTGATTTTCTCAGGTTTTATATATATGTCGG |
| | ACA | AGAAA | CTTAAGA (SEQ ID NO: 370) |
| | (SEQ ID NO: 260) | (SEQ ID NO: 315) | |
| No | cagagacttggtctCTCATCT | gacatggttctacaAGtAGTA | CTCATCTGTCCAGCATCTCCAGAACCAGCGACAAACACAGAGAAACGGCAGCGCTCTGGCTG |
| | aTCCAaCATCTCC | tTGGATttAtAGtAGAAA | TCAGAGCTGGTGGAGGAGCCCGTCATGTCCAGCACATGTGTTTCTGCTGTGGATCCAGTACT |
| | (SEQ ID NO: 261) | (SEQ ID NO: 316) | GCT (SEQ ID NO: 371) |
| No | cagagacttggtctCAAaAaa | gacatggttctacatAtTNgT | CAAGAGGCCTTTCCAAAAAAAAAATGTAATCATATTAATCCGCATGTAGCTTACATCAGCAC |
| | CCTTTCCAAAAAAAAAATaTA | TTGTGtTTGTGTGTG | ACAAACACAACACAAACGTCCTCTTTTTTGATGCGCGCACACACACACAAGCACAAACGAGT |
| | ATC | (SEQ ID NO: 317) | G (SEQ ID NO: 372) |
| | (SEQ ID NO: 262) | | |
| No | cagagacttggtctCTTaTTC | gacatggttctacaGGNgttT | ACTATTGTCTGTTTTAGGCGCCATTGTGCAGTTATTCATTTGTTACGTTTAGTAACAACTCG |
| | ATCAAaCCCCTATC | AAAAtAGATAATAGTG | ACCACGTCGTCTGTCCACAAAAATACCTCTTCTGCTTTCTCTGCCATCGTGTTCATTGTTCC |
| | (SEQ ID NO: 263) | (SEQ ID NO: 318) | ACCGGCGTTTGTTGACT (SEQ ID NO: 373) |
| No | cagagacttggtctAAGATAt | gacatggttctacaTTTTATC | AAGATACAAACTAAATTAAAGCTGCAAGCAGTGATGAAAGGGATCTCGAACCCGGGCTCACC |
| | AAAtTAAATTAAAGETGtAAG | TTTATCAaCTTAAATTATTAT | GCTGCCTTGTGGCCTTAGGATGACAAAGCACAATGGACAATAATAATTTAAGCTGATAAAGA |
| | tAGTG | TaTCCAT | TAAAA (SEQ ID NO: 374) |
| | (SEQ ID NO: 264) | (SEQ ID NO: 319) | |
| No | gacatggttctacaAATGGTG | cagagacttggtctCATTacN | AATGACACaaaagccccaatcaagtagcgaatcgcggcggccccgcctccgttttcagatgt |
| | ttTAAAAtAGAtAATAGTG | TATCCTTCCCTTAT | ctctgttttttcctcatccacactgaaacggagcagcagcgttttagaatgaaaacggcctc |
| | (SEQ ID NO: 265) | (SEQ ID NO: 320) | tccagcgtttttgaaacgctccgt (SEQ ID NO: 375) |
| No | gacatggttctacaAtTGTGG | cagagacttggtctACCTCAA | ACTGTGGGATTATAATGCTTAGTATGACTGAATTCAACCAATCGCTCCATCTGTGATGATAA |
| | GATTATAATGETTAGTAT | ATATTAAaTCAACCCA | TCTGCGATTTTATCAATAGACCGGAGGCCAGACTGAGAGAGATATGTGGGTTGACTTAATAT |
| | (SEQ ID NO: 266) | (SEQ ID NO: 321) | TTGAGGT (SEQ ID NO: 376) |
| No | gacatggttctacaAATTATT | cagagacttggtctaAaAcNa | AATTATTTATTCAGTGATGGTAAGTAAAAGTTTGTTTTAAATAAAAAGTCTGCGTCTCCCTG |
| | TATTAGTGATGGTAAGTAAA | AaCAaaTCAAAACCCT | TGTTGCTCAGGCTGCACTGCAGTGTCTATTCACAGGTGCGATCCCACTACTGATCGGCACGA |
| | AGT | (SEQ ID NO: 322) | GGGTTTTGACCTGCTCCGTCTC (SEQ ID NO: 377) |
| | (SEQ ID NO: 267) | | |
| No | cagagacttggtctATAtTAA | gacatggttctacaaTCTTCC | ATACTAACACATTATTTACTTACAATTAACAGAAGAAATTGAGCAGATTTTTTTGCCATTTT |
| | tAtATTATTTAtTTAtAATTA | AaTTaAaaAaATaTCTCTC | GTACACAGCCAATCACTTGGCGCCATTTACTAAATAATCTTTCTGTTCTCAGCATCCCGAGA |
| | AtAGAAGAAA | (SEQ ID NO: 323) | GAGACATCTCCTCAACTGGAAGAC (SEQ ID NO: 378) |
| | (SEQ ID NO: 268) | | |
| No | cagagacttggtctCAaATTT | gacatggttctacaAAtTtAG | CAGATTTATGCCAAACTTCCTCCTGTCAGCAGGTGGCGCTATAACTGTGACTTAAGATTGGC |
| | ATaCCAAACTTCCTCC | ttAtAtAATGTtttATGTGtt | ATGTAGATGTCTTCCGGAGAGGAATCTTATCAACCATGTGAAGTTTCAGGCACATGGGACAT |
| | (SEQ ID NO: 269) | TGAAA | TGTGTGGCTGAGTT (SEQ ID NO: 379) |
| | | (SEQ ID NO: 324) | |
| No | gacatggttctacaAAtTtAG | cagagacttggtctTATaCCA | AACTCAGCCACACAATGTCCCATGTGCCTGAAACTTCACATGGTTGATAAGATTCCTCTCCG |
| | ttAtAtAATGTtttATGTGtt | AACTTCCTCCTaTC | GAAGATATCTACATGCCAATCTTAAGTCACAATTATATCGCCACCTGCTGACAGGAGGAAGT |
| | TGAAA | (SEQ ID NO: 325) | TTGGCATA (SEQ ID NO: 380) |
| | (SEQ ID NO: 270) | | |
| No | cagagacttggtctCAaaAAa | gacatggttctacaAAGGttT | CAGGAAGTCGGATATTTTGTACTTCCTGCGGCGAAAAAGTGGCGATTTTGCCATTTCCAGGC |
| | TcNaATATTTTaTACTTCC | TTAGATGAAAAAttTAATTT | GTTGTATTTTAACGAACTCCTCCTAGGAATTTTATCCGATCGACACCAAAATTAGGTTTTGT |
| | (SEQ ID NO: 271) | TGGTG | CATCTAAAGGCCTT (SEQ ID NO: 381) |
| | | (SEQ ID NO: 326) | |
| No | cagagacttggtctTTTATaC | gacatggttctacaATAAtTt | TTTATGCCAAACTTCCTTCTGTCAGCAGGTGGCGCTATAACTGTGACTCAAGATTGGCATGT |
| | CAAACTTCCTTCTaTC | AGttAtAtAATGTtTtATtTG | AGATGTCTTCCGGAGAGGAATCTTATCAACCATGTGAAGTTTCAGGCAGATGAGACATTGTG |
| | (SEQ ID NO: 272) | ttTGAAA | TGGCTGAGTTAT (SEQ ID NO: 382) |
| | | (SEQ ID NO: 327) | |
| No | cagagacttggtctCTCATTT | gacatggttctacaTTGtAAA | CCCTCCTCCCCTGTTCGCTGTTCACTTTCACTTTTTTCCGTGCCTCCCCAACTCTTCACTTT |
| | TACTaATCCCCCTC | NgATAGAGAGtAGG | CGTCCGTAACACCCCAGCCTGCTCTCTATCGTTTGCAACACTCCtccaccatccactgcacc |
| | (SEQ ID NO: 273) | (SEQ ID NO: 328) | atccc (SEQ ID NO: 383) |
| No | gacatggttctacaATGNgGA | cagagacttggtctaTAAATA | ATGCGGAATAATTGACACCAGTGCGTTGAATTATTAGAAAAATTGAACCACTTTAAATTTTG |
| | ATAATTGAtAttAGTG | TaTTaaaaTTTAATCTaTaAT | ACTGCAATTTATTACAGATAAAGTACATGCAGGAGGTTATTATCACAGATTAAACCCCAACA |
| | (SEQ ID NO: 274) | AATAACCTCC | TATTTAC (SEQ ID NO: 384) |
| | | (SEQ ID NO: 329) | |
| No | cagagacttggtcttAAAtTA | gacatggttctacaCACAATC | CAGCAGGTGGCGTTATGACTGACTCAATATTGTTATGTGGATGTGTTCAGGAGCGGACTTTT |
| | tTTTTGttAGtAGGTGG | TCCACACAATCTC | ATCAACCATGTGAAGTTTCAGGCAGATCAGAGATTGTGTGGAGATTGTGTTTTAAGACAAAC |
| | (SEQ ID NO: 275) | (SEQ ID NO: 330) | TA (SEQ ID NO: 385) |
| No | gacatggttctacaGNgAATG | cagagacttggtctAAAaCCT | AGGCTTTGTATATCGGATGGGTGTCATCTGCGTTCTTGTTTGCCGGAGGATGCATCTTCATA |
| | GTTtTGATTATTAGTG | CCCCTAaTTCCC | TGCTGCAGTGGTTCTCTAGACAAGGGTCCGGATCCAAAGTATATGTACTCCAGGAATGCACC |
| | (SEQ ID NO: 276) | (SEQ ID NO: 331) | TGCTCCATATGTGGCCTACCAGCCTC (SEQ ID NO: 386) |
| No | cagagacttggtctAAAtAAA | gacatggttctacaaCATCAa | AAACAAAATGAACAACAGTGAAATACTGTGAAATGATGATTGCTAAAAAGTAAGCGAGTCGA |
| | ATGAAtAAtAGTGAAATAt | aTaCAAaTTTTATAACCC | TGCAATTGACACTAGATTTTTAGCAAGCCACTGCTGACTTGACTCTCTAGGGGTTATAAAAC |
| | TGTGAAA (SEQ ID NO: | (SEQ ID NO: 332) | TTGCACCTGATGC (SEQ ID NO: 387) |
| | 277) | | |
| No | gacatggttctacaAAtTTAG | cagagacttggtctTTaTaaa | AACTTAGCCACACAATGTCTGAACTGCTTGAAACTTCACATGGTTGATTAAATTCCTCTCCT |
| | ttAtAtAATGTtTGAAtTGtT | aCTaAaTTaTAAaCCAAACTA | GAACACATTTACATGCCAATATTGAGTCTGTCATAGCGCCGCCTGCTGGCAGAAGGTAGTTT |
| | TGAAA | CCTTC | GGCTTACAACTCAGCCCCACAA (SEQ ID NO: 388) |
| | (SEQ ID NO: 278) | (SEQ ID NO: 333) | |
| No | gacatggttctacaGtNgGTT | cagagacttggtctTTaAaCT | GCTCCCCCAGGAGCACCATATCGATACCGAACTTAGTGTGGACACCTGATCAACATAATCAC |
| | tAttttTtATTAtAtAAttTG | CAaaaCTTCTaaACTaCA | ACTGCAGTCCAGAAGCCCTGAGCTCAAGCGATCCGCAGCCTCAGCCTCCCAGTAGCTTGGAT |
| | GTGGT | (SEQ ID NO: 334) | TAC (SEQ ID NO: 389) |
| | (SEQ ID NO: 279) | | |

### Barcoding and Sequencing

Oligonucleotide barcodes with universal CS1 and CS2 were ligated to the multiplex PCR products using the GoTaq Hot Start Polymerase (Promega) reaction mixture as described in the manufacture's protocol and using the following cycling conditions: 94° C., 5 min; 12 cycles (97° C., 15 s; 45° C., 30 s; 72° C. 2 min); 72° C., 2 min; 4° C. hold. Barcoding was performed using an Eppendorf ProS 96. The Illumina MiSeq Reagent Kit v2 (300 cycle; PN MS-102-2002) was used for sequencing in accordance with the manufacturer's instructions.

### Data Analysis and Age Estimation

SeqKit v1.2 was used to hard clip the reads by 15 bp at both 5′ and 3′ ends to remove adaptor sequences (Shen et al., 2016). Clipped reads were aligned to a reduced representation genome of each species closest relative genome. Lungfish reads were aligned to the zebrafish genome. Bismark v0.20.0 was used to align reads with the following parameters: --bowtie2 -N 1 -L 15 -bam -p 2 -score L, −0.6, −0.6 -non_directional. Methylation calling was performed using bismark_methylation_extractor function with default parameters (Krueger and Andrews, 2011).

70% of the samples were randomly assigned to a training data set and the remaining into a testing data set. Age in years was natural log transformed and an elastic net regression model was applied on the training data sets. Age was regressed over the methylation of each CpG site that was captured during sequencing. The glmnet function used for the elastic net regression model was set to a 10-fold cross validation with an α-parameter=0 (Friedman et al., 2010). The α-parameter was set to 0 to force all sites to be used in the model, as opposed to Example 1 where it was set to 0.5 to identify the minimum number of sites required (Horvath, 2013; Stubbs et al., 2017; Thompson et al., 2017). All analyses were performed in R using version 3.5.1 (R Core Team, 2013).

31 CpG sites were used to calibrate the age estimator model. These 31 CpG sites are shown in Table 12 and referred to as the Lungfish clock.

TABLE 12
Age associated CpG sites used to estimate age in the Australian
Lungfish. The genomic coordinates are based on the zebrafish
genome (danRer10). The intercept is 2.371135587. The coefficient
is also referred to as weight.

| Chromosome | Position | Coefficient |
|---|---|---|
| chr10 | 11142079 | −0.009645245 |
| chr10 | 11142129 | 0.003096468 |
| chr11 | 12533451 | 0.002383155 |
| chr11 | 16311410 | 0.003118103 |
| chr11 | 18605931 | 0.002383005 |
| chr11 | 4718419 | 0.004295323 |
| chr11 | 4718545 | 0.006309799 |
| chr11 | 4718873 | 0.000000181 |
| chr13 | 40742082 | 0.001309717 |
| chr13 | 40742348 | −0.009898325 |
| chr14 | 42183036 | −0.013993076 |
| chr15 | 20939064 | 0.004516888 |
| chr15 | 20938558 | 0.005115907 |
| chr16 | 53732412 | −0.009898102 |
| chr16 | 53732506 | 0.00238343 |
| chr16 | 8112979 | −0.004591806 |
| chr17 | 30406809 | 0.003567435 |
| chr1 | 17053944 | 0.000056200 |
| chr1 | 58411664 | −0.009806872 |
| chr1 | 58529962 | 0.000047300 |
| chr23 | 2856982 | 0.000001640 |
| chr25 | 5024662 | 0.005095162 |
| chr4 | 72407427 | −0.000252804 |
| chr4 | 72407518 | −0.006362557 |
| chr6 | 57183503 | 0.003501601 |
| chr7 | 21450653 | −0.009898003 |
| chr7 | 53689428 | −0.032923816 |
| chr7 | 69800198 | 0.000006860 |
| chr7 | 73184405 | 0.001813072 |
| chr8 | 50244781 | 0.005366123 |
| chr8 | 50245443 | −0.000091100 |

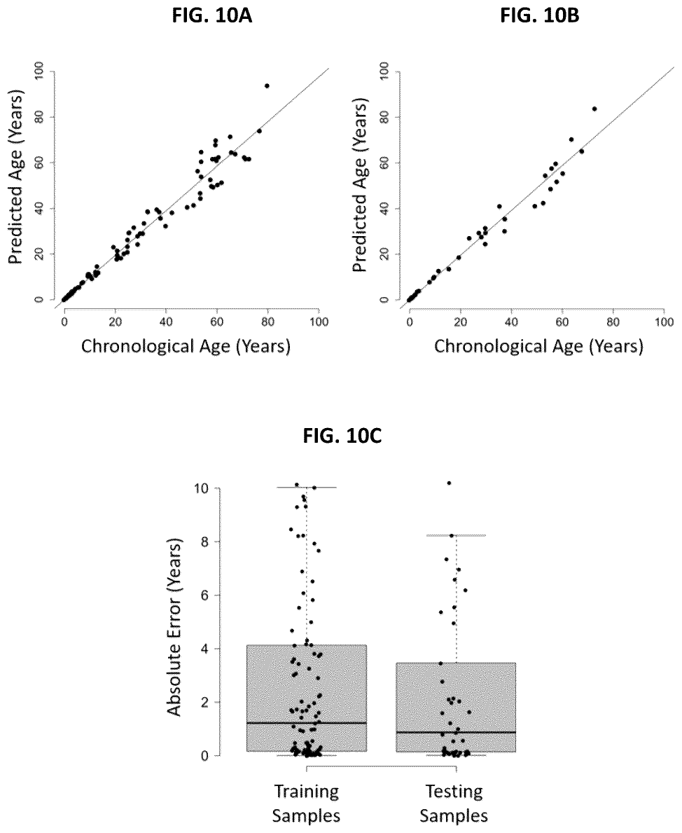

For the Lungfish clock, the inventors found a high correlation between the chronological age and the predicted age in the training data set (Pearson correlation=0.98, p-value=2.92×10⁻⁷⁶) and the testing data set (Pearson correlation=0.98, p-value=1.39×10⁻³²) (FIG. 10A-B). The median absolute error (MAE) in the testing data set was found to be 0.86 years (FIG. 10C). No significant difference in MAE was found between the training and testing data sets (p-value=0.67, t-test, two-tailed). The similar correlation in chronological and predicted age and no significant difference in MAE suggests a lack of overfitting in the model. A higher performance of the model was observed at younger ages (Table 13). The Pearson correlation between the chronological and predicted age decreased and the MAE and relative error increased at higher ages. The performance of the model broken down into age intervals suggest it is better suited towards younger individuals. The inventors also tested if the epigenetic clock was performed better with samples of known age or bomb radiocarbon age. A one-way ANOVA was used to test if the absolute error rate was higher with samples from known age or bomb radiocarbon age. Chronological age was used as a blocking factor as most younger ages were of known age (Table 10). The inventors found no significant difference between the error rate of samples from known or bomb radiocarbon age for both the training (p-value=0.413) and testing data set (p-value=0.803).

TABLE 13
Performance of the Lungfish clocks at increasing
age intervals in the testing data set.

| | | MAE | Median Relative |
|---|---|---|---|
| Age Range | Correlation | (Years) | error (%) |
| ≤20 | 0.99 | 0.16 | 8.44 |
| 21-40 | 0.85 | 2.65 | 7.82 |
| 41-60 | 0.71 | 6.90 | 12.52 |
| >60 | 0.60 | 6.09 | 9.30 |

### Grandad Age Estimation

Grandad, an Australian Lungfish was transported from either the Mary or Burnett River in 1933 for the 1933-34 Chicago world fair. Grandad spent 83 years in captivity before being euthanized in 2017, making it the longest-lived fish in a zoo. When captured in 1933, Grandad was already an adult and so the true age has never been determined. Using the Lungfish clock, the inventors predicted the age of Grandad to be 108 years at death. This suggests that, in captivity, Australian Lungfish can live more than 100 years.

### Discussion

In this study, the inventors have developed a DNA methylation age estimator for Australian lungfish, a threatened fish species. This study has used conserved age associated DNA methylation at CpG sites in zebrafish to develop an epigenetic clock for lungfish. This study demonstrates age associated CpG methylation at sites in one fish species can be predictive of age in other species.

### Example 8—Age Estimation for the Murray Cod (Maccullochella peelii) and Mary River Cod (Maccullochella mariensis)

Otolith ageing is also undesirable in other threatened freshwater fish including the threatened Murray cod (Maccullochella peelii) and Mary River cod (Maccullochella mariensis) (Couch et al., 2016; Espinoza et al., 2019). Another limitation of otoliths is the difficulty in ageing both the youngest and oldest fish (Campana, 2001a). The difficulty in ageing otolith can also introduce reader bias, potentially having an impact on any population management (Campana, 2001b). Where otoliths or other ageing methods are not applicable or too expensive, an alternative non-lethal approach to age estimation is required to better manage wild populations.

In this example, the inventors use the age-associated sites of DNA methylation in zebrafish to develop an epigenetic clock for the threatened Murray cod (Maccullochella peelii) and Mary River cod. This study again demonstrates age associated CpG methylation at sites in one fish species can be predictive of age in other species.

### Animal Ethics and Tissue Collection

Collection of fin tissue from known age Mary River cod was approved under General Fisheries Permit 94765 and Animal Ethics Permit CA 2008/03/253 from wild and captive raised fish. Murray cod otolith and fin tissue were collected from multiple rivers along the Queensland and New South Wales border within the Border Rivers region of the Northern Murray-Darling Basin. Collection of fin tissue was approved by CA 2019/04/1276 and the otolith under NSW Animal Research Authority 10/04. Otolith age of Murray cod was conducted using a previous validated method (Gooley, 1992). Table 14 lists the total number and age ranges used for both Murray cod and Mary River cod. DNA extraction and bisulfite treatment.

TABLE 14
Total number of samples and age ranges
used for Mary River cod and Murray Cod.

| | Total | Age Range |
|---|---|---|
| Species | Samples | (Years) |
| Mary River cod | 37 | 0.5-2.88 |
| (Maccullochella mariensis) | (37 known age) | |
| Murray Cod | 33 | 1.1-12.1 |
| (Maccullochella peelii) | (33 otolith age) | |

### DNA Extraction and Bisulfite Treatment

DNA was extracted using the DNeasy Blood & Tissue Kit (QIAGEN) as instructed in the manufacture's protocol. Extracted DNA was bisulfite converted using the protocol as previously described (Clark et al., 2006).

### Identification of Age-Associated CpG Sites and Primer Design

Multiplex PCR was used to develop an assay for age estimation using sites known to be age associated in zebrafish. Primers were designed targeting CpG sites with methylation levels that are both known to significantly correlate with age in zebrafish and are conserved between species. The Mary River cod and Murray cod have a median divergence time of 1.09 million years ago (MYA) (Nock et al., 2010). Due to the low evolutionary divergence time between the species and the Murray cod being the only one with a reference genome, the primers were designed using the Murray cod genome (GCA002120245.1 mcod v1) but were also used on the Mary River cod (Austin et al., 2017). CpG sites conserved between the zebrafish and Murray cod genomes were identified with LASTZ v1.04.00 with the following conditions: [multiple] -notransition -step=20 -nogapped (Harris, 2007). Conserved DNA sequences between the two genomes with methylation-age associated CpG sites were targeted for primer design (primers shown in Table 15).

TABLE 15
Primers used to amplify conserved age associated CpG sites in the Murray cod and Mary River cod. X = Validated for multiplex PCR

| X | Forward primer | Reverse primer | gDNA amplicon sequence |
|---|---|---|---|
| Yes | cagagacttggtctGTttT | gacatggttctacaAAACT | GTCCTCAGTGCTCTGCTTGTCTCAGCAGCGCGAGCCCGCCGCCACAGCTGCAGCCTATATAAC |
| | tAGTGtTtTGtTTGTtTtA | TCAaTTCAaaTCCCAaATT | CATCGGTGACGTTGCGCTGCAGGCCACGCCCCCTCAGATGGACCGAAATCTGGGACCTGAACT |
| | G (SEQ ID NO: 390) | T | GAAGTTT (SEQ ID NO: 486) |
| | | (SEQ ID NO: 438) | |
| Yes | gacatggttctacaGGTGA | cagagacttggtctcNACA | GGTGACTGTAGGCTGCAAAAGGCCATTTTCAGCCTGAAGCGCGTCTTATTGATCGGTGTGACG |
| | tTGTAGGTGtAAAAG | aCAACTCTCCATCC | GTAAAACCTGGACTAACCCCACCGCCGCGCTCCTTCATGTCCCGGGATGGAGAGTTGCTGTCG |
| | (SEQ ID NO: 391) | (SEQ ID NO: 439) | (SEQ ID NO: 487) |
| Yes | gacatggttctacaGTGGt | cagagacttggtctCAAAA | GTGGCTTTATCATAGGCAACTTTAGCAGAAATCCCAATGGCGATGATGAGCGCTATGACCCCG |
| | TTTATtATAGGtAAtTTTA | CCCAaCCCCCTTC | CAGGTGATGTACACCGTGGTGTTGGTCTGGTCCAGCTCCGGGTCGTACGTATCACCTGTTGGC |
| | G | (SEQ ID NO: 440) | GCCGGTGAAGGGGGCTGGGTTTTG (SEQ ID NO: 488) |
| | (SEQ ID NO: 392) | | |
| Yes | gacatggttctacaGGtTG | cagagacttggtctTTCTa | GGCTGAACGGGTTTCTAAAGGGGTTTCCTCGACTCGAAGGCAAGGACCGGAAGAGGAAAGGGA |
| | AANgGGTTTtTAAAGG | CATTaTTTCATTTAaaTTA | GTGCCGGGTTTTGTCATCACTGCCCCGTTGGAGCTCCACTAACCTAAATGAAACAATGCAGAA |
| | (SEQ ID NO: 393) | aTaaAaCTCCA | (SEQ ID NO: 489) |
| | | (SEQ ID NO: 441) | |
| Yes | cagagacttggtctTGGTN | gacatggttctacaCCCCC | CTATCAGTACGTGGCCCCCGGGGTGATCAATTTAGGCTCCCCGCACGGTTATTTCACGGAGGA |
| | gAGAGGAGtAGtAA | TcNTCTTCCTC | AGACGAGGGGGACATCTTCCCGACTCCGGACCCCCACTACGTTAAGAAATACTACTTCCCCGT |
| | (SEQ ID NO: 394) | (SEQ ID NO: 442) | GAGAGACCTGGA (SEQ ID NO: 490) |
| Yes | gacatggttctacaGGANg | cagagacttggtctTaCAa | GCACCTGGTCCAGAATGTCCGTCTGGAGGTCCCCTGCGACTGCAGACCGGGACAGAAGAAGTG |
| | ATTGATTTttAAATtAAAG | TcNCAaaaaACCTCCA | TACCTGCTACCGGCCGAACCGCAAGGAGACCTGGCTCTTCTCCCGGTTCTCCACCGGCTGGAG |
| | G | (SEQ ID NO: 443) | (SEQ ID NO: 491) |
| | (SEQ ID NO: 395) | | |
| Yes | gacatggttctacaGTAAG | cagagacttggtctTCTaC | GTAAGTAGTTAATGTGCACCAGATTTACGCACGGACCTCATTTGACGCAGTGTGGTCAGCGTG |
| | TAGTTAATGTGtAttAGAT | TcNaTCATTCTCTaTC | CACCCACAAACCTCACATGGTAATTTGAGTAATTTAAAGCATTTGACAGAGAATGACCGAGCA |
| | T | (SEQ ID NO: 444) | GA (SEQ ID NO: 492) |
| | (SEQ ID NO: 396) | | |
| Yes | gacatggttctacaCCTCT | cagagacttggtctNgTAG | CCTCTCCGGAGAAGCTTCCAGTTCCAGCCGGTTACGTCCCCGAGCTCGTCCATCGCTGCCCCC |
| | cagagacttggtctNgTAG | GTtTTttTtTTtTGtTttA | GCTGTGGTGAGGCGTTTGGCCAGGCCAGCAGCCTGCGCCTCCACCTGGAGCAGAAGAGGAAGA |
| | (SEQ ID NO: 397) | G (SEQ ID NO: 445) | CCTACG (SEQ ID NO: 493) |
| Yes | gacatggttctacaTTANg | cagagacttggtctAAATA | TTACGTACACATACTGAGGTGTGAGCTGTCAGGGAAGACACTCACTCGGGGAAAACGTGCTCT |
| | TAtAtATAtTGAGGTGTG | CCCATTTCCTCTaTCAAA | CCGTTATGGCCCGATTTCAACTCTATAAACCATACAGAAGGATACATCTCTGTTTGACAGAGG |
| | (SEQ ID NO: 398) | (SEQ ID NO: 446) | AAATGGGTATTT (SEQ ID NO: 494) |
| Yes | gacatggttctacaAAGtt | cagagacttggtctACcNT | CCGCCCAGGGACATGCTCTCAAACCCGGGGCTCGGGTTCGGGTCGATCCGCTCGTCTTCAAAC |
| | tATGtNgtttAGGGAtAT | CAaCCTCTCACTC | TCTTCATCTGATGACGAGGATGACACAGATGAGGAAGAGTGAGAGGCTGACGGTGTTGGAGAG |
| | (SEQ ID NO: 399) | (SEQ ID NO: 447) | (SEQ ID NO: 495) |
| Yes | gacatggttctacagGGGG | cagagacttggtctCTaCC | GGGGGATCTTAATGAAGCAGTCGTTGAAGGAGAGGTTGTGCCGCAGGGAGTTCTGCCACCGCT |
| | ATtTTAATGAAGLAGT | cNaAaAAaATaCTCCC | GCGTGTTCTCCCGGTAGTACGGGAACCGGTCCATGATGAACTTGTAAATCTCACTGAGGGGGA |
| | (SEQ ID NO: 400) | (SEQ ID NO : 448) | GCATCTTCTCCGGGCAG (SEQ ID NO: 496) |
| Yes | gacatggttctacacNaAa | cagagacttggtctGAtAN | CGGAGGACCTCTTTCACCCCGCAGGCGCTCGAGATCCTCAACAGCCACTTTGAGAAGAACACA |
| | aACCTCTTTCACCC | gtAttAttTtttTGTtATA | CACCCCTCCGGACAGGAAATGACGGAAATAGCGGAGAAACTGAACTATGACAGGGAGGTGGTG |
| | (SEQ ID NO: 401) | GTTLAGT | CGTGTC (SEQ ID NO: 497) |
| | | (SEQ ID NO: 449) | |
| Yes | gacatggttctacaCAaCC | cagagacttggtctGGAGT | CAGCCCCAGATCTCCTATCTTGACGGAGCCGGTGGGGCCGGTGATGAAGATGTTGTCGCACTT |
| | CCAaATCTCCTATC | NgTtAGATttTtAAAGG | GAGGTCCCGGTGGATGATGGGAGGAGTGCGCGTGTGCAGGAAGTGAAGCCCTTTGAGGATCTG |
| | (SEQ ID NO: 402) | (SEQ ID NO: 450) | ACGACTCC (SEQ ID NO: 498) |
| Yes | gacatggttctacaCCCcN | cagagacttggtctgtTtA | CCCCGCTGGTTCTCCTTCAAGTCCAGGTAGTATTTGCGGTTTTCCCGGACTAGAAACTCGCTC |
| | CTaaTTCTCCTTC | GtTGGGGtttAGtAGT | TTGAGAGCTCTCCTGGGCCCGCCGTCCTCACCCGCGCTGGCCTGGGCTATCTGCTCTGGACTG |
| | (SEQ ID NO: 403) | (SEQ ID NO: 451) | CTGGGCCCCAGCTGAGC (SEQ ID NO: 499) |
| Yes | gacatggttctacaNgGTA | cagagacttggtctacNaT | TCCCCTCTGAAGCAGATAAGGCTACACGTCAGGTCAGGGCTGGTTCACCGCAGTTCGGCTATT |
| | TttAGTTATTGAAGtAGT | aAACCAaCCCTaACC | GGGGTgtcaagtgtgtttgtgcatgtgtgtgtatttgtgtacgtttgtgtttgtttgactgtg |
| | (SEQ ID NO: 404) | (SEQ ID NO: 452) | catgtgtgt (SEQ ID NO: 500) |
| Yes | cagagacttggtctGAtAt | gacatggttctacaCCTCT | CCAGTTTTGGGCCTGCGGATCAAGAAGGAGAGTCCCGAGCAGAGGAGACAGCGGGAGAAGTCG |
| | AAtAAAtAGTtAGTGGGtA | aCTcNaaACTCTCC | TCTGCTCCCGCCGGGAATCAGCCGCTGGGAGAGTTCATCTGCCAACTCTGTAAAGAGGAGTAC |
| | A | (SEQ ID NO: 453) | CCCGA (SEQ ID NO: 501) |
| | (SEQ ID NO: 405) | | |
| Yes | cagagacttggtctCTTaA | gacatggttctacaGAtTA | CTTGAAAACCCTGCACTCCCATGACGGTGTTGTTGAGCCTGTGTGTCTGGCCGGGAGCGGGCT |
| | AAACCCTaCACTCCC | GTTAGtTTGttTGAAGTG | CTGAATACGTTCTGTACGCCGTGTTTGTAATGTGATGTGGGGCGAGACAGTAGCTAGCTATGC |
| | (SEQ ID NO: 406) | (SEQ ID NO: 454) | ACTTCAGGCAAGCTAACTAGTC (SEQ ID NO: 502) |
| Yes | gacatggttctacaGTGAG | cagagacttggtctTAAAT | GTGAGCGAGTGTTCCCAAAGGGACTTTTTAAGGAGGAACGCTAACCCGCACTCCTCACATATG |
| | NgAGTGTTtttAAAGG | aCTTCcNaTTaTCCTATC | TAGCTCTTCGGCTTCCCGGCGTGAGTCCGCCTGTGTTTCTTCATGTGATAGGACAACCGGAAG |
| | (SEQ ID NO: 407) | (SEQ ID NO: 455) | CATTTA (SEQ ID NO: 503) |
| Yes | gacatggttctacaCACCT | cagagacttggtctgGttT | AGGGCGGAAGGCCGAGCCATAGCTGGGCTTCCTGTTTTGGAGGGTTGAGGTGTCCAGAAGTTT |
| | CCACTTTCACTTCC | TtNgtttTGTtATTAAGG | GAGCCAGCCCTCCGATGTCACAGGGCGAAGGTCAGGGGTTACTGTTGTGCACTCTGGGTCAAA |
| | (SEQ ID NO: 408) | (SEQ ID NO: 456) | TAGCCCGGTTCTGGGTGTTGTAGT (SEQ ID NO: 504) |
| Yes | gacatggttctacacNaaC | cagagacttggtctGAGtt | GGCCCTGTGCTTCGGGCTAGCAGCTGCCACCCTCATCCAGTCTATTGGCCACATCAGCGGCGG |
| | CTCATCATaTCCTTC | AAtAAGGTAGGtAAAGG | CCACATCAATCCTGCCGTCACCTTTGCCTACCTTGTTGGCTCACAGATGTCTCTTTTCCGCGC |
| | (SEQ ID NO: 409) | (SEQ ID NO: 457) | CAT (SEQ ID NO: 505 |
| Yes | gacatggttctacaCACAC | cagagacttggtctAtTGT | TCTACTATGTCACTGGTTTCTTCATCGCCATCTCGGTCATCACTAATGTGGTGGAGACGGTGC |
| | CTCCACCATaaCC | GTAGNgTTtAttAtATGG | CCTGTGGTTCCACCGCCAACCAGAAAGACATGCCATGTGGTGAACGCTACACAGTGGCATTCT |
| | (SEQ ID NO: 410) | (SEQ ID NO: 458) | TCTGCATGGACACCGCC (SEQ ID NO: 506) |
| Yes | gacatggttctacatATtA | cagagacttggtctCTaAT | CACCCAAACAAATACAACCCAAACGAATCGGATAGATGCCAACAACCCGCCATCAGTGGACTA |
| | GTGtTGATtTtTGtAGtAG | aacNaaTTaTTaaCATCTA | ATTTCTCGCGGGTCGGGGCCTGTAACATCCACCGACGAAGTGCTGGAGTCCAGTCAAGAGGCG |
| | T | TC | CTGCACGTCACGGAGCGTC (SEQ ID NO: 507) |
| | (SEQ ID NO: 411) | (SEQ ID NO: 459) | |
| Yes | cagagacttggtctaaaTC | gacatggttctacaTTAtT | TAAACATCCTTGACTGGAAGCTGGGGTTTGGACAACGAGGGCTTTCAGTCGGATGTTTGCAGC |
| | TTCATCTCAaaTaTTTTTC | GTTGGtTGTtAGAGGtAA | ATCTTATTGCCTCTGACAGCCAACAGTAACCCCAGCGATGACACTAGTTTGTAACATTTGTAA |
| | C | (SEQ ID NO: 460) | CAGAAAAGCAGAACT (SEQ ID NO: 508) |
| | (SEQ ID NO: 412) | | |
| Yes | cagagacttggtcttANgG | gacatggttctacaAaaTC | AACCACCTGCAGTTCCAGGCTGATCCGGACGTGCTGCACAACAGCTACGCCCTGAGAGGGATC |
| | AGttATGtAGGGtATG | CTaaTTaTAaTaaATCCCT | CACTACAACCAGGACCTCATTAACCTGGCGGTGCTGCTGGACATGGAGGGGAAGCCTTTTCTT |
| | (SEQ ID NO: 413) | C | CACGTGTC (SEQ ID NO: 509) |
| | | (SEQ ID NO: 461) | |
| Yes | cagagacttggtctGtAAt | gacatggttctacaACcNT | GCAACCAGGACATGCTGTGCATGGACTACAACCGCAGCCAGACCACCACTGCATCTCCCGTGG |
| | tAGGAtATGtTGTGtATG | AaCTaACCTTCTTCC | TGGCGAAGCCGACCAACCGGCCACTGAAGCCGTACAACCCCAGGAAGAAGGTCAGCTACGGT |
| | (SEQ ID NO: 414) | (SEQ ID NO: 462) | (SEQ ID NO: 510) |
| Yes | cagagacttggtctAGAGt | gacatggttctacaTTaAT | CCCTCTGATACTCTTTCATGTCTCATTGTATCTCTCAGGGAGTCTTTGGAGGCCCTGCTCCAA |
| | TGtNgGtAttAGGTAA | aAAaCATaTaAaAaTAAAa | AGGGCCGTGGCTCACTGTCCCAAGGCAGAGGTCCTGTGGCTGATGGGGGCCAAGTCCAAGTGG |
| | (SEQ ID NO: 415) | aCAaACTCTTA | CTGGCT (SEQ ID NO: 511) |
| | | (SEQ ID NO: 463) | |
| No | gacatggttctacaGtTTA | cagagacttggtctAAcNA | ACCATCATCCAGCTTGGGAAGGAGAAATACTCGACCTGCGTGGTGGAGAAGACCACCGAGCCG |
| | NgGGGtAAGGGtAA | aCACTCTTCCCTCC | GAGTGGAGGGAAGAGTGCTCGTTCGAGCTGCAGCCCGGCGTGCTGGAGAGCAACGGGCGGAGC |
| | (SEQ ID NO: 416) | (SEQ ID NO: 464) | G (SEQ ID NO: 512) |
| No | cagagacttggtctTGAAN | gacatggttctacaCATCT | CTCGGCCCCCTAAATAGCCACAAAAGCGTCGGATGAGTGAAATCGGAGATGCTGCGCACCTCG |
| | gGATTGTGGGATAtAA | CcNATTTCACTCATC | GCCAAAATCCTGGAGCTTTTTTGAACAGGACTGTGGGTGTGTGCGCGTCGGGCTCGCCGTTA |
| | (SEQ ID NO: 417) | (SEQ ID NO: 465) | (SEQ ID NO: 513) |
| No | cagagacttggtctTCTTC | gacatggttctacatTTGT | TCTTCCGCCACATCCTCAACTTCTACCGCACCGGGAAGCTGCACTACCCGCGGCAGGAGTGCA |
| | CNCCACATCCTCA | AGTttTNgTAGtAGtAGT | TCTCCGCGTACGACGAGGAGCTCGCGTTCTTCGGCATCATCCCGGAGATCATCGGGGACTGCT |
| | (SEQ ID NO: 418) | (SEQ ID NO: 466) | GCTACGAGGACTACAAG (SEQ ID NO: 514) |
| No | cagagacttggtctAaTAA | gacatggttctacaGNgGt | ATTCCCGCCGGTGCCATCACGTTCACGCGTAGCCGCACTTGTGCAGCTTCAGGTTGCGTTGGT |
| | CTaTCAAATCAACAaaATT | AAGtAGTTtATtTGTG | CCGAGTATCTCTTCCCACACTTGTCACAGATGAACTGCTTGCCGCCGGCGTGGACGTGCCTCT |
| | CC | (SEQ ID NO: 467) | TGT (SEQ ID NO: 515) |
| | (SEQ ID NO: 419) | | |
| No | gacatggttctacaGAGTt | cagagacttggtctAaTCC | TGCTGCATTCCAGGAGAAAGGTCGTAATGTCTAGATGTTCGAAGTACCCATTTTGCGTGACGT |
| | TTTtAAAAGAGATGtAATA | ACCTaATTTTATTaaATTa | CAAATTTCTTCATGCAAAAATCGCGCGAAGCTCtacaaaaatgcacaattgcataaaaatgct |
| | G | TCATTCT | gtatttgtgCAAATCACTTC (SEQ ID NO: 516) |
| | (SEQ ID NO: 420) | (SEQ ID NO: 468) | |
| No | gacatggttctacaNACTT | cagagacttggtcttATtA | GACTTCCTGTCCTACCTCAGCCTCGAGAGACTGCAGGTTTGGTGGTTGTTGTTTGTACGCTTA |
| | CCTaTCCTACCTCA | tAATTAATttAAtAAtAtA | ACGCAGCAGATTGCTAACAGAAAAGTTAGAGCTATAAATGCAAACACTTCTGGAAGTTTGTGT |
| | (SEQ ID NO: 421) | AAtTTttAGAAGTG | TGTTGGATTAATTGTGATG |
| | | (SEQ ID NO: 469) | (SEQ ID NO: 517) |
| No | cagagacttggtctACCCc | gacatggttctacagGNgG | ACCCCGATGATGTTCTCTATACTGAAAGACGGTCTGTTGGAAGGCTCTGATTTGATCATGGAC |
| | NATaATaTTCTCTATAC | AGtTGAAtAGAAAAG | GCCGTGCTCAGACTGTTGAGCTGAAGCTGAAGGCTCGGGCTGAGCTGGGAGTTGAACGCTTTT |
| | (SEQ ID NO: 422) | (SEQ ID NO: 470) | CTGTTCAGCTCCGCC (SEQ ID NO: 518) |
| No | gacatggttctacagGATA | cagagacttggtctCTaTc | GTTGTGGTCCAGAGGGTTATCGCAGTTGATTATGATGGAGGAAAAAACACCATCGACAGAGGA |
| | tTGTGTTttAAATGtAAtA | NATaaTaTTTTTTCCTCC | TTGGGAGGTGTGGGGGGTGCACCCGAACTGAAATGACATCATCCCCTCCACTCTTCGCCCAaa |
| | TG | (SEQ ID NO: 471) | g (SEQ ID NO: 519) |
| | (SEQ ID NO: 423) | | |
| No | gacatggttctacaaACTC | cagagacttggtctGNgTA | TTTCTGTCATTAACTTTTCTCATTATTTTAGCAGATGATTCTTTTTGCACTTTGCTTTAAAGT |
| | CTaACCACCCTaTC | TAGTAGAATATATTTAAGG | TGTTCTAATAACCTGTAACAGAGTTCAGGTCCAGGATCAGTTAGCAGCTGGTGGTCTGAGAGG |
| | (SEQ ID NO: 424) | (SEQ ID NO: 472) | GTT (SEQ ID NO: 520) |
| No | cagagacttggtctTaATT | gacatggttctacaTAGAA | CGCAGGTTGCAAACTATATTGTGCGTCTTTTATCATCTTCTCAGTGTTTCTATGTCTTGgctt |
| | aaCACATAAATTaAAaTTA | AtAtTGAGAAGATGATAAA | tcactttcctcattatttttcctccctctcctcctgtgtctcaccatctctttctctgtctaa |
| | aaTaATTTTCTC | AG | aGTAAGCTAATGCACGGGAGACACTC (SEQ ID NO: 521) |
| | (SEQ ID NO: 425) | (SEQ ID NO: 473) | |
| No | gacatggttctacaCCcNC | cagagacttggtctGGATT | CGTGGTTTACTTGGTTTACTCGGTGGTGAATCCCCCGTACGTGCATGGAGAGAATGAATGCCA |
| | AaaCCCTCTCC | tAttAtNgAGTAAAttAAG | AACTCCATTGAAAAATCTGGCGATTTAATATGTCGTGATATACACACTATACAACGTGAGGC |
| | (SEQ ID NO: 426) | T | (SEQ ID NO: 522) |
| | | (SEQ ID NO: 474) | |
| No | gacatggttctacatATAt | cagagacttggtctAATCC | CATACACTAACGTAGCTGTAGTATGCGTTATAGCCATTTATCCTGCCCTACCCTGCAGATGTT |
| | AtTAANgTAGtTGTAGTAT | TATaAcNAAAACTATTCC | TAATAGTGTTACTAAACTTTTACTTTGGTACCGAAGCCCTCGCTTTCTAAGTTGTTCCGGTTC |
| | G | (SEQ ID NO: 475) | AGTGGAATAGTTTTCGTCATAGGATT (SEQ ID NO: 523) |
| | (SEQ ID NO: 427) | | |
| No | gacatggttctacaCTcNT | cagagacttggtctANgAT | CTCGTATTTCTGAACCAGCAGTTCAAACTCCCGGTAGTTCTCCTCCGCCTGCTGGTCAAGACG |
| | ATTTCTaAACCAaCAaTT | TTGAATTGtAtttTGGATA | CTGATAAGCCCGGACACACTCGCTGCATCCCCCCATGGCCATATCCAGGGTGCAATTCAAATC |
| | (SEQ ID NO: 428) | T | GT (SEQ ID NO: 524) |
| | | (SEQ ID NO: 476) | |
| No | cagagacttggtctTTATA | gacatggttctacatTGAA | TCCTTTGGGGGCCACCTGGTTCAGGAGGTTGTAGTACGCCCTGGAGTcctgaaacacatttaa |
| | AaaTTAATACAaaaAaaTT | ttAGGTGGtttttAAAGG | tgtcCAGTGTTGaagtcagacagagaggcaccgttataattattattaaagtcAAAAATCACC |
| | AaAaaAATATTTCA | (SEQ ID NO: 477) | TGACTGATCAGTCCAag (SEQ ID NO: 525) |
| | (SEQ ID NO: 429) | | |
| No | gacatggttctacaNgGTG | cagagacttggtctAacNA | CGGCCTCGAGGTTCTTCACCAGACAGTTCTTCTTGGCCATTCGCTTGGCAGTGAGAGTCAGAC |
| | GTGTtAGttTtATGG | ATaaCCAAaAAaAACTaTC | ACACCTGGGAGGACGGCAGGAggtgagggggaggaagagTAGGGGGggagtgaaggagggagg |
| | (SEQ ID NO: 430) | T | aaagaggggaagagaggaaa (SEQ ID NO: 526) |
| | | (SEQ ID NO: 478) | |
| No | cagagacttggtctgAGAt | gacatggttctacaaTaTa | ACCTCTGTATACTGCATAAAGTGTCTATACTTAAACCTACATGTTAATGGAGTTCTGTTGGGC |
| | tTtTGTATAtTGtATAAAG | ATaCCTCCTTCAaTTCAC | CTCAGCGAGCCGTAGTTTCTTAGCGTGGTTCTTGCCCTGGTAGTGGGCCTGTGCAACTGCCGG |
| | TG | (SEQ ID NO: 479) | TGAACTGAAGGAGGCATCACACAGC (SEQ ID NO : 527) |
| | (SEQ ID NO: 431) | | |
| No | gacatggttctacaAAGAt | cagagacttggtctCTCTc | GCAAAGCTTAACGCTGAAAGCTACTTCCTGCCGAGAGCTGGAGTCCTTAACCCGAGCATTATG |
| | tTGTATGTGGtAAAAAGG | NaCAaaAAaTAaCTTTCA | GGATATGGTCCTGGCTCT (SEQ ID NO: 528) |
| | (SEQ ID NO: 432) | (SEQ ID NO: 480) | TCTATGACAATTGCAGATACATTTACAAGCAACATATACATAACAAAGCTCAACTGATTGCCT |
| No | gacatggttctacaGTGGT | cagagacttggtctCATAT | ATTGTAAAACCAGATTTTCGCTGATTTTAATGGACGTGCATTGCTAGCAACACCTTACAGCAT |
| | AGAGGtAAtTAGGTAA | TCTTTACATTATTaCTaAC | AGTGAATGTTTTAAGAGAATGGTCAGCAATAATGTAAAGAATATGCAATTTATAATTAgtgct |
| | (SEQ ID NO: 433) | C | aaaacatgc (SEQ ID NO: 529) |
| | | (SEQ ID NO: 481) | |
| No | gacatggttctacaTTAaA | cagagacttggtcttTGAA | ACGTAAGTCTTCAGAGGCTTGTGCTGATCTAAGgaggaaataatgaaaaacagaatattaggc |
| | AACTacNCTCTaAaCTaTC | GAtTTANgTTGATttAtAt | taaataataattaaattaacagTTACTGAATTATTATTGCATGTACGGAATGGTTTTCTAACC |
| | (SEQ ID NO: 434) | ATGTATG | TGTTGAAAAGTAGGGATCTTCAG (SEQ ID NO: 530) |
| | | (SEQ ID NO: 482) | |
| No | cagagacttggtctTATGT | gacatggttctacaAaCAa | CATATCTTCCAGAAGGAGGGGGTTACTGCTTTCTACAAGGGCTACGTGCCCAACATGCTGGGC |
| | ATGTTAGTtTtTGTATGTA | TAACCCCCTCCTTC | ATCATTCCCTATGCTGGCATCGACCTGGCTGTCtatgaggtgtgtgtttgttgtcaaAGCATA |
| | TG | (SEQ ID NO: 483) | ACATATCTTTGTGTTTACTgg (SEQ ID NO: 531) |
| | (SEQ ID NO: 435) | | |
| No | gacatggttctacaATtTA | cagagacttggtctaTCAA | AACTAAGTCCAGAACCTTCTTGTCCAGGTATGAGATCCGGGACCCACACATGGTGGAGGAAAA |
| | TGTGtTTttTtATGTttAt | AaTcNaCCCTCTCC | TGTCCTGCAGATCCTGAAGGAGAGGGCCGACTTTGACAACTATAAGCCCCGCCCCTTCAACAT |
| | ATTAAtTAAGT | (SEQ ID NO: 484) | G (SEQ ID NO: 532) |
| | (SEQ ID NO: 436) | | |
| No | IgacatggttctacaNgGtt | cagagacttggtctAATTa | CAGAAACAGTTAGACAGTCAGGTTGCTGTTCCAATTTTCGTTTTATTAATACGAAGATAATTA |
| | TTTTGAGTtAtTTGAAAAG | aAACAaCAACCTaACTaTC | AATAATAGTTTTGACTCCCTCTATAATGCTTTGTAAGTGGCGGAGtgtcttttaaaaacagaa |
| | (SEQ ID NO: 437) | T | aacatgagaTGAT (SEQ ID NO: 533) |
| | | (SEQ ID NO: 485) | |

### Data Analysis and Age Estimation

SeqKit v1.2 was used to hard clip the reads by 15 bp at both 5′ and 3′ ends to remove adaptor sequences (Shen et al., 2016). Clipped reads were aligned to a reduced representation genome of each species closest relative genome. Both the Murray cod and Mary River cod were aligned to the Murray cod genome (GCA002120245.1 mcod v1). Bismark v0.20.0 was used to align reads with the following parameters: --bowtie2 -N 1 -L 15 -bam -p 2 -score L, −0.6, −0.6 -non_directional. Methylation calling was performed using bismark_methylation_extractor function with default parameters (Krueger and Andrews, 2011).

70% of the samples were randomly assigned to a training data set and the remaining into a testing data set. Age in years was natural log transformed and an elastic net regression model was applied on the training data sets. Age was regressed over the methylation of each CpG site that was captured during sequencing. The glmnet function used for the elastic net regression model was set to a 10-fold cross validation with an α-parameter=0 (Friedman et al., 2010). The α-parameter was set to 0 to force all sites to be used in the model, as opposed to Example 1 where it was set to 0.5 to identify the minimum number of sites required (Horvath, 2013; Stubbs et al., 2017; Thompson et al., 2017). All analyses were performed in R using version 3.5.1 (R Core Team, 2013).

### Data Analysis and Age Estimation

SeqKit v1.2 was used to hard clip the reads by 15 bp at both 5′ and 3′ ends to remove adaptor sequences (Shen et al., 2016). Clipped reads were aligned to a reduced representation genome of each species closest relative genome. Both the Murray cod and Mary River cod were aligned to the Murray cod genome (GCA002120245.1 mcod v1). Bismark v0.20.0 was used to align reads with the following parameters: --bowtie2 -N 1 -L 15 -bam -p 2 -score L, −0.6, −0.6 -non_directional. Methylation calling was performed using bismark_methylation_extractor function with default parameters (Krueger and Andrews, 2011).

70% of the samples were randomly assigned to a training data set and the remaining into a testing data set. Age in years was natural log transformed and an elastic net regression model was applied on the training data sets. Age was regressed over the methylation of each CpG site that was captured during sequencing. The glmnet function used for the elastic net regression model was set to a 10-fold cross validation with an α-parameter=0 (Friedman et al., 2010). The α-parameter was set to 0 to force all sites to be used in the model, as opposed to Example 1 where it was set to 0.5 to identify the minimum number of sites required (Horvath, 2013; Stubbs et al., 2017; Thompson et al., 2017). All analyses were performed in R using version 3.5.1 (R Core Team, 2013).

For the Murray and Mary River cod 26 CpG sites were used to calibrate the model. These sites are provided in Table 16 and are referred to herein as the Maccullochella clock.

TABLE 16
Age associated CpG sites used to estimate age in the Murray
and Mary river cod. The genomic locations are the Murray
cod genome (GCA002120245.1 mcod v1). The intercept is 0.224753778.
The coefficient is also referred to as weight.

| Chromosome | Position | Coefficient |
|---|---|---|
| LKNJ01000042.1 | 402010 | 0.004093597 |
| LKNJ01000204.1 | 197441 | −0.002050386 |
| LKNJ01000233.1 | 18565 | −0.000112642 |
| LKNJ01000243.1 | 177861 | 0.000969599 |
| LKNJ01000303.1 | 226022 | −0.001422143 |
| LKNJ01000303.1 | 226104 | −0.001687785 |
| LKNJ01000579.1 | 97711 | 0.007701688 |
| LKNJ01000579.1 | 97721 | 0.055978558 |
| LKNJ01000579.1 | 97748 | 0.011710677 |
| LKNJ01000579.1 | 97772 | 0.048485488 |
| LKNJ01000596.1 | 130919 | 0.006567998 |
| LKNJ01000596.1 | 130941 | 0.000497042 |
| LKNJ01000626.1 | 64260 | −0.000013002 |
| LKNJ01000626.1 | 64267 | 0.000000000 |
| LKNJ01001186.1 | 128826 | 0.001434368 |
| LKNJ01001658.1 | 66030 | 0.001204590 |
| LKNJ01001980.1 | 9541 | −0.055750015 |
| LKNJ01001980.1 | 9568 | −0.262247410 |
| LKNJ01002086.1 | 21365 | 0.314310992 |
| LKNJ01002218.1 | 37956 | 0.030189498 |
| LKNJ01002551.1 | 35928 | 0.003941312 |
| LKNJ01002551.1 | 35950 | 0.059712306 |
| LKNJ01003084.1 | 25417 | 0.001623630 |
| LKNJ01003347.1 | 28914 | 0.025396344 |
| LKNJ01003347.1 | 28938 | 0.004647240 |
| LKNJ01003347.1 | 28958 | 0.026382203 |

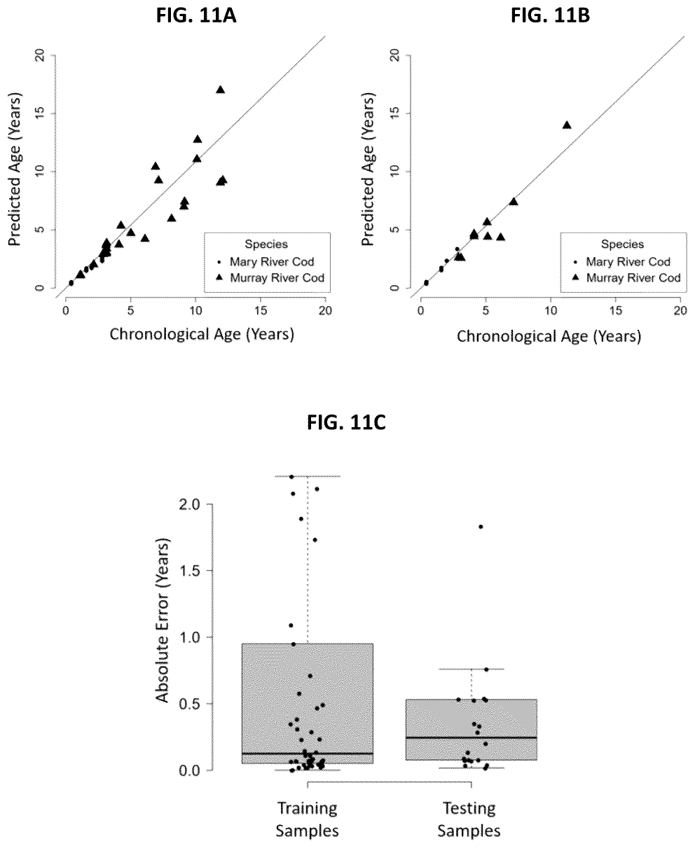

The inventors found a high correlation between the chronological and predicted age in both the training data set (Pearson correlation=0.92, p-value=1.36×10⁻²) and the testing data set (Pearson correlation=0.92, p-value=1.36×10⁻¹³) (FIG. 11A-B). A low MAE of 0.34 years was observed in the testing data with no significant difference in the training data set (p-value=0.53, t-test, two-tailed) (FIG. 11C). As described above, the similar correlation values and low MAE in the training and testing data sets suggests a lack of overfitting by the model.

To test if the model was performing better on either the Murray cod or Mary River cod, a one-way ANOVA was used with chronological age as a blocking factor. A blocking factor was used to reduce bias between the age of samples as all samples above 2.9 years were Murray cod. No difference was found between the species in both the training (p-value=0.139) and testing data set (p-value=0.185). This suggests the model performance is not biased towards one species. Similarly, to the Lungfish clock, the inventors found the performance of the Maccullochella clock to be highest with younger individuals (Table 17).

TABLE 17
Performance of the Maccullochella clocks at increasing
age intervals in the testing data set.

| | | MAE | Median Relative |
|---|---|---|---|
| Age Range | Correlation | (Years) | error (%) |
| ≤5 | 0.98 | 0.35 | 9.16 |
| 6-10 | 0.25 | 1.99 | 28.10 |
| >10 | 0.08 | 2.86 | 24.04 |

### Discussion

In this study, the inventors have developed a DNA methylation age estimator for two threatened fish species. This study has used conserved age associated DNA methylation at CpG sites in zebrafish to develop an epigenetic clock for Murray cod and Mary River cod. This study demonstrates age associated CpG methylation at sites in one fish species can be predictive of age in other species.

One of the advantages of developing the Maccullochella clock with two species is the potential use of age estimation for other members of the Maccullochella genus. The time separating the last common ancestor for the Maccullochella genus ranges between 4.35 and 9.99 MYA (Nock et al., 2010). The Maccullochella genus comprises four species; Murray cod, Mary River cod, Eastern freshwater cod (Maccullochella ikei), and Trout cod (Maccullochella macquariensis) (Nock et al., 2010). The Maccullochella clock therefore has the potential to be used in the Eastern freshwater cod and the Trout cod despite the model not being calibrated with these species.

### Example 9—Age Estimation for Marine Turtles

In this example, the inventors identify CpG sites that were conserved in all species of marine turtle included in the study and significantly correlated with age. The inventors also provide a universal epigenetic clock that can be used to predict the age of all marine turtles thereby providing a non-lethal methodology to predict age in marine turtles.

### Animal Ethics and Tissue Collection

Skin biopsy samples from green sea turtles of known age were collected from a turtle population on Cayman Island and a turtle population from Kélonia Reunion. In addition to known age samples, two wild turtles with paired samples of known time intervals were collected at Ningaloo reef, Western Australia.

In addition, one skin biopsy from each of the following species was included in the reduced representation bisulfite sequencing (RRBS): Flatback turtle (Natator depressus), Hawksbill turtle (Eretmochelys imbricata), Leatherback turtle (Dermochelys coriacea), Loggerhead turtle (Caretta caretta), and Olive Ridley turtle (Lepidochelys olivacea). One sample from each species was used to identify CpG sites conserved within all marine turtle species to develop a universal epigenetic clock for marine turtles. The collection of these samples was approved by the appropriate animal ethics committee.

### DNA Extraction and Bisulfite Treatment

DNA was extracted using the DNeasy Blood & Tissue Kit (QIAGEN) as instructed in the manufacture's protocol. Extracted DNA was bisulfite converted using the protocol as previously described (Clark et al., 2006).

### Reduced Representation Bisulfite Sequencing

A total of 72 marine turtle skin biopsy samples were used for RRBS (Table 18). RRBS libraries were prepared using MspI digestion as previously described (Smallwood et al., 2011). Libraries were sequenced on an Illumina NovaSeq at the Australian Genome Research Facility (AGRF).

TABLE 18
Sample sizes by locations of turtle skin biopsies used
for reduced representation bisulfite sequencing.

| | Species | Age | |
|---|---|---|---|
| Sample | and total | Range | Sex |
| origin | samples | (Years) | distribution |
| Cayman Turtle | Green sea | 1-43 | Female: 31 |
| Centre, Cayman | turtle: 51 | | Male: 10 |
| Islands | | | Unknown: 10 |
| Centre D'Etude Et De | Green sea | 1-34 | Unknown: 12 |
| Découverte Des | turtle: 12 | | |
| Tortues Marines, La | | | |
| Réunion, France | | | |
| Ningaloo Reef, | Flatback turtle: 1 | NA | Unknown: 5 |
| Western Australia, | Hawksbill turtle: 1 | | |
| Australia | Leatherback turtle: 1 | | |
| | Loggerhead turtle: 1 | | |
| | Olive Ridley | | |
| | turtle: 1 | | |

### Sequencing Data Analysis

Demultiplexed fastq files were quality checked using FastQC v0.11.8 (www.bioinformatics.babraham.ac.uk/projects/fastqc/) and were trimmed using trimmomatic v0.38 with the following options SE -phred33 ILLUMINACLIP:TruSeq3-SE:2:30:10 LEADING:3 TRAILING:3 SLIDINGWINDOW:4:15 MINLEN:36). Trimmed reads were aligned using BS-Seeker2 v 2.0.3 with default settings and bowtie2 v2.3.4 to the green sea turtle genome (assembly: rCheMyd1.pri) (Wang et al., 2013; Rhie et al., 2020; Guo et al., 2013; Langmead and Salzberg, 2012). BS-Seeker2 call methylation module with default settings was used for methylation calling. CpG sites with a mean inadequate coverage of <2 reads or a clustering of >100 reads was removed from downstream analysis as previously described (Stubbs et al., 2017; Mayne et al., 2020).

### Universal Marine Turtle Epigenetic Clock

CpG sites that were captured in all species and had adequate coverage, as described above, were included in model generation. Green sea turtle samples of known age were randomly assigned in either a training data set (46 samples) or a testing data set (17 samples). Age was transformed to a natural log to fit a linear model. Using an elastic net regression model, the age of the turtles was regressed over the methylation of CpG sites. The glmnet function in the glmnet R package was used to apply the elastic net regression model (Friedman et al., 2010). The glmnet function was set to a 10-fold cross validation and an α-parameter of 0.5 (optimal between a ridge and lasso model). The glmnet function returned a minimum λ-value of 0.0831635 based on the training data. The performance of the model was assessed using Pearson correlations, absolute error, and relative error rates. All statistical analyses were carried out in R v3.5.1 (R Team, 2013).

### Universal Marine Turtle Age Markers

On average, 45.3 million reads per RRBS library were aligned to the green sea turtle genome with an alignment rate 88.6%. This resulted in a total of 1,261,168 CpG sites with an average coverage of 6 reads per CpG site. Global methylation was found to be 65.5% and was not found to significantly associate with age (Pearson correlation=0.10, p-value=0.67). However, the inventors identified 8,225 CpG sites exclusively in green sea turtles that correlate with age (Pearson correlation, p-value<0.05). A total of 844 CpG sites were found to have full methylation values in all samples and were conserved in all species. Of the 844 conserved CpG sites, 119 significantly correlated with age (Table 19).

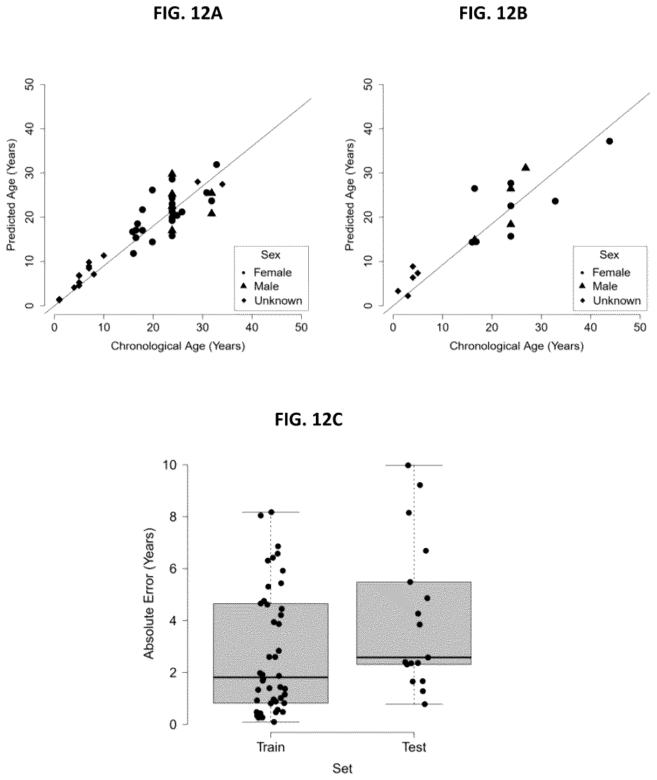

The elastic net regression model was used to identify the minimum number of sites to predict age. The regression model returned 29 CpG sites that are conserved in all marine turtle species and could be used to predict age (Table 20). Using the 29 CpG sites, the inventors found a high correlation between the chronological and predicted age (FIG. 12a,b) in both the training (Pearson correlation=0.93, p-value<2.20×10⁻¹⁶) and testing (Pearson correlation=0.90, p-value=7.54×10⁻⁷) data sets. The inventors also found a low median absolute error rate (MAE) of 2.57 years in the testing data set (FIG. 12c). No statistical significance in absolute error rate was found between training and testing data sets (t-test, two-tailed, p-value=0.10). From herein, the model with the 29 CpG sites conserved across marine turtle species will be referred to as the universal marine turtle clock.

The inventors also performed the elastic net regression model using CpG sites that are found in green sea turtles but not necessarily in other species. The inventors found a similar correlation and MAE in the testing data set compared to the universal marine turtle clock (Pearson correlation=0.90; MAE=3.29 years). However, there was an increase to 38 CpG sites for this specific green sea turtle epigenetic clock. Of the 38 CpG sites; 29 are the universal marine turtle clock. Importantly, there was little to no difference between age prediction models.

TABLE 19
Age associated CpG sites predictive of age in marine turtles. The genomic coordinates
are based on the green sea turtle genome (assembly: rCheMyd1.pri).

| CpG site | | | Associate with age | | CpG site | | | Association with age | |
|---|---|---|---|---|---|---|---|---|---|
| chr | position | strand | Correlation | p-value | chr | position | strand | Correlation | p-value |
| NC_051253.1 | 4789859 | + | −0.62938 | 0.00000003 | NC_051247.1 | 29577068 | + | 0.310867 | 0.01314567 |
| NC_051260.1 | 18752232 | + | −0.57933 | 0.00000065 | NC_051241.1 | 190794258 | + | −0.31062 | 0.01322332 |
| NC_051268.1 | 1438203 | + | 0.57155 | 0.00000100 | NC_051254.1 | 35262262 | + | 0.309567 | 0.01355347 |
| NC_051247.1 | 122622449 | + | 0.571322 | 0.00000101 | NC_051241.1 | 324938896 | + | 0.30863 | 0.01385310 |
| NC_051241.1 | 125974418 | + | −0.56564 | 0.00000136 | NC_051247.1 | 122619902 | + | −0.3078 | 0.01412662 |
| NC_051245.1 | 2887366 | + | 0.55533 | 0.00000231 | NC_051242.1 | 59603651 | + | −0.30718 | 0.01433104 |
| NC_051244.1 | 31108776 | + | −0.50973 | 0.00001982 | NW_023618369.1 | 115306 | + | −0.30674 | 0.01447893 |
| NC_051242.1 | 104251871 | + | −0.48756 | 0.00005059 | NC_051241.1 | 114662020 | + | −0.30649 | 0.01456067 |
| NC_051266.1 | 5261789 | + | 0.483929 | 0.00005864 | NC_051243.1 | 130867428 | + | −0.30649 | 0.01456067 |
| NW_023618337.1 | 683814 | + | −0.47817 | 0.00007385 | NC_051243.1 | 142954596 | + | −0.30649 | 0.01456067 |
| NC_051246.1 | 404436 | + | −0.47264 | 0.00009181 | NC_051244.1 | 112335735 | + | −0.30649 | 0.01456067 |
| NC_051242.1 | 5214346 | + | 0.4696 | 0.00010330 | NC_051249.1 | 48918401 | + | −0.30649 | 0.01456067 |
| NC_051266.1 | 1137469 | + | 0.45902 | 0.00015444 | NC_051250.1 | 26971832 | + | −0.30649 | 0.01456067 |
| NC_051241.1 | 163147016 | + | −0.45607 | 0.00017231 | NC_051250.1 | 67552049 | + | −0.30649 | 0.01456067 |
| NC_051262.1 | 4837160 | + | −0.44361 | 0.00027112 | NC_051251.1 | 17839349 | + | −0.30649 | 0.01456067 |
| NC_051252.1 | 29640955 | − | 0.429837 | 0.00043848 | NC_051255.1 | 26174984 | + | −0.30649 | 0.01456067 |
| NC_051267.1 | 11665443 | + | 0.428604 | 0.00045731 | NC_051259.1 | 2448192 | + | −0.30649 | 0.01456067 |
| NC_051242.1 | 257449886 | + | −0.42207 | 0.00056994 | NC_051260.1 | 114280 | + | −0.30649 | 0.01456067 |
| NC_051246.1 | 252327 | + | −0.41997 | 0.00061131 | NC_051245.1 | 87828097 | + | 0.303613 | 0.01556344 |
| NC_051250.1 | 46230416 | + | −0.41791 | 0.00065429 | NC_051245.1 | 25274841 | + | −0.29863 | 0.01743546 |
| NC_051266.1 | 12851945 | + | 0.41722 | 0.00066936 | NC_051249.1 | 83427054 | + | −0.29778 | 0.01777479 |
| NC_051265.1 | 1469699 | + | −0.41191 | 0.00079586 | NC_051262.1 | 34491 | + | −0.29675 | 0.01819255 |
| NC_051245.1 | 1493239 | + | 0.40881 | 0.00087947 | NC_051259.1 | 7359308 | + | −0.29645 | 0.01831634 |
| NC_051244.1 | 39827463 | + | −0.39637 | 0.00130027 | NC_051252.1 | 35828717 | + | −0.29404 | 0.01932819 |
| NC_051248.1 | 95968272 | + | −0.39296 | 0.00144346 | NC_051253.1 | 1546469 | + | −0.29393 | 0.01937831 |
| NC_051247.1 | 122622791 | + | −0.38923 | 0.00161658 | NC_051243.1 | 20561299 | + | 0.29276 | 0.01988958 |
| NC_051246.1 | 64204711 | + | −0.38699 | 0.00172905 | NC_051267.1 | 5485734 | + | −0.2926 | 0.01995707 |
| NC_051247.1 | 123209497 | + | 0.385726 | 0.00179580 | NW_023618336.1 | 3158677 | + | 0.28668 | 0.02273112 |
| NC_051261.1 | 15509425 | + | −0.3837 | 0.00190720 | NC_051246.1 | 95762557 | + | −0.28476 | 0.02369669 |
| NC_051243.1 | 25798703 | + | −0.38271 | 0.00196382 | NC_051244.1 | 136659670 | + | 0.28361 | 0.02429251 |
| NC_051244.1 | 1391582 | + | −0.38238 | 0.00198337 | NC_051242.1 | 235367452 | + | 0.282658 | 0.02479448 |
| NC_051247.1 | 22362197 | + | 0.382249 | 0.00199101 | NC_051250.1 | 71632357 | + | −0.28208 | 0.02510123 |
| NC_051244.1 | 105404767 | + | −0.38218 | 0.00199481 | NC_051268.1 | 7015372 | + | −0.27878 | 0.02693154 |
| NC_051241.1 | 333468941 | + | −0.36285 | 0.00347016 | NC_051241.1 | 217749260 | + | 0.27714 | 0.02788248 |
| NC_051241.1 | 207956449 | + | −0.35872 | 0.00388971 | NC_051243.1 | 194226771 | + | −0.27668 | 0.02815112 |
| NC_051264.1 | 2208687 | + | −0.35852 | 0.00391061 | NC_051247.1 | 87996095 | + | −0.27583 | 0.02866133 |
| NC_051246.1 | 128332276 | + | 0.35764 | 0.00400632 | NC_051242.1 | 80491050 | + | −0.27366 | 0.02999088 |
| NC_051242.1 | 23178920 | + | −0.35744 | 0.00402862 | NC_051245.1 | 111539015 | + | −0.27366 | 0.02999088 |
| NC_051261.1 | 14546832 | + | −0.35374 | 0.00445374 | NC_051255.1 | 33315256 | − | −0.27262 | 0.03064832 |
| NC_051262.1 | 4837245 | + | −0.35163 | 0.00471424 | NC_051241.1 | 212587650 | + | −0.27195 | 0.03107369 |
| NC_051265.1 | 565064 | + | −0.34819 | 0.00516664 | NC_051252.1 | 27667687 | + | −0.27067 | 0.03190639 |
| NC_051241.1 | 261342954 | + | −0.34226 | 0.00603971 | NC_051250.1 | 80017181 | + | −0.27055 | 0.03198552 |
| NC_051254.1 | 15652959 | + | −0.34214 | 0.00605714 | NC_051259.1 | 1959061 | + | −0.26989 | 0.03242094 |
| NC_051265.1 | 737857 | + | −0.33634 | 0.00703602 | NC_051241.1 | 284697977 | + | −0.26918 | 0.03289629 |
| NC_051244.1 | 117601530 | + | −0.33482 | 0.00731359 | NC_051261.1 | 809116 | + | −0.26664 | 0.03464777 |
| NC_051243.1 | 104492044 | + | −0.3344 | 0.00739271 | NC_051247.1 | 21524700 | + | −0.26603 | 0.03507955 |
| NC_051249.1 | 76127746 | + | −0.33244 | 0.00776813 | NC_051242.1 | 156838930 | + | 0.26574 | 0.03529289 |
| NC_051252.1 | 6896371 | + | 0.331459 | 0.00796238 | NC_051243.1 | 146433309 | + | −0.26446 | 0.03621892 |
| NC_051253.1 | 34859711 | + | −0.33141 | 0.00797123 | NC_051261.1 | 18427159 | + | −0.26054 | 0.03917994 |
| NC_051248.1 | 55305215 | + | −0.32903 | 0.00846134 | NC_051245.1 | 10133556 | + | −0.25884 | 0.04052081 |
| NC_051241.1 | 218171637 | + | −0.32842 | 0.00859287 | NC_051249.1 | 53076204 | + | −0.25874 | 0.04060299 |
| NC_051242.1 | 261130905 | + | −0.32645 | 0.00902316 | NC_051241.1 | 203793846 | + | −0.25655 | 0.04239577 |
| NC_051242.1 | 242755994 | + | 0.325004 | 0.00935096 | NC_051241.1 | 215618275 | + | −0.25576 | 0.04305681 |
| NC_051253.1 | 11964470 | + | −0.3247 | 0.00942182 | NC_051244.1 | 54234475 | + | −0.25461 | 0.04403332 |
| NC_051262.1 | 4823175 | + | −0.32232 | 0.00998865 | NC_051241.1 | 28109409 | + | −0.2538 | 0.04473368 |
| NC_051261.1 | 5886341 | + | 0.32209 | 0.01004385 | NC_051248.1 | 98471267 | + | −0.25087 | 0.04734700 |
| NC_051247.1 | 122616669 | + | −0.32034 | 0.01048207 | NC_051247.1 | 103027749 | + | −0.24953 | 0.04858088 |
| NC_051242.1 | 74564651 | + | 0.317315 | 0.01127584 | NC_051267.1 | 15044585 | + | −0.24927 | 0.04882553 |
| NC_051242.1 | 233760431 | + | −0.31469 | 0.01200851 | NC_051251.1 | 38317747 | + | −0.24894 | 0.04913652 |

TABLE 20
The 29 CpG sites in the universal marine turtle epigenetic clock. The locations of the sites are based on the green sea turtle genome
(assembly: rCheMyd1.pri). The intercept is 8.840734456. The coefficient is also referred to as weight.

| CpG site | | | Association with age | | | Amplicon comprising CpG site. The CpG site of interest is in the located |
|---|---|---|---|---|---|---|
| chromosome | position | strand | Coefficient | Corr. | p-value | within the amplicon that can be used to design primers for multiplex PCR. |
| NC_051241.1 | 114662020 | + | −5.273583789 | −0.20134 | 0.113571 | CCTTAAAGGCTTCAAGCCCTGATGACAAATCAACGCCACCTCCACCCACCCCCGCTTA |
| | | | | | | TCACTCGTGTGAGATCATGGCTGGCAAATACGCGACCCCGGTCTCCGAGGCTCTGCCC |
| | | | | | | AGCTGGCTCTGCCCGACCGCCTGGCTCAGCCCCGCggggccccgccccagccccgcgc |
| | | | | | | CGCGCCGCGCATGCTCCTTCCCCCGCTGCGGCCGCCGGCGCCGGGCCTGGCTGGAGCA |
| | | | | | | TGGGGGCCTGGGAGCCGCCGCCAGCCCCACGGCCGGGCCCGGCGGCTCCTCCTGCTCC |
| | | | | | | GCGCGGGGCCGGGCTCAGCCGCTTCGTGCGGTGCCTCTACCTGGTGGGCTTCCTGGTG |
| | | | | | | AGTGCGGGGTGCAGCATCAGGGACCTGCCGGGCGGGGGCTCGGTCCCCCGCTTCCTgc |
| | | | | | | cgtgtgtgggtgtgtgtgtcccGGTTCCTGCCCTGGCcggcgggggtggggtgtgtgt |
| | | | | | | CCCGGTTCCTGCCCTggccggcgggggcggggggggtgtcccGGTTCCTGCcctggcc |
| | | | | | | ggcgggggggggggggggggggtgtcccggtTCCTGCCCTggccggcgggggtggggg |
| | | | | | | ggtgtgtgtcccGGTTCCTG (SEQ ID NO: 534) |
| NC_051241.1 | 125974418 | + | −2.188538854 | −0.56285 | 0.000002 | Accaactcatggtcactgcctcccagattcccatccacttttgcttcccccactaatt |
| | | | | | | ctacctggtttgtgagcagcaggtcaagaaaagcgccccccctagttggctcccctag |
| | | | | | | cacttgcaccaggaaattgtcccctacgctttccaaaaacttcctggattgtctatgc |
| | | | | | | accgctgtattgctctcccagcagatatcaggaaaattaaagtcacccatgagaatca |
| | | | | | | gggcatgcgatctagtagcttccgtgagttgccggaagaaagcctcatccacctcatc |
| | | | | | | cccctggtccggtggtctatagcagactcccaccactacatcactcttgttgcacaca |
| | | | | | | cttctaaacttaatccagagacactcaggtttttccacagtttcgtaccgttATATCC |
| | | | | | | CTGGCTTCATCTTATCAATGTTATacatcttttttgggggggggcacacaAGCTTTGG |
| | | | | | | AAGCAATCCCGTTACACATATGTATCTGAAAAATGAATTAGATATGAAAGATTAATCA |
| | | | | | | TATTTCAGCACATAAGTAAAAAAGAAGACTAATTGCCTAGGATTCTCCCATAGGAATG |
| | | | | | | CTATTGCATAATTGTTCGTT (SEQ ID NO: 535) |
| NC_051241.1 | 217749260 | + | −0.28179239 | 0.27166 | 0.031261 | ccccccccccccagaacccctcttctgtcccctgactgcccccagaaccggacaggag |
| | | | | | | ggtctcgtgggccaccgtagtgggtgcctaccccacccctaagagccagaggcacctg |
| | | | | | | ccagggggcgaggtggggagtcctggcagtgcttacctggggcggctcccaggaagca |
| | | | | | | cctggcaggttcctctggctcctaggggCggggcaGCGTAGCTAGGTGGGGAgtaggg |
| | | | | | | ggagcagctgctccccccactgatcacatcaaaagtggcgccataggcgccgactccc |
| | | | | | | tgggtgatccggggctggagcacccacggggaaaatttggtgggtgcagagcacccac |
| | | | | | | cgtcagctccccaccccgcccccatctcagttcacctctgctccgcctccgcctcctc |
| | | | | | | ccctgaacgtaccgccccgctctgcttctctgcaccttcccccaccaccacccccccc |
| | | | | | | cccccccccgccggcttcccgcaaatcagctgttcggagggaagccggggagggctga |
| | | | | | | gaagcaggccgcggcttcccactcaggccaagggtggtggaggtgagctggggcaagg |
| | | | | | | agcggttcccctgcgtgtcc (SEQ ID NO: 536) |
| NC_051241.1 | 261342954 | + | −0.292522455 | −0.36529 | 0.003242 | gtttcaggagatcaaggccctagatctgaccccggtcacccagggggaggatgacctt |
| | | | | | | ttgccagcaaacctcgatctGGGCGATCTCACTCCACCCCTCtattccccatgctccc |
| | | | | | | tccccctaactgctgcttctgctcccacctccgaggagcccctggactcctccatcaa |
| | | | | | | cccagccactgatggcaccccgctgacgaccaccaagcctgctcaggtgacagccggc |
| | | | | | | gccacgtGGCTAGGACAggagtcgccaggggcaccccccgttggtgcggagcaatcga |
| | | | | | | cttccttcccgggcgggggccctatagaagataatccacctcctgatgctgtggccgc |
| | | | | | | taaatccaccatagagcctgtGCCCgctatcactgagagctccctccccaccccttta |
| | | | | | | accctcgagcctgatcaGGAGGCGCCatcatccagctgcttgcctcctgaaacccaga |
| | | | | | | acctcgcctctgcccctgccccggcccttACCTCTATccagtttacctcctgcaatgt |
| | | | | | | tattgccacccccggggctgtctccttcccttttccaactgatgacccccagggagtg |
| | | | | | | gcctttgtgttctcctaccc ( (SEQ ID NO: 537) |
| NC_051242.1 | 242755994 | + | 0.47277516 | 0.28381 | 0.024187 | TTCCCTCCCTTCACTATTTTAAATGCACACATCTAACCCAAATCCGGGCTCGTATCTT |
| | | | | | | ATTTGCTCCATCCCACACCCAGCACATATAAATCAGCGCCGAAAGACTCTCCTTCCCG |
| | | | | | | GTATAATGCCACGTACAGCACAGCACCAAGGCGTGGGAACGATAGCGAAAAGACGGAG |
| | | | | | | ATGCGGCCCTGCAGCGAGCCGGATTGAAGTACCAATTCCATTGTGAACTGCCCCCAGT |
| | | | | | | TAAAAGGAAGCAATTGGGTTATTAGTGTTCAAGTGGGAAAAATTAAAACCCAACCGGC |
| | | | | | | TGAAAGCCCCGGCGCAGGAAATTAGAAAGCGGTGCTAATTTACAGGCATTGTCTTAAT |
| | | | | | | TCAAGGGCTAACAATGTGGAAAGTTGTTGTTAGCCCCGGCAAGCAAAGTGGAAAGGAA |
| | | | | | | TAAAATGACCGACCGAAACGGCCCTTTCAGGAGATTTCATTATGAAACTCATTCCCAC |
| | | | | | | TAAATTGTCTTTAACTATTGAATAATGAAAAAGGTAGAACGTTATTTGCTATTTAAGC |
| | | | | | | TTCCCACTTAGAGCCCGCCCGTTAGAATAGTGCTGCGTTTGGGAGCCTCCCGGCAGAT |
| | | | | | | TTCTGGTGGAGTCAGCATAA (SEQ ID NO: 538) |
| NC_051242.1 | 5214346 | + | −0.198160546 | −0.48958 | 0.000047 | ATGAGCCAGAAGAAAAATTAGAAGGTCCCAAGagttagtattttttaaaagctcatcT |
| | | | | | | TTTAAACCCAATTTCACAACTCAGGGCTGTGCGGGGCGGGGCACTCAGTGGTGTGTGC |
| | | | | | | CCCAAGTACTGAGGTACACACaagaattatgacgtgattggaataacagagacttggt |
| | | | | | | gggacaactcacatgactggagtactgtcatggatggatataagctgttcaggaagga |
| | | | | | | caggcagggcagaaaaggtgggggagtagcactgtatgtaagggagcagtatgacagc |
| | | | | | | tcagagctccggtacgaaactgcagaaaaacctgagagtctctggattaagtttagaa |
| | | | | | | gcgtgagcaacaagggtgatgtcgtggtgggggtctgctatagaccaccggaccaggg |
| | | | | | | ggatgaggtggacgaagctttcttccggcaactcacagaagttactagatcgcacgcc |
| | | | | | | ctggttctcatgggagacttcaatcatcctgatatctgctgggagagcaatacagcgg |
| | | | | | | tgcacagacgatccaggaagtttttggaaagtgtaggggacaatttcctggtgcaagt |
| | | | | | | gctggaggaaccaactaggg (SEQ ID NO: 539) |
| NC_051243.1 | 25798703 | + | −0.328460678 | −0.46450 | 0.000126 | ctgggggggggggaagtcagataataattaaaacacatttaacTTAGCACATAGCTCA |
| | | | | | | AAAagctaaaataattaatttttgagGGAGCGGGGGAAAGAGCAGAGGGTAGAATTTG |
| | | | | | | TTTCTGAGGCTGAACTCTTCCAAATACGCAGGTGATGTCGGCTTTGGATTCGTTGACC |
| | | | | | | ATGGAATgttgttccaagaaggaggagtgctaggcagggacaggctccacctaaagaa |
| | | | | | | gagagggaagagcatcttcgcaagaaggctggctaacctagtgaggagggctttaaac |
| | | | | | | taggttcaccgggggaaggagaccaaagccctgaggtaggtgggaaagtgggataccg |
| | | | | | | ggaggaagcacgagcaggagcgcgcaagagggcagggctcctgcctcgtactgagaaa |
| | | | | | | gagggacgatcagcgagttatctcaagtgcctgtacacaaatgcaagaagcctgggtc |
| | | | | | | ctatacacaaatgcaagagcctgagaaacaagcagggagaactggaagtcctggcaca |
| | | | | | | gtcaaggaattatgatgtgattggaataacagagacttggtgggataactcacatgac |
| | | | | | | tggagtactgtcacggatgg (SEQ ID NO: 540) |
| NC_051244.1 | 112335735 | + | −5.731226229 | −0.20134 | 0.113571 | ccccactcccaggGTCCGAGTGGCTTCCCCAGCGCTCTCCCCCATGCGGAGGGGCTCC |
| | | | | | | CCGCTGGCGGCCGCCCCGAAGCTCCCGGGCCCCGCACCTGGTGGTGGTTGTAGGAGTT |
| | | | | | | CCTCTGCTGCAGGAAGGCGGCCGCGGCCGCCGCCGCCTGGTGttggtgctggagctgg |
| | | | | | | gggctCACAGGGGATCTCcggttctgctgctgctgctgctgctgaggtaaATTCATCG |
| | | | | | | GCGGCGGCGGGGCGGCGGCCGAGAAGGGGCTGCTGAAGGGCCCGGCGCAGGGGTTGTG |
| | | | | | | AGGCGACACCGGCGAGAAGCTCTGGAAGAAAGCGGGGTTGATCGAAGACGGGATCCCC |
| | | | | | | GGGTAGAAGCTGGTCTCGGAGTCCGGGCTCGGCGGAGGCATCGCGCTGATCTGGCtcc |
| | | | | | | cgccgccgctgctgctggAGACCGGAGGCTGCTGCGTTTGCGGCGGAGGCGGCGACGA |
| | | | | | | GGTCTGCACCGACCAAGGGGTGCCGAAGCCGGGCAGCGGCGGCGGAGGAGGGGAAGCC |
| | | | | | | GCCGAGGAGGAGCCGCCCCCGCTCTGGTGATGGGGGCTGGGGATCTCCGAGCTCTGCA |
| | | | | | | GGCTGCTGAAGCCGGAGCCG (SEQ ID NO: 541) |
| NC_051244.1 | 54234475 | + | −5.737056225 | −0.13374 | 0.296046 | CCCTCCCGCCCCTCTCAAGTCCCAGCGCTGCCGGGGTGGAACTTTGCCGGGCTCCACG |
| | | | | | | TTCCAGCCCAGCCGGGAGGGGGAAGCGAGGGACACAAGCGCTGCCCGGCCgattcccg |
| | | | | | | cccccctcccagcgCAGCAGCTGAGAAGGAGGAAGGAGGCGCGCTAGGCAGCGGCGCT |
| | | | | | | GAGATTtgccaggcaggcagcagaggAAGAGCAGCAAGGGAAGCCCCCGTGGTCCGTC |
| | | | | | | CTCGCGAGCCGGCTCTTGCGGCAGCCCGCAGGAGCGAGCCTGGCAGCGCTGCGGCGGG |
| | | | | | | GTTGTTCCCCGGGACGCGGGCGCTGAAGTTGCGGTGGCCCAGGCGGGGCGGCGgCccc |
| | | | | | | tgctgctcctctgcctgcTGTTTGTGTGCCTCGGTGGCCGGAGGGGGAGAGCCGCCCA |
| | | | | | | CCTCCGTCCAGCTTCCCACTCCGCTCCCCCGGCTCGGGCTGTTTGTGTGGGAGACACC |
| | | | | | | CACTCCTCCTTCGTTCCCTCCCCCCGGCcgcctcctcccctttccccagagCCGGAGG |
| | | | | | | AGGCCGGAGCGGGCGAGGTGCGGTTGCTGTTGTGGCTCTGGGCTCTCTCGGCCCGGCA |
| | | | | | | CGGCCGCGCCGCTGCTACGG (SEQ ID NO: 542) |
| NC_051245.1 | 2887366 | + | −0.522776785 | −0.53680 | 0.000006 | GGTCCTGCACGATGTACCTGGGGGGGGCGGCGCCCCGAGGGGGGGCAGGCATGGGGGT |
| | | | | | | CAAGGGGCATAACATGATTGGTAAGGGGATGCGGCACCTGTGGGGTGGCGGGCCGGGC |
| | | | | | | ACACGGGGACCCCTCCTCCAGTGCGGGGCCGGGACTCCCCTGGGGGGGGGCCGGGTCC |
| | | | | | | CCGGGGCCGGGACTCCCCTGGGGCGGGGCCGGGTCCCCCCGGGGCACGGCGGAGGATA |
| | | | | | | CACGTCGCGCATGTGGTCGATGGCCGCGGCCCGCAGGATGTCGTTGATCCCGATGATG |
| | | | | | | TTCTCGTGCCGGAAGCGGAGCAGGATTTTGATCTCGCGCAGGGTGCGCTGACAGTACG |
| | | | | | | TCTGGTGCTCGAAGGGGCTGATCTTCTTGATGGCCGCCCGGAGCTTGTTCGCGTGGTC |
| | | | | | | GTAGGCCGAGCTGCGCAGAGAGGGGAGGCCATGGGGCACGGGGGGCACGGGGGGCCCG |
| | | | | | | GCCCCCAGCTTCCCGCGGCAGAGCCCCCTCGGCACCACGGGGacccagaacccaggag |
| | | | | | | tccggcgggcagcccctccccccgcgcgCCTCCCGAGCCGGGACAGGACTGTACCCCG |
| | | | | | | GGGGCGGCGGCGCTGcgagc (SEQ ID NO: 543) |
| NC_051246.1 | 252327 | + | −1.284344404 | −0.44656 | 0.000244 | ATTATGCAGCCCCATGATGGGGCACCTCCTGACAAATAGTGAAGCTATTTGCAGAGAT |
| | | | | | | TCTGGCTCAGGGAGTCATTTGCTAACCCTAGCCAGGACAGGATATTGACCAGCCCTGT |
| | | | | | | TCCTTTATGTGGgccaagccctggtctacactaggacttgaggtcgaatttagcagca |
| | | | | | | ttaaatcgatgtaaacctgcacccgtccacacgatgaagccatttttttttgacttaa |
| | | | | | | agggctcttaaaatcgatttctttactccacccctgacaagtggattagcgcttaaat |
| | | | | | | cggccttgccgggtcgaatttggggtactgtggacacaattcgatggtattggcctcc |
| | | | | | | gagagctatcccagagtgctccattgtgaccgctctggacagcactctcaactcagat |
| | | | | | | gcactggccaggtagacaggaaaagaaccgcgaacttttgaatctcatttcctgtttg |
| | | | | | | gccagcgtggcaagctgcaggtgaccatgcagagctcatcagcagaggtgaccatgat |
| | | | | | | ggagtcccagaatcgcaaaagagctccagcatggactgaacgggaggtacgggatctg |
| | | | | | | atcgctgtatggggagagga (SEQ ID NO: 544) |
| NC_051246.1 | 404436 | + | −0.295417225 | −0.34151 | 0.006158 | aGCCCAGTCCGGTCAGCATGCCAGCCTGGGCAGATAGACTTGTGCGAGCAGGGCCTTC |
| | | | | | | ACGCCCGGCCGACGTTGGCTCTGAGCTCAGGGGGCCGCAGCCTCCACACTGCTCGTTT |
| | | | | | | AGTGCCCGACCTCGGGCCCCACCAGCCCACGACTGTCCCGGGCTCGCAGGCTGGCTCC |
| | | | | | | CCGCTGCAGGGCAGACACAGCCCAGGAGCCCCCCACACGCCCCTGTGAGCCAGAGCCC |
| | | | | | | AGCCCGGTCAGTCCTGGCTGGCCCCCACCGGCCCGCTGAGCCCGGCGAGGGAGGTGGC |
| | | | | | | AGTCTTCCCCGGCAGGGTGGTGCGGGGCAGCGTTTGGGTTTGCAGAGCGGTGAGTAAA |
| | | | | | | GGAGCAGGAATGCCCGGCACAGATTCACTCCCAAAAAGGAGATTGGAACAACTTGTGG |
| | | | | | | GTTTCCTGTTTACTGAAGCCAGCAAGGCTGCCCGTGCggggtcccctcccccaccccg |
| | | | | | | gctgcCGGAGGCAGCactggccaggctggggaggggacctCGAGGCTGGGGGGCCTGC |
| | | | | | | GGATGCCCTCGGGGCCCCGGTCACTCTCTGCCACCTCTGGCCGGAGATTGGCTGGGCT |
| | | | | | | CCTCagccgggggggggcag (SEQ ID NO: 545) |
| NC_051247.1 | 122622449 | + | 0.718999798 | 0.59246 | 0.000000 | CCGCACCTGTCGGCGTAGGGTAGACACACGCTGAGCCAGTCAGTGTAGCGCGCGTGCA |
| | | | | | | GCCCCGGACATCTAAGGGCATCACAGACCTGTTATTGCTCAATCTCGGGTGGCTGAAC |
| | | | | | | GCCACTTGTCCCTCTAAGAAGTTGGACGCCGACCGCTCGGGGGTCGCATAACTAGTTA |
| | | | | | | GCATGCCAGAGTCTCGTTCGTTATCGGAATTAACCAGACAAATCGCTCCACCAACTAA |
| | | | | | | GAACGGCCATGCACCACCACCCACAGAATCGAGAAAGAGCTATCAATCTGTCAATCCT |
| | | | | | | TTCCGTGTCCGGGCCGGGTGAGGTTTCCCGTGTTGAGTCAAATTAAGCCGCAGGCTCC |
| | | | | | | ACTCCTGGTGGTGCCCTTCCGTCAATTCCTTTAAGTTTCAGCTTTGCAACCATACTCC |
| | | | | | | CCCCGGAACCCAAAGACTTTGGTTTCCCGTAAGCTGCCCGGCGGGTCATGGGAATAAC |
| | | | | | | GCCGCCGGATCGCTAGTCGGCATCGTTTATGGTCGGAACTACGACGGTATCTGATCGT |
| | | | | | | CTTCGAACCTCCGACTTTCGTTCTTGATTAATGAAAACATTCTTGGCAAATGCTTTCG |
| | | | | | | CTTTGGTCCGTCTTGCGCCG (SEQ ID NO: 546) |
| NC_051247.1 | 123209497 | + | 1.335074269 | 0.31624 | 0.011571 | GCTGCTCGCTGGAGGCAGACTGGAGCCCAGCGCAGCTCTCCGCGCCGCTTTGCTCAGG |
| | | | | | | GCGGGAGCTGCAAACACCGGCCAGCGCGATGGCCCAGCCTCCTCCCCGGCGGGGCAAC |
| | | | | | | TGAGGGCCGCTGCGCTCCGCGGAGCCCCAGTCCCGGCCCCTCTGTGCccctggcgggg |
| | | | | | | gcgggggaccGGCGCAGCAGCTGTTGGGGCCGCAGGGCAGAAGGGGGCTCGGCCCAAA |
| | | | | | | GCCTGTCCGATCCCCCACGGCGGCCGGGGATGGGAACGCGTCCCCCGGCTCTGGGTGG |
| | | | | | | CTCGGCTGCCGGGCTGGGCATGGAGCTGGAATCCCCGCACCGTCCAAGCAGCTTCGCT |
| | | | | | | TGTAAAAAGCCAAAGTGTCGGGGTCTGGGGGAGGCAGCCGCGCGGAGAGCCGGGCCCC |
| | | | | | | TGTATCTACTCTGTATCCGCTGTAAGTGCCAGCCCGCGCAGGAGCGAGGGACTTAATA |
| | | | | | | AACCAGGTGCAATAGCCCCCGCGCCTCATTGTTAGCGGCCCGGGAGCCCGTGGCCTGG |
| | | | | | | AGCAGGGACCGGAGCGGACCGCCGGCGGGGGATTTCCGTTCGCCCCAGCCCACGGGAT |
| | | | | | | GTTGTACGTTAGGGGTGAAG (SEQ ID NO: 547) |
| NC_051247.1 | 22362197 | + | 0.100105839 | 0.32096 | 0.010324 | CCAATGCTGGCTGCATGGCCCCTGTGCGTGGTTCTGAGCAGGCGGCCCCTGCGGTGAA |
| | | | | | | GTTGTGGAGCAGTTTCCGACCCGTGCCCTCTTCCCATGCCTCTCCTGGGTGGCTGCAT |
| | | | | | | ATGCTCCCTCAGCTGAAATCCAGAGCCCTCGCCTGGTGAGACATGGCACTCATGGGCT |
| | | | | | | CTTGTGATTCAGGCCTGAGTCCTAGGTGTCTTTTCCAATTAGGCCATTCACTGCTCAG |
| | | | | | | GCCTGTGCCTGCCCTGAGCACTTGGGATTGCCATGGCAACCAGGAGCGCCAGCATTTA |
| | | | | | | TTAATCATCCGGGTGGCCTTTGGGTTGGCATGGCAACTGCAGACAAGAATAGAAACAC |
| | | | | | | AGGAGTGGGAAAGTGGCAGAGGAGCTGCCAGGCAATGGCACATGTCAGCCGGAGCTGG |
| | | | | | | GGCCCCCCTGTCCCGGTGCCGGGTGCTGCGAGAATTGAGGCCTTCCCTTCAGATCTGG |
| | | | | | | CATTTTTTCTTGGCTTCTGAAACAATTTTACTTTAGGCTAAGAAAACAGCTCTGCACA |
| | | | | | | CGGGAGTGATTCTGGGATCCCTgcctgctgcagggaggggctcagTTGAGGCTGAAGG |
| | | | | | | ATGCTACAAAGAGAAACCCA (SEQ ID NO: 548) |
| NC_051248.1 | 95968272 | + | −6.513613347 | −0.25138 | 0.046883 | AAAATCTAAAGTAGTTCCACTTTTGATTGTAATTTCAACTAAAATACTTCTACAGCAA |
| | | | | | | ATAAACCCTCTTCTGCAAGCGTTTGAAAGCGTAAGTTGAAAGTGTAAAAGAGGAGAAA |
| | | | | | | CCTAATTTTCCATCGTCCAAactgaaagaaatgcaaaatgaaaatcCAACCGTATTAA |
| | | | | | | ATAGTGAAAGAGTTTTAGATGCTCACTAAGCGAAGACATTACTTGTAAGCAAACGTTT |
| | | | | | | GCTACAACATACACCCTTTAAGTTCACAGAATGCCCCCTAAAATACAGAATCGCCGCA |
| | | | | | | AAAGCAGCCCGGTATTGTTCGAGCAGCAAAAGAAAGTCATTTTCCCAGGAGGTCCTGC |
| | | | | | | GAACTCCTCGAGCGAAGGAAACGCTGGAGCCCGGTTTTTACGCCCCCTCCGTGCGGGG |
| | | | | | | CCAGCAGCGCTCGCCGCTTGTTGTGAGCGCCCGGGCTGAGCTGGCGGAAGCCTGCGCG |
| | | | | | | CTGCCAGCAGGCGAATCACCGCCACTCGCGGGCCGCGCGCGTGGACTGGCGACACGAG |
| | | | | | | CAGCGCCAGCCCGTCCCGGCGGGTCTCCGCGAGCAGAGCGGGCCGCCGAGAcgcgggc |
| | | | | | | agggggaggagctgcgCGCC (SEQ ID NO: 549) |
| NC_051249.1 | 48918401 | + | −8.861229161 | −0.20134 | 0.113571 | TCCCACAGCGCCGCCACCCTGCAAATTCTGTCGGCGCGGTGACAGACGGACGAAGCGA |
| | | | | | | GGGTCGAAGGGTTTTCTCGAAAGCGGCTCCGGGATGCTGAGACGGTGGGCTGCAGGTG |
| | | | | | | CAGTAAAGCCCTAGATGGCCCGGCCTGTTGGTCCCACCCTGGCCCACATGCTCCCGGA |
| | | | | | | CCCCAGCACCCCGCCTGCAGGCCCGGCCCTGTGCCCCACACCACGGGCACCGGGCGCT |
| | | | | | | GGCGGCAGCACCACGAGCCTGGGGAGCCCCCGGGCGCTCGCGCCCAGCTCTGGTGACC |
| | | | | | | CGGGGGTCCCGGGCCCACAGCGAGAGCGGCGCCGTCAGGCACGGGGGCGAGATGTGGG |
| | | | | | | CGGCTCCCGCAGCAGGGAGGAGCCGCTTCCCCGGGCCAGAGAGGGCAGGACCGGAGCC |
| | | | | | | gagctgggggagagggggctgggcccGGCCGTCACCCACCTGCCGGCCCCGAGCGGGC |
| | | | | | | CCCCCGGGCGCAGCCGCGCCGACAGCAGGTAGAGGCCGCAGGAAGCGGCCCCGAGCAG |
| | | | | | | CGCCAGGAAACCGCAGAGCGACCGCATCCCGGCGCGGGAGCTACAGCGCCAAGAGCGC |
| | | | | | | CGGGTACCGGCGGTTCGCGG (SEQ ID NO: 550) |
| NC_051250.1 | 46230416 | + | −1.753160894 | −0.47821 | 0.000074 | caaatgttttcgcatttcgaaaactggaacagagttctgatagtaCAGATTCCTCTCC |
| | | | | | | CTATACAGCGATCAGAGCCCGTACCttccgttcagtccatgctggagctcttttgcga |
| | | | | | | ttctgggactccatcatggtcacctctgctgatgagctctgcactcacctgcagcttg |
| | | | | | | ccacgctggccaaacaggaaatgaaattcaaaagttcactggccttttcctgtctacc |
| | | | | | | tggccagtgcatctgagttgagagtgctgtccagagcggtcacaatggagcactctgg |
| | | | | | | gatagctcccggaggccaataccgtctaattgcgtccacagtaccccaaattcaacct |
| | | | | | | gcaaggccgatttcagcactaatccccttgtcgggggtggagtaaagaaatcgatttt |
| | | | | | | aagagcgctttaagtcgaaaaaaagggcttcgtcatgtggacggttgcagggttaaat |
| | | | | | | caatttaacgctgctaaattcgacctcaactcctaaagtgtagaccagggcttaggct |
| | | | | | | tCTGCAGCTGCAAACAACAAGGGATCAtgaaaattaaactaaacaaaGAGAATACATG |
| | | | | | | TTGGCAGCCTACTGGAGACC (SEQ ID NO: 551) |
| NC_051252.1 | 35828717 | + | −0.297612289 | −0.21671 | 0.088018 | tctccatcctggGCCATGTGTGAAGCCGGGCGGGAGGTTTGTCTAGAAATCTGCCTAC |
| | | | | | | gttcccatccccccacccgcCAGGTGATCCCAGTCTCCCCGTCCCTCCTTCCAGCTGG |
| | | | | | | GAATTCTCCAGGGGGCTGGAGTGGTGGGGAGCAGAAACGAGGCTCGGAGAGTGGGCTT |
| | | | | | | GGGAGCCCGTCTCGCTCACTTCAGCTCTGGCGTTCCTCGCCCCCATCCCACCCTCAGC |
| | | | | | | tccccgctcctctgcggagacTCCCTGCGCGCCTCAGAGCCAGATTTCTGCTGCGGGA |
| | | | | | | GAAATCAACCGGCTGCCGTCCTCTTACCCCGTCGTAAGGCAGCAAATCCAAGGCCCAG |
| | | | | | | GCCGAACGGCTCGGGGGCGTGGAGCAAGAACGCCGGGAGCCCCTGGGCGCGGCCAAGG |
| | | | | | | GCTGGATTTCAGCCccgttggggcgggggggtctatGGCGAACTGCTCCATCCTGGGG |
| | | | | | | TGAAAGCTGAGTAAATAGGCAGGGCCGTGTCCCTGCCAAGAACAGCCAGGAGCTGGAG |
| | | | | | | CCTGCTCCACAAACCTCCGCTTCTCTGCAGGGCGACAGCGGGGAAGAAGGGCCTAGCT |
| | | | | | | GGGTTAACAACCCCCCAGTG (SEQ ID NO: 552) |
| NC_051253.1 | 11964470 | + | −7.17774885 | −0.26461 | 0.036109 | CCGTGCCCGCCAGACACCACACGCCGGCGGGGCCAAGCCGAGCAGCCCCCGGTCCCCG |
| | | | | | | CGACTGCTCGGCACCTTTACCGCCGCCCCTGCGCCCCGCCAGGCCCCCGCGCGGACTC |
| | | | | | | GCCCCGCTCACCCCCGTGGCGCCGGGCCGCCCTCGAGAGCCGCCGGGCCGGCGGGCCC |
| | | | | | | CCGAGCCCGCGCAGCCCCGGCGACGTCGCCGCcctccgcagcagcagcagcatcccgg |
| | | | | | | CAACAGCCAGGCGCGGAAACAGCCCCTGCCCTTTCACCCCGACCCGCGTGTCGTCATC |
| | | | | | | GCCGCGCGCCGGAAGTGACGGAGAGACTGGAGCGTGCTGGGCGGAGGCGAGGAGCGAG |
| | | | | | | GTGAGGGGTCCCCCGGCGCCCCCGGGAGCGGGCCGCGGAGAGCGTGGGATCAGCCGGG |
| | | | | | | CCCCGCGGAGCTTCCCTAGCCGGCGGCCGCAGGGTCCCGGCTCGCGCGGTGCAGGCGC |
| | | | | | | CAgggcttgccccagccctgccaagcTGGGGGCCGCACCAGTGTCCCGTGGCCGTGAG |
| | | | | | | TTCCGCAGGCTCCTCTACAGCCTCCGAGCCCCCGTGCTCTGTGACGGAGTGAGCCGGG |
| | | | | | | CTCTGGCCCCCTCCAGGGCG (SEQ ID NO: 553) |
| NC_051253.1 | 4789859 | + | -0.531908053 | -0.61576 | 0.000000 | AGTCTCTGTTTCATGATGGCAGCATATCGCAACAGGTGGCAGATGGCAACATAGCGGT |
| | | | | | | CAAAGGACATGACCATAAGAAGGATGAACTCCATAGCTCCCAGGGTGAAATAGAAGTA |
| | | | | | | GGATTGGGCCATGCAGGCAAGGAATGAGATGGTTTTGCTGTCTGAGAGAAAGTTCAAC |
| | | | | | | AGCATCTTGGGGTTTGTGACCGAGGTGAACCAGATCTCCAAGAAGGACAGATTGCTGA |
| | | | | | | TGAAAAAGTACATGGGGGGTATGGAGTCGGTGATCCACCCACACTATGAAAATGATTA |
| | | | | | | ATGAGTTCCCGGTTAGTGTGACCAAGTAGGTCAGTAAAAGAACAAAGAAGAGAAACAT |
| | | | | | | CTGGAGTTTGTCATGAACTCCGGAAAACCCCAAGAGCCTGAATTCAGCCACTGTGGTT |
| | | | | | | TCATTTGCTTCCTCCATTTCCGATTTCAGTTTCCCCTGTAGAAAGagcaacaataaaa |
| | | | | | | aaataagagcATGAACCTTTATCTATCACATGTATGGATTCAACAGCAATGTTCTGGA |
| | | | | | | GGGTTGTGAGACAGAAAAGCTGAACACTCGAGATCACACACTAAGGAAGGAGACAGAA |
| | | | | | | TTAGTGAGATTGACAGAGAA (SEQ ID NO: 554) |
| NC_051255.1 | 33315256 | − | −0.893274231 | −0.36204 | 0.003549 | AGGCTGGCTGTCGAATAAATTTTCCTGCCACacagggtttcttcaggtatgttagcaa |
| | | | | | | caagaagaaagtcaaggaaagtgtgggccccttactgaatgagggaggcaacctagtg |
| | | | | | | acagaggatgtggaaaaagctaatatactcaatgctttttttgcctctgtcttcacga |
| | | | | | | acaaggtcagctcccacactactgcactgggcagcaagcatggggaggaggtgaccag |
| | | | | | | cctctgtggagaaagaagtggtttgggactatttagaaaagctgaacgagcaaaagtc |
| | | | | | | catggggccggaggcgctgcatccgagagtgctaaaggagttggcggatgtgattgca |
| | | | | | | gagccattggccattatctttgaaaactcatggcgatcgggggaagtcccggacgact |
| | | | | | | ggaaaaaggctaatgtagtgcccatctttaaaaaagggaagaaggaggatcctgggaa |
| | | | | | | ctacaggccagtcagccacacctcagtccctggaaaaatcatggagcaggtcctcaag |
| | | | | | | gaatcaattctgaagcacttagaagagaggaaagtgattaggaacagtcagcttggat |
| | | | | | | tcaccaagggcaagtcatga (SEQ ID NO: 555) |
| NC_051259.1 | 1959061 | + | −0.078729836 | −0.24997 | 0.048170 | ggtggcggagatgagctggggcgggaACCGAttcccctgcacccctgccccgggttac |
| | | | | | | ctgctgcggcgcaggcgaccctcctcgtgcccccccctcccccccagctcccctccgc |
| | | | | | | tccgcctccctgggcctgagcgcgaagccgccccctgcttctcagcccccggcttccc |
| | | | | | | acgcgaacagctgattcgcgggaagcggtgggggggaggcggagaagcagagcggggc |
| | | | | | | ggagcATAACTCAGGGGCGGaagcggagcagaggtgagctggggccagggctggggcg |
| | | | | | | gggagctgccggtgggtgctctgcacccaccaaattttccccgtgggtgctccagcct |
| | | | | | | cgaAGCACCCATGGGgtcggcacctaaggcaccacttttggctggttgttacatttag |
| | | | | | | aagcccttttagaacatgacggacaaccggttctaaaagggcttctaaatttaacaac |
| | | | | | | cggttctagtgaactggtgcgaaccggctccagctcaccactgcccttGCCGCACCCT |
| | | | | | | CTGCCTCGCCACTTCCCCGAGGCCtcgaccctgccctgccccttctctgaggcccctg |
| | | | | | | ccctgctcactccatccccc (SEQ ID NO: 556) |
| NC_051260.1 | \|114280 | + | −5.929687527 | −0.20134 | 0.113571 | CTGAGGAGGGATGAAAGCCCCTCCCTTTCATGCCACTTTATAGGCAACCCCGATTTTG |
| | | | | | | TTCTTTGGAGGCCAATTTCACACAAGCTGGGGGAGGCAGAGAATGGGGTTAACAGCAG |
| | | | | | | CTATTCAAATGACATTTATGTGAGGTGTACGAACACCCGACCCCCTTACACGCTGACC |
| | | | | | | CCATAATCCAGTTGGAGTTACTGCCCCATGCCCGGCTCCGGCCCAGCACCTCCTCCCT |
| | | | | | | GCCTCTTACCCCGTGCCTGCAGGCCTGCTCGTTGAGGGCTCGCACCCAAGCTCGCTGC |
| | | | | | | CAGTTCTCCCGGAAGGATCTGAAGGCGAAGAGCGAAGCAAGGAGCCCTCTGGCCCCCG |
| | | | | | | CTACCGCCTCCTCGGGAGCTCCGTCCGGGGCCACCCGCAGTCTCAGTAAGGACTTCCA |
| | | | | | | GATGCCTGCTTCCCTTAGAGCTCCTCCCGAGGTCTCCGGCTTTCCCCGGCCGCTCCAC |
| | | | | | | ACGCCCCGGGAATACTGCAACAGCCAGGCCGAGACGGTGAGCAGGGAGGCGGCGAAGA |
| | | | | | | GCAGCACCAGAgccgcccagcccagctccagctccagctccatggTGCTCTCTGCTCA |
| | | | | | | GCAACTTCGGGGAGGCTAGT (SEQ ID NO: 557) |
| NC_051265.1 | 1469699 | + | −0.102712233 | −0.46298 | 0.000133 | Accatctcatggtcactgcctcccaggttcccatccactttagcttcccctactaatt |
| | | | | | | cttcccggtttctgagcagcagatcaagaagagctctgcccctagttggttcttccag |
| | | | | | | cacttgcaccaggaaattgtcccctaccctttccaaaaacttcctggattgtctgtgc |
| | | | | | | accgctgtattgctctcccagcagatatcaggatgattgaagtctcccatgagaacca |
| | | | | | | gggtctgcgatctagtaacttccgtgagttgccggaagaaagcctcgtccacctcatc |
| | | | | | | cccctggtccggtggtctatagcagactcccatcacgacatcacccttgttgctcaca |
| | | | | | | cttctaaacttaatccagagacactcaggtttttctgcagtttcataccgaagctctg |
| | | | | | | agcattcatactgctctcttacatacagtgcaactctgcCACCTTttttgccctgcct |
| | | | | | | gtccttcctgaacagtttatatccatccatgacagcattccagtcatatgagttatcc |
| | | | | | | caccaagtctctgttgttccaatcaaatcataattccttgactgtgccaggacttcca |
| | | | | | | gttctccctgcttgtttccc (SEQ ID NO: 558) |
| NC_051265.1 | 565064 | + | −0.152218372 | −0.32387 | 0.009614 | Tcactgcggactacgtggctctgggaagaaggataaaggagttggaggcgcaagtggt |
| | | | | | | gttctcgtccatcctccccgtggaaggaaaaggcctgggtagggaccgtcgaatcgtg |
| | | | | | | gaagtcaacgaatggctacgcaggtggtgtcggagagaaggctttggattctttgacc |
| | | | | | | atgggatggtgttccatgaaggaggagtgctgggcagagatgggctccatcttacgaa |
| | | | | | | gagagggaagagcatctttgcgagcaggctggctaacctagtgaggagggctttaaac |
| | | | | | | taggttcaccgggggaaggagaccaaagccctgagataagtgggaaagcgggataccg |
| | | | | | | ggaggaagcacaggcaggaatgtctgtgaggggagggctcctgcctcatactgggaat |
| | | | | | | gaggggcgatcaacaggttatctcaagtgcttatatacgaatgcacaaagccttggaa |
| | | | | | | acaagcagggagaactggaggtcctggtgatgtcaaggaactatgacgtgatcggaat |
| | | | | | | aacagagacttggtgggataactcacatgactggagcactgtcatggatggttataaa |
| | | | | | | ctgttcaggaaggacaggca (SEQ ID NO: 559) |
| NC_051266.1 | 5261789 | + | 1.114350619 | 0.45100 | 0.000208 | acctctgctcccctcctggctggagggctggttccccagctggctgctttcccctctc |
| | | | | | | tgccccatgcCTTCCCTGGAGGACCCCTGGAAGCCAGTAGTGATGATGACAGGACCAC |
| | | | | | | TTGATGGTGCCAGGGACTTTGTTTACAATCATCAGCTATACAAACACATTGTTTCTGT |
| | | | | | | GCAAGAAGCTACCAGGCTAAGGTGTGGGGGGGCCGGGGATAGTGGACTGGGGagagcc |
| | | | | | | ccttccccctccctgctcatcAGCCAGGAGCTGTTGACATCTGTTTTCATTGGGGATG |
| | | | | | | CTTTGATGCCGGCTGTTCTTGATTGAAGGCAAACAGAGCCCTGGAGGCGAGTGAGGAC |
| | | | | | | AGGTTTCCAATCCTCGGGGGCTCCGGTGTCGTCTCGGCACAGCCATCAATAATGGTCC |
| | | | | | | GAGGGGCCTCAGCTTCATCCTGGCCCTTGCAGGGGGCTCCAGCTCCTACAGACAGCTA |
| | | | | | | TTTGTGCTTTGTTGGAAAGACCCAAGTACCCAGACGGGCTTGCTGACTGAACGGTAAC |
| | | | | | | GCTGCAGGGGCAGGCAAGGGACACATGTATGTGGGATCAGAGCAGGGGGCACATCTAG |
| | | | | | | GCCAGTGTAGCCCCCTTCCT (SEQ ID NO: 560) |
| NC_051267.1 | \|11665443 | + | 0.212431685 | 0.40292 | 0.001060 | AGAaccggctgctggccccttgcccgTGACAGAGCACAGGGCCACACACCTCATTAAG |
| | | | | | | GCAGGAGATGAATTTAACAATTGTACCTGGATTCGAGGGCACGCTGAATTTTGTGGCT |
| | | | | | | TCGTTGTTTTCAAATCTGTGACCTGCTTTGGCTTTTCACAGCCAGGCACCCAAAtaga |
| | | | | | | gattgggggggggggggcgggggaatagcTCGCCTTGGCTAGAATGAATACATTTGCt |
| | | | | | | ttgctgcggggggggggggtagttgggagacttttttttttaagtgtgtgtctACATA |
| | | | | | | TGCACGCCCCGGGAGAGAGAGGGTGTGATCGTTGCTTGGAAAGCAGAAGGGGATTGGA |
| | | | | | | GTGAAAACTCCAGGGCTGGGCACAAAGATAATAAGCACCGGGCAGAATTTTTAGTGGC |
| | | | | | | GGAAAAACATGACTTGTCTGCAAGGGGCAGttcccatgccctcccccccccccccata |
| | | | | | | cacacacgaAAGAAGACAAAACGGAAAGGGGAAAAACAACGGGTTTTGCCAACATTTT |
| | | | | | | ATCCCGAGTTTGAGACCCTGAGCGGGATGTTTGCTGCACGAAATAAAGTGGGGTTACA |
| | | | | | | ATCTAGTCCCCCTGGGAAAT (SEQ ID NO: 561) |
| NC_051268.1 | 1438203 | + | −1.770073119 | −0.50049 | 0.0000295 | CTCTGGATTTACCATTTCCTGGCTGCGGAAAGACgcacttcccctccctcctgggccA |
| | | | | | | GGGCATGGGGGGTGTCCCTgcatcctcccccccacccccccagctggcAATCTAcacc |
| | | | | | | ccccagggctccctctcctccctactgcggcggggggggggctttgcTTTGGCAGGCT |
| | | | | | | TCagtggggagaacccaggagtcctggctcccatcagCCCGTGGCTGTCCTGCCGGGG |
| | | | | | | TGGCACTGGGGCTGTGTGGCACCTTGGCCCCGTACTGCCCCAGGCTGAGGGCGATTCT |
| | | | | | | CCGCGCGCCCGGCTGCTGAGGGCGGCTCTCCTGGTACAAAGGGGCCCTGTGCGAGCCC |
| | | | | | | CGCTGTGAGTCACCGGGGCTGGTGGGGAGCCGCGGCTTGGCTGGAGTTCCCAGCCGGC |
| | | | | | | CGCCCGCACAAAGGGCCTTTGAGAGAGGGGGCGGGCCCAGAgccgggagggggttggg |
| | | | | | | ggactATGGGGGccctggctctgagctggggggggagcagcttTGGGGGGGACTTTGG |
| | | | | | | AGGGGGGAGCAGCCTTTTCCAACCACTAGCCCGGTTCACCGCCCCCTCTGTGTATAGC |
| | | | | | | CTGAAGGGGGGGCAAGCCCC (SEQ ID NO: 562) |

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU2020903422 filed 23 Sep. 2020, the entire contents of which are incorporated by reference herein. The present application also claims priority from AU2021900750 filed 16 Mar. 2021, the entire contents of which are incorporated by reference herein.

### REFERENCES

Adam et al. (2019) PLOS ONE 14:e0220934.
Austin et al. (2017) GigaScience 6:1-6.
Biscotti et al. (2016) Scientific Reports 6:21571.
Bolger et al. (2014) Bioinformatics 30:2114-2120.
Cadrin and Friedland (1999) Fisheries Research 43:129-139.
Campana (2001a) Journal of fish biology 59:197-242.
Campana (2001b) Journal of fish biology 59:197-242.
Campana and Thorrold S (2001) Canadian Journal of Fisheries and Aquatic Sciences 58:30-38.
Caughley (1977) Analysis of vertebrate populations: Wiley.
Clark et al. (2006) Nature Protocols 1:2353-2364.
Couch et al. (2016) PeerJ 4:e2593-e2593.
Espinoza et al. (2019) Maccullochella mariensis. The IUCN Red List of Threatened Species 2019: e.T122906177A123382286 (Available online at dx.doi.org/10.2305/IUCN.UK.2019-3.RLTS.T122906177A123382286.en)
Falisse et al. (2018) Environmental Pollution 243:1867-1877.
Fallon et al (2015). Radiocarbon 57:195-196.
Fallon et al. (2019) PLOS ONE 14:e0210168.
Fowler (2009) in Tropical Fish Otoliths: Information for Assessment, Management and Ecology. eds, Green et al., (Springer) pp. 55-92.
Friedman et al. (2010) Journal of Statistical Software 33:1-22.
Gauldie et al. (1986) New Zealand Journal of Marine and Freshwater Research 20:81-92.
Gooley (1992) Marine and Freshwater Research 43:1091-1102.
Guo et al. (2013) BMC Genomics 14:article number 774.
Harris (2007) Improved pairwise Alignment of genomic DNA PhD Thesis Pennsylvania State University.
Harris (2007) Improved pairwise alignment of genomic DNA. Ph.D. thesis, Pennsylvania State University.
Herman et al. (1996) Proceedings of the National Academy of Sciences of the United States of America 93:9821-9826.
Horvath (2013) Genome Biol 14:R115.
Huang et al. (2013) in Ovarian Cancer: Methods and Protocols. eds Malek et al: (Humana Press) pp. 75-82.
James et al. (2010) Radiocarbon 52:1084-1089.
Kim et al. (2015) Nature methods 12:357-360.
Korbie et al. (2015) Clinical Epigenetics 7:article number 28.
Krueger and Andrews (2011) Bioinformatics 27:1571-1572.
Kuhn (2008) Journal of Statistical Software 28:1-26.
Kuleshov et al. (2016) Nucleic Acids Research 44:W90-W97.
Langmead and Salzberg (2012) Nature Methods 9:357-359.
Le et al. (2008) Journal of Statistical Software, 25:1-18.
Li and Dahiya (2002) Bioinformatics 18:1427-1431.
Lu et al. (2017) Scientific Reports 7:1-12.
Mayne et al. (2020) Aging (Albany NY) 12:24817-24835.
Nock et al. (2010). Marine and Freshwater Research 61:980-991.
Ortega-Recalde et al. (2019) Nature Communications 10:article number 3053.
Picard and Cook (1984) Journal of the American Statistical Association 79:575-583.
R Core Team (2018) R: A language and environment for statistical computing. (Available online at www.R-project.org/).
Ralser et al., (2006) Biochemical and Biophysical Research Communications 347:747-75.
Rhie et al. (2020) bioRxiv 2020:2020.2005.2022.110833.
Shen et al. (2016) PLOS ONE 11:e0163962-e0163962.
Smallwood et al. (2011) Nature Genetics 43:811-814.
Stubbs et al. (2017) Genome Biol 18:68.
Thompson et al. (1994) Nucleic Acids Research 22:4673-4680.
Thompson et al. (2017) Aging (Albany NY) 9:1055-1068.
Wang et al. (2013) Nature Genetics 45:701.
Worthington et al. (2011) Canadian Journal of Fisheries and Aquatic Sciences 52:2320-2326.
Xi et al. (2012) Bioinformatics 28:430-432.

1-56. (canceled)

57. A method for estimating the age of a fish comprising:
analysing DNA obtained from a fish for the presence of a methylated cytosine at age-associated CpG sites; and
estimating the age of the fish based on methylated cytosine levels at the age-associated CpG sites.

58. (canceled)

59. The method of claim 57, wherein the age-associated cpg sites are selected from:
(i) Table 8 or 9 or a homolog of one or more thereof,
(ii) Table 1, 2 or 3 or a homolog of one or more thereof;
(iii) Table 12 or a homolog of one or more thereof, or
(iv) Table 16 or a homolog of one or more thereof.

60. The method of claim 57, wherein the age-associated CpG sites are selected from Table 8 or 9 or a homolog of one or more thereof.

61. The method of claim 57, wherein the age-associated CpG sites are selected from Table 1, 2 or 3 or a homolog of one or more thereof.

62. The method of claim 57, wherein the age-associated CpG sites are comprised within one or more of the amplicons listed in Table 5.

63. The method of claim 57, wherein the presence of methylated cytosine is analysed at five or more, 10 or more, 15 or more, 20 or more, or 25 or more of the age-associated CpG sites.

64. The method of claim 57, wherein analysing DNA comprises multiplex PCR and DNA sequencing.

65. The method of claim 64, wherein the multiplex PCR uses two or more primer pairs configured to amplify a region of the DNA comprising the age-associated CpG sites.

66. The method of claim 65, wherein at least one of the primers (i) is selected from Table 4; and/or (ii) can be used to amplify the same CpG site as the primers of (i); and/or (iii) hybridizes to a region of the DNA within 100 or 50 or 20 base-pairs of a primer of (i).

67. The method of claim 57, wherein analysing DNA comprises determining the methylation beta value of the age associated CpG sites.

68. The method of claim 57, wherein the DNA analysed is from caudal fin and/or a skin biopsy.

69. The method of claim 57, wherein the fish is a member of the subclass Elasmobranchii.

70. The method of claim 69, wherein the fish is a shark.

71. The method of claim 57, wherein the fish is a member of the infraclass Teleostei.

72. The method of claim 71, wherein the fish is a Grouper, Tuna, Cobia, Sturgeon, Mahi-mahi, Bonito, Dhufish, Murray cod, Barramundi, Herring, Tra catfish, Mekong giant catfish, Cod, Pilchard, Pollock, Turbot, Hake, Anchovy, Haddock, Black carp, Grass carp, Eels, Koi Carp, Giant gourami, zebrafish, Mackerel, Australian lungfish, Mary river cod, Salmon or Trout.

73. The method of claim 57, wherein the age-associated CpG sites are identified by:
analysing DNA obtained from the species of fish of different chronological ages for the presence of methylated cytosine at CpG sites; and
using a statistical algorithm to identify age-associated CpG sites.

74. A method of identifying an age-associated CpG site for a second species of fish comprising
(i) analysing DNA of the second fish species for a candidate age-associated CpG site corresponding to an age-associated CpG site identified for a first species of fish;
(ii) analysing the methylation patterns of a candidate age-associated CpG site identified in (i) in different ages of the second species of fish to determine if it is an age-associated CpG site in that second fish species.

75. The method of claim 74, wherein the first fish species is zebrafish and step (i) comprises analysing DNA of the second fish species for a candidate age-associated CpG site corresponding to an age-associated CpG site listed in Table 1, 2 or 3.

76. The method of claim 74, wherein the first fish species is a shark species and step (i) comprises analysing DNA of the second fish species for a candidate age-associated CpG site corresponding to an age-associated CpG site listed in Table 8 or 9.

77. The method of claim 1, wherein the age-associated cpg sites are identified by the method according to claim 74.


SEQUENCE LISTING (562 sequences)

SEQ ID NO: 1 - DNA, 37 bp, Artificial Sequence
  Primer
gacatggttctacatgagtgtttgtttggttaagtat

SEQ ID NO: 2 - DNA, 33 bp, Artificial Sequence
  Primer
cagagacttggtctcaaaacaattcctccaccc

SEQ ID NO: 3 - DNA, 33 bp, Artificial Sequence
  primer
  misc_feature (21)..(21): n is a, c, g, or t
gacatggttctacaaaccccnaaaaaccactac

SEQ ID NO: 4 - DNA, 45 bp, Artificial Sequence
  primer
cagagacttggtctaataagatagttgaaatttttaaggtgttta

SEQ ID NO: 5 - DNA, 45 bp, Artificial Sequence
  primer
cagagacttggtctttacatctaaataaatatttccctttataat

SEQ ID NO: 6 - DNA, 39 bp, Artificial Sequence
  primer
gacatggttctacatttttgtattgtgaggagtttataa

SEQ ID NO: 7 - DNA, 37 bp, Artificial Sequence
  primer
gacatggttctacagattgaggtagttttgaagataa

SEQ ID NO: 8 - DNA, 39 bp, Artificial Sequence
  primer
cagagacttggtcttccttaaaacataaccattatttct

SEQ ID NO: 9 - DNA, 35 bp, Artificial Sequence
  primer
  misc_feature (17)..(17): n is a, c, g, or t
gacatggttctacatcnaatcacaaatctccaatc

SEQ ID NO: 10 - DNA, 37 bp, Artificial Sequence
  primer
  misc_feature (25)..(25): n is a, c, g, or t
cagagacttggtcttagtagatgtngttttagattag

SEQ ID NO: 11 - DNA, 35 bp, Artificial Sequence
  primer
  misc_feature (15)..(15): n is a, c, g, or t
gacatggttctacanggtttgtgtatgtgaaagtg

SEQ ID NO: 12 - DNA, 33 bp, Artificial Sequence
  primer
cagagacttggtctccacctcaaatcattctcc

SEQ ID NO: 13 - DNA, 37 bp, Artificial Sequence
  primer
cagagacttggtcttaaacatctcctaaatctctaca

SEQ ID NO: 14 - DNA, 39 bp, Artificial Sequence
  primer
gacatggttctacagtattgaatattaaagttgaatgtg

SEQ ID NO: 15 - DNA, 35 bp, Artificial Sequence
  primer
  misc_feature (18)..(18): n is a, c, g, or t
gacatggttctacaaacntactttactatctcacc

SEQ ID NO: 16 - DNA, 38 bp, Artificial Sequence
  primer
  misc_feature (20)..(20): n is a, c, g, or t
cagagacttggtctgttgtnggtttaaattttaatagg

SEQ ID NO: 17 - DNA, 35 bp, Artificial Sequence
  primer
gacatggttctacatatgtttttgtgggtggagtt

SEQ ID NO: 18 - DNA, 32 bp, Artificial Sequence
  primer
  misc_feature (17)..(17): n is a, c, g, or t
cagagacttggtctccnccaccatcttaacca

SEQ ID NO: 19 - DNA, 37 bp, Artificial Sequence
  primer
gacatggttctacaatagttgtattagtgtttgtgtg

SEQ ID NO: 20 - DNA, 30 bp, Artificial Sequence
  primer
cagagacttggtctcccttcctaccccctc

SEQ ID NO: 21 - DNA, 36 bp, Artificial Sequence
  primer
  misc_feature (16)..(16): n is a, c, g, or t
gacatggttctacacnaccaaataaaacaaaaaccc

SEQ ID NO: 22 - DNA, 37 bp, Artificial Sequence
  primer
  misc_feature (24)..(24): n is a, c, g, or t
cagagacttggtctaaggagatangttgtttttgaag

SEQ ID NO: 23 - DNA, 32 bp, Artificial Sequence
  primer
cagagacttggtctctctaacccctacctccc

SEQ ID NO: 24 - DNA, 39 bp, Artificial Sequence
  primer
  misc_feature (17)..(17): n is a, c, g, or t
gacatggttctacagtnggtttataatttgatatgttaa

SEQ ID NO: 25 - DNA, 37 bp, Artificial Sequence
  primer
gacatggttctacaaattccaactcaaatcttcttct

SEQ ID NO: 26 - DNA, 37 bp, Artificial Sequence
  primer
  misc_feature (19)..(19): n is a, c, g, or t
cagagacttggtctaaaangtgtaaatgagagagaaa

SEQ ID NO: 27 - DNA, 39 bp, Artificial Sequence
  primer
cagagacttggtcttattgttttaagtgtgtaatttgtg

SEQ ID NO: 28 - DNA, 39 bp, Artificial Sequence
  primer
  misc_feature (19)..(19): n is a, c, g, or t
gacatggttctacatatcntcaacaataatactacaatt

SEQ ID NO: 29 - DNA, 37 bp, Artificial Sequence
  primer
  misc_feature (22)..(22): n is a, c, g, or t
gacatggttctacatttacccnattttataaataccc

SEQ ID NO: 30 - DNA, 40 bp, Artificial Sequence
  primer
cagagacttggtctgattagattgttagatatttagtatg

SEQ ID NO: 31 - DNA, 38 bp, Artificial Sequence
  primer
  misc_feature (16)..(16): n is a, c, g, or t
gacatggttctacatngttagatttggagttatttatg

SEQ ID NO: 32 - DNA, 33 bp, Artificial Sequence
  primer
cagagacttggtcttaaacccaaacctcctccc

SEQ ID NO: 33 - DNA, 47 bp, Artificial Sequence
  primer
cagagacttggtctgttttttttatatgttatgaaattttagatatg

SEQ ID NO: 34 - DNA, 35 bp, Artificial Sequence
  primer
gacatggttctacacccctaacatctatatctaca

SEQ ID NO: 35 - DNA, 39 bp, Artificial Sequence
  primer
gacatggttctacattattagatagtggtaaataaaggt

SEQ ID NO: 36 - DNA, 38 bp, Artificial Sequence
  primer
cagagacttggtctcaaattaatcaaactatcaacacc

SEQ ID NO: 37 - DNA, 35 bp, Artificial Sequence
  primer
cagagacttggtctgtgtttggaagaatagagagg

SEQ ID NO: 38 - DNA, 39 bp, Artificial Sequence
  primer
gacatggttctacactatataaattcccttcatatcaat

SEQ ID NO: 39 - DNA, 36 bp, Artificial Sequence
  primer
gacatggttctacagagtagagttgaggattaatag

SEQ ID NO: 40 - DNA, 35 bp, Artificial Sequence
  primer
cagagacttggtctctcctacactcatcaaatcaa

SEQ ID NO: 41 - DNA, 37 bp, Artificial Sequence
  primer
cagagacttggtctaaaagttaaagtagatagggagt

SEQ ID NO: 42 - DNA, 35 bp, Artificial Sequence
  primer
gacatggttctacacctttactctttaacttccca

SEQ ID NO: 43 - DNA, 37 bp, Artificial Sequence
  primer
  misc_feature (22)..(22): n is a, c, g, or t
cagagacttggtctcaaaaccnataacattctacatc

SEQ ID NO: 44 - DNA, 39 bp, Artificial Sequence
  primer
  misc_feature (16)..(16): n is a, c, g, or t
gacatggttctacaangtagatttgtaaagtgaataaaa

SEQ ID NO: 45 - DNA, 38 bp, Artificial Sequence
  primer
cagagacttggtctttatattttatttcattcccaccc

SEQ ID NO: 46 - DNA, 35 bp, Artificial Sequence
  primer
  misc_feature (23)..(23): n is a, c, g, or t
gacatggttctacaataggtatnggttgaagtgaa

SEQ ID NO: 47 - DNA, 37 bp, Artificial Sequence
  primer
cagagacttggtctggttaaatatgtgtttttgtgtg

SEQ ID NO: 48 - DNA, 37 bp, Artificial Sequence
  primer
  misc_feature (24)..(24): n is a, c, g, or t
gacatggttctacaatttaacccnaaactactcaatt

SEQ ID NO: 49 - DNA, 38 bp, Artificial Sequence
  primer
cagagacttggtctatttcatctacaataaccacatac

SEQ ID NO: 50 - DNA, 39 bp, Artificial Sequence
  primer
gacatggttctacatttaaaatagaggtgtgtttgaaaa

SEQ ID NO: 51 - DNA, 37 bp, Artificial Sequence
  primer
cagagacttggtctttaaatgaagttatggttgtgtg

SEQ ID NO: 52 - DNA, 39 bp, Artificial Sequence
  primer
gacatggttctacaaaacaattctaacacctatctatat

SEQ ID NO: 53 - DNA, 146 bp, Danio rerio
tgagtgtttgtttggtcaagcatcggctctctctctccctcgtgcacgcgcagatatctgcacagctgtgacccacatagcggacaagacaggtgctgctgctcggccgggcaggtggtcatcctgtgggtggaggaactgtcctg

SEQ ID NO: 54 - DNA, 143 bp, Danio rerio
agccccggagaaccactacaggttactttccaggccttcttcaacaaatgagctgttgccggcggctggtttccctgtgagccctctgaataattatgtgtaccgccggctctagacaccttgaggatttcagctgtcttgtt

SEQ ID NO: 55 - DNA, 135 bp, Danio rerio
ttacatctgaataggtgtttccctttgtgatgtcgccgcttttgttaagtggggaaggcgcgaagccggagacaatggcgttcgtgtttggtgcggaaaagcggctctgtttatgaactcctcacaatgcagggg

SEQ ID NO: 56 - DNA, 127 bp, Danio rerio
gattgaggcagttctgaagacaaaagggggtccaacacggtactcataaggtgtacctaataaagtggccggtgagtgtaaatggggaaaaaacgaagcgctagaaacaatggttatgctttaagga

SEQ ID NO: 57 - DNA, 133 bp, Danio rerio
tcggatcacaaatctccaatcatttctgaaagtcgagaccgcattaatttattcacacgaaacacacacttgttttccacacacatacagcatttaatgccgggaacacgctgatctaaagcggcatctgctg

SEQ ID NO: 58 - DNA, 149 bp, Danio rerio
cggtctgtgtatgtgaaagtgcgaggcactccggcttcaaatagcagggacagggagacggacggatttattaatggccaggaaccagggtgcaggggcgggggggatccgagatccggagaggaagactggagaatgatttgaggtgg

SEQ ID NO: 59 - DNA, 136 bp, Danio rerio
tgaacatctcctggatctctgcatctctgcttgctgtctgctttgagagtaaatatatgatataaaggtgctgtagaggagccgtgccggcctgtagagcgagcgccggtgcacattcagctttgatattcagtgc

SEQ ID NO: 60 - DNA, 130 bp, Danio rerio
agcgtactttactgtctcaccaccgctgccgctccggcgtgagggcacactcccacacacatgcgcgtgtatgttaagtcgctcttgatgtggtcatatttatgttcctgttaaagtttgagccggcagc

SEQ ID NO: 61 - DNA, 134 bp, Danio rerio
catgtccctgtgggtggagttcatcaccgcctccggttatctatcggcacgaaagattcgctcccgctttcagacgctggttgcccaggccgtggataaatgcagctaccgggacgtggttaagatggtggcgg

SEQ ID NO: 62 - DNA, 141 bp, Danio rerio
atagctgtaccagtgtttgtgtgtgtgtaccggaggaaggttttgtgctggtatgccgttgttgggaccaagacccggcacagatgggtcctgctgcagtggagggatctgagcggggttaatctgagggggcaggaaggg

SEQ ID NO: 63 - DNA, 123 bp, Danio rerio
cggccaggtggagcagagaccctgccgcctccttcatctcctcgtcctcctcctcttcctcctcctcttctttgaccaggatgttttcggcgcgcctcttcttcaggggcagcgtgtctcctt

SEQ ID NO: 64 - DNA, 147 bp, Danio rerio
ctctgacccctgcctccccaggagccttggctctgtcggagacgattcaaatcacagggactgtggctatcaatcaaacacggggaccggagctcaaccgaagaatatgtcaagaagccattttaacatgtcaggttgtaggccggc

SEQ ID NO: 65 - DNA, 149 bp, Danio rerio
aattccagctcaaatcttcttctaaacgagtgataacaaccctaatccagtttggtccggaacctgcagacaacacggaaactcatctggttaagcctggtattttatctctgctaaactggatgctttctctctcatttacacgtttt

SEQ ID NO: 66 - DNA, 129 bp, Danio rerio
tattgcttcaagtgtgcaacttgtgcgcggttccaaacaggaagtggcgcgctggcaaccgggaagatatacatcactgcacagcgctgataagtaaaaactataattgcagtattattgctgacgata

SEQ ID NO: 67 - DNA, 149 bp, Danio rerio
tttacccggttttataaatgcccaacaatgcaaagtttgcgaagcaagaacactctgtcgtgctgctgataggccaaactctgttccaacgcccggggagaactttaaataaacaattgtcttcatactaaatgtctgacaatctagtc

SEQ ID NO: 68 - DNA, 122 bp, Danio rerio
ccgtcagatttggagccacctatggaggcaaccgtctgttctctggagcccggagtggtggaggcgccagctcggctctgtcccgttcactcggcctggcaaggggaggaggtttgggttta

SEQ ID NO: 69 - DNA, 148 bp, Danio rerio
gctcttcctacatgctatgaaatttcagacatgtgcgggcattgaaaggagtcaaaggcaatacccagaacaaatgtgttgatagagatcccggatattgtggtgccaacaacaaaacggagggcaatgtagacatagatgttagggg

SEQ ID NO: 70 - DNA, 129 bp, Danio rerio
ttatcagacagtggtaaataaaggtctggcccgggttaccgcaggctgtcagcaggcccgtcccggaggggaaataaaactcttattaacatgcttctgctcattggtgctgacagcttgatcaatctg

SEQ ID NO: 71 - DNA, 152 bp, Danio rerio
gtgtttggaagaatagagaggcctaggtctgggttagaggagattacactgcaggcacgtcgggtgaagactggctggagaaactgcacaccggctcactgtcaccatattgtccttaaagcaatagattgacatgaagggaatttacacag

SEQ ID NO: 72 - DNA, 153 bp, Danio rerio
gagcagagctgaggattaacagcagtctctgacctacagctgcgtttctgagcagcaccgcggagtccagagccatgagatgatggagatcctgacctgaagtgagtctgagtgtgaatgagcggatccggattgatctgatgagtgcaggag

SEQ ID NO: 73 - DNA, 146 bp, Danio rerio
aaaagtcaaagcagacagggagtgggtttttatgaacatgcatttctgagcgccgatgaaattttggtcctggacagatggatgaatatctgccgggagatggcgaacatgaagagagtcagaaatgggaagccaaagagcaaagg

SEQ ID NO: 74 - DNA, 137 bp, Danio rerio
cagggccggtgacattctgcatcccaggcgctcgctgttctttaaacacactccgatcagtggagcaaaactgtgtgagccggcttccgcgaattaaccgcacatgccaatgttttattcactttgcaagtctgcgt

SEQ ID NO: 75 - DNA, 150 bp, Danio rerio
ttatgttttatttcattcccaccccagcggagagcagcggtggtgagaaaagccctccgggttctgcagcttccaagagagcacgcaccgcttacaccagcgcacagctggtggagctcgagaaggagtttcacttcaaccgatacctgt

SEQ ID NO: 76 - DNA, 130 bp, Danio rerio
ggctaaacatgtgtttttgtgtggcagacgactgataaagaggctccgggattggtccgcatgcacactctggcctacttgagcggctttccagcatcgctgaaggaaactgagcagctccgggtcaaac

SEQ ID NO: 77 - DNA, 139 bp, Danio rerio
atttcatctgcagtgaccacatacacacacacactttgcatccacggcctacctgggaatccgcctggtagagagataattaccggatcacaatccccatctcctttttttcgattttcagacacacctctgttttgaa

SEQ ID NO: 78 - DNA, 149 bp, Danio rerio
ccaaatgaagccatggctgtgtgattgaggtttatagtgggaggtcagcagtctgggctcaggagtcctccgggcaccataaatcaccacaggccaataaacacagacagcagaatttctcagtatatagacaggtgtcagaactgctc

SEQ ID NO: 79 - DNA, 24 bp, Artificial Sequence
  primer
atatatataaacggatggtttcgg

SEQ ID NO: 80 - DNA, 26 bp, Artificial Sequence
  primer
ttatataaaactaaacgaacctaacg

SEQ ID NO: 81 - DNA, 24 bp, Artificial Sequence
  primer
ggataagataggtgttgttgttcg

SEQ ID NO: 82 - DNA, 24 bp, Artificial Sequence
  primer
gtataacctctttctatcatcccg

SEQ ID NO: 83 - DNA, 28 bp, Artificial Sequence
  primer
ttgtgagttaataaagaaaagaatagac

SEQ ID NO: 84 - DNA, 26 bp, Artificial Sequence
  primer
aaatcctcaaaatatctaaaaccgac

SEQ ID NO: 85 - DNA, 23 bp, Artificial Sequence
  primer
gagaagaaatgaagatgattacg

SEQ ID NO: 86 - DNA, 26 bp, Artificial Sequence
  primer
acctataactacgtaaaaacaacgca

SEQ ID NO: 87 - DNA, 25 bp, Artificial Sequence
  primer
tgagttattatggtaagaagagtgc

SEQ ID NO: 88 - DNA, 24 bp, Artificial Sequence
  primer
tatattacaaaaactaatttcgca

SEQ ID NO: 89 - DNA, 23 bp, Artificial Sequence
  primer
gataaaagggggtttaatacggt

SEQ ID NO: 90 - DNA, 26 bp, Artificial Sequence
  primer
ataaaatacctaaaacaaattaatcg

SEQ ID NO: 91 - DNA, 27 bp, Artificial Sequence
  primer
gcgaatatataaaagtagaagaaacgc

SEQ ID NO: 92 - DNA, 21 bp, Artificial Sequence
  primer
taccgctttaaatcaacgtat

SEQ ID NO: 93 - DNA, 22 bp, Artificial Sequence
  primer
tagatagatgtaacgttgcgag

SEQ ID NO: 94 - DNA, 29 bp, Artificial Sequence
  primer
cttaatctcacaatataaaacgataaacg

SEQ ID NO: 95 - DNA, 23 bp, Artificial Sequence
  primer
aggtgttgtagaggagtcgtgtc

SEQ ID NO: 96 - DNA, 25 bp, Artificial Sequence
  primer
taattctctatactctaaaacccga

SEQ ID NO: 97 - DNA, 22 bp, Artificial Sequence
  primer
agatttgtaaaagtgttggtgc

SEQ ID NO: 98 - DNA, 28 bp, Artificial Sequence
  primer
gcgcatatatataaaaatataccctcac

SEQ ID NO: 99 - DNA, 21 bp, Artificial Sequence
  primer
gcggtgtttaagtttagcgac

SEQ ID NO: 100 - DNA, 21 bp, Artificial Sequence
  primer
gaatacgcaatttcacttcgc

SEQ ID NO: 101 - DNA, 23 bp, Artificial Sequence
  primer
gggttttaatgaggaagacgatt

SEQ ID NO: 102 - DNA, 21 bp, Artificial Sequence
  primer
caaaacccatctataccgaat

SEQ ID NO: 103 - DNA, 25 bp, Artificial Sequence
  primer
ttgaatagaagtatttagatttgcg

SEQ ID NO: 104 - DNA, 21 bp, Artificial Sequence
  primer
ctctactccacctaaccgacg

SEQ ID NO: 105 - DNA, 22 bp, Artificial Sequence
  primer
tggttattaattaaatacgggg

SEQ ID NO: 106 - DNA, 24 bp, Artificial Sequence
  primer
aaatcttacgaaacgtatctcgct

SEQ ID NO: 107 - DNA, 27 bp, Artificial Sequence
  primer
agtaggatgattaaagaatgttagcga

SEQ ID NO: 108 - DNA, 25 bp, Artificial Sequence
  primer
aacttaaccaaataaatttccgtat

SEQ ID NO: 109 - DNA, 29 bp, Artificial Sequence
  primer
agttgtatatataataaaataaagacgtt

SEQ ID NO: 110 - DNA, 30 bp, Artificial Sequence
  primer
cacaataatataaaaacaataattataccg

SEQ ID NO: 111 - DNA, 24 bp, Artificial Sequence
  primer
atagaagcggagttattaagcgaa

SEQ ID NO: 112 - DNA, 25 bp, Artificial Sequence
  primer
aaacttataaaaccaatactcgaaa

SEQ ID NO: 113 - DNA, 23 bp, Artificial Sequence
  primer
atgatagagttaagtttgcggat

SEQ ID NO: 114 - DNA, 21 bp, Artificial Sequence
  primer
cgaataaacgaaacaaaaccg

SEQ ID NO: 115 - DNA, 24 bp, Artificial Sequence
  primer
tatgaaattttagatatgtgcggg

SEQ ID NO: 116 - DNA, 25 bp, Artificial Sequence
  primer
atctatatctacattaccctccgtt

SEQ ID NO: 117 - DNA, 20 bp, Artificial Sequence
  primer
gattgatcggtaaatcgaga

SEQ ID NO: 118 - DNA, 23 bp, Artificial Sequence
  primer
tccaaacaaacactcctaacgat

SEQ ID NO: 119 - DNA, 25 bp, Artificial Sequence
  primer
tagtggtaaataaaggtttggttcg

SEQ ID NO: 120 - DNA, 20 bp, Artificial Sequence
  primer
tttcaacctccatcaaaacg

SEQ ID NO: 121 - DNA, 27 bp, Artificial Sequence
  primer
agaggagattatattgtaggtacgtcg

SEQ ID NO: 122 - DNA, 27 bp, Artificial Sequence
  primer
tatataaacaatctaaactacacgacc

SEQ ID NO: 123 - DNA, 20 bp, Artificial Sequence
  primer
agtttgagtgtgaatgagcg

SEQ ID NO: 124 - DNA, 20 bp, Artificial Sequence
  primer
aactctcgaacgaaaccgtc

SEQ ID NO: 125 - DNA, 25 bp, Artificial Sequence
  primer
ggtaatggtttaaatatgtggttcg

SEQ ID NO: 126 - DNA, 24 bp, Artificial Sequence
  primer
acgttaaattaaatcaacacgtta

SEQ ID NO: 127 - DNA, 20 bp, Artificial Sequence
  primer
tgcgtatcgtagggatgttc

SEQ ID NO: 128 - DNA, 24 bp, Artificial Sequence
  primer
atatacgattaattcgcgaaaacc

SEQ ID NO: 129 - DNA, 21 bp, Artificial Sequence
  primer
gttttattagtgggaacgatg

SEQ ID NO: 130 - DNA, 24 bp, Artificial Sequence
  primer
cgattaaaataaaactccttctcg

SEQ ID NO: 131 - DNA, 21 bp, Artificial Sequence
  primer
gttttattagtgggaacgatg

SEQ ID NO: 132 - DNA, 24 bp, Artificial Sequence
  primer
cgattaaaataaaactccttctcg

SEQ ID NO: 133 - DNA, 20 bp, Artificial Sequence
  primer
aatcgacgagtgagacggtt

SEQ ID NO: 134 - DNA, 27 bp, Artificial Sequence
  primer
aaacaaaaatatatctaaaaatcgaaa

SEQ ID NO: 135 - DNA, 23 bp, Artificial Sequence
  primer
aaaaggttgttgaggttgatacg

SEQ ID NO: 136 - DNA, 24 bp, Artificial Sequence
  primer
ttaaccttaaaccttataccgaaa

SEQ ID NO: 137 - DNA, 22 bp, Artificial Sequence
  primer
atataaatggatggttttggga

SEQ ID NO: 138 - DNA, 29 bp, Artificial Sequence
  primer
ttatataaaactaaacaaacctaacattc

SEQ ID NO: 139 - DNA, 23 bp, Artificial Sequence
  primer
ataagataggtgttgttgtttgg

SEQ ID NO: 140 - DNA, 26 bp, Artificial Sequence
  primer
tcatataacctctttctatcatccca

SEQ ID NO: 141 - DNA, 27 bp, Artificial Sequence
  primer
tgtgagttaataaagaaaagaatagat

SEQ ID NO: 142 - DNA, 27 bp, Artificial Sequence
  primer
aaaatcctcaaaatatctaaaaccaac

SEQ ID NO: 143 - DNA, 23 bp, Artificial Sequence
  primer
gagaagaaatgaagatgattatg

SEQ ID NO: 144 - DNA, 30 bp, Artificial Sequence
  primer
ctacctataactacataaaaacaacacaac

SEQ ID NO: 145 - DNA, 25 bp, Artificial Sequence
  primer
gagttattatggtaagaagagtgtg

SEQ ID NO: 146 - DNA, 25 bp, Artificial Sequence
  primer
tatattacaaaaactaatttcacaa

SEQ ID NO: 147 - DNA, 26 bp, Artificial Sequence
  primer
aagataaaagggggtttaatatggta

SEQ ID NO: 148 - DNA, 26 bp, Artificial Sequence
  primer
aaatacctaaaacaaattaatcattc

SEQ ID NO: 149 - DNA, 30 bp, Artificial Sequence
  primer
gtgaatatataaaagtagaagaaatgtgta

SEQ ID NO: 150 - DNA, 27 bp, Artificial Sequence
  primer
aataccactttaaatcaacatattccc

SEQ ID NO: 151 - DNA, 24 bp, Artificial Sequence
  primer
ggtagatagatgtaatgttgtgag

SEQ ID NO: 152 - DNA, 25 bp, Artificial Sequence
  primer
tcacaatataaaacaataaacaaaa

SEQ ID NO: 153 - DNA, 20 bp, Artificial Sequence
  primer
gtagaggagttgtgttggtt

SEQ ID NO: 154 - DNA, 25 bp, Artificial Sequence
  primer
aattctctatactctaaaacccaat

SEQ ID NO: 155 - DNA, 20 bp, Artificial Sequence
  primer
tgtaaaagtgttggtgtgtg

SEQ ID NO: 156 - DNA, 30 bp, Artificial Sequence
  primer
acacacatatatataaaaatataccctcac

SEQ ID NO: 157 - DNA, 23 bp, Artificial Sequence
  primer
gtggtgtttaagtttagtgatgg

SEQ ID NO: 158 - DNA, 25 bp, Artificial Sequence
  primer
ccaaatacacaatttcacttcactc

SEQ ID NO: 159 - DNA, 23 bp, Artificial Sequence
  primer
ggggttttaatgaggaagatgat

SEQ ID NO: 160 - DNA, 23 bp, Artificial Sequence
  primer
aacaaaacccatctataccaaat

SEQ ID NO: 161 - DNA, 24 bp, Artificial Sequence
  primer
tgaatagaagtatttagatttgtg

SEQ ID NO: 162 - DNA, 22 bp, Artificial Sequence
  primer
ctctactccacctaaccaacat

SEQ ID NO: 163 - DNA, 24 bp, Artificial Sequence
  primer
tgtggttattaattaaatatgggg

SEQ ID NO: 164 - DNA, 30 bp, Artificial Sequence
  primer
ctttcaaatcttacaaaacatatctcacta

SEQ ID NO: 165 - DNA, 30 bp, Artificial Sequence
  primer
gaagtaggatgattaaagaatgttagtgag

SEQ ID NO: 166 - DNA, 24 bp, Artificial Sequence
  primer
aaacttaaccaaataaatttccat

SEQ ID NO: 167 - DNA, 22 bp, Artificial Sequence
  primer
ttaaataggaagtggtgtgttg

SEQ ID NO: 168 - DNA, 25 bp, Artificial Sequence
  primer
aaactattaatatacaaatccacaa

SEQ ID NO: 169 - DNA, 27 bp, Artificial Sequence
  primer
tggatagaagtggagttattaagtgaa

SEQ ID NO: 170 - DNA, 26 bp, Artificial Sequence
  primer
aaaacttataaaaccaatactcaaaa

SEQ ID NO: 171 - DNA, 26 bp, Artificial Sequence
  primer
aaaatgatagagttaagtttgtggat

SEQ ID NO: 172 - DNA, 25 bp, Artificial Sequence
  primer
accaaataaacaaaacaaaaccaaa

SEQ ID NO: 173 - DNA, 24 bp, Artificial Sequence
  primer
tatgaaattttagatatgtgtggg

SEQ ID NO: 174 - DNA, 28 bp, Artificial Sequence
  primer
aacatctatatctacattaccctccatt

SEQ ID NO: 175 - DNA, 27 bp, Artificial Sequence
  primer
gaaatgtagattgattggtaaattgag

SEQ ID NO: 176 - DNA, 22 bp, Artificial Sequence
  primer
caaacaaacactcctaacaatc

SEQ ID NO: 177 - DNA, 20 bp, Artificial Sequence
  primer
aataaaggtttggtttgggt

SEQ ID NO: 178 - DNA, 21 bp, Artificial Sequence
  primer
tcaacctccatcaaaacatcc

SEQ ID NO: 179 - DNA, 26 bp, Artificial Sequence
  primer
ggagattatattgtaggtatgttggg

SEQ ID NO: 180 - DNA, 28 bp, Artificial Sequence
  primer
ctatataaacaatctaaactacacaacc

SEQ ID NO: 181 - DNA, 22 bp, Artificial Sequence
  primer
gtttgagtgtgaatgagtggat

SEQ ID NO: 182 - DNA, 27 bp, Artificial Sequence
  primer
aaactaaaactctcaaacaaaaccatc

SEQ ID NO: 183 - DNA, 23 bp, Artificial Sequence
  primer
aatggtttaaatatgtggtttgg

SEQ ID NO: 184 - DNA, 25 bp, Artificial Sequence
  primer
cacattaaattaaatcaacacatta

SEQ ID NO: 185 - DNA, 23 bp, Artificial Sequence
  primer
gtgtattgtagggatgtttgtag

SEQ ID NO: 186 - DNA, 30 bp, Artificial Sequence
  primer
aaaacattaacatatacaattaattcacaa

SEQ ID NO: 187 - DNA, 22 bp, Artificial Sequence
  primer
attttagtggagagtagtggtg

SEQ ID NO: 188 - DNA, 27 bp, Artificial Sequence
  primer
caattaaaataaaactccttctcaaac

SEQ ID NO: 189 - DNA, 21 bp, Artificial Sequence
  primer
gtgtttttgtgtggtagatga

SEQ ID NO: 190 - DNA, 27 bp, Artificial Sequence
  primer
aaaactactcaatttccttcaacaata

SEQ ID NO: 191 - DNA, 26 bp, Artificial Sequence
  primer
gttatgaattgatgagtgagatggtt

SEQ ID NO: 192 - DNA, 28 bp, Artificial Sequence
  primer
aaaacaaaaatatatctaaaaatcaaaa

SEQ ID NO: 193 - DNA, 26 bp, Artificial Sequence
  primer
taaaaggttgttgaggttgatatgta

SEQ ID NO: 194 - DNA, 25 bp, Artificial Sequence
  primer
cttaaccttaaaccttataccaaaa

SEQ ID NO: 195 - DNA, 300 bp, Carcharodon carcharias
acagataccctggagtgagttacagactggaatctaatcgaggtgtttgggttggtttatatatagaataacagataaccgggagtgagttacagactggaatctaatcgagggtgtaggggtggtttatatatagaataacagatacccgggagtgagttacagactggaatctaatcgaggggttcggggtggtttatatatagaataacagatacccgccagtgagtcacaggctggaatctaatcgaggtgttcggggtggtttatatatagagtaacagataccctggagtgagt

SEQ ID NO: 196 - DNA, 300 bp, Carcharodon carcharias
acagatacccgggagtgagttacagactggaatctaatcgaggggttcgggggggtttatatatagaataacagatacccgggagtgagttacagactggaatctaatcgaggggttcggggtggtttatatatagaataacagatacccgggagtgagttacaggctggaatctaatcgaggggttcagggtgggtttacatatagaataacagatacccgaaagtgagttacagactggaatcttatcaaggggttcgggggggtttatatatgtaataacagatatccgggagtgag

SEQ ID NO: 197 - DNA, 300 bp, Carcharodon carcharias
actcactcccgggtacctgttattctatatataaacccccccccgaacccctcgattacattccagtctgtaacttcactcccgggtatctgttattctatatgtaaaccacccctaaccctcgattagtttccagtctgtaactcactcgcgggtatctgttattctatatataaaccaccccgaacccctcgattagattccagtctgtaactcactcctgggtatctgttattctgtatataaaccgccccgaacccctcgattagattccagtctgtaactcactcccgggtatct

SEQ ID NO: 198 - DNA, 300 bp, Carcharodon carcharias
aaataggtgggaaaagaaaaatctatataaattattgggaaaaacaaggagggggaagaaacaaaaagtgggtggggacgaaggagagagttcaagatctaaaattgttgaactcagtattcagtccggaaggctgtaaagtgcttagtcggaagatgagatgctgttcctccagtttgtgttgagcttcactggaacaatgcagcaagccaagggtagacatgtgggcatgggagcagggtggagtgttgaaatggcaagcgagagggaggtctgggtaatgcttacggacagaccgaa

SEQ ID NO: 199 - DNA, 300 bp, Carcharodon carcharias
accttatccacctgcgttgccactttcagtgacctgtggacctgtacacccagatccctctgcctgtcgatgcacttaagggttctgccatttactgtataattcctgcctgtattagaccttccaaaatgcattacctcgcatttgtccggattaaactccatctgccatttctgcgcccaagtctccaaccaatctatatcccgttgtatcctttgacaatcctcttcaccatctgcaactcctccaaccttagtgttgtctgcaaacttactaattaatccagttacattttcctcc

SEQ ID NO: 200 - DNA, 300 bp, Carcharodon carcharias
tccccatttacacacaccgggcagtctgttccctatcggagggggattaaccctctcacccacctccccccatttacacacaccgggcagtctgttccctatcggagggggattaaccctctcacccacctctccccatttacacacaccgggcagtctgttcccctatcggagggggattaaccctctcacccacctctccccatttacacacaccgggcagtctgttccctatcggagggggattaaccctctcacccacctgtccacatttacacacatcgggcaaccttttcccta

SEQ ID NO: 201 - DNA, 300 bp, Carcharodon carcharias
ttttagtaaaacacaaatttaataatgggggcaacgtggtggtttggtgctgctgcctcacagctctatggacctgggttcgatcttggcctcaggtgcctgtctgcttggagtttgtacgtttcccccatgtctgcgtgggtctccctcgggtgctttggtttcttcccactgcccaaagacatgctggttaggtgtattgactaaggtaaattgtcccccagtgtgtgtatgtctacatgtgagagtgtgccctgtgatggattggtttcccatccttgatgtgtcctgcttaatgcc

SEQ ID NO: 202 - DNA, 300 bp, Carcharodon carcharias
aaatgctattggtttctgtgggggtagtacggttgcgcagtagctagcactgctgccttgcagttgcaaggacccaggcttgattctgaccttggatgtctgtctgcgtggagtttgcacgttccccgtgtgtccacgtgggtttctgccgggtgcttcagtttcctcccaccatccaaagacgtgctggttagatggattggctacgcctatattgccccttagagtgtgcttatgtgcatctgagtgtgtgccctgtgataggctggcatcccatcctgggtgtaatggccattgcct

SEQ ID NO: 203 - DNA, 300 bp, Carcharodon carcharias
ataaatagaataacagatacccaggagagacttacagactggaatataattaagggtgtcagggtggtttatatagagaattagagatagctacagagtggaatctaatcgagtgtttcgcagtgtttataaatggaataacagatacacgggagtgagttacagactggaatctgatcgaggggttcggggtggtttatatatagaataacagatatcctggcgtgagttacaaacaggaattttatcgaggttttcaggggctttatatataaaataacagatacccgggagtgagtt

SEQ ID NO: 204 - DNA, 300 bp, Carcharodon carcharias
ccaccattggaacatccttcctgcatctaccctgtctagtcctgttagaattttataggtttctatgagctctctcctctttcttctgaactccagtgaatataatcctaaccaactcaatctcttctcatatgtcagtcccaccatcccggaatcagtctggtaaaccttcgctgcactccctctatagcaagaacatccttcctcagataaggagaccaaaactgcacataatattccaggtgtggcctcaccaaggccctgtataattgcagcaagacatccctgctcctgtactcg

SEQ ID NO: 205 - DNA, 300 bp, Carcharodon carcharias
gcacactaaggggaaatatactgtagctaatccaactaaccagcacgtctttggtcagtggaaggaaactggagcacctgacagaaacccaagcaaacacaagccgaacgtgtgatctccacacagacatccgaggtcagaattgaacccgggtccctggagctgtgaggcagcagcactaaccactgtgccaccatgccgcccaaaatttataaacactaataaatagatttactaaaattagaatgttaaagttaattttattgcagagttgatattctccttaatgaattgttatat

SEQ ID NO: 206 - DNA, 300 bp, Carcharodon carcharias
acagggatggggagcaggaacccgggctgattcacaccccctccctaacccaggggtcagtggacagggatggggagcaggaacccgggctgattcacacccccctccctaacccaggggtcagtggacagggatggggagcaggaacccgggctgattcaccccccctccctaacccaggggtcagtggacagggactgggagcaggaacccgggctgattcacaccctccctaacccaggggtcagtggacagggatggggagcaggaacccgggctgattcacacccccctccctaa

SEQ ID NO: 207 - DNA, 300 bp, Carcharodon carcharias
cacccggcccggacacggaaaggattgacagattgatagctctttctcgattctgtgggtggtggtgcatggccgttcttagttggtggagcgatttgtctggttaattccgataacgaacgagactcctccatgctaaatagttacgcgacccccgagcggtccgcgtccaacttcttagagggacaagtggcgtacagccacacgagattgagcaataacaggtctgtgatgcccttagatgtccggggctgcacgcgcgctacactgaatggatcagcgtgtgtctacccttcgccg

SEQ ID NO: 208 - DNA, 300 bp, Carcharodon carcharias
ttttatggcgtgcctgggcacgccggggccgcgccttcggatggggcttcccggcagatgtcggcgagcgtggggtgcggtccgtgcgcggcttcctcgcgggaggatccgaccgaaagctctgtacaactcttagcggtggatcactcggctcgtgcgtcgatgaagaacgcagctagctgcgagaattaatgtgaattgcaggacacattgatcatcgacactttgaacgcactttgcggccccgggttcctcccggggctacgcctgtctgagggtcgcttgacgatcaatcgcact

SEQ ID NO: 209 - DNA, 300 bp, Carcharodon carcharias; identical to SEQ ID NO: 207

SEQ ID NO: 210 - DNA, 300 bp, Carcharodon carcharias
tccttttgtcttcttagtctcatgcactgcccctttcatgagctacaaggactaccccctccccctccctggctccagttgttgactacacccatgttttgacaccgtgctgcagtttccacacttgctccctcaccctaatctctctccggcgtgctcactctctctctttctcttctcttctctctctctctttctctctctctttcgctctctcagacattcttctgttctctctttttaagccatctgctctctcattacttactactttcttgcactccctttgtctcccttaca

SEQ ID NO: 211 - DNA, 300 bp, Carcharodon carcharias
tatttacaggggagtccctggggagtgtgtcagtatttacagggggtccccggggagtgtgtcagtatttacaggggggtccccagggagtgtgtcagtatttacaggggtgtccccgggggagtgtgtcagtatttacagggggaatccggggggagtgtgtcagtatttacaggggggggtccccggggagtgtgtcagtatttacaggggtccccggggagtgtgtcagtatttacaggggggtctccggggagtgagtcagtatttacagggggtccccagggagtgagtcagtat

SEQ ID NO: 212 - DNA, 300 bp, Carcharodon carcharias
cacggtgctccgagtgtggtctaaccgagggggatacggagaccggaactgtgcacggtgctccgagtgtggtctaaccgagggggatacggagaccggaactgtgcacggtgctccgagtgtggtctaaccgagggggatacggagaccggaactgtgcacggcgctccgagtgtgggtctaaccgagggatatggagaccggaactgtgcacggtgctccgagtgtgggctaacccagggggatacggagactgggactgtgcacggtgctccgagtgtggtctaaccgagggggata

SEQ ID NO: 213 - DNA, 300 bp, Carcharodon carcharias
cggtctcctatcctcggttagaccacactcggagcaccgtgcacagttccggtctccgtatccctcgattgaccacactcggagcaccgtgcacagttccggtctcctatccctcggttagaccacactcggagcaccgtgcacagttccggtctccctatccctcggttagacccactcggagcaccgtgcacagttccggtctccgtatcccctcggttagaccacactcggagcaccgtgcacagttcggtctccgtatccctcgattagaccacactcggagcaccgtgcacagtt

SEQ ID NO: 214 - DNA, 300 bp, Carcharodon carcharias
tgctgtagaggctgctatgggaagtgtcaatgctgctgctatctcaaggcggcatgtagatggtgtagcttaaaaaataccataacttagttattatctcagggtctggtcaattgttctcaattttaaatttctcaccctgggcagcacggtgacacagtggttagcactgctgcctcagctccagggacccgggttcaatcctgacctctggtctctgtctgtatttagagtctccatgtctgcgtgggcttctgccaggtgctccgattctcttttccaccccccaccatccaaaga

SEQ ID NO: 215 - DNA, 300 bp, Carcharodon carcharias
ttgtaggacaattttaccgtagccgatccacctaaccagtatgtctttggatggtgggaggaaaccggagcacccagtggaaactcacgcagacacggggagaatgtgcaatctccacatagacagacacctgaggtcaagatcaaacccgggtgcctggagctgtgaggcagcagcactaaccactccgccaccgtgctgcctcaagaatcaatttattgcaattatggaagaacttgtaatgtgcaatggatttaacattttgtctgaaatcaagaagcgagatggtattaatgatgg

SEQ ID NO: 216 - DNA, 300 bp, Carcharodon carcharias
gtcagtatttacagggggtccccggggagtgtgtcagtatttacagggggaccaggggagtgtgtcagtatttacagggaattacttggtagtgtgtcagtatttacagtgggtcccgggggagtgtgtcagtatttacagggggtccccggggagtgtgtcagtatttacagggggtccccggggagtgtgtcagtatttacagggggtaccgaggagtgtgtcagtatttacagggggtccccggggagtgtgtcagtatttacagggggtcccggggagtgtgtcagtatttacagg

SEQ ID NO: 217 - DNA, 300 bp, Carcharodon carcharias
tcagtatttacagtgtttccccggtgagtgtgtcagtatttacagtgtttccccggggagtgtgtcagtatttacagggggtccccgggcagtgtgtcagtatttacagtgtttccccggtgagtgtgtcagtatttacagtgtttccccggggagtgtgtcagtatttacagtgtttccccggtgagtgtgtcagtatttacagtgtttccccggggagtgtgtcagtatttacagggggtccccggggagtgtgtcagtatttacaggggagtccctggggagtgtgtcagtatttac

SEQ ID NO: 218 - DNA, 300 bp, Carcharodon carcharias
catgaggaccctacaaagaggttctgggaactggtcatagttgcgaccacactaaaaaattgtactgtacagataggtaggtagaaaggtgggcaggtaggtgggtagataggaaggtagatagataagtaggtaggcagccagataggcggatagataggtaagcaggtaggcaggcaggttgatgggtagagaggcagcgaggtagttttataggcagggtgggcaggtgggcaggtaggtgggtaggtaggtaggtaggtagataagtaggcaggttggtaggcaggcaagtaggca

SEQ ID NO: 219 - DNA, 300 bp, Carcharodon carcharias
caagatgttaactcttcttgtgatacatttccacaagtggtgccaactccacagcactttgcctaagtggccattcttcatttctgagactgacactaagagttgacaaattattgaattgtgaaaggtgatacaactgagtcctatcccgggttcactctctgtccacatatcgatactttcaagcaagattcacaaattagtgagcaggaactgggaccatgactgagatctagcttgtcaaaagtacttagaccaacttttgggcctgagtgagatcagttggttcagtgcaggcca

SEQ ID NO: 220 - DNA, 300 bp, Carcharodon carcharias
gagataattgatgaacaaatcaaagttagagatgatgaagcctctggccctttcagattgcttccggcaatttaaaagaagttgatggcaacttctgtgtggagtttgcatattctccccatgtctgcgtgggtttctgccaggtgcttcggtttcctcccaccatccaaagacgtgctggttaggtggattggctacgataaattgtctcctagtgtgtgcgtgtctgcgtgtgtatgtgtgagtatgtgccctgtgatggactgatgtcctgtcttgggtgtaccctgcctagcaccc

SEQ ID NO: 221 - DNA, 300 bp, Carcharodon carcharias
gagggcagagatggacaagatgggctcatggttcacgaattacaaaaaactcaaacacaagcatgaaataacagataccggggagtgagttacagactggaatctaatcgaaaggttcggggtggtttatatatagaattacagatacccgggaatgagttacagactggaatctgatcgaggggttcggggtggtttatatatagaataacagatacccgggagtgagttacagactggaatctaattgaagggtttaggggtggggtttaaacgtagaatagcatgataacaactgag

SEQ ID NO: 222 - DNA, 300 bp, Carcharodon carcharias
tgtgacacccggtcccagagggggaaaggacagtgtatctaaagccctgtgtacctggtcccagagggggaaaggacagtgtatctaacgcactgtgacacctggtccgagagggggaaaggacagtgtatctaacgcactgtgacacccggtcccagagggggaaaggacagtgtatcgaacgcactgtgacacccggtcccagagggggaaaggacagtgtatctaacgcaatgttacaaccggtcccagtgggggaaaggacagtgtatctaacgcactgtgacacccggtcccaga

SEQ ID NO: 223 - DNA, 300 bp, Carcharodon carcharias
tgccttttttccagcgagtagaagaagggggagccgcggtccatctccttgaggaaccggatccgcgacctcacgaacgcgccccgagacccgaccagttgcaggtcccgcagcgcgcccttcttctcatcgtacaccgaccgcagggccgggtccgcgtcgggctgaccgagacgtgcctccaggtcgagcacctccttctccaactcctcgaccctggatttgcgtcgctttgtcgaccccctcacgtactcctgacagaaaactcggacgtgagtcttgcccacgtcccaccagagc

SEQ ID NO: 224 - DNA, 300 bp, Carcharodon carcharias
cccggaatgtgtcagtatttacaggggtccccgggagtgtgtcagtatttacaggggtcccgaagtgagtcagtatttacagggtttccccgggagtgtgtcagtatttacagggtccccgggagtgtgtcagtattacagggggtccccggggggagtgtgtcagtatttacagggggtccccgggagtgtgtcagtatttacagggtgtcccgggaggtgtcagtatttacagggggtcccgggagtgtgtcagtatttacagggggtccccgggagtgtcagtatttacaggggtcc

SEQ ID NO: 225 - DNA, 40 bp, Neoceratodus forsteri
gacatggttctacatgtttattaatgaagtgttatttgtg

SEQ ID NO: 226 - DNA, 37 bp, Neoceratodus forsteri
  misc_feature (17)..(17): n is a, c, g, or t
gacatggttctacaggngtttaaaatagataatagtg

SEQ ID NO: 227 - DNA, 35 bp, Neoceratodus forsteri
cagagacttggtctcttattttatccatccccctc

SEQ ID NO: 228 - DNA, 39 bp, Neoceratodus forsteri
gacatggttctacaatggtgtttaaaatagataatagtg

SEQ ID NO: 229 - DNA, 47 bp, Neoceratodus forsteri
cagagacttggtcttcaatcttaattttttttattccttttttttcc

SEQ ID NO: 230 - DNA, 32 bp, Neoceratodus forsteri
cagagacttggtctcccattctcacaactccc

SEQ ID NO: 231 - DNA, 35 bp, Neoceratodus forsteri
  misc_feature (21)..(21): n is a, c, g, or t
gacatggttctacacattacntatccttcccttat

SEQ ID NO: 232 - DNA, 45 bp, Neoceratodus forsteri
gacatggttctacaatcaaaattctaattactaacttatctaatc

SEQ ID NO: 233 - DNA, 37 bp, Neoceratodus forsteri
  misc_feature (17)..(17): n is a, c, g, or t
gacatggttctacattngtttattttgagtatgtgtg

SEQ ID NO: 234 - DNA, 35 bp, Neoceratodus forsteri
cagagacttggtctccttatactaaaactccctcc

SEQ ID NO: 235 - DNA, 45 bp, Neoceratodus forsteri
  misc_feature (19)..(19): n is a, c, g, or t
gacatggttctacaatganggtaattgtttattataaaatgtgaa

SEQ ID NO: 236 - DNA, 33 bp, Neoceratodus forsteri
gacatggttctacaaccccacatctaattctcc

SEQ ID NO: 237 - DNA, 39 bp, Neoceratodus forsteri
gacatggttctacataaattattttttgttagtaggtgg

SEQ ID NO: 238 - DNA, 37 bp, Neoceratodus forsteri
  misc_feature (18)..(18): n is a, c, g, or t
gacatggttctacagttngatatttggttattgttag

SEQ ID NO: 239 - DNA, 35 bp, Neoceratodus forsteri
  misc_feature (19)..(19): n is a, c, g, or t
gacatggttctacatattnggtggttttaggatga

SEQ ID NO: 240 - DNA, 35 bp, Neoceratodus forsteri
  misc_feature (21)..(21): n is a, c, g, or t
cagagacttggtctcattacntatccttcccttat

SEQ ID NO: 241 - DNA, 45 bp, Neoceratodus forsteri
cagagacttggtctctctaaaatctaatttaacattttactaatc

SEQ ID NO: 242 - DNA, 40 bp, Neoceratodus forsteri
  misc_feature (18)..(18): n is a, c, g, or t
gacatggttctacagtgngaataatatttttatttttgtg

SEQ ID NO: 243 - DNA, 47 bp, Neoceratodus forsteri
cagagacttggtctttattaagtttaatagtgtttaatttgtgaatt

SEQ ID NO: 244 - DNA, 39 bp, Neoceratodus forsteri
  misc_feature (16)..(16): n is a, c, g, or t
cagagacttggtctcnaaatacattattctacttatacc

SEQ ID NO: 245 - DNA, 37 bp, Neoceratodus forsteri; identical to SEQ ID NO: 226
  misc_feature (17)..(17): n is a, c, g, or t

SEQ ID NO: 246 - DNA, 39 bp, Neoceratodus forsteri
gacatggttctacaatcatataacactcaatcatattac

SEQ ID NO: 247 - DNA, 35 bp, Neoceratodus forsteri
  misc_feature (15)..(15): n is a, c, g, or t
cagagacttggtctnggatgtgatgtattttagtg

SEQ ID NO: 248 - DNA, 37 bp, Neoceratodus forsteri
  misc_feature (20)..(20): n is a, c, g, or t
gacatggttctacaaatccnaataccaattaatttcc

SEQ ID NO: 249 - DNA, 50 bp, Neoceratodus forsteri
gacatggttctacaatttattataatttattattagtgtttaatttgtga

SEQ ID NO: 250 - DNA, 34 bp, Neoceratodus forsteri
cagagacttggtcttcattttaccaatccccctc

SEQ ID NO: 251 - DNA, 33 bp, Neoceratodus forsteri
  misc_feature (17)..(17): n is a, c, g, or t
gacatggttctacatcnaaactccactcaactc

SEQ ID NO: 252 - DNA, 35 bp, Neoceratodus forsteri
  misc_feature (19)..(19): n is a, c, g, or t
gacatggttctacaaaccntatatccttcccttat

SEQ ID NO: 253 - DNA, 32 bp, Neoceratodus forsteri
  misc_feature (16)..(16): n is a, c, g, or t
cagagacttggtctcnctctactacccccttc

SEQ ID NO: 254 - DNA, 35 bp, Neoceratodus forsteri
  misc_feature (16)..(16): n is a, c, g, or t
cagagacttggtctcntcattatatccttccctta

SEQ ID NO: 255 - DNA, 37 bp, Neoceratodus forsteri
gacatggttctacatagattggtatagattgatgtga

SEQ ID NO: 256 - DNA, 35 bp, Neoceratodus forsteri; identical to SEQ ID NO: 231
  misc_feature (21)..(21): n is a, c, g, or t

SEQ ID NO: 257 - DNA, 35 bp, Neoceratodus forsteri
  misc_feature (24)..(24): n is a, c, g, or t
gacatggttctacatatcattacntatccttccct

SEQ ID NO: 258 - DNA, 37 bp, Neoceratodus forsteri
gacatggttctacattgagtagtttttttagatgtgg

SEQ ID NO: 259 - DNA, 39 bp, Neoceratodus forsteri
  misc_feature (21)..(21): n is a, c, g, or t
gacatggttctacattttttngtatgttttggtatgttt

SEQ ID NO: 260 - DNA, 45 bp, Neoceratodus forsteri
gacatggttctacataattaaattcctctcctaaaaaaatctaca

SEQ ID NO: 261 - DNA, 34 bp, Neoceratodus forsteri
cagagacttggtctctcatctatccaacatctcc

SEQ ID NO: 262 - DNA, 45 bp, Neoceratodus forsteri
cagagacttggtctcaaaaaacctttccaaaaaaaaaatataatc

SEQ ID NO: 263 - DNA, 35 bp, Neoceratodus forsteri
cagagacttggtctcttattcatcaaacccctatc

SEQ ID NO: 264 - DNA, 47 bp, Neoceratodus forsteri
cagagacttggtctaagatataaattaaattaaagttgtaagtagtg

SEQ ID NO: 265 - DNA, 40 bp, Neoceratodus forsteri
gacatggttctacaaatggtgtttaaaatagataatagtg

SEQ ID NO: 266 - DNA, 39 bp, Neoceratodus forsteri
gacatggttctacaattgtgggattataatgtttagtat

SEQ ID NO: 267 - DNA, 45 bp, Neoceratodus forsteri
gacatggttctacaaattatttatttagtgatggtaagtaaaagt

SEQ ID NO: 268 - DNA, 52 bp, Neoceratodus forsteri
cagagacttggtctatattaatatattatttatttataattaatagaagaaa

SEQ ID NO: 269 - DNA, 37 bp, Neoceratodus forsteri
cagagacttggtctcaaatttataccaaacttcctcc

SEQ ID NO: 270 - DNA, 47 bp, Neoceratodus forsteri
gacatggttctacaaatttagttatataatgttttatgtgtttgaaa

SEQ ID NO: 271 - DNA, 40 bp, Neoceratodus forsteri
  misc_feature (24)..(24): n is a, c, g, or t
cagagacttggtctcaaaaaatcnaatattttatacttcc

SEQ ID NO: 272 - DNA, 37 bp, Neoceratodus forsteri
cagagacttggtcttttataccaaacttccttctatc

SEQ ID NO: 273 - DNA, 35 bp, Neoceratodus forsteri
cagagacttggtctctcattttactaatccccctc

SEQ ID NO: 274 - DNA, 37 bp, Neoceratodus forsteri
  misc_feature (18)..(18): n is a, c, g, or t
gacatggttctacaatgnggaataattgatattagtg

SEQ ID NO: 275 - DNA, 39 bp, Neoceratodus forsteri
cagagacttggtcttaaattattttttgttagtaggtgg

SEQ ID NO: 276 - DNA, 37 bp, Neoceratodus forsteri
  misc_feature (16)..(16): n is a, c, g, or t
gacatggttctacagngaatggttttgattattagtg

SEQ ID NO: 277 - DNA, 47 bp, Neoceratodus forsteri
cagagacttggtctaaataaaatgaataatagtgaaatattgtgaaa

SEQ ID NO: 278 - DNA, 47 bp, Neoceratodus forsteri
gacatggttctacaaatttagttatataatgtttgaattgtttgaaa

SEQ ID NO: 279 - DNA, 47 bp, Neoceratodus forsteri
  misc_feature (17)..(17): n is a, c, g, or t
gacatggttctacagtnggtttattttttattatataatttggtggt

SEQ ID NO: 280 - DNA, 37 bp, Neoceratodus forsteri
  misc_feature (21)..(21): n is a, c, g, or t
cagagacttggtctctaaccnaaaatattcaactaca

SEQ ID NO: 281 - DNA, 35 bp, Neoceratodus forsteri
  misc_feature (22)..(22): n is a, c, g, or t
cagagacttggtctcttattcntcaaacccctatc

SEQ ID NO: 282 - DNA, 34 bp, Neoceratodus forsteri
gacatggttctacaggatgaaagtgaagagtagg

SEQ ID NO: 283 - DNA, 34 bp, Neoceratodus forsteri
  misc_feature (21)..(21): n is a, c, g, or t
cagagacttggtctctattcntcaaacccctatc

SEQ ID NO: 284 - DNA, 33 bp, Neoceratodus forsteri
  misc_feature (19)..(19): n is a, c, g, or t
gacatggttctacaggatnggtaaaatgaggtg

SEQ ID NO: 285 - DNA, 33 bp, Neoceratodus forsteri
  misc_feature (15)..(15): n is a, c, g, or t
gacatggttctacangttggttagtgattggtg

SEQ ID NO: 286 - DNA, 37 bp, Neoceratodus forsteri
  misc_feature (17)..(17): n is a, c, g, or t
cagagacttggtctggngtttaaaatagataatagtg

SEQ ID NO: 287 - DNA, 35 bp, Neoceratodus forsteri
cagagacttggtctgtgaatgttttagtgtgtgtg

SEQ ID NO: 288 - DNA, 49 bp, Neoceratodus forsteri
cagagacttggtctacaaataatataaaaatcaaaaatttaacttaatc

SEQ ID NO: 289 - DNA, 37 bp, Neoceratodus forsteri
gacatggttctacagtttgtattattttgagtgtgtg

SEQ ID NO: 290 - DNA, 36 bp, Neoceratodus forsteri
cagagacttggtctcatttacccaattctcaattcc

SEQ ID NO: 291 - DNA, 33 bp, Neoceratodus forsteri
  misc_feature (19)..(19): n is a, c, g, or t
cagagacttggtctggaangtgtttgtgtggtg

SEQ ID NO: 292 - DNA, 37 bp, Neoceratodus forsteri
  misc_feature (16)..(16): n is a, c, g, or t
cagagacttggtctcnctataaaacaaaaaatccttc

SEQ ID NO: 293 - DNA, 49 bp, Neoceratodus forsteri
cagagacttggtctatacaattattcatttattacatttaataacaact

SEQ ID NO: 294 - DNA, 45 bp, Neoceratodus forsteri
cagagacttggtctatctacataccaatattaaattacaatcata

SEQ ID NO: 295 - DNA, 40 bp, Neoceratodus forsteri
  misc_feature (25)..(25): n is a, c, g, or t
gacatggttctacagtgtaaataangtttttatttttgtg

SEQ ID NO: 296 - DNA, 33 bp, Neoceratodus forsteri
  misc_feature (16)..(16): n is a, c, g, or t
gacatggttctacagnggatgaaagtgagtagg

SEQ ID NO: 297 - DNA, 35 bp, Neoceratodus forsteri; identical to SEQ ID NO: 240
  misc_feature (21)..(21): n is a, c, g, or t

SEQ ID NO: 298 - DNA, 30 bp, Neoceratodus forsteri
  misc_feature (16)..(16): n is a, c, g, or t
gacatggttctacacnactccccaaccctc

SEQ ID NO: 299 - DNA, 35 bp, Neoceratodus forsteri
  misc_feature (21)..(21): n is a, c, g, or t
gacatggttctacagtgatgngtaagaaattagtg

SEQ ID NO: 300 - DNA, 36 bp, Neoceratodus forsteri
  misc_feature (23)..(23): n is a, c, g, or t
cagagacttggtctccttattcntcaaattcctatc

SEQ ID NO: 301 - DNA, 39 bp, Neoceratodus forsteri
  misc_feature (25)..(25): n is a, c, g, or t
cagagacttggtcttatattggatngttttaaagaatag

SEQ ID NO: 302 - DNA, 49 bp, Neoceratodus forsteri
gacatggttctacaataaaaaaaaaatattaattttactactcatttat

SEQ ID NO: 303 - DNA, 50 bp, Neoceratodus forsteri
cagagacttggtcttattatttatattatgtatttaaatatatagaagtg

SEQ ID NO: 304 - DNA, 33 bp, Neoceratodus forsteri
cagagacttggtctttccataactcctcaaccc

SEQ ID NO: 305 - DNA, 37 bp, Neoceratodus forsteri
  misc_feature (18)..(18): n is a, c, g, or t
gacatggttctacattgnggataaagaataaaagtga

SEQ ID NO: 306 - DNA, 49 bp, Neoceratodus forsteri
cagagacttggtcttttatttgtttttattattatattggatttaaagt

SEQ ID NO: 307 - DNA, 39 bp, Neoceratodus forsteri
cagagacttggtctatggtgtttaaaatagataatagtg

SEQ ID NO: 308 - DNA, 37 bp, Neoceratodus forsteri
  misc_feature (21)..(21): n is a, c, g, or t
gacatggttctacattatttngaatagtagaagtgtg

SEQ ID NO: 309 - DNA, 39 bp, Neoceratodus forsteri
gacatggttctacagtgttgtttaaaatagataatagtg

SEQ ID NO: 310 - DNA, 47 bp, Neoceratodus forsteri
  misc_feature (17)..(17): n is a, c, g, or t
cagagacttggtctacncttcaaataaaaaaaaaaataaaaaaactc

SEQ ID NO: 311 - DNA, 39 bp, Neoceratodus forsteri
cagagacttggtcttgtttatgttgttgtttatagtgaa

SEQ ID NO: 312 - DNA, 45 bp, Neoceratodus forsteri
cagagacttggtctttatttttgtgtttatgttgttgtttatagt

SEQ ID NO: 313 - DNA, 37 bp, Neoceratodus forsteri
  misc_feature (25)..(25): n is a, c, g, or t
cagagacttggtctcattaatcacnaaatacatacct

SEQ ID NO: 314 - DNA, 39 bp, Neoceratodus forsteri
  misc_feature (22)..(22): n is a, c, g, or t
cagagacttggtctaataaacntaacaaaataactcaac

SEQ ID NO: 315 - DNA, 47 bp, Neoceratodus forsteri
  misc_feature (23)..(23): n is a, c, g, or t
cagagacttggtctttttaagtngatatatatataaaatttgagaaa

SEQ ID NO: 316 - DNA, 39 bp, Neoceratodus forsteri
gacatggttctacaagtagtattggatttatagtagaaa

SEQ ID NO: 317 - DNA, 36 bp, Neoceratodus forsteri
  misc_feature (19)..(19): n is a, c, g, or t
gacatggttctacatattngtttgtgtttgtgtgtg

SEQ ID NO: 318 - DNA, 37 bp, Neoceratodus forsteri; identical to SEQ ID NO: 226
  misc_feature (17)..(17): n is a, c, g, or t

SEQ ID NO: 319 - DNA, 49 bp, Neoceratodus forsteri
gacatggttctacattttatctttatcaacttaaattattattatccat

SEQ ID NO: 320 - DNA, 35 bp, Neoceratodus forsteri; identical to SEQ ID NO: 240
  misc_feature (21)..(21): n is a, c, g, or t

SEQ ID NO: 321 - DNA, 37 bp, Neoceratodus forsteri
cagagacttggtctacctcaaatattaaatcaaccca

SEQ ID NO: 322 - DNA, 37 bp, Neoceratodus forsteri
  misc_feature (20)..(20): n is a, c, g, or t
cagagacttggtctaaaacnaaacaaatcaaaaccct

SEQ ID NO: 323 - DNA, 40 bp, Neoceratodus forsteri
gacatggttctacaatcttccaattaaaaaaatatctctc

SEQ ID NO: 324 - DNA, 47 bp, Neoceratodus forsteri; identical to SEQ ID NO: 270

SEQ ID NO: 325 - DNA, 35 bp, Neoceratodus forsteri
cagagacttggtcttataccaaacttcctcctatc

SEQ ID NO: 326 - DNA, 47 bp, Neoceratodus forsteri
gacatggttctacaaaggtttttagatgataaaatttaattttggtg

SEQ ID NO: 327 - DNA, 49 bp, Neoceratodus forsteri
gacatggttctacaataatttagttatataatgttttatttgtttgaaa

SEQ ID NO: 328 - DNA, 35 bp, Neoceratodus forsteri
  misc_feature (22)..(22): n is a, c, g, or t
gacatggttctacattgtaaangatagagagtagg

SEQ ID NO: 329 - DNA, 52 bp, Neoceratodus forsteri
cagagacttggtctataaatatattaaaatttaatctataataataacctcc

SEQ ID NO: 330 - DNA, 34 bp, Neoceratodus forsteri
gacatggttctacacacaatctccacacaatctc

SEQ ID NO: 331 - DNA, 33 bp, Neoceratodus forsteri
cagagacttggtctaaaacctcccctaattccc

SEQ ID NO: 332 - DNA, 39 bp, Neoceratodus forsteri
gacatggttctacaacatcaaatacaaattttataaccc

SEQ ID NO: 333 - DNA, 47 bp, Neoceratodus forsteri
cagagacttggtctttataaaactaaattataaaccaaactaccttc

SEQ ID NO: 334 - DNA, 39 bp, Neoceratodus forsteri
cagagacttggtctttaaactcaaaacttctaaactaca

SEQ ID NO: 335 - DNA, 125 bp, Neoceratodus forsteri
tgtctactaatgaagtgttacctgtgggcgcgagggtggtgggggccctgagcgggtctatctccccggggatgcagagctcgggtccgtcggtaaaccggctgcagttgaacatctccggccag

SEQ ID NO: 336 - DNA, 129 bp, Neoceratodus forsteri
ctaaaacagacaatagtgcaaacaacgctttcatttcggtgtccatgttgttgtttacagtactgtcttccggtagcgttaaagccatgtgttatagtcatgtgataggggcttgacgaataagggaag

SEQ ID NO: 337 - DNA, 128 bp, Neoceratodus forsteri
cttattttatccatccccctcctcaactctccttctgccctgtccgctgttcactttcactttcttccgcgactccccatccctctttactttcgtctgcaacaaccccctgctcttcactttcatcc

SEQ ID NO: 338 - DNA, 142 bp, Neoceratodus forsteri
cctaaaacagacaatagtgcaaacaacgctcccatttctgtgcccatgttattgtttacagtgaaggctggtctgctgtcttccggtagcgttaaagtcatgtgttatagtcatgtgataggggcttgacgaatagggaaag

SEQ ID NO: 339 - DNA, 146 bp, Neoceratodus forsteri
gtgccggaacatgccagatctggaacctctgaaatctgatccggcacctcattttaccgatcccccttctaaatcaccctcctcccccgtccactttcactttcttctgcgactccccaacccgctgttgtccgagacaacccccc

SEQ ID NO: 340 - DNA, 147 bp, Neoceratodus forsteri
cccattctcacaactcccacccagtccgttcactttcgtttgtgacacagcaaccctctggcctccccaccgctcttcactttcgtccatgaaagccaccaccccccgctcttcaccttcgtctatgacaccaatcactggccaacg

SEQ ID NO: 341 - DNA, 148 bp, Neoceratodus forsteri
acacatggctttatcgctaccggaagacagtactgtaaacaacaacatggacacagaaatgggagcgttgtttgcactattgtctgttttaggcgccattgtgcagttattcatttgatacatttagtaacaacttgacctccttgtc

SEQ ID NO: 342 - DNA, 142 bp, Neoceratodus forsteri
gtcagagttctggttgctggcttgtctggtcagtgcagcgctgccgactgaagcttcatcctccctcctcttcttcatcgccatggagacctgccatcatcataacgcattagcaactacacacacacactaaaacattcac

SEQ ID NO: 343 - DNA, 141 bp, Neoceratodus forsteri
ctcgctcactctgagcatgtgtgcagatctgacggtcaagtcttttttttcttcatttattcccaaatacttacagccattaaagataccactctgtcaccacgaagaccaagtcaaattcctgatttttatactatctgt

SEQ ID NO: 344 - DNA, 149 bp, Neoceratodus forsteri
ctccctcctgaagtgtcttcatttcatctcgtcttttctcaagcacttgttcaataaatgctgaagtttgagagccgtgttgagtccgtcattactgtgttcagtgtttcctccacacactcagagtaatgcagacctgaccatcaaaa

SEQ ID NO: 345 - DNA, 139 bp, Neoceratodus forsteri
atgacggcaactgttcatcataaaatgtgaaaagttctcaaaattaaacaccctggttaatgatattaacaatgacttcatgtgctcataaacattcaaactcattaacgtaacatgggaactgagaattgggtaaatg

SEQ ID NO: 346 - DNA, 142 bp, Neoceratodus forsteri
accccacatctagttctcctgtatgtccacacatgtagaagacagacaataaagccggatttgactcacatgatggtctggacgacgcgcgggtggcggctcatgcccagatagtcattactgcaccacacagacacgttcc

SEQ ID NO: 347 - DNA, 151 bp, Neoceratodus forsteri
caaactaccttctgccagcaggtggcgttatgactgtgactcaatattgttatgtggatgtgttcaggagcggacttttatcaatcatgtgaagtttcaggcagatcagatattgtgtggttgagttagaaggacttcctgttccatagcg

SEQ ID NO: 348 - DNA, 137 bp, Neoceratodus forsteri
gctcgacatttggttattgttagtcaacaaacgccggatgaacaatgaatgcgatggcaaagaaagcaagagaggtatttttgtggacaggcgacgaggtcgagttgttactaaatgtaacaaatgaacaactgcac

SEQ ID NO: 349 - DNA, 152 bp, Neoceratodus forsteri
cacccggtggtcttaggatgacagagaatagtgaacaataataatttaagctgataaatattttaaaaatcagagaaaatcactgatctatgccaaacttccttctgtcagcaggtggcggtatgactgtaactcaatattggtatgtagat

SEQ ID NO: 350 - DNA, 137 bp, Neoceratodus forsteri
cattgcgtatccttcccttattcgtcaagcccttatcacatgactataacatacggctttaacgctaccggaagacagcagaccagccttcactgtgaacaacaacacgggcacagaaataagagcgttgtttgcac

SEQ ID NO: 351 - DNA, 126 bp, Neoceratodus forsteri
ctctgagatctgatttggcattttactgatcctccttcaaccccgtttactttcactttcttccgcgactctccaacccccatcactgctgtccgtgacacccccgccctgctcactttcatccgc

SEQ ID NO: 352 - DNA, 137 bp, Neoceratodus forsteri
gtccatgttgttgtttacagtgaaggctggtctgctgtcttccagtagtgttaaagccatgtgttatagtcatgtgataggggcttgacgaataagggaaggatacgcaatgacacaaaagccccaatcaggtagcg

SEQ ID NO: 353 - DNA, 127 bp, Neoceratodus forsteri
agtgcttaatttgtgaattgcgaggtcccggaacagatcggggtaacggatccagaatataactcaaggatggagaggtgtcgcggttgaaagtgaagagggttggggagtcgcagaagaaagtgaa

SEQ ID NO: 354 - DNA, 132 bp, Neoceratodus forsteri
cgaagtacattattctgcttataccacagttcccacagctaaatccactgttcagatgttgtattttatacatttgtcaggtttttgttctcaagctgttcctgtgtgcagcactagtttcttacgcatcac

SEQ ID NO: 355 - DNA, 139 bp, Neoceratodus forsteri
aacagacaatagtgcaaacaacgctcccatttctgtgtccatgttgttgtttacagtgaaggctggtctgctgtcttccggtagcgttaaagccatgtgcagtcatgtgataggaacttgacgaataagggaaggatac

SEQ ID NO: 356 - DNA, 136 bp, Neoceratodus forsteri
gtcatgtgacactcagtcatgttactaattcgtttactttattttcgttgtgagtatgattttagtgatgtcgtccttactgtgagggtaagcagcgtcatcagcaggatactgttctttaaagcggtccagtata

SEQ ID NO: 357 - DNA, 153 bp, Neoceratodus forsteri
cggatgtgatgtatttcagtgtttaagcaggaccgtgcatgcgagataagaaattgtagttttactggttattgtttatatcagaaaaagtctatattaaaataaaacatttctattcataaatgagtagtaaaactaatattttttttttat

SEQ ID NO: 358 - DNA, 143 bp, Neoceratodus forsteri
agtccggataccaattaatttcctatgacggtcacttttgaccgggaacaccacaggtgtaacaaagttgattaaaacactcaaaattcaatgaaagagatgataatcacttctatatatttaggtgcataatgtggatgatg

SEQ ID NO: 359 - DNA, 146 bp, Neoceratodus forsteri
attcattataactcattaccagtgcttaatttgtgaattgcgaggtcccggaacagatcggggtaacaaggacgtggacgaaggacgaaagggaagagcagaggtttgtcgcggacaaaagtaaagagggttgaggagtcatggaa

SEQ ID NO: 360 - DNA, 138 bp, Neoceratodus forsteri
tcattttaccaatccccctcttcaaccgcccttctcccctgtccgctgtttactttccctttcttctgcgactccccaaccctcttcacttttttccacgacaaccttccgctcttcacttttattctttgtccgcaa

SEQ ID NO: 361 - DNA, 147 bp, Neoceratodus forsteri
aataataagagcaagtggatttttaaaaatctttataaaaagaacaaacaaaaacatattcagcccccttttttatatttattatctaaattttatttttttcaatggtttccagatcttatttcaatctcaataaatatagatttt

SEQ ID NO: 362 - DNA, 153 bp, Neoceratodus forsteri
cccttattcgtcaaggccctattacttgactaaaaataaattactttaatgctggaagacagaccagccttcactgtaaacaacaacacagacacagaaatgggagcttgtttgcactattgtctgttttaggcaccattgtgaatgtattca

SEQ ID NO: 363 - DNA, 153 bp, Neoceratodus forsteri
cgctctgctgcccccttcacgacagtcccgttatcggttgtcaggacaacagcccgttctgtgcgcgggcacgtatcacaaaagaccgccaagcgtgtgcccgagaacaccaaaagagcacgcgcacagacacacttctgctgttcggaatga

SEQ ID NO: 364 - DNA, 148 bp, Neoceratodus forsteri
gcttcaacgctaccgggagacagcactgtaaacaacaacatgggcacagaaaagggatcgttgtttgcactattgtctgttttaggcagcacagttattcatttgttacgtttagtaacaactcgtcctcgtcgtctgtccacaaaaa

SEQ ID NO: 365 - DNA, 130 bp, Neoceratodus forsteri
cagactggtacagactgatgtgacttgagtggggtgggggtgttttatttctacatatatacgcctttttttaggtgagggaatggaagttttgagagagttcccccactttttcccccacttgaagcgc

SEQ ID NO: 366 - DNA, 133 bp, Neoceratodus forsteri
cattgcgtatccttcccttattcgtcatctatacccctataaccacgtacccctatcacatgactataacacatggctttaacgctaccggaagacagcagaccagccttcactgtaaacaacaacatggaca

SEQ ID NO: 367 - DNA, 125 bp, Neoceratodus forsteri
tgtcattgcgtatccttcccttattcgtcaagcccatatcacatgactataacacatggctttaatgctaccggaagacagcagaccagccttcactgtaaacaacaacatggacacagaaatgg

SEQ ID NO: 368 - DNA, 129 bp, Neoceratodus forsteri
ctgagtagctttctcagatgtgggcataacttatgttcgctcatttaatatgttacgtgtcgccagatggttttactcactcgtttccagtggtttttggattgccaggtatgtattccgtgatcaatg

SEQ ID NO: 369 - DNA, 128 bp, Neoceratodus forsteri
ctttttcgtatgttttggcatgtttgaggtgtgtgccgattttcttgcatgtgcgtgattcgtggatcgggggcttgtccggttaatttttctaggtggcgctgttgagtcattttgccacgcccatt

SEQ ID NO: 370 - DNA, 131 bp, Neoceratodus forsteri
tgattaaattcctctcctgaagaaatctacattgcaatattgagtcacggtcatagcgccacctgctgacagaaggaagtttggcataaatctgtgattttctcaggttttatatatatgtcggcttaaga

SEQ ID NO: 371 - DNA, 127 bp, Neoceratodus forsteri
ctcatctgtccagcatctccagaaccagcgacaaacacagagaaacggcagcgctctggctgtcagagctggtggaggagcccgtcatgtccagcacatgtgtttctgctgtggatccagtactgct

SEQ ID NO: 372 - DNA, 125 bp, Neoceratodus forsteri
caagaggcctttccaaaaaaaaaatgtaatcatattaatccgcatgtagcttacatcagcacacaaacacaacacaaacgtcctcttttttgatgcgcgcacacacacacaagcacaaacgagtg

SEQ ID NO: 373 - DNA, 141 bp, Neoceratodus forsteri
actattgtctgttttaggcgccattgtgcagttattcatttgttacgtttagtaacaactcgaccacgtcgtctgtccacaaaaatacctcttctgctttctctgccatcgtgttcattgttccaccggcgtttgttgact

SEQ ID NO: 374 - DNA, 129 bp, Neoceratodus forsteri
aagatacaaactaaattaaagctgcaagcagtgatgaaagggatctcgaacccgggctcaccgctgccttgtggccttaggatgacaaagcacaatggacaataataatttaagctgataaagataaaa

SEQ ID NO: 375 - DNA, 148 bp, Neoceratodus forsteri
aatgacacaaaagccccaatcaagtagcgaatcgcggcggccccgcctccgttttcagatgtctctgttttttcctcatccacactgaaacggagcagcagcgttttagaatgaaaacggcctctccagcgtttttgaaacgctccgt

SEQ ID NO: 376 - DNA, 131 bp, Neoceratodus forsteri
actgtgggattataatgcttagtatgactgaattcaaccaatcgctccatctgtgatgataatctgcgattttatcaatagaccggaggccagactgagagagatatgtgggttgacttaatatttgaggt

SEQ ID NO: 377 - DNA, 146 bp, Neoceratodus forsteri
aattatttattcagtgatggtaagtaaaagtttgttttaaataaaaagtctgcgtctccctgtgttgctcaggctgcactgcagtgtctattcacaggtgcgatcccactactgatcggcacgagggttttgacctgctccgtctc

SEQ ID NO: 378 - DNA, 148 bp, Neoceratodus forsteri
atactaacacattatttacttacaattaacagaagaaattgagcagatttttttgccattttgtacacagccaatcacttggcgccatttactaaataatctttctgttctcagcatcccgagagagacatctcctcaactggaagac

SEQ ID NO: 379 - DNA, 138 bp, Neoceratodus forsteri
cagatttatgccaaacttcctcctgtcagcaggtggcgctataactgtgacttaagattggcatgtagatgtcttccggagaggaatcttatcaaccatgtgaagtttcaggcacatgggacattgtgtggctgagtt

SEQ ID NO: 380 - DNA, 132 bp, Neoceratodus forsteri
aactcagccacacaatgtcccatgtgcctgaaacttcacatggttgataagattcctctccggaagatatctacatgccaatcttaagtcacaattatatcgccacctgctgacaggaggaagtttggcata

SEQ ID NO: 381 - DNA, 138 bp, Neoceratodus forsteri
caggaagtcggatattttgtacttcctgcggcgaaaaagtggcgattttgccatttccaggcgttgtattttaacgaactcctcctaggaattttatccgatcgacaccaaaattaggttttgtcatctaaaggcctt

SEQ ID NO: 382 - DNA, 136 bp, Neoceratodus forsteri
tttatgccaaacttccttctgtcagcaggtggcgctataactgtgactcaagattggcatgtagatgtcttccggagaggaatcttatcaaccatgtgaagtttcaggcagatgagacattgtgtggctgagttat

SEQ ID NO: 383 - DNA, 129 bp, Neoceratodus forsteri
ccctcctcccctgttcgctgttcactttcacttttttccgtgcctccccaactcttcactttcgtccgtaacaccccagcctgctctctatcgtttgcaacactcctccaccatccactgcaccatccc

SEQ ID NO: 384 - DNA, 131 bp, Neoceratodus forsteri
atgcggaataattgacaccagtgcgttgaattattagaaaaattgaaccactttaaattttgactgcaatttattacagataaagtacatgcaggaggttattatcacagattaaaccccaacatatttac

SEQ ID NO: 385 - DNA, 126 bp, Neoceratodus forsteri
cagcaggtggcgttatgactgactcaatattgttatgtggatgtgttcaggagcggacttttatcaaccatgtgaagtttcaggcagatcagagattgtgtggagattgtgttttaagacaaacta

SEQ ID NO: 386 - DNA, 150 bp, Neoceratodus forsteri
aggctttgtatatcggatgggtgtcatctgcgttcttgtttgccggaggatgcatcttcatatgctgcagtggttctctagacaagggtccggatccaaagtatatgtactccaggaatgcacctgctccatatgtggcctaccagcctc

SEQ ID NO: 387 - DNA, 137 bp, Neoceratodus forsteri
aaacaaaatgaacaacagtgaaatactgtgaaatgatgattgctaaaaagtaagcgagtcgatgcaattgacactagatttttagcaagccactgctgacttgactctctaggggttataaaacttgcacctgatgc

SEQ ID NO: 388 - DNA, 146 bp, Neoceratodus forsteri
aacttagccacacaatgtctgaactgcttgaaacttcacatggttgattaaattcctctcctgaacacatttacatgccaatattgagtctgtcatagcgccgcctgctggcagaaggtagtttggcttacaactcagccccacaa

SEQ ID NO: 389 - DNA, 127 bp, Neoceratodus forsteri
gctcccccaggagcaccatatcgataccgaacttagtgtggacacctgatcaacataatcacactgcagtccagaagccctgagctcaagcgatccgcagcctcagcctcccagtagcttggattac

SEQ ID NO: 390 - DNA, 39 bp, Maccullochella peelii
cagagacttggtctgtttttagtgttttgtttgttttag

SEQ ID NO: 391 - DNA, 35 bp, Maccullochella peelii
gacatggttctacaggtgattgtaggttgtaaaag

SEQ ID NO: 392 - DNA, 39 bp, Maccullochella peelii
gacatggttctacagtggttttattataggtaattttag

SEQ ID NO: 393 - DNA, 35 bp, Maccullochella peelii
  misc_feature (22)..(22): n is a, c, g, or t
gacatggttctacaggttgaangggtttttaaagg

SEQ ID NO: 394 - DNA, 33 bp, Maccullochella peelii
  misc_feature (19)..(19): n is a, c, g, or t
cagagacttggtcttggtngagaggagtagtaa

SEQ ID NO: 395 - DNA, 39 bp, Maccullochella peelii
  misc_feature (18)..(18): n is a, c, g, or t
gacatggttctacaggangattgatttttaaattaaagg

SEQ ID NO: 396 - DNA, 39 bp, Maccullochella peelii
gacatggttctacagtaagtagttaatgtgtattagatt

SEQ ID NO: 397 - DNA, 33 bp, Maccullochella peelii
  misc_feature (22)..(22): n is a, c, g, or t
gacatggttctacacctctccnaaaaaacttcc

SEQ ID NO: 398 - DNA, 37 bp, Maccullochella peelii
  misc_feature (18)..(18): n is a, c, g, or t
gacatggttctacattangtatatatattgaggtgtg

SEQ ID NO: 399 - DNA, 37 bp, Maccullochella peelii
  misc_feature (25)..(25): n is a, c, g, or t
gacatggttctacaaagtttatgtngtttagggatat

SEQ ID NO: 400 - DNA, 35 bp, Maccullochella peelii
gacatggttctacagggggattttaatgaagtagt

SEQ ID NO: 401 - DNA, 33 bp, Maccullochella peelii
  misc_feature (16)..(16): n is a, c, g, or t
gacatggttctacacnaaaaacctctttcaccc

SEQ ID NO: 402 - DNA, 33 bp, Maccullochella peelii
gacatggttctacacaaccccaaatctcctatc

SEQ ID NO: 403 - DNA, 32 bp, Maccullochella peelii
  misc_feature (19)..(19): n is a, c, g, or t
gacatggttctacaccccnctaattctccttc

SEQ ID NO: 404 - DNA, 37 bp, Maccullochella peelii
  misc_feature (15)..(15): n is a, c, g, or t
gacatggttctacanggtatttagttattgaagtagt

SEQ ID NO: 405 - DNA, 39 bp, Maccullochella peelii
cagagacttggtctgatataataaatagttagtgggtaa

SEQ ID NO: 406 - DNA, 34 bp, Maccullochella peelii
cagagacttggtctcttaaaaaccctacactccc

SEQ ID NO: 407 - DNA, 35 bp, Maccullochella peelii
  misc_feature (20)..(20): n is a, c, g, or t
gacatggttctacagtgagngagtgtttttaaagg

SEQ ID NO: 408 - DNA, 33 bp, Maccullochella peelii
gacatggttctacacacctccactttcacttcc

SEQ ID NO: 409 - DNA, 34 bp, Maccullochella peelii
  misc_feature (16)..(16): n is a, c, g, or t
gacatggttctacacnaacctcatcatatccttc

SEQ ID NO: 410 - DNA, 32 bp, Maccullochella peelii
gacatggttctacacacacctccaccataacc

SEQ ID NO: 411 - DNA, 39 bp, Maccullochella peelii
gacatggttctacatattagtgttgatttttgtagtagt

SEQ ID NO: 412 - DNA, 39 bp, Maccullochella peelii
cagagacttggtctaaatcttcatctcaaatatttttcc

SEQ ID NO: 413 - DNA, 35 bp, Maccullochella peelii
  misc_feature (17)..(17): n is a, c, g, or t
cagagacttggtcttanggagttatgtagggtatg

SEQ ID NO: 414 - DNA, 37 bp, Maccullochella peelii
cagagacttggtctgtaattaggatatgttgtgtatg

SEQ ID NO: 415 - DNA, 35 bp, Maccullochella peelii
  misc_feature (23)..(23): n is a, c, g, or t
cagagacttggtctagagttgtnggtattaggtaa

SEQ ID NO: 416 - DNA, 33 bp, Maccullochella peelii
  misc_feature (20)..(20): n is a, c, g, or t
gacatggttctacagtttanggggtaagggtaa

SEQ ID NO: 417 - DNA, 35 bp, Maccullochella peelii
  misc_feature (19)..(19): n is a, c, g, or t
cagagacttggtcttgaanggattgtgggatataa

SEQ ID NO: 418 - DNA, 32 bp, Maccullochella peelii
  misc_feature (21)..(21): n is a, c, g, or t
cagagacttggtcttcttccnccacatcctca

SEQ ID NO: 419 - DNA, 40 bp, Maccullochella peelii
cagagacttggtctaataactatcaaatcaacaaaattcc

SEQ ID NO: 420 - DNA, 39 bp, Maccullochella peelii
gacatggttctacagagttttttaaaagagatgtaatag

SEQ ID NO: 421 - DNA, 33 bp, Maccullochella peelii
  misc_feature (15)..(15): n is a, c, g, or t
gacatggttctacanacttcctatcctacctca

SEQ ID NO: 422 - DNA, 36 bp, Maccullochella peelii
  misc_feature (20)..(20): n is a, c, g, or t
cagagacttggtctaccccnataatattctctatac

SEQ ID NO: 423 - DNA, 40 bp, Maccullochella peelii
gacatggttctacaggatattgtgttttaaatgtaatatg

SEQ ID NO: 424 - DNA, 33 bp, Maccullochella peelii
gacatggttctacaaactcctaaccaccctatc

SEQ ID NO: 425 - DNA, 50 bp, Maccullochella peelii
cagagacttggtcttaattaacacataaattaaaattaaataattttctc

SEQ ID NO: 426 - DNA, 30 bp, Maccullochella peelii
  misc_feature (18)..(18): n is a, c, g, or t
gacatggttctacacccncaaaccctctcc

SEQ ID NO: 427 - DNA, 39 bp, Maccullochella peelii
  misc_feature (25)..(25): n is a, c, g, or t
gacatggttctacatatatattaangtagttgtagtatg

SEQ ID NO: 428 - DNA, 37 bp, Maccullochella peelii
  misc_feature (18)..(18): n is a, c, g, or t
gacatggttctacactcntatttctaaaccaacaatt

SEQ ID NO: 429 - DNA, 52 bp, Maccullochella peelii
cagagacttggtctttataaaattaatacaaaaaaattaaaaaaatatttca

SEQ ID NO: 430 - DNA, 34 bp, Maccullochella peelii
  misc_feature (15)..(15): n is a, c, g, or t
gacatggttctacanggtggtgttagttttatgg

SEQ ID NO: 431 - DNA, 40 bp, Maccullochella peelii
cagagacttggtctgagatttttgtatattgtataaagtg

SEQ ID NO: 432 - DNA, 37 bp, Maccullochella peelii
gacatggttctacaaagatttgtatgtggtaaaaagg

SEQ ID NO: 433 - DNA, 35 bp, Maccullochella peelii
gacatggttctacagtggtagaggtaattaggtaa

SEQ ID NO: 434 - DNA, 38 bp, Maccullochella peelii
  misc_feature (26)..(26): n is a, c, g, or t
gacatggttctacattaaaaactacnctctaaactatc

SEQ ID NO: 435 - DNA, 40 bp, Maccullochella peelii
cagagacttggtcttatgtatgttagtttttgtatgtatg

SEQ ID NO: 436 - DNA, 49 bp, Maccullochella peelii
gacatggttctacaatttatgtgtttttttatgtttatattaattaagt

SEQ ID NO: 437 - DNA, 38 bp, Maccullochella peelii
  misc_feature (15)..(15): n is a, c, g, or t
gacatggttctacanggttttttgagttatttgaaaag

SEQ ID NO: 438 - DNA, 39 bp, Maccullochella peelii
gacatggttctacaaaacttcaattcaaatcccaaattt

SEQ ID NO: 439 - DNA, 33 bp, Maccullochella peelii
  misc_feature (16)..(16): n is a, c, g, or t
cagagacttggtctcnacaacaactctccatcc

SEQ ID NO: 440 - DNA, 32 bp, Maccullochella peelii
cagagacttggtctcaaaacccaacccccttc

SEQ ID NO: 441 - DNA, 49 bp, Maccullochella peelii
cagagacttggtctttctacattatttcatttaaattaataaaactcca

SEQ ID NO: 442 - DNA, 30 bp, Maccullochella peelii
  misc_feature (22)..(22): n is a, c, g, or t
gacatggttctacaccccctcntcttcctc

SEQ ID NO: 443 - DNA, 35 bp, Maccullochella peelii
  misc_feature (22)..(22): n is a, c, g, or t
cagagacttggtcttacaatcncaaaaaacctcca

SEQ ID NO: 444 - DNA, 35 bp, Maccullochella peelii
  misc_feature (22)..(22): n is a, c, g, or t
cagagacttggtcttctactcnatcattctctatc

SEQ ID NO: 445 - DNA, 39 bp, Maccullochella peelii
  misc_feature (15)..(15): n is a, c, g, or t
cagagacttggtctngtaggttttttttttttgttttag

SEQ ID NO: 446 - DNA, 37 bp, Maccullochella peelii
cagagacttggtctaaatacccatttcctctatcaaa

SEQ ID NO: 447 - DNA, 32 bp, Maccullochella peelii
  misc_feature (18)..(18): n is a, c, g, or t
cagagacttggtctaccntcaacctctcactc

SEQ ID NO: 448 - DNA, 35 bp, Maccullochella peelii
  misc_feature (21)..(21): n is a, c, g, or t
cagagacttggtctctacccnaaaaaaatactccc

SEQ ID NO: 449 - DNA, 45 bp, Maccullochella peelii
  misc_feature (19)..(19): n is a, c, g, or t
cagagacttggtctgatangtattatttttttgttatagtttagt

SEQ ID NO: 450 - DNA, 36 bp, Maccullochella peelii
  misc_feature (20)..(20): n is a, c, g, or t
cagagacttggtctggagtngttagatttttaaagg

SEQ ID NO: 451 - DNA, 35 bp, Maccullochella peelii
cagagacttggtctgtttagttggggtttagtagt

SEQ ID NO: 452 - DNA, 34 bp, Maccullochella peelii
  misc_feature (17)..(17): n is a, c, g, or t
cagagacttggtctacnataaaccaaccctaacc

SEQ ID NO: 453 - DNA, 33 bp, Maccullochella peelii
  misc_feature (24)..(24): n is a, c, g, or t
gacatggttctacacctctactcnaaactctcc

SEQ ID NO: 454 - DNA, 37 bp, Maccullochella peelii
gacatggttctacagattagttagtttgtttgaagtg

SEQ ID NO: 455 - DNA, 37 bp, Maccullochella peelii
  misc_feature (26)..(26): n is a, c, g, or t
cagagacttggtcttaaatacttccnattatcctatc

SEQ ID NO: 456 - DNA, 37 bp, Maccullochella peelii
  misc_feature (22)..(22): n is a, c, g, or t
cagagacttggtctggtttttngttttgttattaagg

SEQ ID NO: 457 - DNA, 36 bp, Maccullochella peelii
cagagacttggtctgagttaataaggtaggtaaagg

SEQ ID NO: 458 - DNA, 37 bp, Maccullochella peelii
  misc_feature (24)..(24): n is a, c, g, or t
cagagacttggtctattgtgtagngtttattatatgg

SEQ ID NO: 459 - DNA, 40 bp, Maccullochella peelii
  misc_feature (23)..(23): n is a, c, g, or t
cagagacttggtctctaataacnaattattaacatctatc

SEQ ID NO: 460 - DNA, 37 bp, Maccullochella peelii
gacatggttctacattattgttggttgttagaggtaa

SEQ ID NO: 461 - DNA, 39 bp, Maccullochella peelii
gacatggttctacaaaatcctaattataataaatccctc

SEQ ID NO: 462 - DNA, 34 bp, Maccullochella peelii
  misc_feature (18)..(18): n is a, c, g, or t
gacatggttctacaaccntaactaaccttcttcc

SEQ ID NO: 463 - DNA, 49 bp, Maccullochella peelii
gacatggttctacattaataaaacatataaaaataaaaacaaactctta

SEQ ID NO: 464 - DNA, 33 bp, Maccullochella peelii
  misc_feature (18)..(18): n is a, c, g, or t
cagagacttggtctaacnaacactcttccctcc

SEQ ID NO: 465 - DNA, 34 bp, Maccullochella peelii
  misc_feature (22)..(22): n is a, c, g, or t
gacatggttctacacatctccnatttcactcatc

SEQ ID NO: 466 - DNA, 37 bp, Maccullochella peelii
  misc_feature (26)..(26): n is a, c, g, or t
gacatggttctacatttgtagttttngtagtagtagt

SEQ ID NO: 467 - DNA, 35 bp, Maccullochella peelii
  misc_feature (16)..(16): n is a, c, g, or t
gacatggttctacagnggtaagtagtttatttgtg

SEQ ID NO: 468 - DNA, 45 bp, Maccullochella peelii
cagagacttggtctaatccacctaattttattaaattatcattct

SEQ ID NO: 469 - DNA, 52 bp, Maccullochella peelii
cagagacttggtcttattataattaatttaataatataaatttttagaagtg

SEQ ID NO: 470 - DNA, 34 bp, Maccullochella peelii
  misc_feature (17)..(17): n is a, c, g, or t
gacatggttctacaggnggagttgaatagaaaag

SEQ ID NO: 471 - DNA, 37 bp, Maccullochella peelii
  misc_feature (20)..(20): n is a, c, g, or t
cagagacttggtctctatcnataatattttttcctcc

SEQ ID NO: 472 - DNA, 38 bp, Maccullochella peelii
  misc_feature (16)..(16): n is a, c, g, or t
cagagacttggtctgngtatagtagaatatatttaagg

SEQ ID NO: 473 - DNA, 40 bp, Maccullochella peelii
gacatggttctacatagaaatattgagaagatgataaaag

SEQ ID NO: 474 - DNA, 39 bp, Maccullochella peelii
  misc_feature (26)..(26): n is a, c, g, or t
cagagacttggtctggatttattatngagtaaattaagt

SEQ ID NO: 475 - DNA, 37 bp, Maccullochella peelii
  misc_feature (26)..(26): n is a, c, g, or t
cagagacttggtctaatcctataacnaaaactattcc

SEQ ID NO: 476 - DNA, 39 bp, Maccullochella peelii
  misc_feature (16)..(16): n is a, c, g, or t
cagagacttggtctangatttgaattgtattttggatat

SEQ ID NO: 477 - DNA, 37 bp, Maccullochella peelii
gacatggttctacattgaattaggtggtttttaaagg

SEQ ID NO: 478 - DNA, 39 bp, Maccullochella peelii
  misc_feature (18)..(18): n is a, c, g, or t
cagagacttggtctaacnaataaccaaaaaaaactatct

SEQ ID NO: 479 - DNA, 37 bp, Maccullochella peelii
gacatggttctacaatataatacctccttcaattcac

SEQ ID NO: 480 - DNA, 37 bp, Maccullochella peelii
  misc_feature (20)..(20): n is a, c, g, or t
cagagacttggtctctctcnacaaaaaataactttca

SEQ ID NO: 481 - DNA, 39 bp, Maccullochella peelii
cagagacttggtctcatattctttacattattactaacc

SEQ ID NO: 482 - DNA, 45 bp, Maccullochella peelii
  misc_feature (26)..(26): n is a, c, g, or t
cagagacttggtctttgaagatttangttgatttatatatgtatg

SEQ ID NO: 483 - DNA, 33 bp, Maccullochella peelii
gacatggttctacaaacaataaccccctccttc

SEQ ID NO: 484 - DNA, 33 bp, Maccullochella peelii
  misc_feature (24)..(24): n is a, c, g, or t
cagagacttggtctatcaaaatcnaccctctcc

SEQ ID NO: 485 - DNA, 39 bp, Maccullochella peelii
cagagacttggtctaattaaaacaacaacctaactatct

SEQ ID NO: 486 - DNA, 133 bp, Maccullochella peelii
gtcctcagtgctctgcttgtctcagcagcgcgagcccgccgccacagctgcagcctatataaccatcggtgacgttgcgctgcaggccacgccccctcagatggaccgaaatctgggacctgaactgaagttt

SEQ ID NO: 487 - DNA, 126 bp, Maccullochella peelii
ggtgactgtaggctgcaaaaggccattttcagcctgaagcgcgtcttattgatcggtgtgacggtaaaacctggactaaccccaccgccgcgctccttcatgtcccgggatggagagttgctgtcg

SEQ ID NO: 488 - DNA, 150 bp, Maccullochella peelii
gtggctttatcataggcaactttagcagaaatcccaatggcgatgatgagcgctatgaccccgcaggtgatgtacaccgtggtgttggtctggtccagctccgggtcgtacgtatcacctgttggcgccggtgaagggggctgggttttg

SEQ ID NO: 489 - DNA, 126 bp, Maccullochella peelii
ggctgaacgggtttctaaaggggtttcctcgactcgaaggcaaggaccggaagaggaaagggagtgccgggttttgtcatcactgccccgttggagctccactaacctaaatgaaacaatgcagaa

SEQ ID NO: 490 - DNA, 138 bp, Maccullochella peelii
ctatcagtacgtggcccccggggtgatcaatttaggctccccgcacggttatttcacggaggaagacgagggggacatcttcccgactccggacccccactacgttaagaaatactacttccccgtgagagacctgga

SEQ ID NO: 491 - DNA, 126 bp, Maccullochella peelii
gcacctggtccagaatgtccgtctggaggtcccctgcgactgcagaccgggacagaagaagtgtacctgctaccggccgaaccgcaaggagacctggctcttctcccggttctccaccggctggag

SEQ ID NO: 492 - DNA, 128 bp, Maccullochella peelii
gtaagtagttaatgtgcaccagatttacgcacggacctcatttgacgcagtgtggtcagcgtgcacccacaaacctcacatggtaatttgagtaatttaaagcatttgacagagaatgaccgagcaga

SEQ ID NO: 493 - DNA, 132 bp, Maccullochella peelii
cctctccggagaagcttccagttccagccggttacgtccccgagctcgtccatcgctgcccccgctgtggtgaggcgtttggccaggccagcagcctgcgcctccacctggagcagaagaggaagacctacg

SEQ ID NO: 494 - DNA, 138 bp, Maccullochella peelii
ttacgtacacatactgaggtgtgagctgtcagggaagacactcactcggggaaaacgtgctctccgttatggcccgatttcaactctataaaccatacagaaggatacatctctgtttgacagaggaaatgggtattt

SEQ ID NO: 495 - DNA, 126 bp, Maccullochella peelii
ccgcccagggacatgctctcaaacccggggctcgggttcgggtcgatccgctcgtcttcaaactcttcatctgatgacgaggatgacacagatgaggaagagtgagaggctgacggtgttggagag

SEQ ID NO: 496 - DNA, 143 bp, Maccullochella peelii
gggggatcttaatgaagcagtcgttgaaggagaggttgtgccgcagggagttctgccaccgctgcgtgttctcccggtagtacgggaaccggtccatgatgaacttgtaaatctcactgagggggagcatcttctccgggcag

SEQ ID NO: 497 - DNA, 132 bp, Maccullochella peelii
cggaggacctctttcaccccgcaggcgctcgagatcctcaacagccactttgagaagaacacacacccctccggacaggaaatgacggaaatagcggagaaactgaactatgacagggaggtggtgcgtgtc

SEQ ID NO: 498 - DNA, 134 bp, Maccullochella peelii
cagccccagatctcctatcttgacggagccggtggggccggtgatgaagatgttgtcgcacttgaggtcccggtggatgatgggaggagtgcgcgtgtgcaggaagtgaagccctttgaggatctgacgactcc

SEQ ID NO: 499 - DNA, 143 bp, Maccullochella peelii
ccccgctggttctccttcaagtccaggtagtatttgcggttttcccggactagaaactcgctcttgagagctctcctgggcccgccgtcctcacccgcgctggcctgggctatctgctctggactgctgggccccagctgagc

SEQ ID NO: 500 - DNA, 135 bp, Maccullochella peelii
tcccctctgaagcagataaggctacacgtcaggtcagggctggttcaccgcagttcggctattggggtgtcaagtgtgtttgtgcatgtgtgtgtatttgtgtacgtttgtgtttgtttgactgtgcatgtgtgt

SEQ ID NO: 501 - DNA, 131 bp, Maccullochella peelii
ccagttttgggcctgcggatcaagaaggagagtcccgagcagaggagacagcgggagaagtcgtctgctcccgccgggaatcagccgctgggagagttcatctgccaactctgtaaagaggagtaccccga

SEQ ID NO: 502 - DNA, 148 bp, Maccullochella peelii
cttgaaaaccctgcactcccatgacggtgttgttgagcctgtgtgtctggccgggagcgggctctgaatacgttctgtacgccgtgtttgtaatgtgatgtggggcgagacagtagctagctatgcacttcaggcaagctaactagtc

SEQ ID NO: 503 - DNA, 132 bp, Maccullochella peelii
gtgagcgagtgttcccaaagggactttttaaggaggaacgctaacccgcactcctcacatatgtagctcttcggcttcccggcgtgagtccgcctgtgtttcttcatgtgataggacaaccggaagcattta

SEQ ID NO: 504 - DNA, 150 bp, Maccullochella peelii
agggcggaaggccgagccatagctgggcttcctgttttggagggttgaggtgtccagaagtttgagccagccctccgatgtcacagggcgaaggtcaggggttactgttgtgcactctgggtcaaatagcccggttctgggtgttgtagt

SEQ ID NO: 505 - DNA, 129 bp, Maccullochella peelii
ggccctgtgcttcgggctagcagctgccaccctcatccagtctattggccacatcagcggcggccacatcaatcctgccgtcacctttgcctaccttgttggctcacagatgtctcttttccgcgccat

SEQ ID NO: 506 - DNA, 143 bp, Maccullochella peelii
tctactatgtcactggtttcttcatcgccatctcggtcatcactaatgtggtggagacggtgccctgtggttccaccgccaaccagaaagacatgccatgtggtgaacgctacacagtggcattcttctgcatggacaccgcc

SEQ ID NO: 507 - DNA, 145 bp, Maccullochella peelii
cacccaaacaaatacaacccaaacgaatcggatagatgccaacaacccgccatcagtggactaatttctcgcgggtcggggcctgtaacatccaccgacgaagtgctggagtccagtcaagaggcgctgcacgtcacggagcgtc

SEQ ID NO: 508 - DNA, 141 bp, Maccullochella peelii
taaacatccttgactggaagctggggtttggacaacgagggctttcagtcggatgtttgcagcatcttattgcctctgacagccaacagtaaccccagcgatgacactagtttgtaacatttgtaacagaaaagcagaact

SEQ ID NO: 509 - DNA, 134 bp, Maccullochella peelii
aaccacctgcagttccaggctgatccggacgtgctgcacaacagctacgccctgagagggatccactacaaccaggacctcattaacctggcggtgctgctggacatggaggggaagccttttcttcacgtgtc

SEQ ID NO: 510 - DNA, 125 bp, Maccullochella peelii
gcaaccaggacatgctgtgcatggactacaaccgcagccagaccaccactgcatctcccgtggtggcgaagccgaccaaccggccactgaagccgtacaaccccaggaagaaggtcagctacggt

SEQ ID NO: 511 - DNA, 132 bp, Maccullochella peelii
ccctctgatactctttcatgtctcattgtatctctcagggagtctttggaggccctgctccaaagggccgtggctcactgtcccaaggcagaggtcctgtggctgatgggggccaagtccaagtggctggct

SEQ ID NO: 512 - DNA, 127 bp, Maccullochella peelii
accatcatccagcttgggaaggagaaatactcgacctgcgtggtggagaagaccaccgagccggagtggagggaagagtgctcgttcgagctgcagcccggcgtgctggagagcaacgggcggagcg

SEQ ID NO: 513 - DNA, 125 bp, Maccullochella peelii
ctcggccccctaaatagccacaaaagcgtcggatgagtgaaatcggagatgctgcgcacctcggccaaaatcctggagcttttttgaacaggactgtgggtgtgtgcgcgtcgggctcgccgtta

SEQ ID NO: 514 - DNA, 143 bp, Maccullochella peelii
tcttccgccacatcctcaacttctaccgcaccgggaagctgcactacccgcggcaggagtgcatctccgcgtacgacgaggagctcgcgttcttcggcatcatcccggagatcatcggggactgctgctacgaggactacaag

SEQ ID NO: 515 - DNA, 129 bp, Maccullochella peelii
attcccgccggtgccatcacgttcacgcgtagccgcacttgtgcagcttcaggttgcgttggtccgagtatctcttcccacacttgtcacagatgaactgcttgccgccggcgtggacgtgcctcttgt

SEQ ID NO: 516 - DNA, 146 bp, Maccullochella peelii
tgctgcattccaggagaaaggtcgtaatgtctagatgttcgaagtacccattttgcgtgacgtcaaatttcttcatgcaaaaatcgcgcgaagctctacaaaaatgcacaattgcataaaaatgctgtatttgtgcaaatcacttc

SEQ ID NO: 517 - DNA, 145 bp, Maccullochella peelii
gacttcctgtcctacctcagcctcgagagactgcaggtttggtggttgttgtttgtacgcttaacgcagcagattgctaacagaaaagttagagctataaatgcaaacacttctggaagtttgtgttgttggattaattgtgatg

SEQ ID NO: 518 - DNA, 141 bp, Maccullochella peelii
accccgatgatgttctctatactgaaagacggtctgttggaaggctctgatttgatcatggacgccgtgctcagactgttgagctgaagctgaaggctcgggctgagctgggagttgaacgcttttctgttcagctccgcc

SEQ ID NO: 519 - DNA, 127 bp, Maccullochella peelii
gttgtggtccagagggttatcgcagttgattatgatggaggaaaaaacaccatcgacagaggattgggaggtgtggggggtgcacccgaactgaaatgacatcatcccctccactcttcgcccaaag

SEQ ID NO: 520 - DNA, 129 bp, Maccullochella peelii
tttctgtcattaacttttctcattattttagcagatgattctttttgcactttgctttaaagttgttctaataacctgtaacagagttcaggtccaggatcagttagcagctggtggtctgagagggtt

SEQ ID NO: 521 - DNA, 152 bp, Maccullochella peelii
cgcaggttgcaaactatattgtgcgtcttttatcatcttctcagtgtttctatgtcttggctttcactttcctcattatttttcctccctctcctcctgtgtctcaccatctctttctctgtctaaagtaagctaatgcacgggagacactc

SEQ ID NO: 522 - DNA, 125 bp, Maccullochella peelii
cgtggtttacttggtttactcggtggtgaatcccccgtacgtgcatggagagaatgaatgccaaactccattgaaaaatctggcgatttaatatgtcgtgatatacacactatacaacgtgaggc

SEQ ID NO: 523 - DNA, 152 bp, Maccullochella peelii
catacactaacgtagctgtagtatgcgttatagccatttatcctgccctaccctgcagatgtttaatagtgttactaaacttttactttggtaccgaagccctcgctttctaagttgttccggttcagtggaatagttttcgtcataggatt

SEQ ID NO: 524 - DNA, 128 bp, Maccullochella peelii
ctcgtatttctgaaccagcagttcaaactcccggtagttctcctccgcctgctggtcaagacgctgataagcccggacacactcgctgcatccccccatggccatatccagggtgcaattcaaatcgt

SEQ ID NO: 525 - DNA, 143 bp, Maccullochella peelii
tcctttgggggccacctggttcaggaggttgtagtacgccctggagtcctgaaacacatttaatgtccagtgttgaagtcagacagagaggcaccgttataattattattaaagtcaaaaatcacctgactgatcagtccaag

SEQ ID NO: 526 - DNA, 146 bp, Maccullochella peelii
cggcctcgaggttcttcaccagacagttcttcttggccattcgcttggcagtgagagtcagacacacctgggaggacggcaggaggtgagggggaggaagagtagggggggagtgaaggagggaggaaagaggggaagagaggaaa

SEQ ID NO: 527 - DNA, 151 bp, Maccullochella peelii
acctctgtatactgcataaagtgtctatacttaaacctacatgttaatggagttctgttgggcctcagcgagccgtagtttcttagcgtggttcttgccctggtagtgggcctgtgcaactgccggtgaactgaaggaggcatcacacagc

SEQ ID NO: 528 - DNA, 144 bp, Maccullochella peelii
tctatgacaattgcagatacatttacaagcaacatatacataacaaagctcaactgattgcctgcaaagcttaacgctgaaagctacttcctgccgagagctggagtccttaacccgagcattatgggatatggtcctggctct

SEQ ID NO: 529 - DNA, 135 bp, Maccullochella peelii
attgtaaaaccagattttcgctgattttaatggacgtgcattgctagcaacaccttacagcatagtgaatgttttaagagaatggtcagcaataatgtaaagaatatgcaatttataattagtgctaaaacatgc

SEQ ID NO: 530 - DNA, 149 bp, Maccullochella peelii
acgtaagtcttcagaggcttgtgctgatctaaggaggaaataatgaaaaacagaatattaggctaaataataattaaattaacagttactgaattattattgcatgtacggaatggttttctaacctgttgaaaagtagggatcttcag

SEQ ID NO: 531 - DNA, 147 bp, Maccullochella peelii
catatcttccagaaggagggggttactgctttctacaagggctacgtgcccaacatgctgggcatcattccctatgctggcatcgacctggctgtctatgaggtgtgtgtttgttgtcaaagcataacatatctttgtgtttactgg

SEQ ID NO: 532 - DNA, 127 bp, Maccullochella peelii
aactaagtccagaaccttcttgtccaggtatgagatccgggacccacacatggtggaggaaaatgtcctgcagatcctgaaggagagggccgactttgacaactataagccccgccccttcaacatg

SEQ ID NO: 533 - DNA, 139 bp, Maccullochella peelii
cagaaacagttagacagtcaggttgctgttccaattttcgttttattaatacgaagataattaaataatagttttgactccctctataatgctttgtaagtggcggagtgtcttttaaaaacagaaaacatgagatgat

SEQ ID NO: 534 - DNA, 600 bp, Chelonia mydas
ccttaaaggcttcaagccctgatgacaaatcaacgccacctccacccacccccgcttatcactcgtgtgagatcatggctggcaaatacgcgaccccggtctccgaggctctgcccagctggctctgcccgaccgcctggctcagccccgcggggccccgccccagccccgcgccgcgccgcgcatgctccttcccccgctgcggccgccggcgccgggcctggctggagcatgggggcctgggagccgccgccagccccacggccgggcccggcggctcctcctgctccgcgcggggccgggctcagccgcttcgtgcggtgcctctacctggtgggcttcctggtgagtgcggggtgcagcatcagggacctgccgggcgggggctcggtcccccgcttcctgccgtgtgtgggtgtgtgtgtcccggttcctgccctggccggcgggggtggggtgtgtgtcccggttcctgccctggccggcgggggcggggggggtgtcccggttcctgccctggccggcgggggggggggggggggggtgtcccggttcctgccctggccggcgggggtgggggggtgtgtgtcccggttcctg

SEQ ID NO: 535 - DNA, 600 bp, Chelonia mydas
accaactcatggtcactgcctcccagattcccatccacttttgcttcccccactaattctacctggtttgtgagcagcaggtcaagaaaagcgccccccctagttggctcccctagcacttgcaccaggaaattgtcccctacgctttccaaaaacttcctggattgtctatgcaccgctgtattgctctcccagcagatatcaggaaaattaaagtcacccatgagaatcagggcatgcgatctagtagcttccgtgagttgccggaagaaagcctcatccacctcatccccctggtccggtggtctatagcagactcccaccactacatcactcttgttgcacacacttctaaacttaatccagagacactcaggtttttccacagtttcgtaccgttatatccctggcttcatcttatcaatgttatacatcttttttgggggggggcacacaagctttggaagcaatcccgttacacatatgtatctgaaaaatgaattagatatgaaagattaatcatatttcagcacataagtaaaaaagaagactaattgcctaggattctcccataggaatgctattgcataattgttcgtt

SEQ ID NO: 536 - DNA, 600 bp, Chelonia mydas
ccccccccccccagaacccctcttctgtcccctgactgcccccagaaccggacaggagggtctcgtgggccaccgtagtgggtgcctaccccacccctaagagccagaggcacctgccagggggcgaggtggggagtcctggcagtgcttacctggggcggctcccaggaagcacctggcaggttcctctggctcctaggggcggggcagcgtagctaggtggggagtagggggagcagctgctccccccactgatcacatcaaaagtggcgccataggcgccgactccctgggtgatccggggctggagcacccacggggaaaatttggtgggtgcagagcacccaccgtcagctccccaccccgcccccatctcagttcacctctgctccgcctccgcctcctcccctgaacgtaccgccccgctctgcttctctgcaccttcccccaccaccacccccccccccccccccgccggcttcccgcaaatcagctgttcggagggaagccggggagggctgagaagcaggccgcggcttcccactcaggccaagggtggtggaggtgagctggggcaaggagcggttcccctgcgtgtcc

SEQ ID NO: 537 - DNA, 600 bp, Chelonia mydas
gtttcaggagatcaaggccctagatctgaccccggtcacccagggggaggatgaccttttgccagcaaacctcgatctgggcgatctcactccacccctctattccccatgctccctccccctaactgctgcttctgctcccacctccgaggagcccctggactcctccatcaacccagccactgatggcaccccgctgacgaccaccaagcctgctcaggtgacagccggcgccacgtggctaggacaggagtcgccaggggcaccccccgttggtgcggagcaatcgacttccttcccgggcgggggccctatagaagataatccacctcctgatgctgtggccgctaaatccaccatagagcctgtgcccgctatcactgagagctccctccccacccctttaaccctcgagcctgatcaggaggcgccatcatccagctgcttgcctcctgaaacccagaacctcgcctctgcccctgccccggcccttacctctatccagtttacctcctgcaatgttattgccacccccggggctgtctccttcccttttccaactgatgacccccagggagtggcctttgtgttctcctaccc

SEQ ID NO: 538 - DNA, 600 bp, Chelonia mydas
ttccctcccttcactattttaaatgcacacatctaacccaaatccgggctcgtatcttatttgctccatcccacacccagcacatataaatcagcgccgaaagactctccttcccggtataatgccacgtacagcacagcaccaaggcgtgggaacgatagcgaaaagacggagatgcggccctgcagcgagccggattgaagtaccaattccattgtgaactgcccccagttaaaaggaagcaattgggttattagtgttcaagtgggaaaaattaaaacccaaccggctgaaagccccggcgcaggaaattagaaagcggtgctaatttacaggcattgtcttaattcaagggctaacaatgtggaaagttgttgttagccccggcaagcaaagtggaaaggaataaaatgaccgaccgaaacggccctttcaggagatttcattatgaaactcattcccactaaattgtctttaactattgaataatgaaaaaggtagaacgttatttgctatttaagcttcccacttagagcccgcccgttagaatagtgctgcgtttgggagcctcccggcagatttctggtggagtcagcataa

SEQ ID NO: 539 - DNA, 600 bp, Chelonia mydas
atgagccagaagaaaaattagaaggtcccaagagttagtattttttaaaagctcatcttttaaacccaatttcacaactcagggctgtgcggggcggggcactcagtggtgtgtgccccaagtactgaggtacacacaagaattatgacgtgattggaataacagagacttggtgggacaactcacatgactggagtactgtcatggatggatataagctgttcaggaaggacaggcagggcagaaaaggtgggggagtagcactgtatgtaagggagcagtatgacagctcagagctccggtacgaaactgcagaaaaacctgagagtctctggattaagtttagaagcgtgagcaacaagggtgatgtcgtggtgggggtctgctatagaccaccggaccagggggatgaggtggacgaagctttcttccggcaactcacagaagttactagatcgcacgccctggttctcatgggagacttcaatcatcctgatatctgctgggagagcaatacagcggtgcacagacgatccaggaagtttttggaaagtgtaggggacaatttcctggtgcaagtgctggaggaaccaactaggg

SEQ ID NO: 540 - DNA, 600 bp, Chelonia mydas
ctgggggggggggaagtcagataataattaaaacacatttaacttagcacatagctcaaaaagctaaaataattaatttttgagggagcgggggaaagagcagagggtagaatttgtttctgaggctgaactcttccaaatacgcaggtgatgtcggctttggattcgttgaccatggaatgttgttccaagaaggaggagtgctaggcagggacaggctccacctaaagaagagagggaagagcatcttcgcaagaaggctggctaacctagtgaggagggctttaaactaggttcaccgggggaaggagaccaaagccctgaggtaggtgggaaagtgggataccgggaggaagcacgagcaggagcgcgcaagagggcagggctcctgcctcgtactgagaaagagggacgatcagcgagttatctcaagtgcctgtacacaaatgcaagaagcctgggtcctatacacaaatgcaagagcctgagaaacaagcagggagaactggaagtcctggcacagtcaaggaattatgatgtgattggaataacagagacttggtgggataactcacatgactggagtactgtcacggatgg

SEQ ID NO: 541 - DNA, 600 bp, Chelonia mydas
ccccactcccagggtccgagtggcttccccagcgctctcccccatgcggaggggctccccgctggcggccgccccgaagctcccgggccccgcacctggtggtggttgtaggagttcctctgctgcaggaaggcggccgcggccgccgccgcctggtgttggtgctggagctgggggctcacaggggatctccggttctgctgctgctgctgctgctgaggtaaattcatcggcggcggcggggcggcggccgagaaggggctgctgaagggcccggcgcaggggttgtgaggcgacaccggcgagaagctctggaagaaagcggggttgatcgaagacgggatccccgggtagaagctggtctcggagtccgggctcggcggaggcatcgcgctgatctggctcccgccgccgctgctgctggagaccggaggctgctgcgtttgcggcggaggcggcgacgaggtctgcaccgaccaaggggtgccgaagccgggcagcggcggcggaggaggggaagccgccgaggaggagccgcccccgctctggtgatgggggctggggatctccgagctctgcaggctgctgaagccggagccg

SEQ ID NO: 542 - DNA, 600 bp, Chelonia mydas
ccctcccgcccctctcaagtcccagcgctgccggggtggaactttgccgggctccacgttccagcccagccgggagggggaagcgagggacacaagcgctgcccggccgattcccgcccccctcccagcgcagcagctgagaaggaggaaggaggcgcgctaggcagcggcgctgagatttgccaggcaggcagcagaggaagagcagcaagggaagcccccgtggtccgtcctcgcgagccggctcttgcggcagcccgcaggagcgagcctggcagcgctgcggcggggttgttccccgggacgcgggcgctgaagttgcggtggcccaggcggggcggcggcccctgctgctcctctgcctgctgtttgtgtgcctcggtggccggagggggagagccgcccacctccgtccagcttcccactccgctcccccggctcgggctgtttgtgtgggagacacccactcctccttcgttccctccccccggccgcctcctcccctttccccagagccggaggaggccggagcgggcgaggtgcggttgctgttgtggctctgggctctctcggcccggcacggccgcgccgctgctacgg

SEQ ID NO: 543 - DNA, 600 bp, Chelonia mydas
ggtcctgcacgatgtacctggggggggcggcgccccgagggggggcaggcatgggggtcaaggggcataacatgattggtaaggggatgcggcacctgtggggtggcgggccgggcacacggggacccctcctccagtgcggggccgggactcccctggggcggggccgggtccccggggccgggactcccctggggcggggccgggtccccccggggcacggcggaggatacacgtcgcgcatgtggtcgatggccgcggcccgcaggatgtcgttgatcccgatgatgttctcgtgccggaagcggagcaggattttgatctcgcgcagggtgcgctgacagtacgtctggtgctcgaaggggctgatcttcttgatggccgcccggagcttgttcgcgtggtcgtaggccgagctgcgcagagaggggaggccatggggcacggggggcacggggggcccggcccccagcttcccgcggcagagccccctcggcaccacggggacccagaacccaggagtccggcgggcagcccctccccccgcgcgcctcccgagccgggacaggactgtaccccgggggcggcggcgctgcgagc

SEQ ID NO: 544 - DNA, 600 bp, Chelonia mydas
attatgcagccccatgatggggcacctcctgacaaatagtgaagctatttgcagagattctggctcagggagtcatttgctaaccctagccaggacaggatattgaccagccctgttcctttatgtgggccaagccctggtctacactaggacttgaggtcgaatttagcagcattaaatcgatgtaaacctgcacccgtccacacgatgaagccatttttttttgacttaaagggctcttaaaatcgatttctttactccacccctgacaagtggattagcgcttaaatcggccttgccgggtcgaatttggggtactgtggacacaattcgatggtattggcctccgagagctatcccagagtgctccattgtgaccgctctggacagcactctcaactcagatgcactggccaggtagacaggaaaagaaccgcgaacttttgaatctcatttcctgtttggccagcgtggcaagctgcaggtgaccatgcagagctcatcagcagaggtgaccatgatggagtcccagaatcgcaaaagagctccagcatggactgaacgggaggtacgggatctgatcgctgtatggggagagga

SEQ ID NO: 545 - DNA, 600 bp, Chelonia mydas
agcccagtccggtcagcatgccagcctgggcagatagacttgtgcgagcagggccttcacgcccggccgacgttggctctgagctcagggggccgcagcctccacactgctcgtttagtgcccgacctcgggccccaccagcccacgactgtcccgggctcgcaggctggctccccgctgcagggcagacacagcccaggagccccccacacgcccctgtgagccagagcccagcccggtcagtcctggctggcccccaccggcccgctgagcccggcgagggaggtggcagtcttccccggcagggtggtgcggggcagcgtttgggtttgcagagcggtgagtaaaggagcaggaatgcccggcacagattcactcccaaaaaggagattggaacaacttgtgggtttcctgtttactgaagccagcaaggctgcccgtgcggggtcccctcccccaccccggctgccggaggcagcactggccaggctggggaggggacctcgaggctggggggcctgcggatgccctcggggccccggtcactctctgccacctctggccggagattggctgggctcctcagccgggggggggcag

SEQ ID NO: 546 - DNA, 600 bp, Chelonia mydas
ccgcacctgtcggcgtagggtagacacacgctgagccagtcagtgtagcgcgcgtgcagccccggacatctaagggcatcacagacctgttattgctcaatctcgggtggctgaacgccacttgtccctctaagaagttggacgccgaccgctcgggggtcgcataactagttagcatgccagagtctcgttcgttatcggaattaaccagacaaatcgctccaccaactaagaacggccatgcaccaccacccacagaatcgagaaagagctatcaatctgtcaatcctttccgtgtccgggccgggtgaggtttcccgtgttgagtcaaattaagccgcaggctccactcctggtggtgcccttccgtcaattcctttaagtttcagctttgcaaccatactccccccggaacccaaagactttggtttcccgtaagctgcccggcgggtcatgggaataacgccgccggatcgctagtcggcatcgtttatggtcggaactacgacggtatctgatcgtcttcgaacctccgactttcgttcttgattaatgaaaacattcttggcaaatgctttcgctttggtccgtcttgcgccg

SEQ ID NO: 547 - DNA, 600 bp, Chelonia mydas
gctgctcgctggaggcagactggagcccagcgcagctctccgcgccgctttgctcagggcgggagctgcaaacaccggccagcgcgatggcccagcctcctccccggcggggcaactgagggccgctgcgctccgcggagccccagtcccggcccctctgtgcccctggcgggggcgggggaccggcgcagcagctgttggggccgcagggcagaagggggctcggcccaaagcctgtccgatcccccacggcggccggggatgggaacgcgtcccccggctctgggtggctcggctgccgggctgggcatggagctggaatccccgcaccgtccaagcagcttcgcttgtaaaaagccaaagtgtcggggtctgggggaggcagccgcgcggagagccgggcccctgtatctactctgtatccgctgtaagtgccagcccgcgcaggagcgagggacttaataaaccaggtgcaatagcccccgcgcctcattgttagcggcccgggagcccgtggcctggagcagggaccggagcggaccgccggcgggggatttccgttcgccccagcccacgggatgttgtacgttaggggtgaag

SEQ ID NO: 548 - DNA, 600 bp, Chelonia mydas
ccaatgctggctgcatggcccctgtgcgtggttctgagcaggcggcccctgcggtgaagttgtggagcagtttccgacccgtgccctcttcccatgcctctcctgggtggctgcatatgctccctcagctgaaatccagagccctcgcctggtgagacatggcactcatgggctcttgtgattcaggcctgagtcctaggtgtcttttccaattaggccattcactgctcaggcctgtgcctgccctgagcacttgggattgccatggcaaccaggagcgccagcatttattaatcatccgggtggcctttgggttggcatggcaactgcagacaagaatagaaacacaggagtgggaaagtggcagaggagctgccaggcaatggcacatgtcagccggagctggggcccccctgtcccggtgccgggtgctgcgagaattgaggccttcccttcagatctggcattttttcttggcttctgaaacaattttactttaggctaagaaaacagctctgcacacgggagtgattctgggatccctgcctgctgcagggaggggctcagttgaggctgaaggatgctacaaagagaaaccca

SEQ ID NO: 549 - DNA, 600 bp, Chelonia mydas
aaaatctaaagtagttccacttttgattgtaatttcaactaaaatacttctacagcaaataaaccctcttctgcaagcgtttgaaagcgtaagttgaaagtgtaaaagaggagaaacctaattttccatcgtccaaactgaaagaaatgcaaaatgaaaatccaaccgtattaaatagtgaaagagttttagatgctcactaagcgaagacattacttgtaagcaaacgtttgctacaacatacaccctttaagttcacagaatgccccctaaaatacagaatcgccgcaaaagcagcccggtattgttcgagcagcaaaagaaagtcattttcccaggaggtcctgcgaactcctcgagcgaaggaaacgctggagcccggtttttacgccccctccgtgcggggccagcagcgctcgccgcttgttgtgagcgcccgggctgagctggcggaagcctgcgcgctgccagcaggcgaatcaccgccactcgcgggccgcgcgcgtggactggcgacacgagcagcgccagcccgtcccggcgggtctccgcgagcagagcgggccgccgagacgcgggcagggggaggagctgcgcgcc

SEQ ID NO: 550 - DNA, 600 bp, Chelonia mydas
tcccacagcgccgccaccctgcaaattctgtcggcgcggtgacagacggacgaagcgagggtcgaagggttttctcgaaagcggctccgggatgctgagacggtgggctgcaggtgcagtaaagccctagatggcccggcctgttggtcccaccctggcccacatgctcccggaccccagcaccccgcctgcaggcccggccctgtgccccacaccacgggcaccgggcgctggcggcagcaccacgagcctggggagcccccgggcgctcgcgcccagctctggtgacccgggggtcccgggcccacagcgagagcggcgccgtcaggcacgggggcgagatgtgggcggctcccgcagcagggaggagccgcttccccgggccagagagggcaggaccggagccgagctgggggagagggggctgggcccggccgtcacccacctgccggccccgagcgggccccccgggcgcagccgcgccgacagcaggtagaggccgcaggaagcggccccgagcagcgccaggaaaccgcagagcgaccgcatcccggcgcgggagctacagcgccaagagcgccgggtaccggcggttcgcgg

SEQ ID NO: 551 - DNA, 600 bp, Chelonia mydas
caaatgttttcgcatttcgaaaactggaacagagttctgatagtacagattcctctccctatacagcgatcagagcccgtaccttccgttcagtccatgctggagctcttttgcgattctgggactccatcatggtcacctctgctgatgagctctgcactcacctgcagcttgccacgctggccaaacaggaaatgaaattcaaaagttcactggccttttcctgtctacctggccagtgcatctgagttgagagtgctgtccagagcggtcacaatggagcactctgggatagctcccggaggccaataccgtctaattgcgtccacagtaccccaaattcaacctgcaaggccgatttcagcactaatccccttgtcgggggtggagtaaagaaatcgattttaagagcgctttaagtcgaaaaaaagggcttcgtcatgtggacggttgcagggttaaatcaatttaacgctgctaaattcgacctcaactcctaaagtgtagaccagggcttaggcttctgcagctgcaaacaacaagggatcatgaaaattaaactaaacaaagagaatacatgttggcagcctactggagacc

SEQ ID NO: 552 - DNA, 600 bp, Chelonia mydas
tctccatcctgggccatgtgtgaagccgggcgggaggtttgtctagaaatctgcctacgttcccatccccccacccgccaggtgatcccagtctccccgtccctccttccagctgggaattctccagggggctggagtggtggggagcagaaacgaggctcggagagtgggcttgggagcccgtctcgctcacttcagctctggcgttcctcgcccccatcccaccctcagctccccgctcctctgcggagactccctgcgcgcctcagagccagatttctgctgcgggagaaatcaaccggctgccgtcctcttaccccgtcgtaaggcagcaaatccaaggcccaggccgaacggctcgggggcgtggagcaagaacgccgggagcccctgggcgcggccaagggctggatttcagccccgttggggcgggggggtctatggcgaactgctccatcctggggtgaaagctgagtaaataggcagggccgtgtccctgccaagaacagccaggagctggagcctgctccacaaacctccgcttctctgcagggcgacagcggggaagaagggcctagctgggttaacaaccccccagtg

SEQ ID NO: 553 - DNA, 600 bp, Chelonia mydas
ccgtgcccgccagacaccacacgccggcggggccaagccgagcagcccccggtccccgcgactgctcggcacctttaccgccgcccctgcgccccgccaggcccccgcgcggactcgccccgctcacccccgtggcgccgggccgccctcgagagccgccgggccggcgggcccccgagcccgcgcagccccggcgacgtcgccgccctccgcagcagcagcagcatcccggcaacagccaggcgcggaaacagcccctgccctttcaccccgacccgcgtgtcgtcatcgccgcgcgccggaagtgacggagagactggagcgtgctgggcggaggcgaggagcgaggtgaggggtcccccggcgcccccgggagcgggccgcggagagcgtgggatcagccgggccccgcggagcttccctagccggcggccgcagggtcccggctcgcgcggtgcaggcgccagggcttgccccagccctgccaagctgggggccgcaccagtgtcccgtggccgtgagttccgcaggctcctctacagcctccgagcccccgtgctctgtgacggagtgagccgggctctggccccctccagggcg

SEQ ID NO: 554 - DNA, 600 bp, Chelonia mydas
agtctctgtttcatgatggcagcatatcgcaacaggtggcagatggcaacatagcggtcaaaggacatgaccataagaaggatgaactccatagctcccagggtgaaatagaagtaggattgggccatgcaggcaaggaatgagatggttttgctgtctgagagaaagttcaacagcatcttggggtttgtgaccgaggtgaaccagatctccaagaaggacagattgctgatgaaaaagtacatggggggtatggagtcggtgatccacccacactatgaaaatgattaatgagttcccggttagtgtgaccaagtaggtcagtaaaagaacaaagaagagaaacatctggagtttgtcatgaactccggaaaaccccaagagcctgaattcagccactgtggtttcatttgcttcctccatttccgatttcagtttcccctgtagaaagagcaacaataaaaaaataagagcatgaacctttatctatcacatgtatggattcaacagcaatgttctggagggttgtgagacagaaaagctgaacactcgagatcacacactaaggaaggagacagaattagtgagattgacagagaa

SEQ ID NO: 555 - DNA, 600 bp, Chelonia mydas
aggctggctgtcgaataaattttcctgccacacagggtttcttcaggtatgttagcaacaagaagaaagtcaaggaaagtgtgggccccttactgaatgagggaggcaacctagtgacagaggatgtggaaaaagctaatatactcaatgctttttttgcctctgtcttcacgaacaaggtcagctcccacactactgcactgggcagcaagcatggggaggaggtgaccagcctctgtggagaaagaagtggtttgggactatttagaaaagctgaacgagcaaaagtccatggggccggaggcgctgcatccgagagtgctaaaggagttggcggatgtgattgcagagccattggccattatctttgaaaactcatggcgatcgggggaagtcccggacgactggaaaaaggctaatgtagtgcccatctttaaaaaagggaagaaggaggatcctgggaactacaggccagtcagccacacctcagtccctggaaaaatcatggagcaggtcctcaaggaatcaattctgaagcacttagaagagaggaaagtgattaggaacagtcagcttggattcaccaagggcaagtcatga

SEQ ID NO: 556 - DNA, 600 bp, Chelonia mydas
ggtggcggagatgagctggggcgggaaccgattcccctgcacccctgccccgggttacctgctgcggcgcaggcgaccctcctcgtgcccccccctcccccccagctcccctccgctccgcctccctgggcctgagcgcgaagccgccccctgcttctcagcccccggcttcccacgcgaacagctgattcgcgggaagcggtgggggggaggcggagaagcagagcggggcggagcataactcaggggcggaagcggagcagaggtgagctggggccagggctggggcggggagctgccggtgggtgctctgcacccaccaaattttccccgtgggtgctccagcctcgaagcacccatggggtcggcacctaaggcaccacttttggctggttgttacatttagaagcccttttagaacatgacggacaaccggttctaaaagggcttctaaatttaacaaccggttctagtgaactggtgcgaaccggctccagctcaccactgcccttgccgcaccctctgcctcgccacttccccgaggcctcgaccctgccctgccccttctctgaggcccctgccctgctcactccatccccc

SEQ ID NO: 557 - DNA, 600 bp, Chelonia mydas
ctgaggagggatgaaagcccctccctttcatgccactttataggcaaccccgattttgttctttggaggccaatttcacacaagctgggggaggcagagaatggggttaacagcagctattcaaatgacatttatgtgaggtgtacgaacacccgacccccttacacgctgaccccataatccagttggagttactgccccatgcccggctccggcccagcacctcctccctgcctcttaccccgtgcctgcaggcctgctcgttgagggctcgcacccaagctcgctgccagttctcccggaaggatctgaaggcgaagagcgaagcaaggagccctctggcccccgctaccgcctcctcgggagctccgtccggggccacccgcagtctcagtaaggacttccagatgcctgcttcccttagagctcctcccgaggtctccggctttccccggccgctccacacgccccgggaatactgcaacagccaggccgagacggtgagcagggaggcggcgaagagcagcaccagagccgcccagcccagctccagctccagctccatggtgctctctgctcagcaacttcggggaggctagt

SEQ ID NO: 558 - DNA, 600 bp, Chelonia mydas
accatctcatggtcactgcctcccaggttcccatccactttagcttcccctactaattcttcccggtttctgagcagcagatcaagaagagctctgcccctagttggttcttccagcacttgcaccaggaaattgtcccctaccctttccaaaaacttcctggattgtctgtgcaccgctgtattgctctcccagcagatatcaggatgattgaagtctcccatgagaaccagggtctgcgatctagtaacttccgtgagttgccggaagaaagcctcgtccacctcatccccctggtccggtggtctatagcagactcccatcacgacatcacccttgttgctcacacttctaaacttaatccagagacactcaggtttttctgcagtttcataccgaagctctgagcattcatactgctctcttacatacagtgcaactctgccaccttttttgccctgcctgtccttcctgaacagtttatatccatccatgacagcattccagtcatatgagttatcccaccaagtctctgttgttccaatcaaatcataattccttgactgtgccaggacttccagttctccctgcttgtttccc

SEQ ID NO: 559 - DNA, 600 bp, Chelonia mydas
tcactgcggactacgtggctctgggaagaaggataaaggagttggaggcgcaagtggtgttctcgtccatcctccccgtggaaggaaaaggcctgggtagggaccgtcgaatcgtggaagtcaacgaatggctacgcaggtggtgtcggagagaaggctttggattctttgaccatgggatggtgttccatgaaggaggagtgctgggcagagatgggctccatcttacgaagagagggaagagcatctttgcgagcaggctggctaacctagtgaggagggctttaaactaggttcaccgggggaaggagaccaaagccctgagataagtgggaaagcgggataccgggaggaagcacaggcaggaatgtctgtgaggggagggctcctgcctcatactgggaatgaggggcgatcaacaggttatctcaagtgcttatatacgaatgcacaaagccttggaaacaagcagggagaactggaggtcctggtgatgtcaaggaactatgacgtgatcggaataacagagacttggtgggataactcacatgactggagcactgtcatggatggttataaactgttcaggaaggacaggca

SEQ ID NO: 560 - DNA, 600 bp, Chelonia mydas
acctctgctcccctcctggctggagggctggttccccagctggctgctttcccctctctgccccatgccttccctggaggacccctggaagccagtagtgatgatgacaggaccacttgatggtgccagggactttgtttacaatcatcagctatacaaacacattgtttctgtgcaagaagctaccaggctaaggtgtgggggggccggggatagtggactggggagagccccttccccctccctgctcatcagccaggagctgttgacatctgttttcattggggatgctttgatgccggctgttcttgattgaaggcaaacagagccctggaggcgagtgaggacaggtttccaatcctcgggggctccggtgtcgtctcggcacagccatcaataatggtccgaggggcctcagcttcatcctggcccttgcagggggctccagctcctacagacagctatttgtgctttgttggaaagacccaagtacccagacgggcttgctgactgaacggtaacgctgcaggggcaggcaagggacacatgtatgtgggatcagagcagggggcacatctaggccagtgtagcccccttcct

SEQ ID NO: 561 - DNA, 600 bp, Chelonia mydas
agaaccggctgctggccccttgcccgtgacagagcacagggccacacacctcattaaggcaggagatgaatttaacaattgtacctggattcgagggcacgctgaattttgtggcttcgttgttttcaaatctgtgacctgctttggcttttcacagccaggcacccaaatagagattgggggggggggggcgggggaatagctcgccttggctagaatgaatacatttgctttgctgcggggggggggggtagttgggagacttttttttttaagtgtgtgtctacatatgcacgccccgggagagagagggtgtgatcgttgcttggaaagcagaaggggattggagtgaaaactccagggctgggcacaaagataataagcaccgggcagaatttttagtggcggaaaaacatgacttgtctgcaaggggcagttcccatgccctcccccccccccccatacacacacgaaagaagacaaaacggaaaggggaaaaacaacgggttttgccaacattttatcccgagtttgagaccctgagcgggatgtttgctgcacgaaataaagtggggttacaatctagtccccctgggaaat

SEQ ID NO: 562 - DNA, 600 bp, Chelonia mydas
ctctggatttaccatttcctggctgcggaaagacgcacttcccctccctcctgggccagggcatggggggtgtccctgcatcctcccccccacccccccagctggcaatctacaccccccagggctccctctcctccctactgcggcggggggggggctttgctttggcaggcttcagtggggagaacccaggagtcctggctcccatcagcccgtggctgtcctgccggggtggcactggggctgtgtggcaccttggccccgtactgccccaggctgagggcgattctccgcgcgcccggctgctgagggcggctctcctggtacaaaggggccctgtgcgagccccgctgtgagtcaccggggctggtggggagccgcggcttggctggagttcccagccggccgcccgcacaaagggcctttgagagagggggcgggcccagagccgggagggggttgggggactatgggggccctggctctgagctggggggggagcagctttgggggggactttggaggggggagcagccttttccaaccactagcccggttcaccgccccctctgtgtatagcctgaagggggggcaagcccc